(19) United States
(12) Patent Application Publication
(10) Pub. No.: US 2024/0361688 A1
(43) Pub. Date: Oct. 31, 2024

(54) ONIUM SALT, RESIST COMPOSITION, AND PATTERN FORMING PROCESS

(71) Applicant: Shin-Etsu Chemical Co., Ltd., Tokyo (JP)

(72) Inventor: Masahiro Fukushima, Joetsu-shi (JP)

(73) Assignee: Shin-Etsu Chemical Co., Ltd., Tokyo (JP)

(21) Appl. No.: 18/626,972

(22) Filed: Apr. 4, 2024

(30) Foreign Application Priority Data
Apr. 12, 2023 (JP) 2023-064766

(51) Int. Cl. G03F 7/004; C07C 25/18; C07C 211/63; C07C 381/12; C07D 207/16; C07D 211/46; C07D 211/54; C07D 211/62; C07D 221/14; C07D 327/08; C07D 333/76; C07D 405/12

(52) U.S. Cl. CPC G03F 7/0045; C07C 25/18; C07C 211/63; C07C 381/12; C07D 207/16; C07D 211/46; C07D 211/54; C07D 211/62; C07D 221/14; C07D 327/08; C07D 333/76; C07D 405/12

(57) ABSTRACT

A chemically amplified resist composition is provided which is excellent in LWR and resolution and can prevent a resist pattern from collapsing in far ultraviolet lithography and EUV lithography, an onium salt for use therein, and a pattern forming process using the resist composition. The chemically amplified resist composition includes an onium salt having the following formula (1).

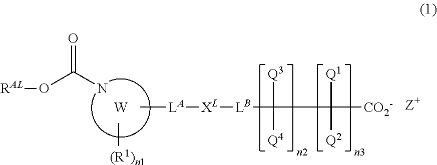

### CROSS-REFERENCE TO RELATED APPLICATIONS

This non-provisional application claims priority under 35 U.S.C. § 119 (a) on Patent Application No. 2023-064766 filed in Japan on Apr. 12, 2023, the entire contents of which are hereby incorporated by reference.

### TECHNICAL FIELD

The present invention relates to an onium salt, a resist composition, and a pattern forming process.

### BACKGROUND ART

To meet the demand for higher integration density and operating speed of LSIs, the effort to reduce the pattern rule is in rapid progress. As the use of 5G high-speed communications and artificial intelligence (AI) is widely spreading, high-performance devices are needed for their processing. As the advanced miniaturization technology, manufacturing of microelectronic devices at the 5-nm node by the lithography using extreme ultraviolet (EUV) having a wavelength of 13.5 nm has been implemented in a mass scale. Studies are made on the application of EUV lithography to 3-nm node devices of the next generation and 2-nm node devices of the next-but-one generation.

As the feature size reduces, image blurs due to acid diffusion become a problem. To insure resolution for fine patterns with a size of 45 nm et seq., not only an improvement in dissolution contrast is important as previously reported, but the control of acid diffusion is also important as reported. However, since chemically amplified resist compositions are designed such that sensitivity and contrast are enhanced by acid diffusion, an attempt to minimize acid diffusion by reducing the temperature and/or time of post exposure bake (PEB) fails, resulting in drastic reductions of sensitivity and contrast.

The triangular tradeoff relationship among sensitivity, resolution, and edge roughness (LER, LWR) has been pointed out. A resolution improvement requires to suppress acid diffusion whereas a short acid diffusion distance leads to a decline of sensitivity.

The addition of an acid generator capable of generating a bulky acid is an effective means for suppressing acid diffusion. It has been then proposed to incorporate repeat units derived from an onium salt having a polymerizable unsaturated bond in a polymer. In this case, this polymer functions as an acid generator (polymer-bound acid generator). Patent Document 1 discloses a sulfonium or iodonium salt having a polymerizable unsaturated bond, capable of generating a specific sulfonic acid. Patent Document 2 discloses a sulfonium salt having a sulfonic acid directly attached to the backbone.

The triangular tradeoff relationship among sensitivity, resolution, and edge roughness has been pointed out. A resolution improvement requires to suppress acid diffusion whereas a short acid diffusion distance leads to a decline of sensitivity.

Various studies have also been made on quenchers (acid diffusion inhibitors). As the quencher, various amines are mainly used, but many problems to be improved in line width roughness (LWR) as an index of pattern roughness, pattern profile, and the like. Studies using a weak acid onium salt as a quencher have also been reported. For example, Patent Document 1 describes that patterns with minimal roughness can be formed using a compound capable of generating a carboxylic acid having a boiling point of 150° C. or higher. Patent Document 2 describes improvements in sensitivity, resolution, and exposure margin by the addition of ammonium salts of sulfonic acids or carboxylic acids. Patent Document 3 describes that a resist composition for KrF or electron beam (EB) lithography comprising a photoacid generator capable of generating a fluorine atom-containing carboxylic acid is improved in resolution and process latitude such as exposure margin and depth of focus. Patent Document 4 describes a positive photosensitive composition for ArF excimer laser lithography comprising a carboxylic acid onium salt. Patent Document 5 describes an onium salt of fluoroalkanesulfonamide as the weak acid onium salt, but even if this onium salt is applied to the upcoming generation of ultrafine processing using ArF lithography or ArF immersion lithography, the LWR as an index of pattern roughness and resolution are yet insufficient, and there is still a need for a weak acid onium salt having better quencher function. Patent Document 6 describes an onium salt of α,α-difluorocarboxylic acid as the carboxylic acid onium salt. On use of this onium salt, it can also act as an acid generator in some cases because the carboxylic acid resulting from proton exchange with strong acid has an acidity which is not fully low. Because of such low quencher capability, LWR and resolution are unsatisfactory. An example of using an aromatic carboxylic acid onium salt, which has not been positively applied in the ArF lithography, in the EUV lithography on which development efforts are recently concentrated, has also been reported.

A carboxylic acid onium salt having a nitrogen-containing structure in the same molecule has also been reported. Patent Documents 7 to 9 describe a carboxylic acid onium salt having indole or indoline as a nitrogen-containing heterocyclic compound and a piperidinecarboxylic acid structure, Patent Document 10 describes a carboxylic acid onium salt having an aminobenzoic acid structure, and Patent Document 11 describes a carboxylic acid onium salt having an amide bond. Although these also act as a quencher, aromatic amines and amide bonds are not highly basic, and thus acid diffusion controllability is not sufficient, and piperidinecarboxylic acid has extremely high water solubility, and there are many problems in industrial production.

These series of weak acid onium salts are based on the mechanism that a salt exchange occurs between a weak acid onium salt and a strong acid (sulfonic acid) which is generated by another photoacid generator upon exposure, to form a weak acid and a strong acid onium salt. That is, the strong acid (α,α-difluorosulfonic acid) having high acidity is replaced by a weak acid (alkanesulfonic acid or carboxylic acid), thereby suppressing acid-aided elimination reaction of an acid labile group and reducing (controlling) the distance of acid diffusion. The onium salt apparently functions as a quencher. However, as the microfabrication technology is currently further advanced, the resist compositions using such weak acid onium salts become unsatisfactory with respect to resolution, roughness, depth of focus, and the like, particularly when processed by the EUV lithography. The alkanesulfonic acid salts have a low quencher capability because their acidity is not fully low. The carboxylic acid salts are not only insufficient in the above-referred properties, but also suffer from a swell problem because they are highly hydrophilic and thus have a high affinity to an alkaline developer so that the developer is sucked in the exposed area. Particularly in forming small size line patterns, the swell causes the resist pattern to collapse down. To comply with the requirement for further miniaturization, it is desired to have a quencher which has favorable sensitivity and excellent acid diffusion controllability, and prevents the resist patterns from collapsing as a result of swelling in an alkaline developer.

### CITATION LIST

Patent Document 1: JP-A H11-125907
Patent Document 2: JP-A H11-327143
Patent Document 3: JP-A 2001-281849
Patent Document 4: JP 4226803
Patent Document 5: JP-A 2012-108447
Patent Document 6: JP-A 2015-54833
Patent Document 7: JP 6217561
Patent Document 8: JP 6874738
Patent Document 9: JP 6512049
Patent Document 10: JP 6323302
Patent Document 11: WO 2019/087626

### SUMMARY OF THE INVENTION

It is an object of the present invention to provide a chemically amplified resist composition which is excellent in LWR and resolution and can prevent a resist pattern from collapsing in far ultraviolet lithography and EUV lithography, an onium salt for use therein, and a pattern forming process using the resist composition.

The present inventors have conducted intensive studies to achieve the above object, and as a result, have found that a resist composition comprising, as a quencher, an onium salt containing an anion having a nitrogen atom-containing aliphatic heterocyclic ring and a fluorocarboxylic acid structure is excellent in resolution of a resist film, has a small LWR, further suppresses swelling during development, and is extremely effective for precise fine processing, thereby completing the present invention.

That is, the present invention provides the following onium salt, resist composition, and pattern forming process.
1. An onium salt having the following formula (1):

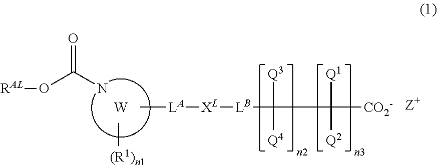

wherein n1 is an integer of 0 to 6, n2 is an integer of 0 to 3, n3 is an integer of 1 to 4,
W is a C₂-C₂₀ nitrogen atom-containing aliphatic heterocyclic ring which may contain a heteroatom,
L^{A} and L^{B} are each independently a single bond, an ether bond, an ester bond, an amide bond, a sulfonic acid ester bond, a carbonate bond, or a carbamate bond,
X^{L} is a single bond or a C₁-C₄₀ hydrocarbylene group which may contain a heteroatom,
R¹ is a C₁-C₂₀ hydrocarbyl group which may contain a heteroatom, when n1≥2, a plurality of R¹'s may bond together to form a ring with the carbon atom on W to which they are attached,
Q¹ to Q⁴ are each independently a hydrogen atom, a fluorine atom, a C₁-C₆ hydrocarbyl group, or a C₁-C₆ fluorinated hydrocarbyl group, provided that at least one of Q¹ to Q⁴ is a fluorine atom or a C₁-C₆ fluorinated hydrocarbyl group, Q³ and Q⁴ may bond together to form a ring with the carbon atom to which they are attached,
R^{AL} is an acid labile group, and
Z⁺ is an onium cation.
2. The onium salt according to 1, wherein R^{AL} is a group having the following formula (AL-1) or (AL-2):

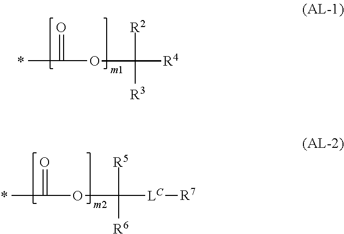

wherein LC is-O— or —S—,
R², R³, and R⁴ are each independently a C₁-C₁₀ hydrocarbyl group, any two of R², R³,
and R⁴ may bond together to form a ring with the carbon atom to which they are attached,
R⁵ and R⁶ are each independently a hydrogen atom or a C₁-C₁₀ hydrocarbyl group, R⁷ is a C₁-C₂₀ hydrocarbyl group, some of —CH₂— in the hydrocarbyl group may be replaced by —O— or —S—, R⁶ and R⁷ may bond together to form a C₃-C₂₀ heterocyclic group with the carbon atom and LC to which they are attached, some of —CH₂— in the heterocyclic group may be replaced by —O— or —S—,
m1 and m2 are each independently 0 or 1, and
* designates a point of attachment to the adjacent —O—.
3. The onium salt according to 1 or 2, wherein Z⁺ is a sulfonium cation having the following formula (cation-1), an iodonium cation having the following formula (cation-2), or an ammonium cation having the following formula (cation-3):

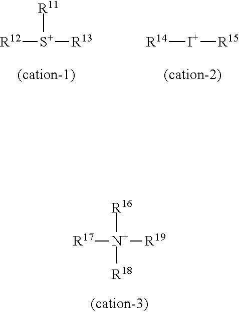

wherein R¹¹ to R¹⁹ are each independently a C₁-C₃₀ hydrocarbyl group which may contain a heteroatom, and R¹¹ and R¹² may bond together to form a ring with the sulfur atom to which they are attached.
4. The onium salt according to any one of 1 to 3, which has the following formula (1A):

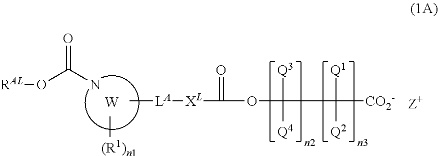

wherein n1 to n3, W, L^{A}, X^{L}, R¹, Q¹ to Q⁴, R^{AL}, and Z⁺ are as defined above.
5. The onium salt according to 4, which has the following formula (1B):

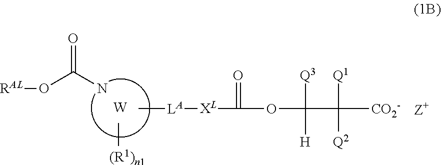

wherein n1, W, L^{A}, X^{L}, R¹, Q¹ to Q³, R^{AL}, and Z⁺ are as defined above.
6. A quencher comprising the onium salt according to any one of 1 to 5.
7. A resist composition comprising the quencher according to 6.
8. The resist composition according to 7, further comprising an organic solvent.
9. The resist composition according to 7 or 8, further comprising a base polymer comprising repeat units having the following formula (a1):

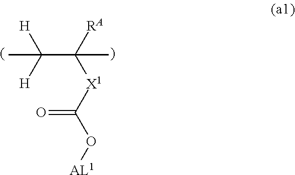

wherein R^{A} is a hydrogen atom, a fluorine atom, a methyl group, or a trifluoromethyl group,
X¹ is a single bond, a phenylene group, a naphthylene group, or *—C(═O)—O—X¹¹—, the phenylene group or the naphthylene group may be substituted with a C₁-C₁₀ alkoxy group which may contain a fluorine atom, or a halogen atom, X¹¹ is a C₁-C₁₀ saturated hydrocarbylene group, a phenylene group, or a naphthylene group, the saturated hydrocarbylene group may contain a hydroxy group, an ether bond, an ester bond, or a lactone ring, * designates a point of attachment to the carbon atom in the backbone, and AL¹ is an acid labile group.
10. The resist composition according to 9, wherein the base polymer further comprises repeat units having the following formula (a2):

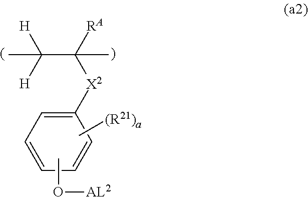

wherein R^{A} is a hydrogen atom, a fluorine atom, a methyl group, or a trifluoromethyl group,
X² is a single bond or *—C(═O)—O—, * designates a point of attachment to the carbon atom in the backbone,
R²¹ is a halogen atom, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
AL² is an acid labile group, and
a is an integer of 0 to 4.
11. The resist composition according to 9 or 10, wherein the base polymer further comprises repeat units having the following formula (b1) or (b2):

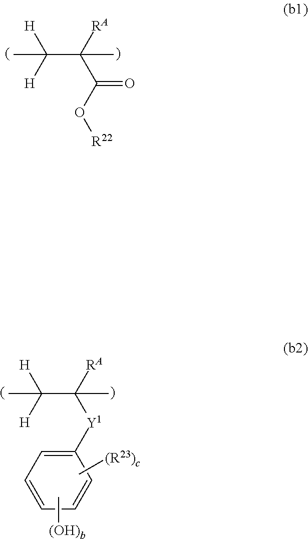

wherein R^{A} is each independently a hydrogen atom, a fluorine atom, a methyl group, or a trifluoromethyl group,
Y¹ is a single bond or *—C(═O)—O—, * designates a point of attachment to the carbon atom in the backbone,
R²² is a hydrogen atom or a C₁-C₂₀ group containing at least one or more structures selected from a hydroxy group other than a phenolic hydroxy group, a cyano group, a carbonyl group, a carboxy group, an ether bond, an ester bond, a sulfonic acid ester bond, a carbonate bond, a lactone ring, a sultone ring, and a carboxylic anhydride (—C(═O)—O—C(═O)—),
R²³ is a halogen atom, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
b is an integer of 1 to 4, and c is an integer of 0 to 4, provided that b+c is from 1 to 5.
12. The resist composition according to any one of 9 to 11, wherein the base polymer further comprises repeat units having any one of the following formulae (c1) to (c4):

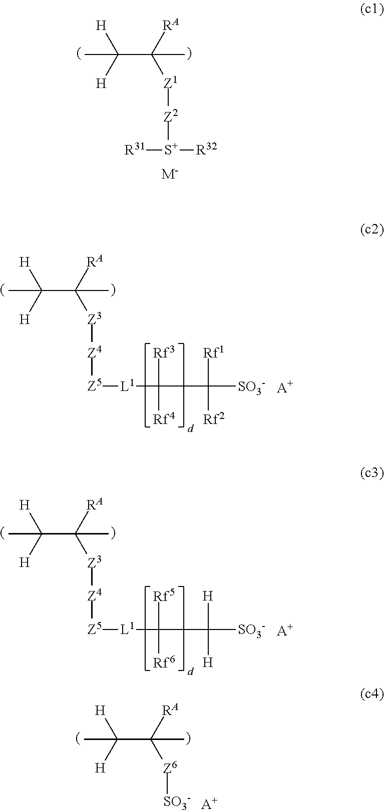

wherein R^{A} is each independently a hydrogen atom, a fluorine atom, a methyl group, or a trifluoromethyl group,
Z¹ is a single bond or a phenylene group,
Z² is *—C(═O)—O—Z²¹—, *—C(═O)—NH—Z²¹—, or *—O—Z²¹, —Z²¹ is a C₁-C₆ aliphatic hydrocarbylene group, a phenylene group, or a divalent group obtained by combining the foregoing, which may contain a carbonyl group, an ester bond, an ether bond, or a hydroxy group,
Z³ is each independently a single bond, a phenylene group, a naphthylene group, or *—C(═O)—O—Z³¹—, Z³¹ is a C₁-C₁₀ aliphatic hydrocarbylene group, a phenylene group, or a naphthylene group, the aliphatic hydrocarbylene group may contain a hydroxy group, an ether bond, an ester bond, or a lactone ring,
Z⁴ is each independently a single bond, *—Z⁴¹—C(═O)—O—, *—C(═O)—NH—Z⁴¹—, or *—O—Z⁴¹—, Z⁴¹ is a C₁-C₂₀ hydrocarbylene group which may contain a heteroatom,
Z⁵ is each independently a single bond, *—Z⁵¹—C(═O)—O—, *—C(═O)—NH—Z⁵¹—, or *—O—Z⁵¹—, Z⁵¹ is a C₁-C₂₀ hydrocarbylene group which may contain a heteroatom,
Z⁶ is a single bond, a methylene group, an ethylene group, a phenylene group, a fluorinated phenylene group, a trifluoromethyl group-substituted phenylene group, *—C(═O)—O—Z⁶¹—, *—C(═O)—N(H)—Z⁶¹—, or *—O—Z⁶¹—, Z⁶¹ is a C₁-C₆ aliphatic hydrocarbylene group, a phenylene group, a fluorinated phenylene group, or a trifluoromethyl group-substituted phenylene group, which may contain a carbonyl group, an ester bond, an ether bond, or a hydroxy group,
* designates a point of attachment to the carbon atom in the backbone,
R³¹ and R³² are each independently a C₁-C₂₀ hydrocarbyl group which may contain a heteroatom, R³¹ and R³² may bond together to form a ring with the sulfur atom to which they are attached,
L¹ is a single bond, an ether bond, an ester bond, a carbonyl group, a sulfonic acid ester bond, a carbonate bond, or a carbamate bond,
Rf¹ and Rf² are each independently a fluorine atom or a C₁-C₆ fluorinated saturated hydrocarbyl group,
Rf³ and Rf⁴ are each independently a hydrogen atom, a fluorine atom, or a C₁-C₆ fluorinated saturated hydrocarbyl group,
Rf⁵ and Rf⁶ are each independently a hydrogen atom, a fluorine atom, or a C₁-C₆ fluorinated saturated hydrocarbyl group, provided that all Rf⁵ and Rf⁶ are not a hydrogen atom at the same time,
M⁻ is a non-nucleophilic counter ion,
A⁺ is an onium cation, and
d is an integer of 0 to 3.
13. The resist composition according to any one of 7 to 12, further comprising a photoacid generator.
14. The resist composition according to any one of 7 to 13, further comprising a quencher other than the quencher according to 6.
15. The resist composition according to any one of 7 to 14, further comprising a surfactant.
16. A pattern forming process comprising the steps of: applying the resist composition according to any one of 7 to 15 onto a substrate to form a resist film thereon; exposing the resist film to high-energy radiation; and developing the exposed resist film in a developer.
17. The pattern forming process according to 16, wherein the high-energy radiation is KrF excimer laser, ArF excimer laser, EB, or EUV of wavelength 3 to 15 nm.

### Advantageous Effects of the Invention

The onium salt of the present invention functions well as a quencher in the resist composition. A resist film obtained from the resist composition comprising an onium salt of the present invention has favorable sensitivity and is excellent in dissolution contrast, and as a result, dimensional uniformity (CDU) of a hole pattern and LWR of a line pattern are improved, and a pattern profile with high resolution and excellent rectangularity can be constructed.

### BRIEF DESCRIPTION OF THE DRAWINGS

FIG. 1 is a ¹H-NMR spectrum of Onium Salt NSQ-1 obtained in Example 1-1.

### DETAILED DESCRIPTION OF THE INVENTION

### [Onium Salt]

An onium salt of the present invention has the following formula (1).

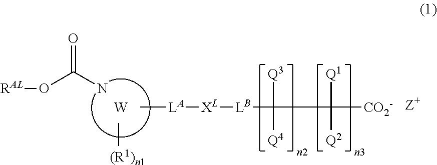

In formula (1), n1 is an integer of 0 to 6. n2 is an integer of 0 to 3, preferably 0 or 1. n3 is an integer of 1 to 4, preferably 1 or 2, more preferably 1.

In formula (1), W is a C₂-C₂₀ nitrogen atom-containing aliphatic heterocyclic ring which may contain a heteroatom. Specific examples of the structure of W are shown below, but not limited thereto. In the following formula, * designates a point of attachment to L^{A}, and ** designates a point of attachment to R^{AL}—O—C(═O)—.

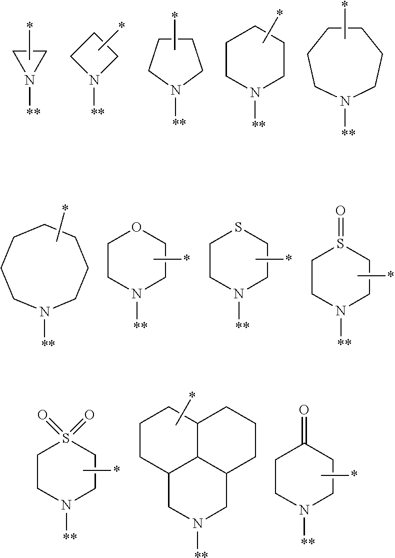

In formula (1), L^{A} and L^{B} are each independently a single bond, an ether bond, an ester bond, an amide bond, a sulfonic acid ester bond, a carbonate bond, or a carbamate bond. Of these, a single bond, an ether bond, an ester bond, or an amide bond is preferable, and a single bond, an ester bond, or an amide bond is more preferable.

In formula (1), X^{L} is a single bond or a C₁-C₄₀ hydrocarbylene group which may contain a heteroatom. The hydrocarbylene group may be straight, branched, or cyclic, and specific examples thereof include an alkanediyl group and a cyclic saturated hydrocarbylene group. Specific examples of the heteroatom include an oxygen atom, a nitrogen atom, and a sulfur atom.

Specific examples of the C₁-C₄₀ hydrocarbylene group, which may contain a heteroatom, represented by X^{L} are shown below, but not limited thereto. In the following formula, * designates a point of attachment to L^{A} and L^{B}.

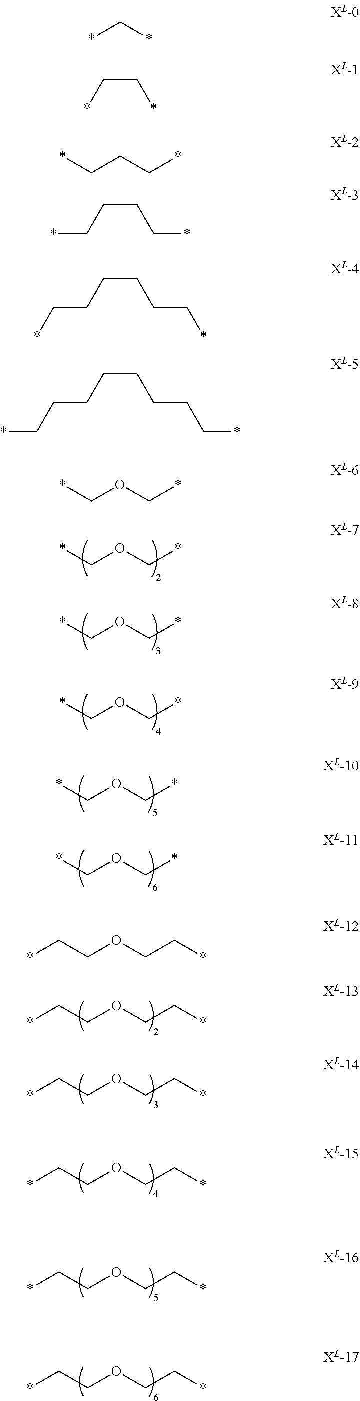

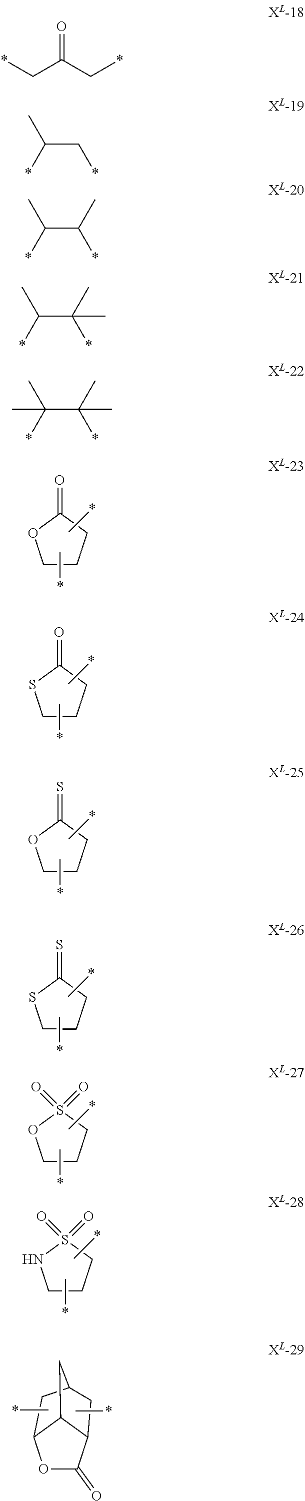

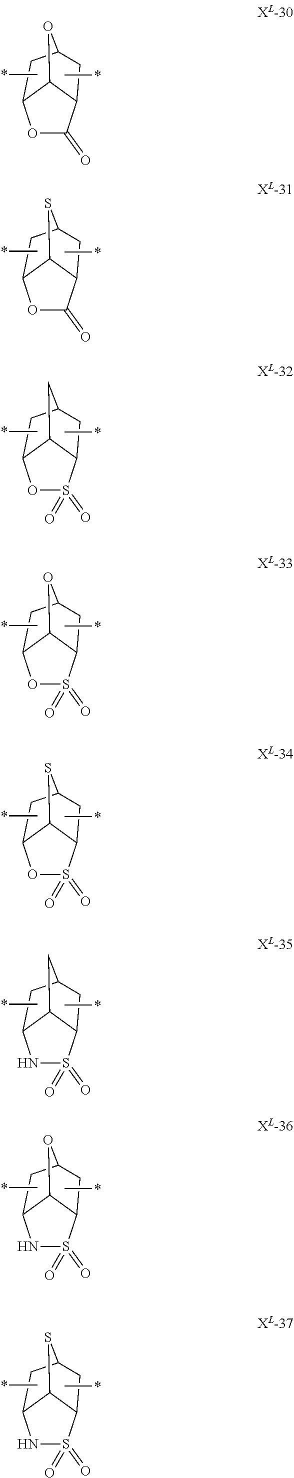

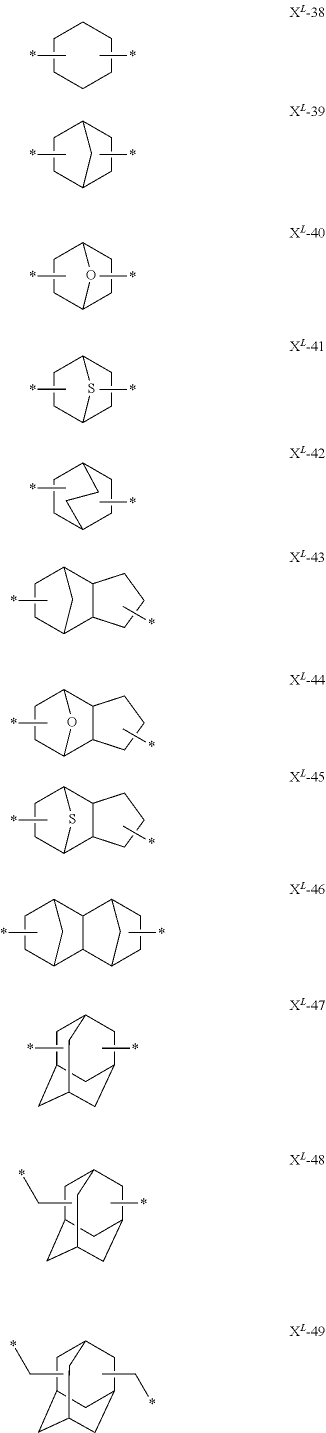

Of these, X^{L}-0 to X^{L}-22 and X^{L}-47 to X^{L}-49 are preferable, and X^{L}-0 to X^{L}-17 are more preferable.

In formula (1), R¹ is a C₁-C₂₀ hydrocarbyl group which may contain a heteroatom. The hydrocarbyl group may be saturated or unsaturated and straight, branched, or cyclic. Specific examples thereof include C₁-C₂₀ alkyl groups such as a methyl group, an ethyl group, a propyl group, an isopropyl group, a n-butyl group, a sec-butyl group, an isobutyl group, a tert-butyl group, a n-pentyl group, a tert-pentyl group, a n-hexyl group, a n-octyl group, a 2-ethylhexyl group, a n-nonyl group, and a n-decyl group; C₃-C₂₀ cyclic saturated hydrocarbyl groups such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.02.6]decyl group, an adamantyl group, and an adamantylmethyl group; C₆-C₂₀ aryl groups such as a phenyl group, a naphthyl group, and an anthryl group; and groups obtained by combining these. Some or all of the hydrogen atoms in the hydrocarbyl group may be substituted with a group containing a heteroatom such as an oxygen atom, a sulfur atom, a nitrogen atom, or a halogen atom, some of —CH₂— in the hydrocarbyl group may be substituted with a group containing a heteroatom such as an oxygen atom, a sulfur atom, or a nitrogen atom, so that the group may contain a hydroxy group, a fluorine atom, a chlorine atom, a bromine atom, an iodine atom, a cyano group, a carbonyl group, an ether bond, an ester bond, a sulfonic acid ester bond, a carbonate bond, a carbamate bond, an amide bond, an imide bond, a lactone ring, a sultone ring, a thiolactone ring, a lactam ring, a sultam ring, a carboxylic anhydride (—C(═O)—O—C(═O)—), a haloalkyl group, or the like.

When n1≥2, a plurality of R¹'s may bond together to form a ring with the carbon atom on W to which they are attached. Specific examples of the ring formed at this time include a cyclopropane ring, a cyclobutane ring, a cyclopentane ring, a cyclohexane ring, a norbornane ring, and an adamantane ring. Some or all of the hydrogen atoms in the ring may be substituted with a group containing a heteroatom such as an oxygen atom, a sulfur atom, a nitrogen atom, or a halogen atom, some of —CH₂— in the ring may be substituted with a group containing a heteroatom such as an oxygen atom, a sulfur atom, or a nitrogen atom, so that the group may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, or the like. Two R¹'s bonded to the same atom forming W may bond together to form a ring, thereby forming a spiro ring.

In formula (1), Q¹ to Q⁴ are each independently a hydrogen atom, a fluorine atom, a C₁-C₆ hydrocarbyl group, or a C₁-C₆ fluorinated hydrocarbyl group. Provided that at least one of Q¹ to Q⁴ is a fluorine atom or a C₁-C₆ fluorinated hydrocarbyl group. The hydrocarbyl group may be saturated or unsaturated and straight, branched, or cyclic. Specific examples thereof include those having 1 to 6 carbon atoms among those exemplified for the C₁-C₂₀ hydrocarbyl group represented by R¹. A C₁-C₆ fluorinated hydrocarbyl group is preferably a trifluoromethyl group. Q³ and Q⁴ may bond together to form a ring with the carbon atom to which they are attached. Specific examples of the ring formed at this time include a cyclopropane ring, a cyclobutane ring, a cyclopentane ring, a cyclohexane ring, a norbornane ring, and an adamantane ring.

In formula (1), R^{AL} is an acid labile group. Specifically, R^{AL} is preferably a group having the following formula (AL-1) or (AL-2).

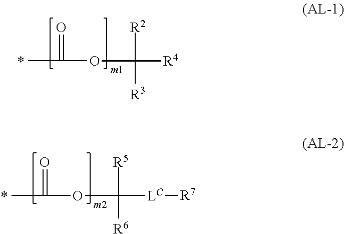

wherein * designates a point of attachment to the adjacent —O—.

In formula (AL-2), LC is-O— or —S—.

In formula (AL-1), R², R³, and R⁴ are each independently a C₁-C₁₀ hydrocarbyl group. Any two of R², R³, and R⁴ may bond together to form a ring with the carbon atom to which they are attached. m1 is 0 or 1.

In formula (AL-2), R⁵ and R⁶ are each independently a hydrogen atom or a C₁-C₁₀ hydrocarbyl group. R⁷ is a C₁-C₂₀ hydrocarbyl group, some of —CH₂— in the hydrocarbyl group may be replaced by —O— or —S—. R⁶ and R⁷ may bond together to form a C₃-C₂₀ heterocyclic group with the carbon atom and LC to which they are attached, some of —CH₂— in the heterocyclic group may be replaced by —O— or —S—. m2 is 0 or 1.

Specific examples of the acid labile group having formula (AL-1) are shown below, but not limited thereto. In the following formula, * designates a point of attachment to the adjacent —O—.

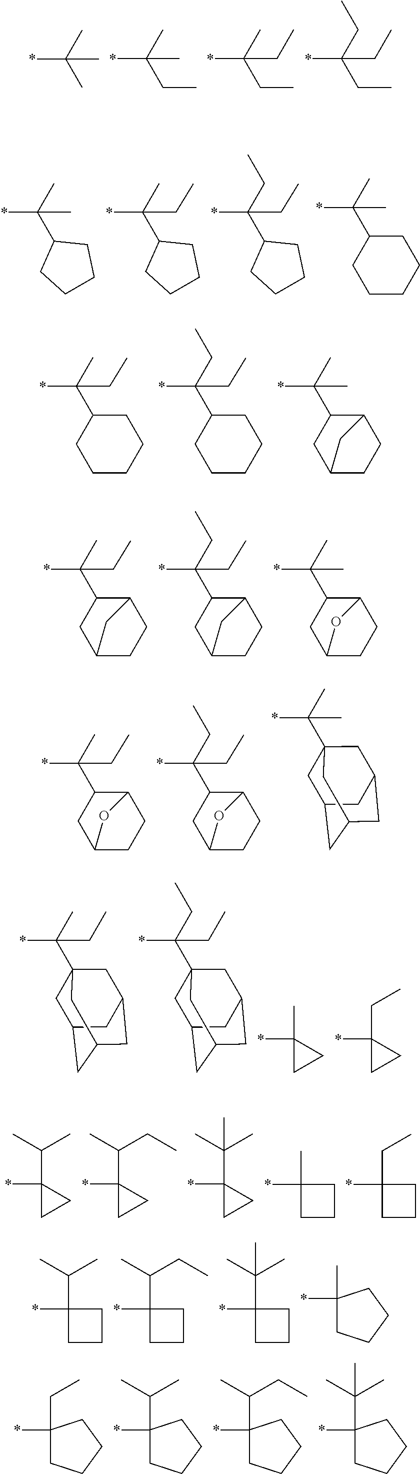

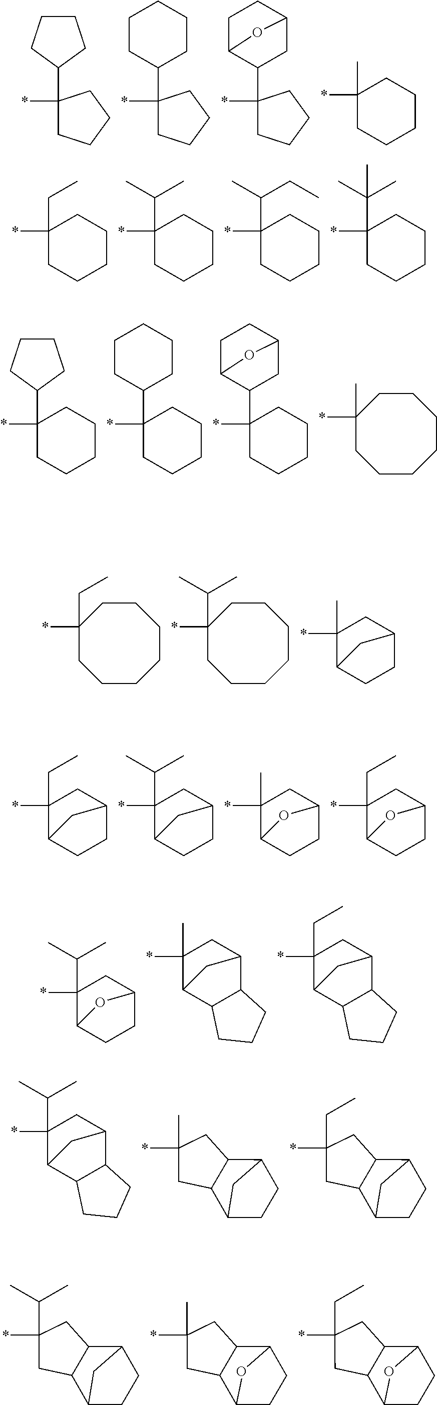

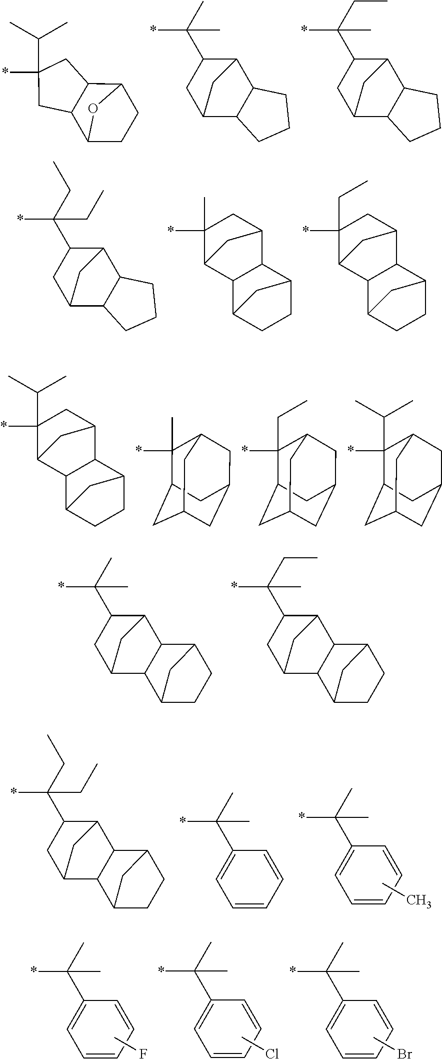

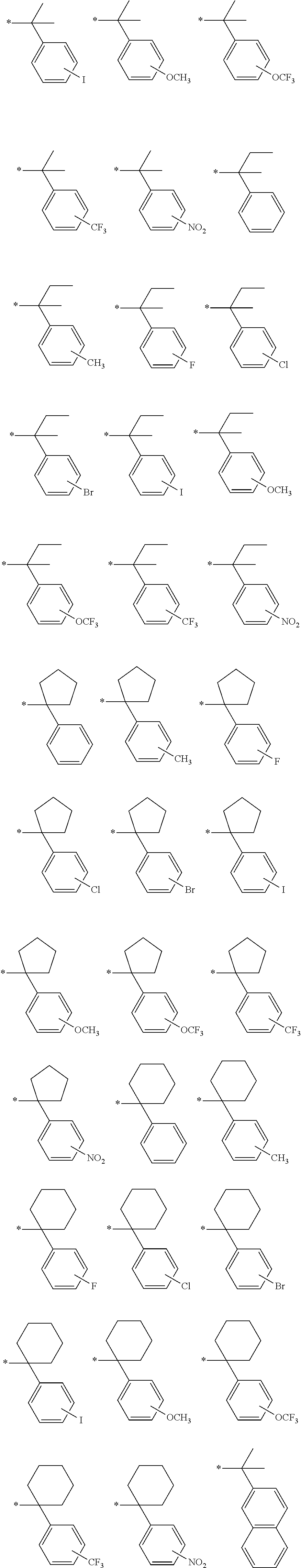

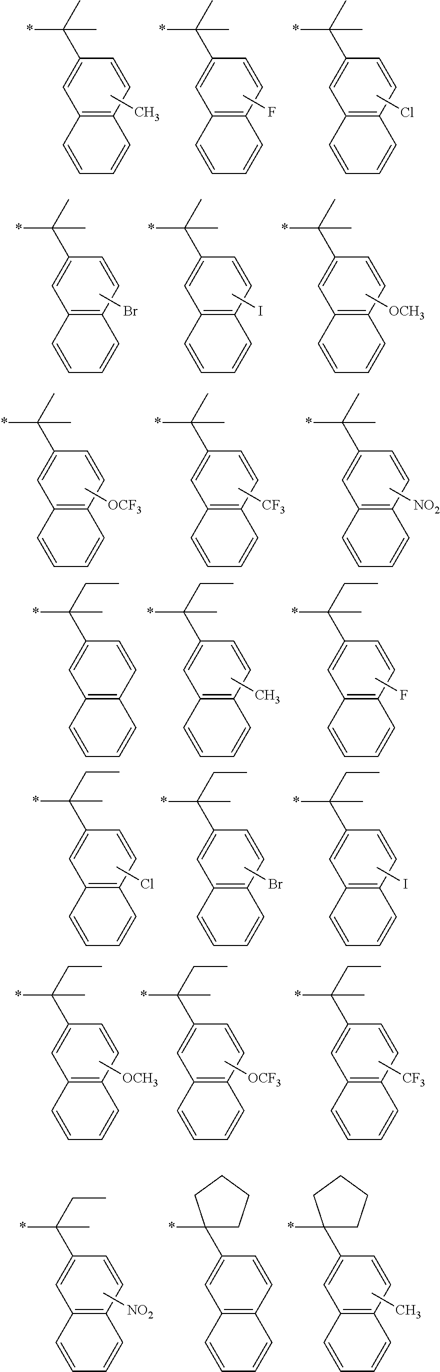

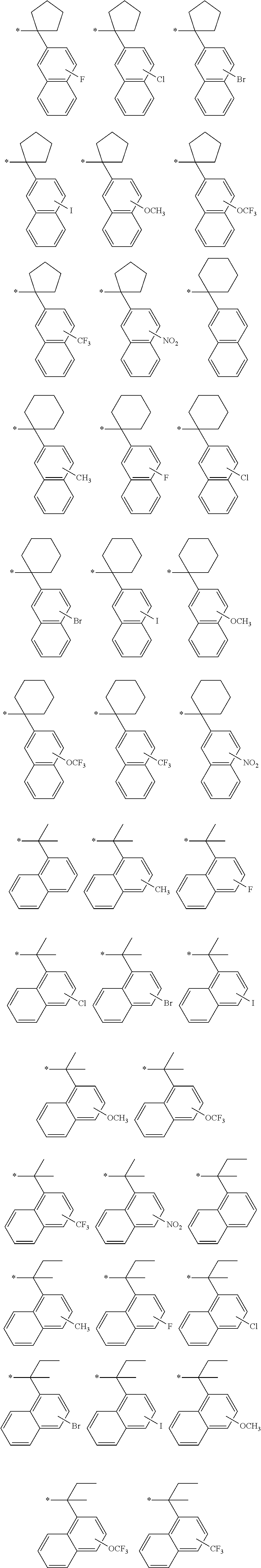

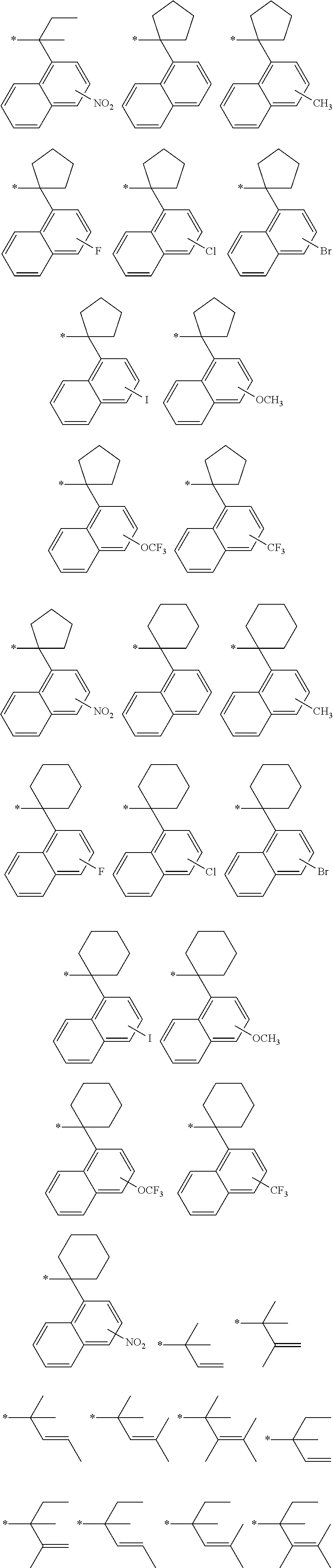

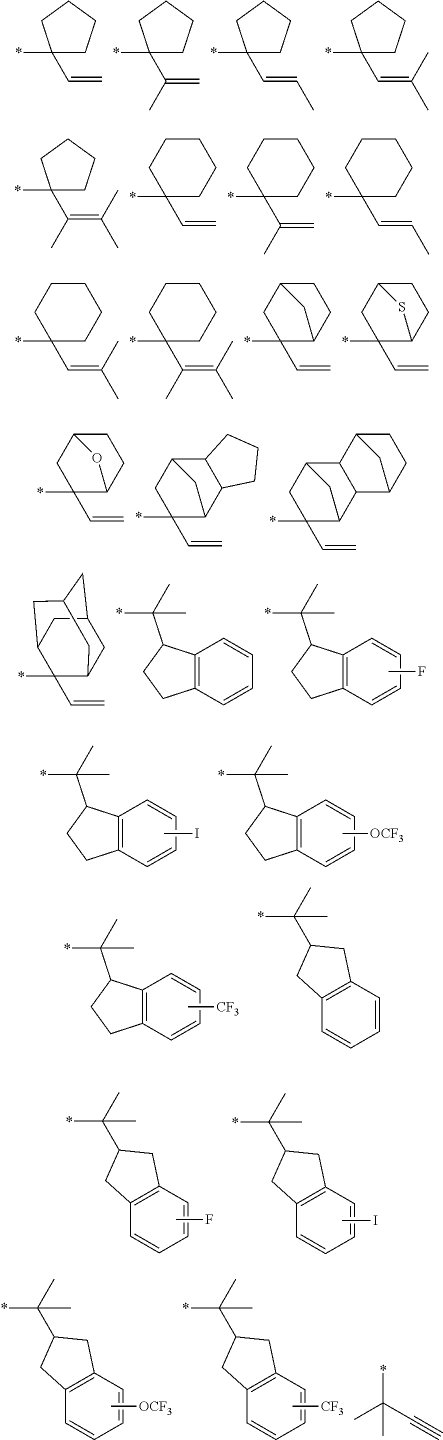

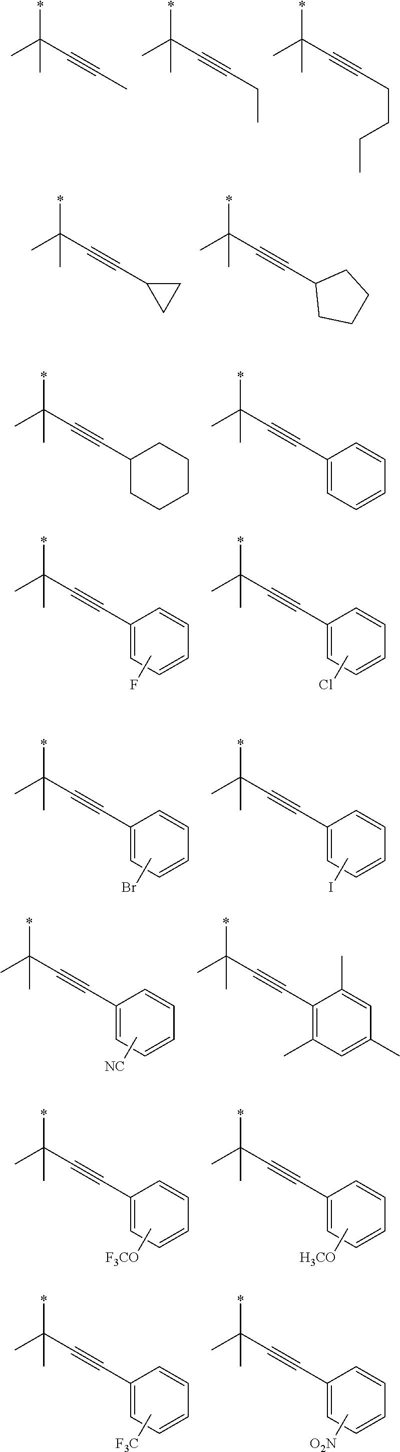

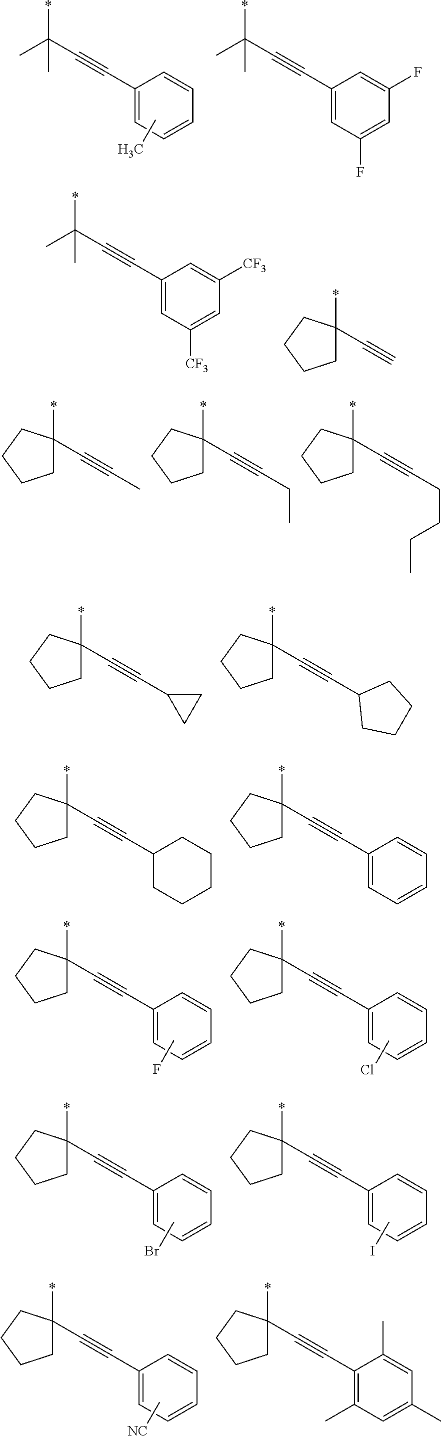

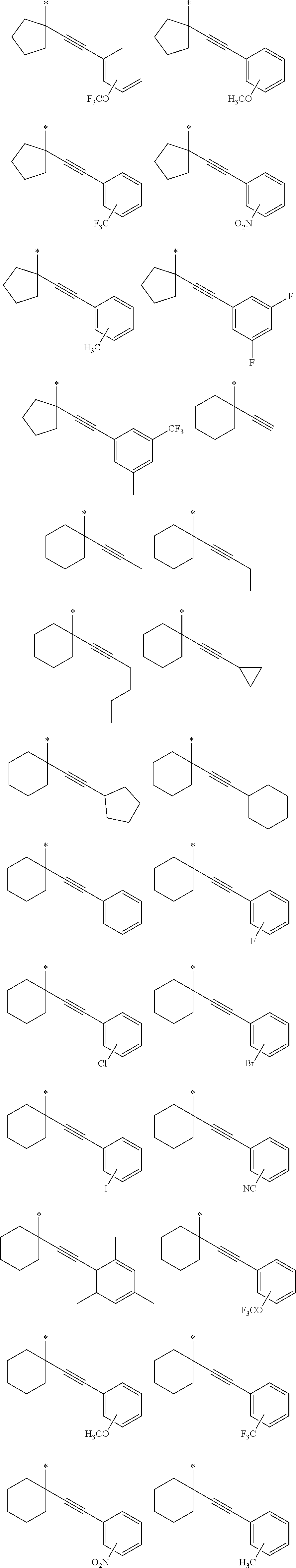

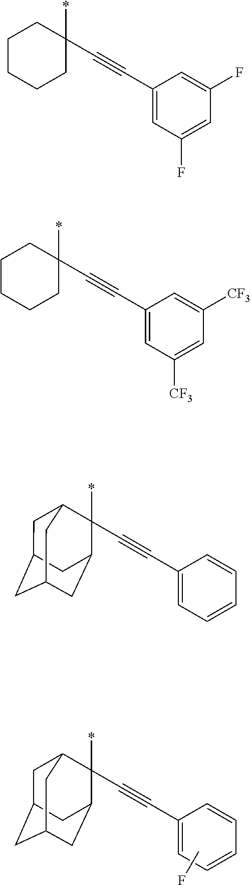

Specific examples of the acid labile group having formula (AL-2) are shown below, but not limited thereto. In the following formula. * designates a point of attachment to the adjacent —O—.

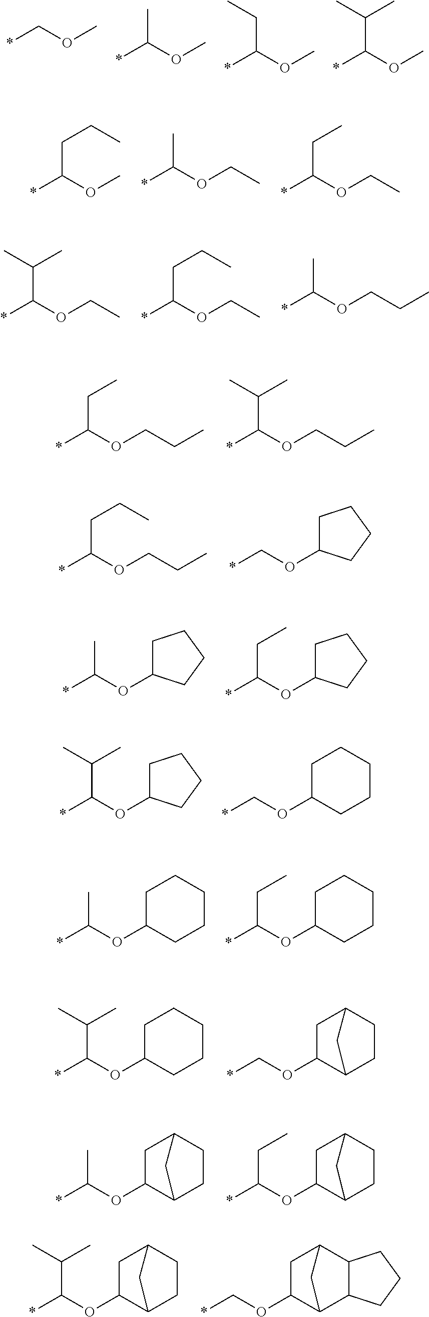

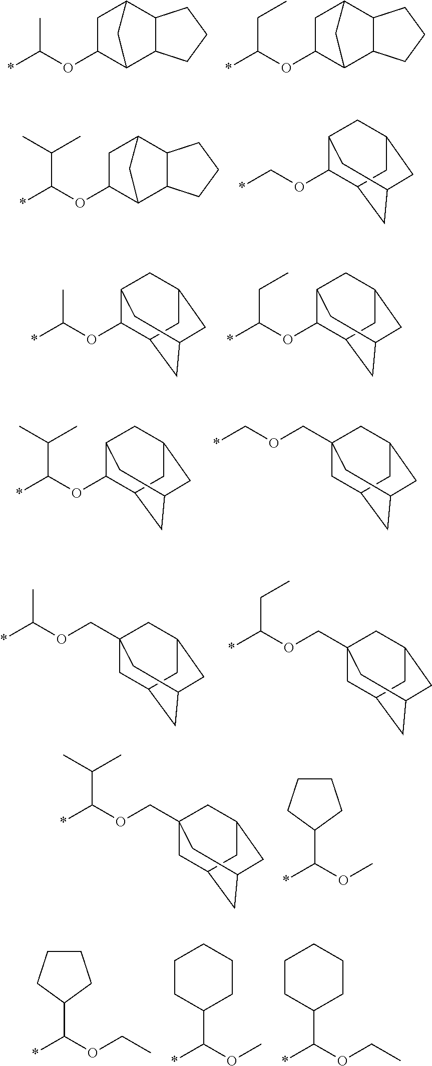

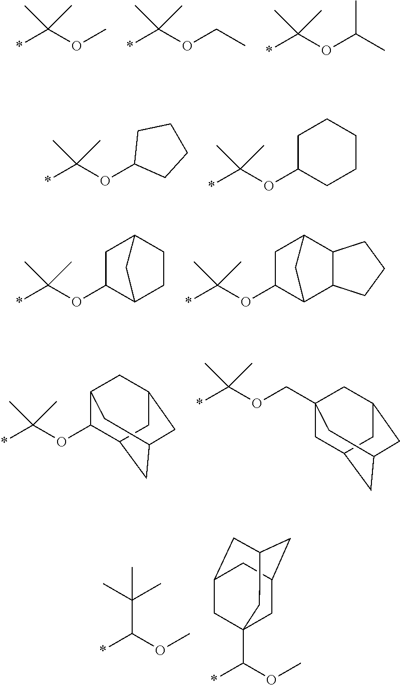

The onium salt having formula (1) preferably has the following formula (1A).

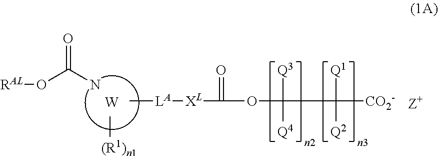

wherein n1 to n3, W, L^{A}, X^{L}, R¹, Q¹ to Q⁴, R^{AL}, and Z⁺ are as defined above.

The onium salt having formula (1A) preferably has the following formula (1B).

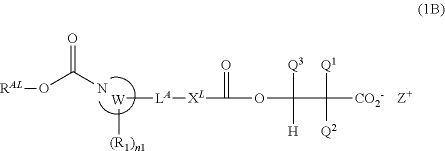

wherein n1, W, L^{A}, X^{L}, R¹, Q¹ to Q³, R^{AL}, and Z⁺ are as defined above.

Specific examples of the anion of the onium salt having formula (1) are shown below, but not limited thereto.

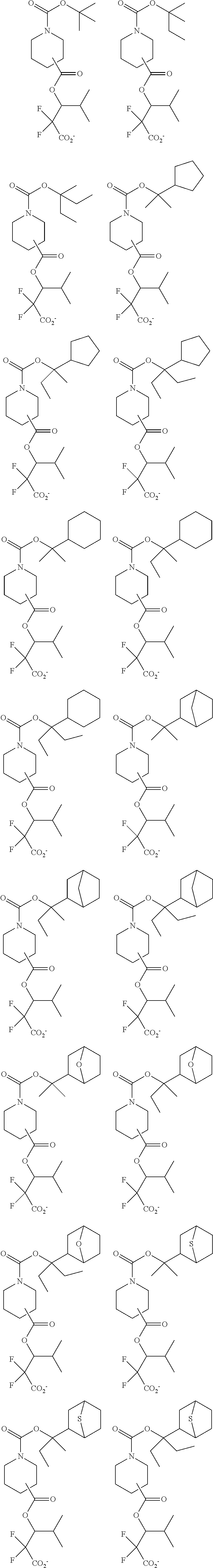

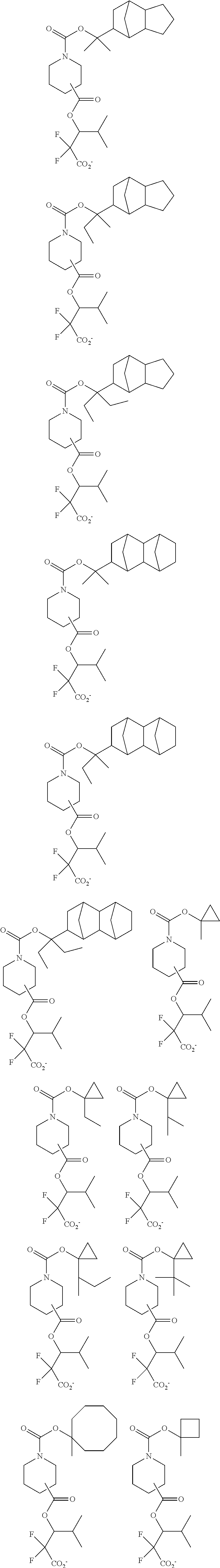

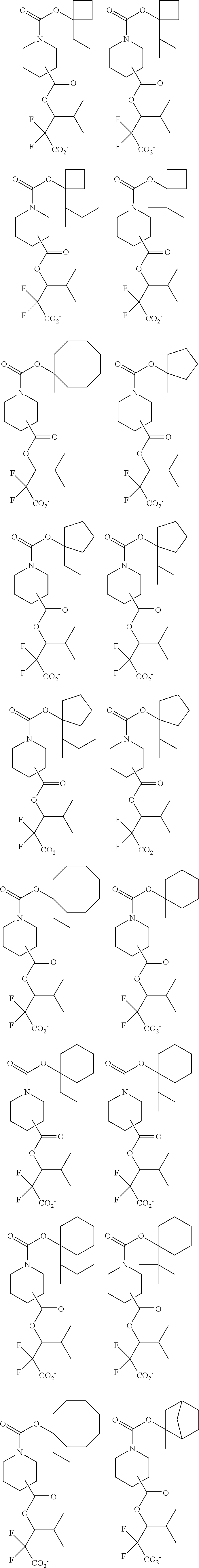

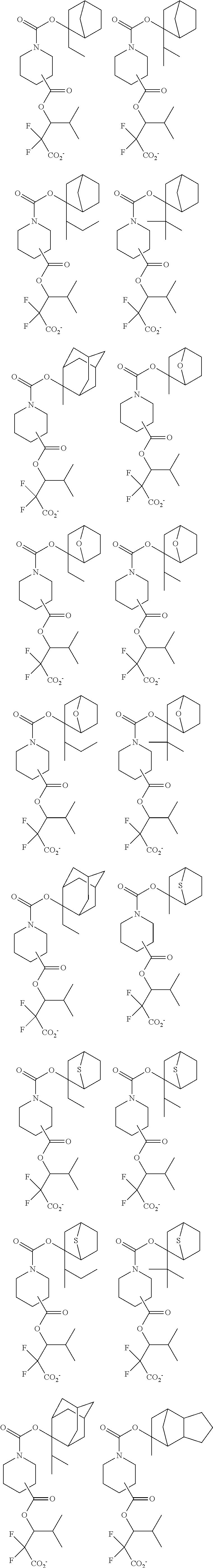

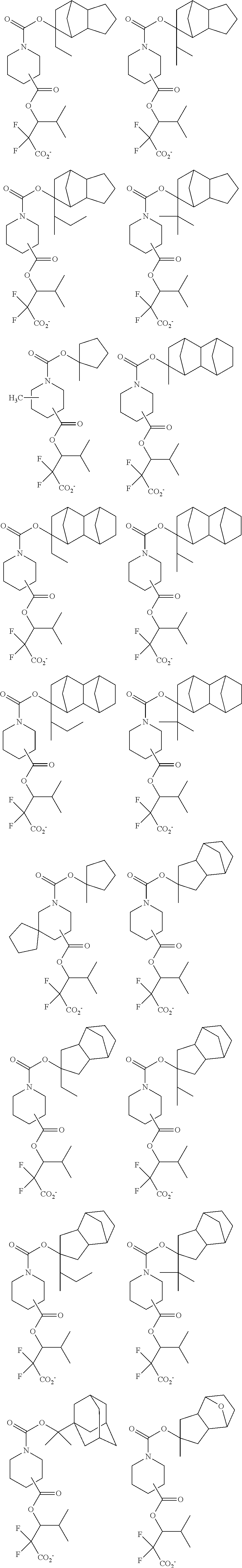

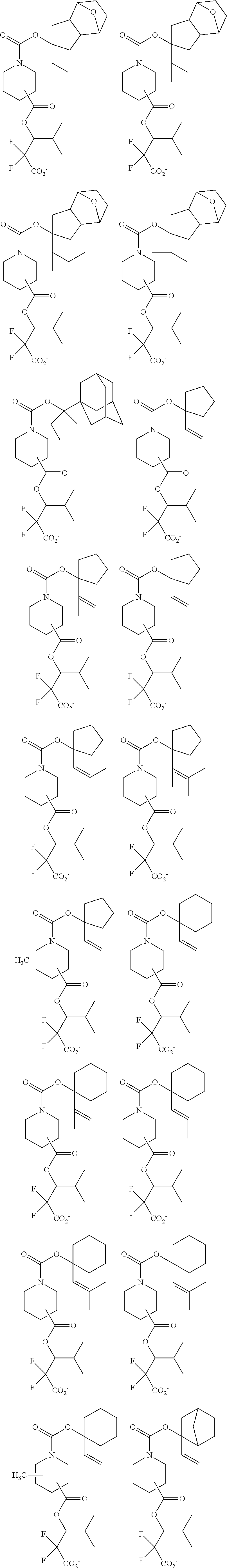

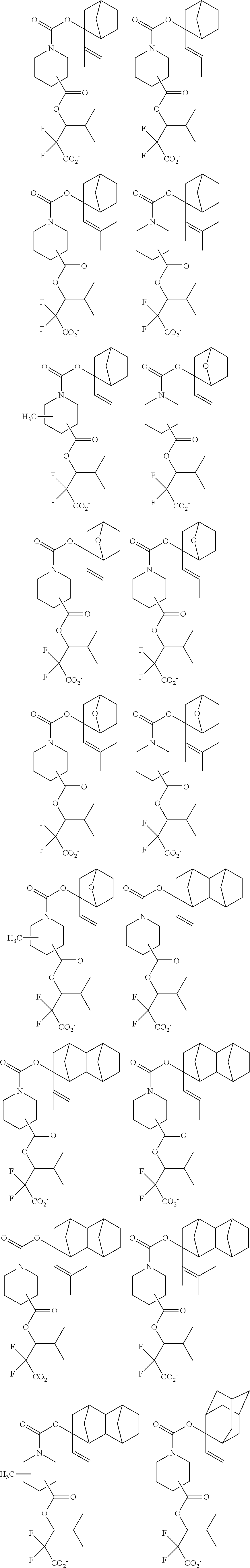

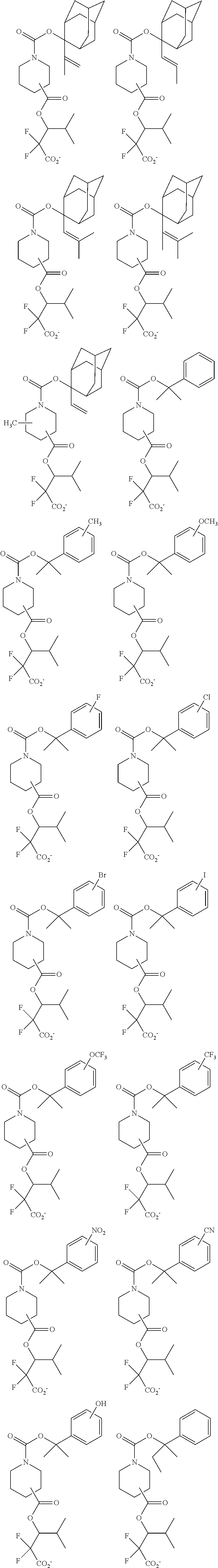

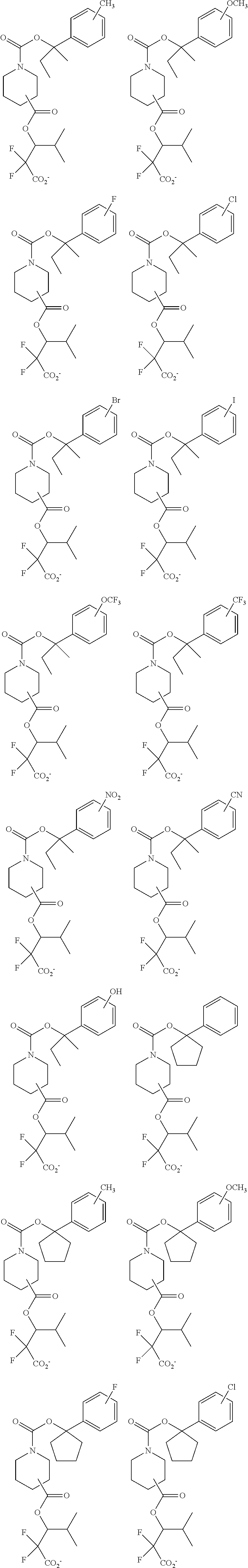

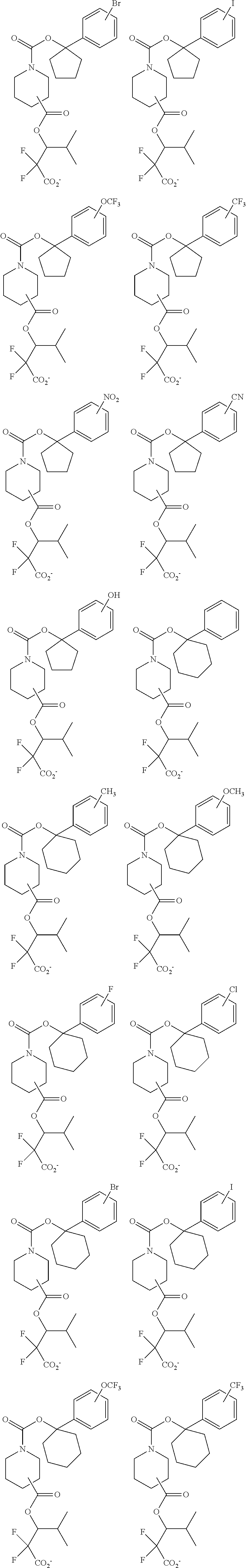

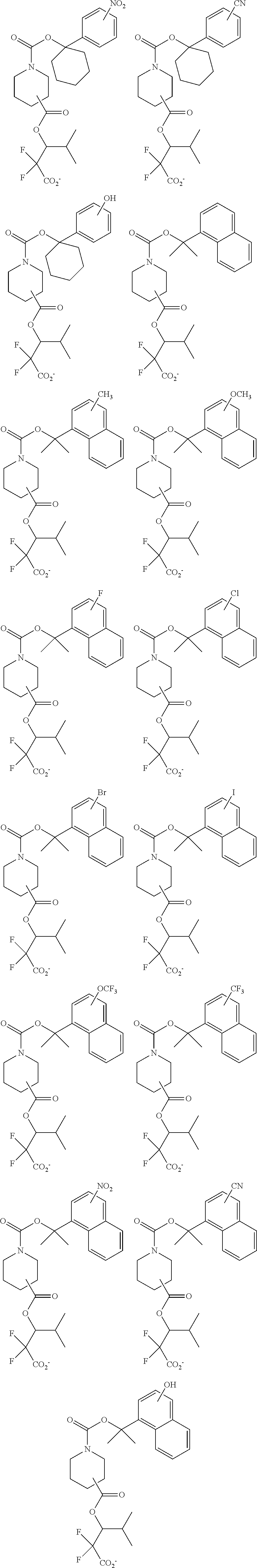

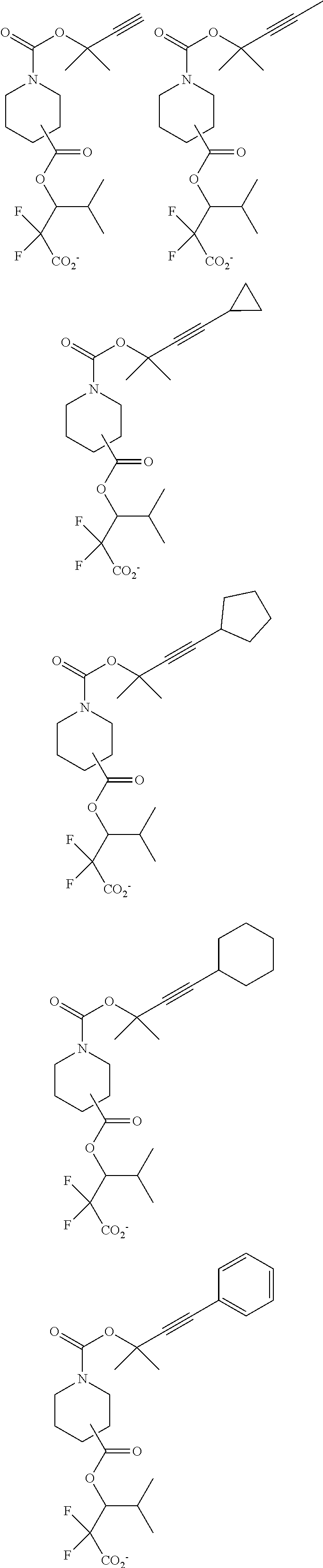

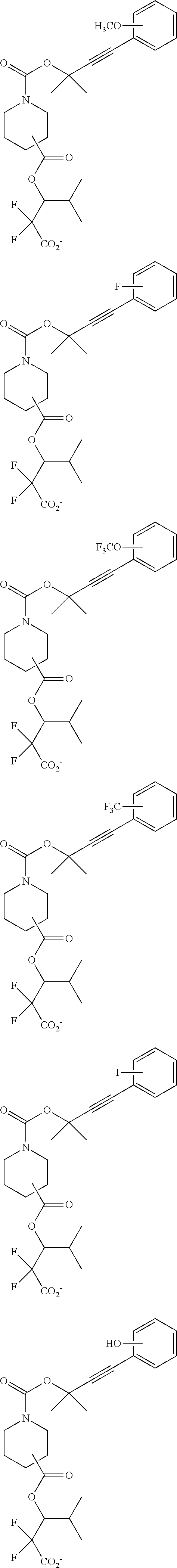

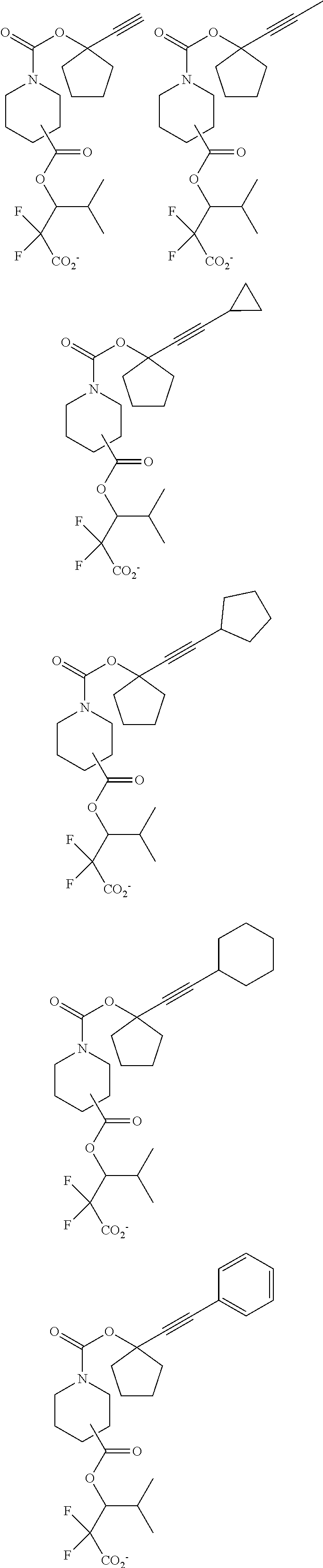

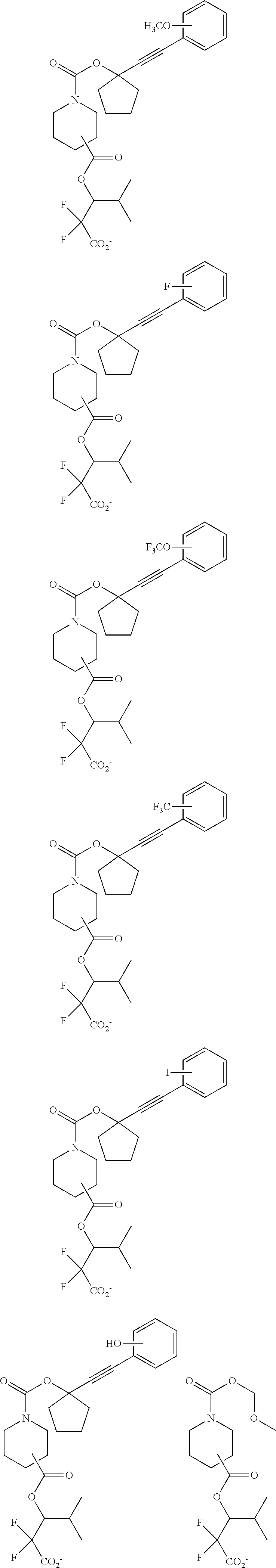

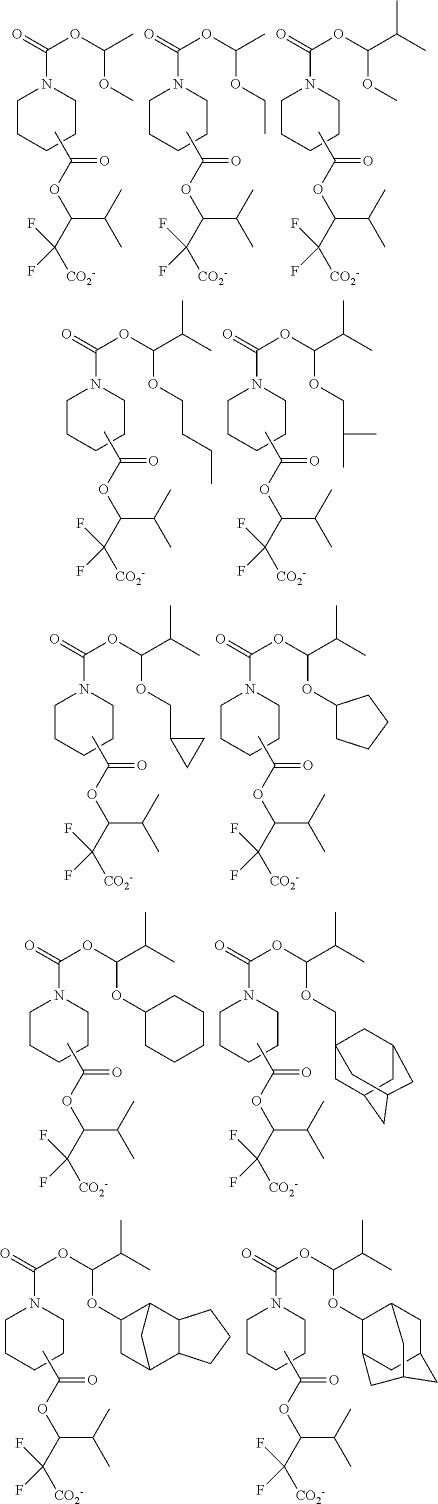

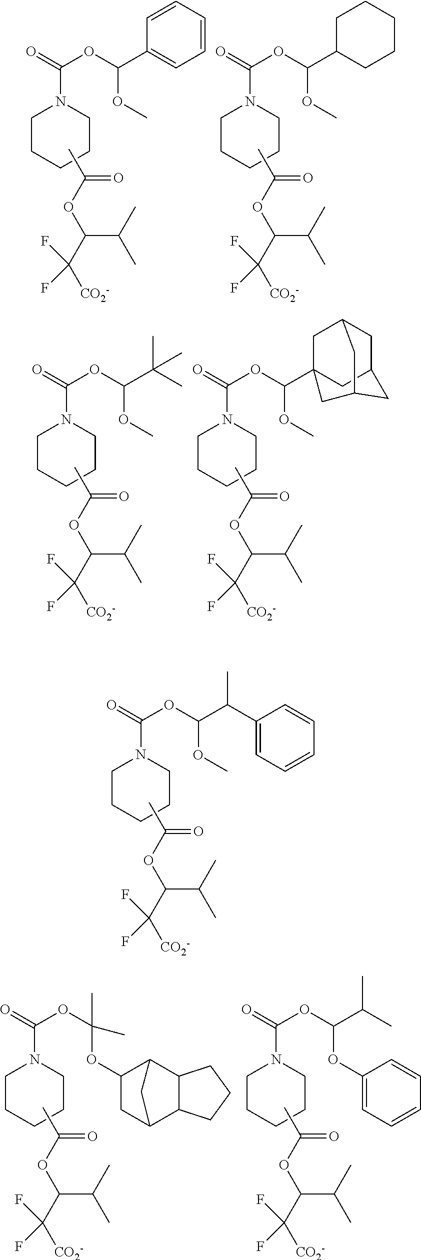

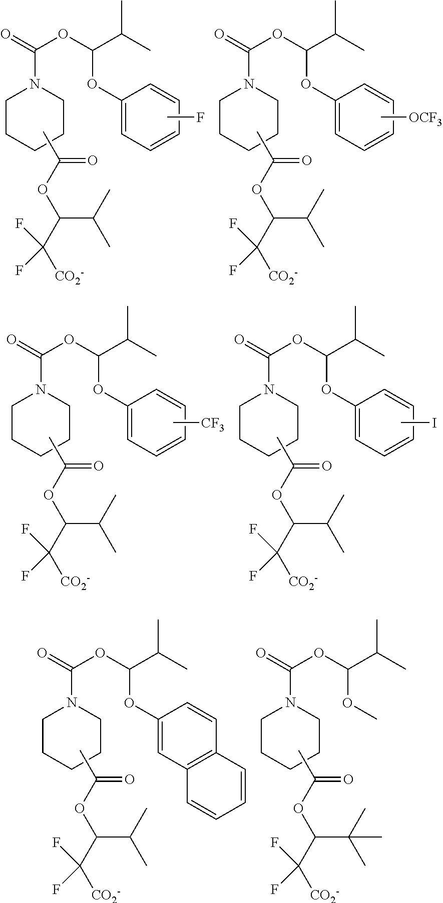

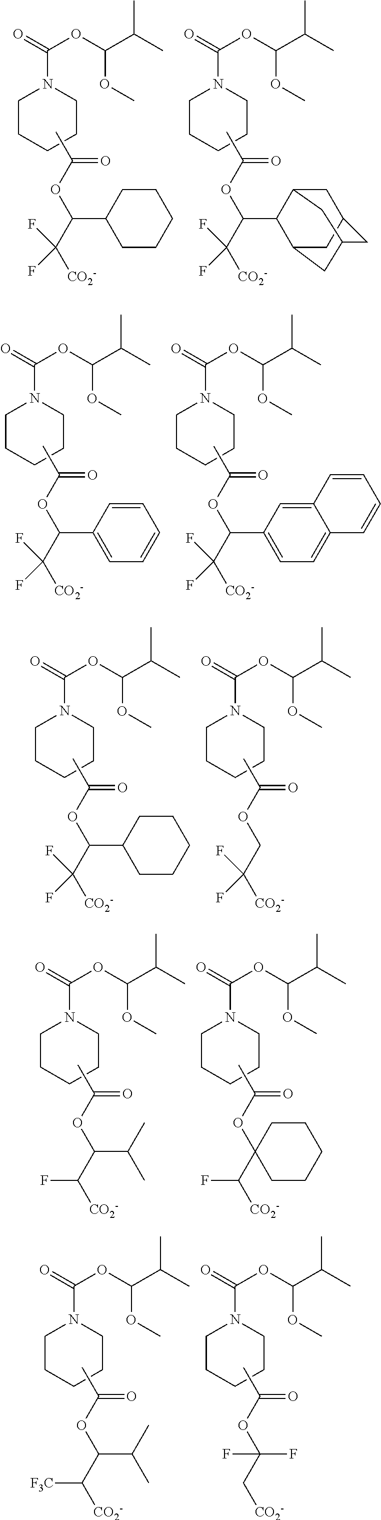

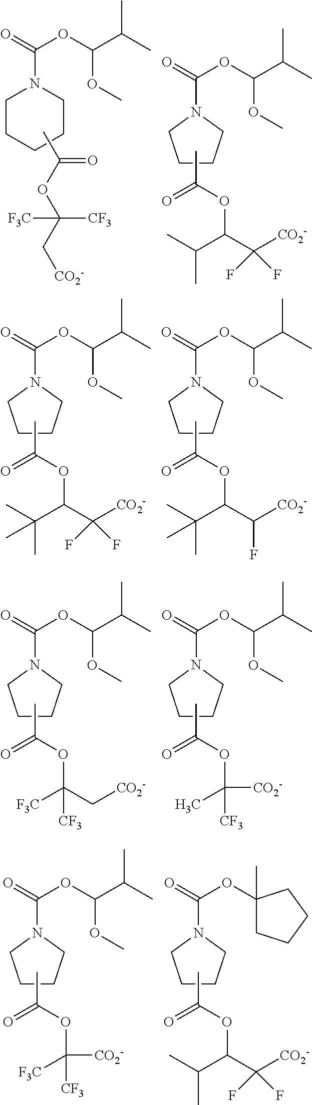

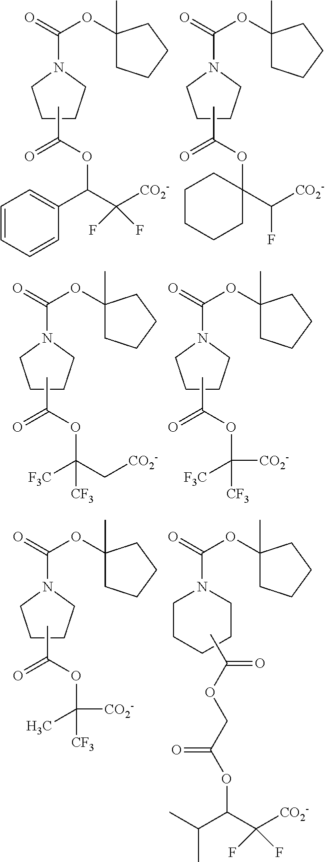

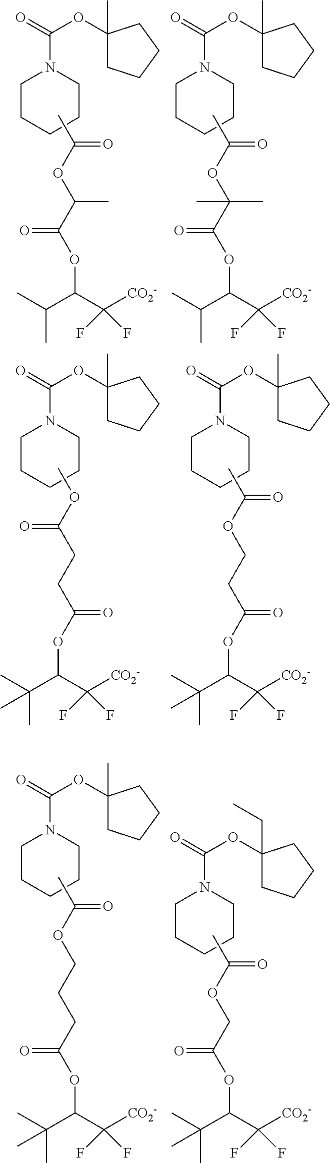

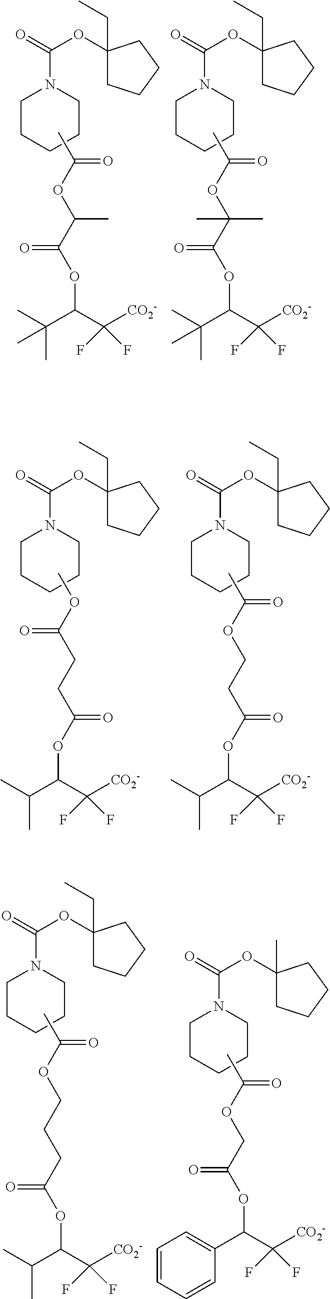

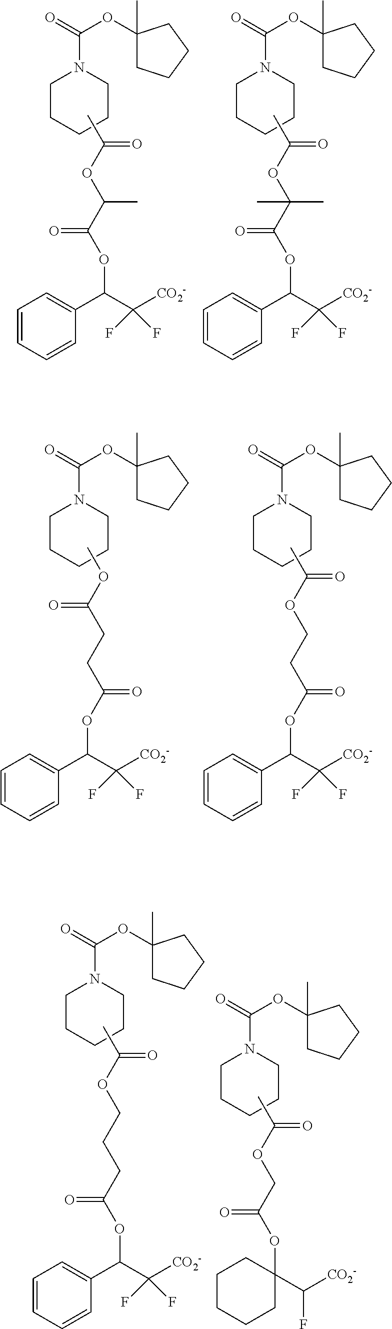

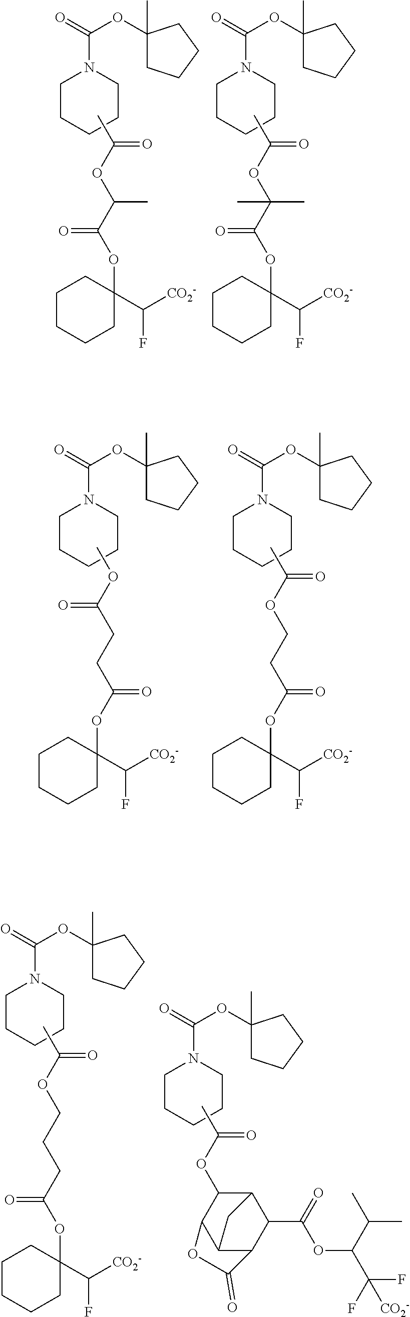

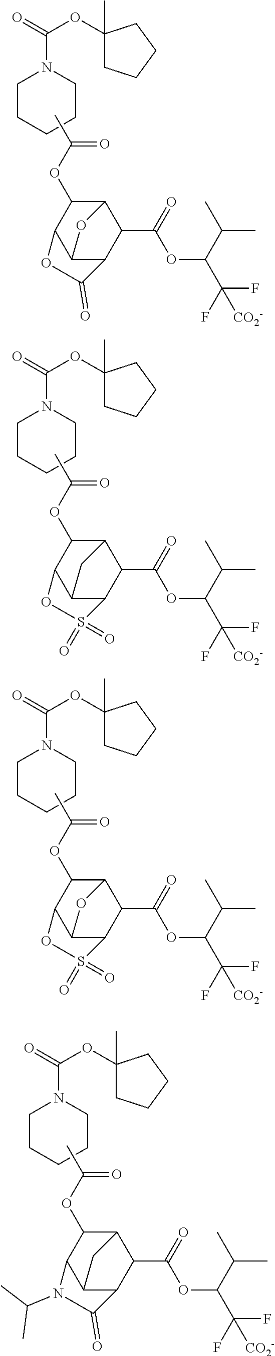

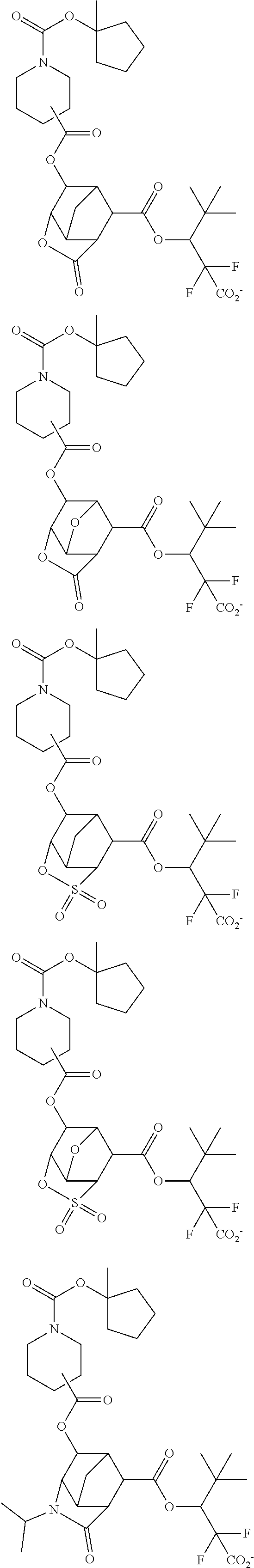

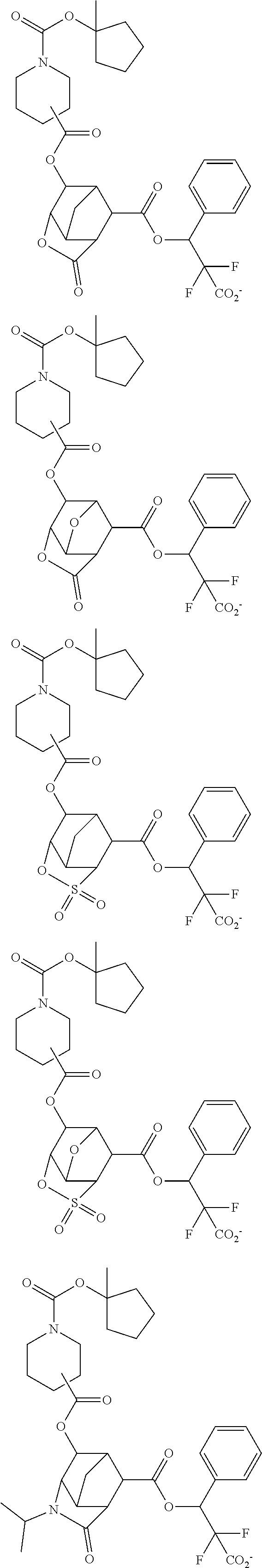

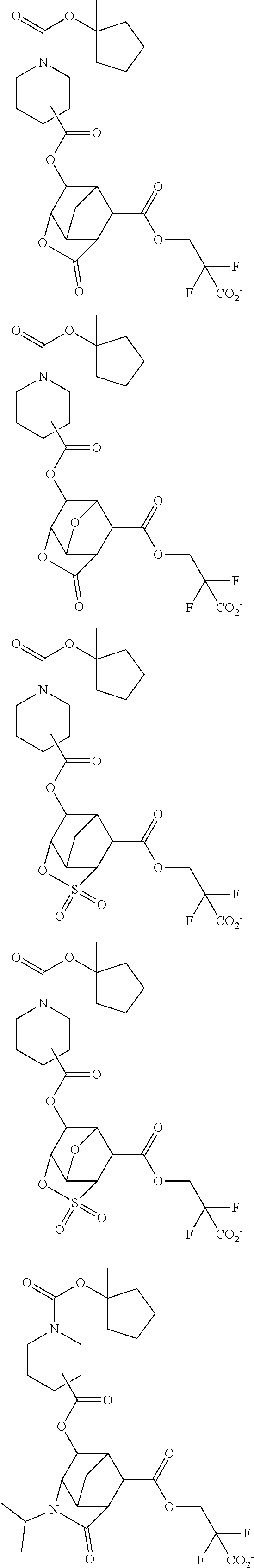

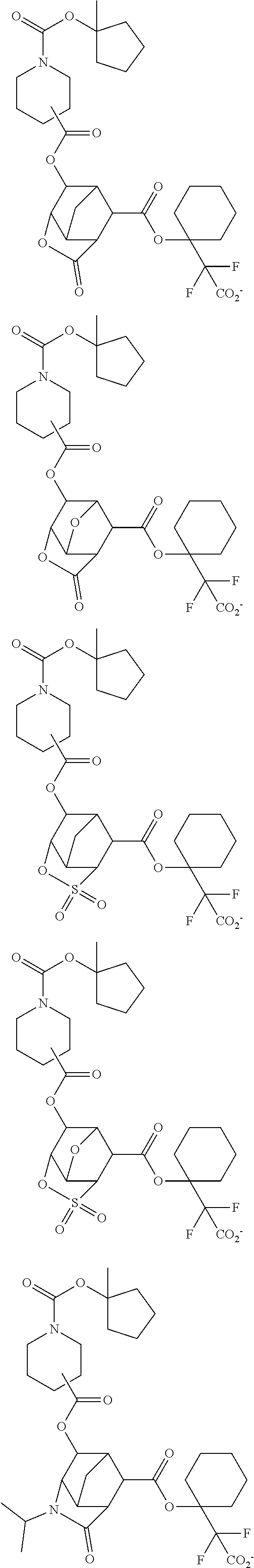

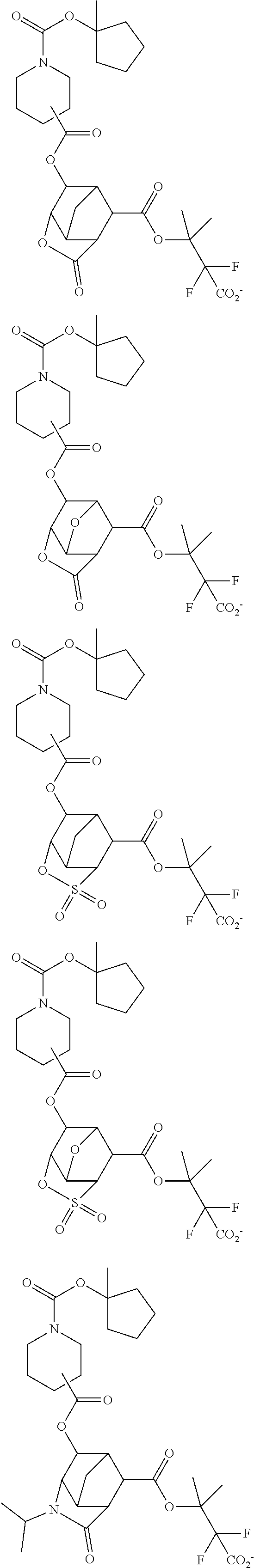

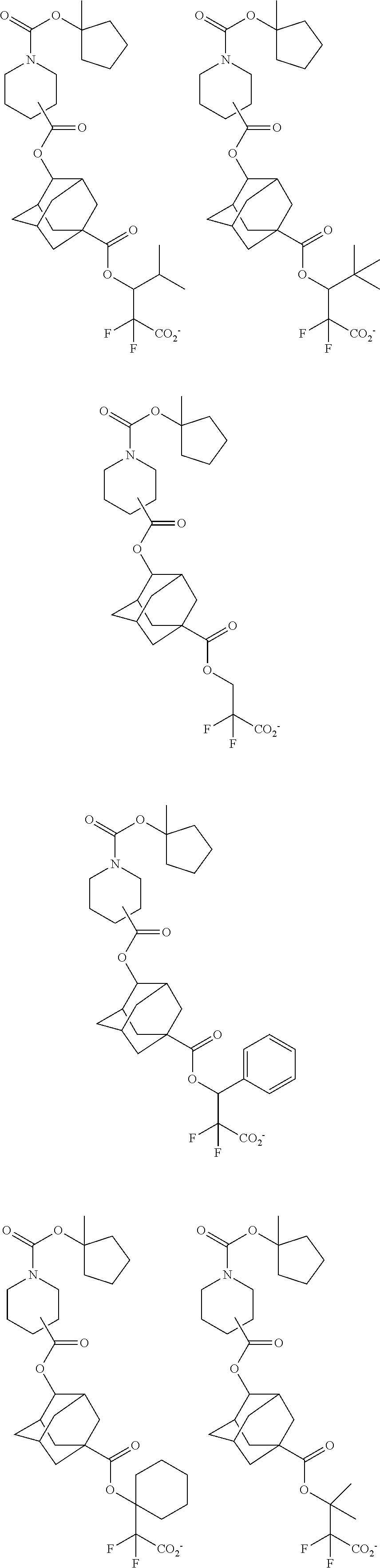

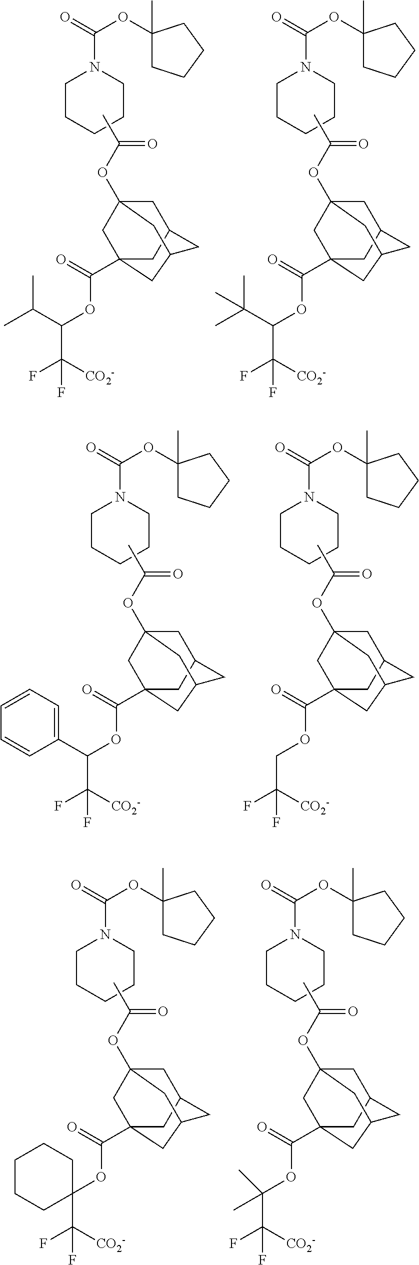

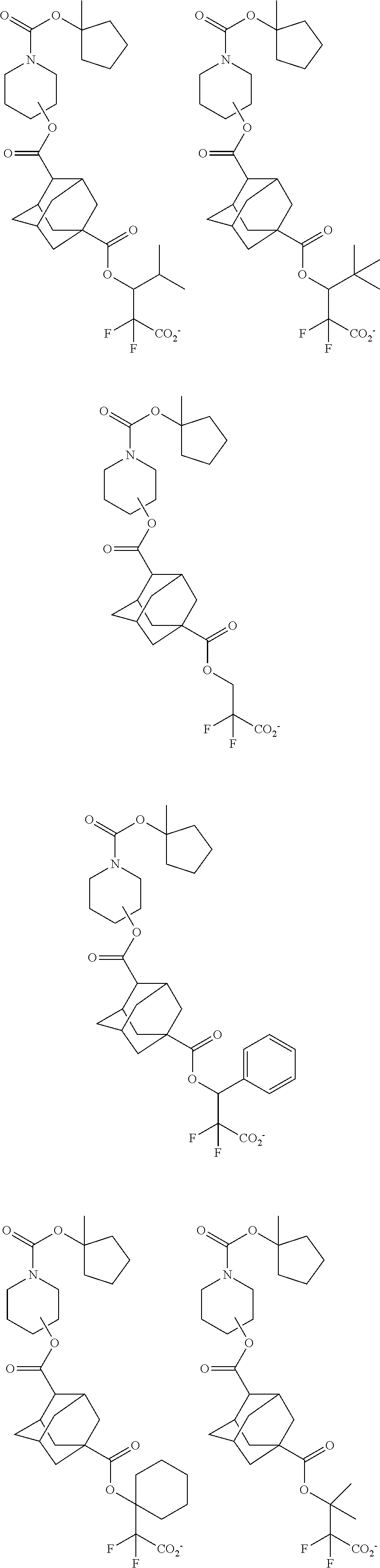

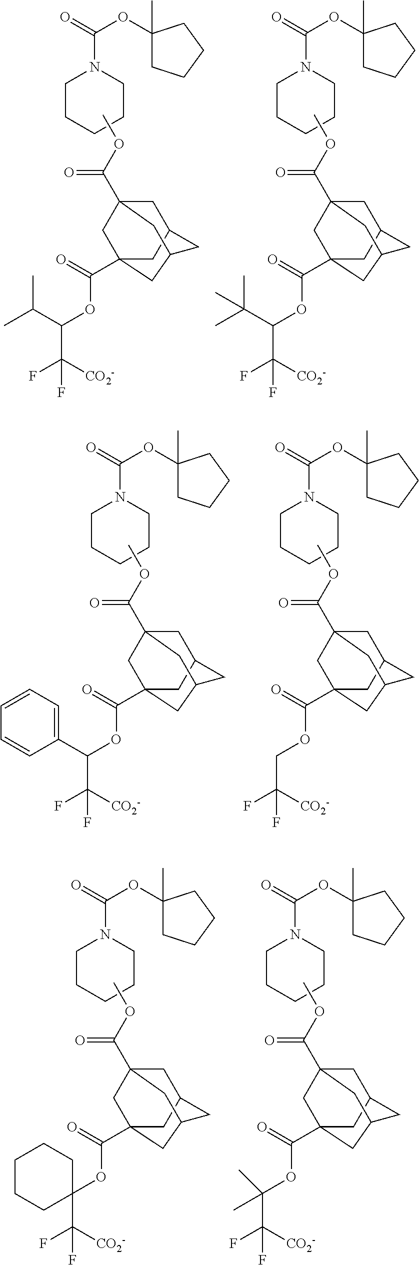

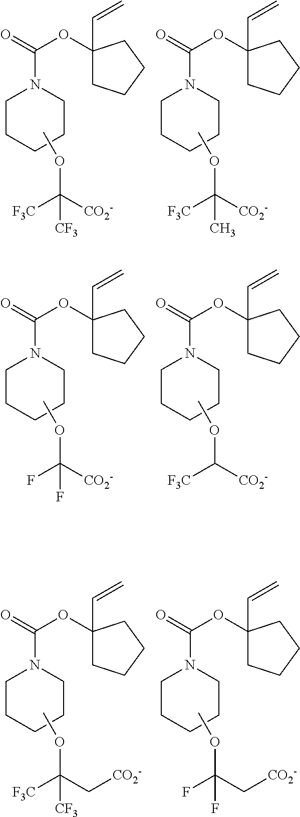

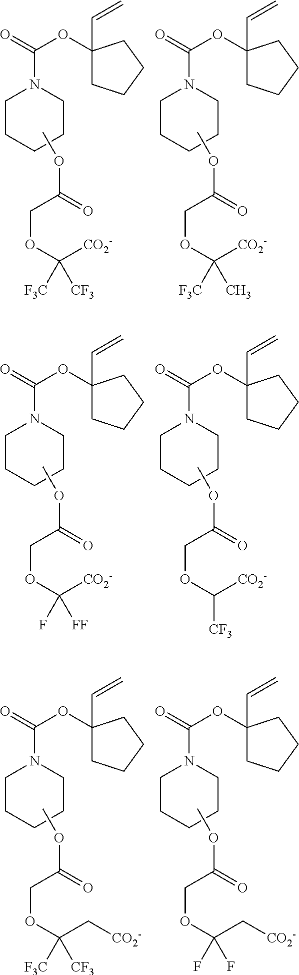

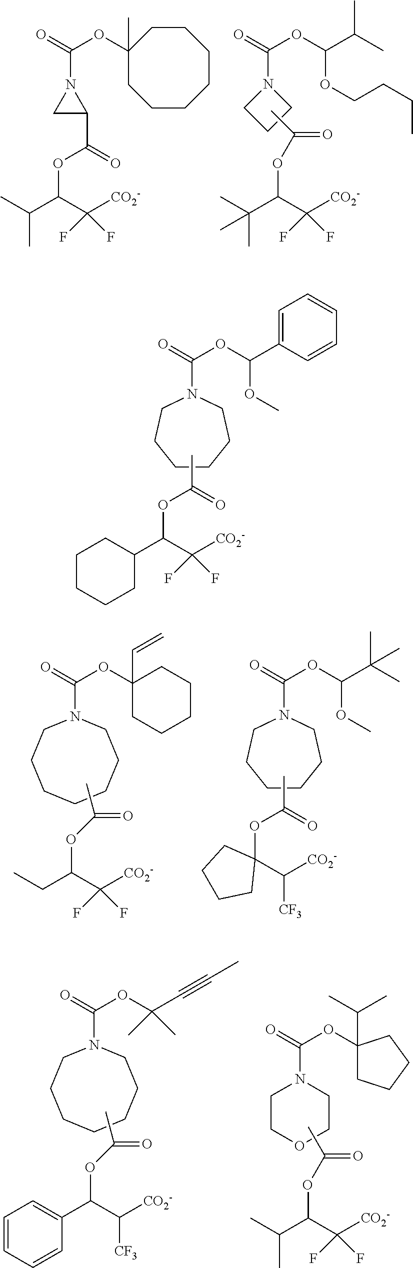

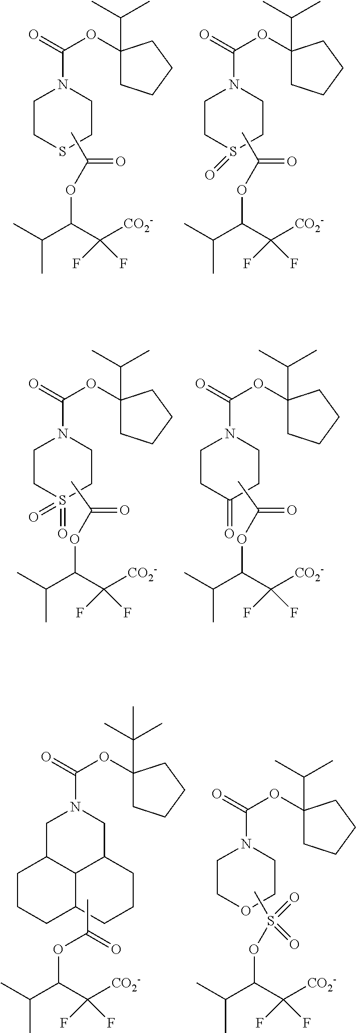

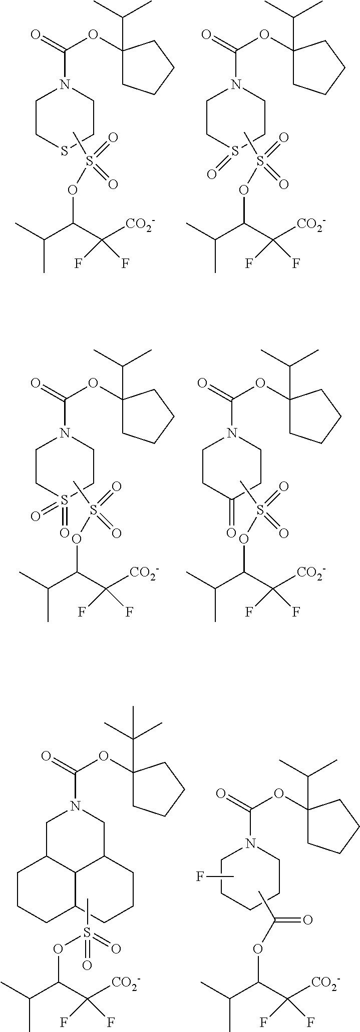

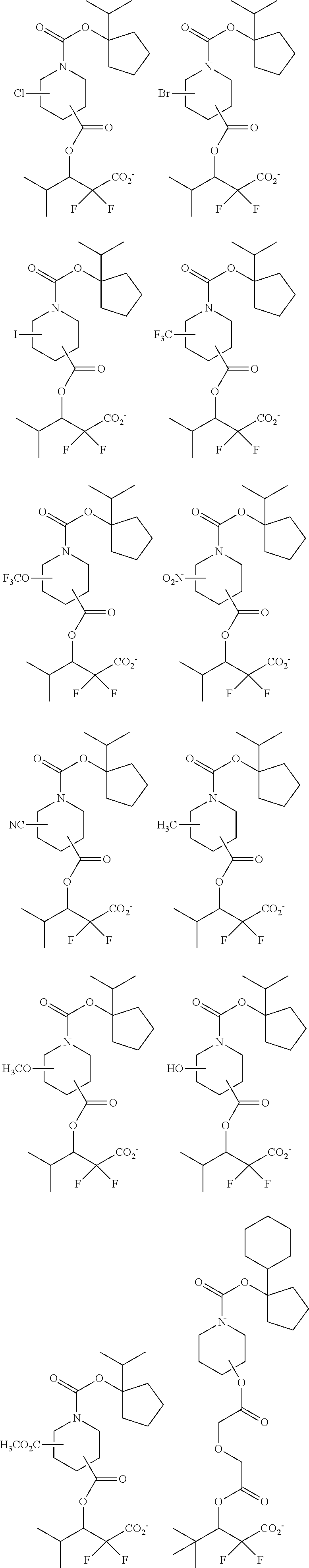

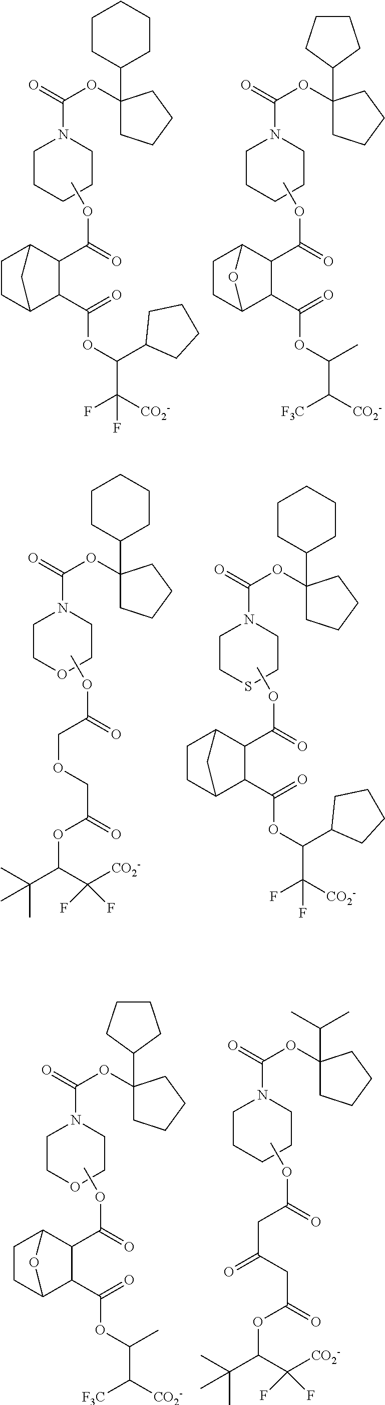

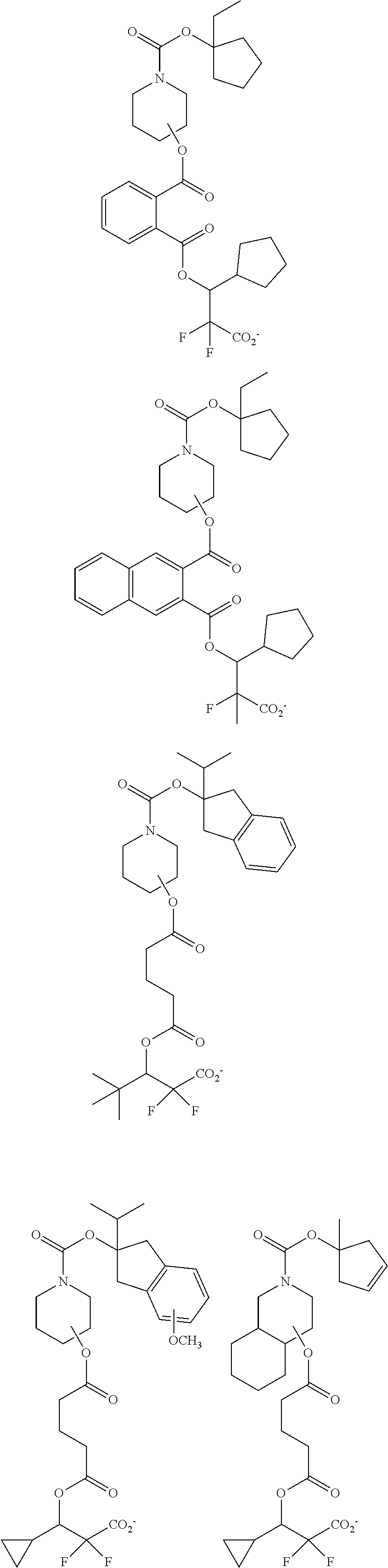

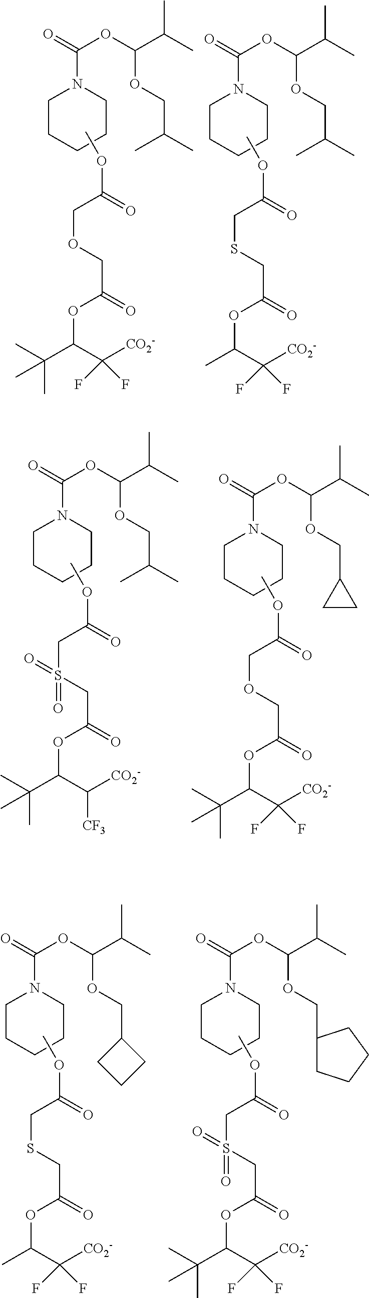

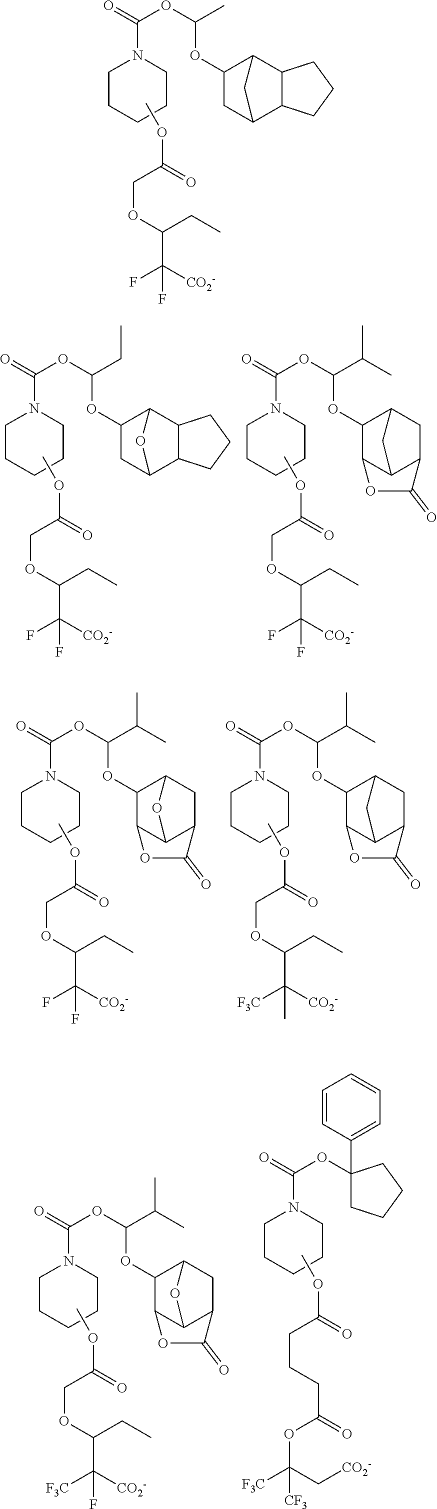

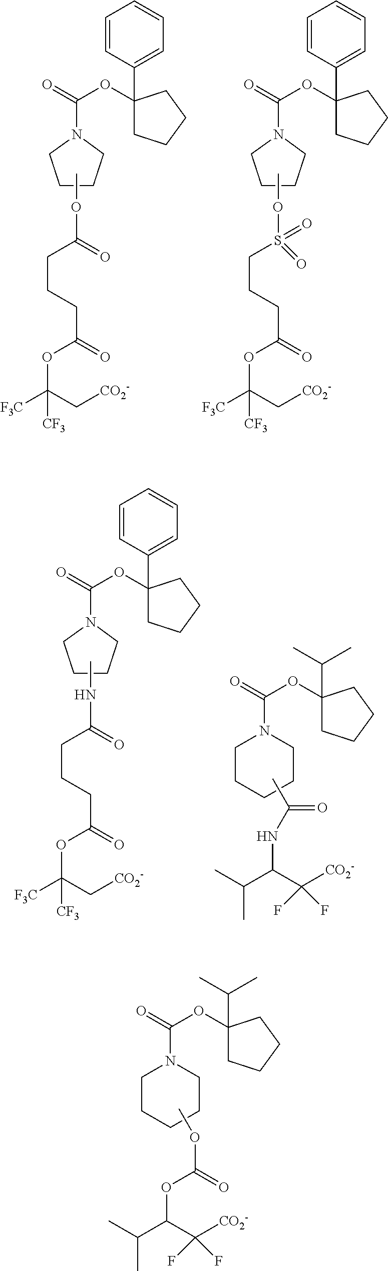

In formula (1). Z⁺ is an onium cation. The onium cation is preferably a sulfonium cation having the following formula (cation-1), an iodonium cation having the following formula (cation-2), or an ammonium cation having the following formula (cation-3):

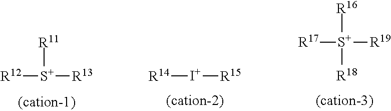

In formulae (cation-1) to (cation-3), R¹¹ to R¹⁹ are each independently a C₁-C₃₀ hydrocarbyl group which may contain a heteroatom. The hydrocarbyl group may be saturated or unsaturated and straight, branched, or cyclic. Specific examples thereof include C₁-C₃₀ alkyl groups such as a methyl group, an ethyl group, a n-propyl group, an isopropyl group, a n-butyl group, and a tert-butyl group; C₃-C₃₀ cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₃₀ alkenyl groups such as a vinyl group, an allyl group, a propenyl group, a butenyl group, and a hexenyl group; C₃-C₃₀ cyclic unsaturated hydrocarbyl groups such as a cyclohexenyl group; C₆-C₃₀ aryl groups such as a phenyl group, a naphthyl group, and a thienyl group; C₇-C₃₀ aralkyl groups such as a benzyl group, a 1-phenylethyl group, and a 2-phenylethyl group; and groups obtained by combining these, and an aryl group is preferable. Some or all of the hydrogen atoms in the hydrocarbyl group may be substituted with a group containing a heteroatom such as an oxygen atom, a sulfur atom, a nitrogen atom, or a halogen atom, some of —CH₂— in the hydrocarbyl group may be substituted with a group containing a heteroatom such as an oxygen atom, a sulfur atom, or a nitrogen atom, so that the group may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, or the like.

R¹¹ and R¹² may bond together to form a ring with the sulfur atom to which they are attached. Specific examples of the sulfonium cation having formula (cation-1) are those having the following formula.

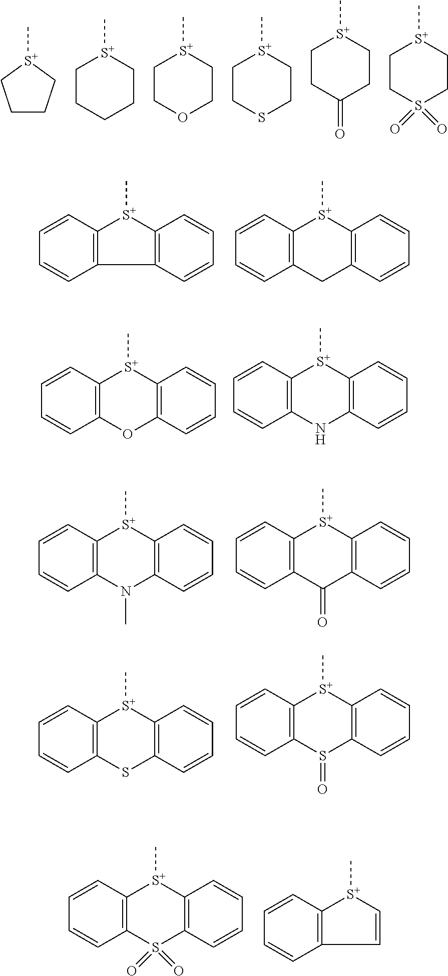

wherein the broken line denotes a point of attachment to R¹³.

Specific examples of the cation of the sulfonium salt having formula (cation-1) are shown below, but not limited thereto.

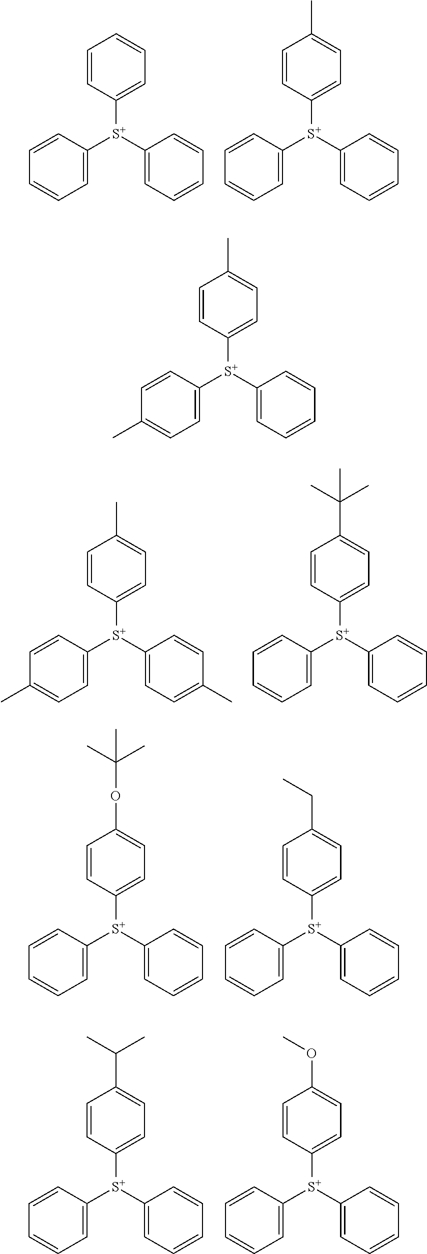

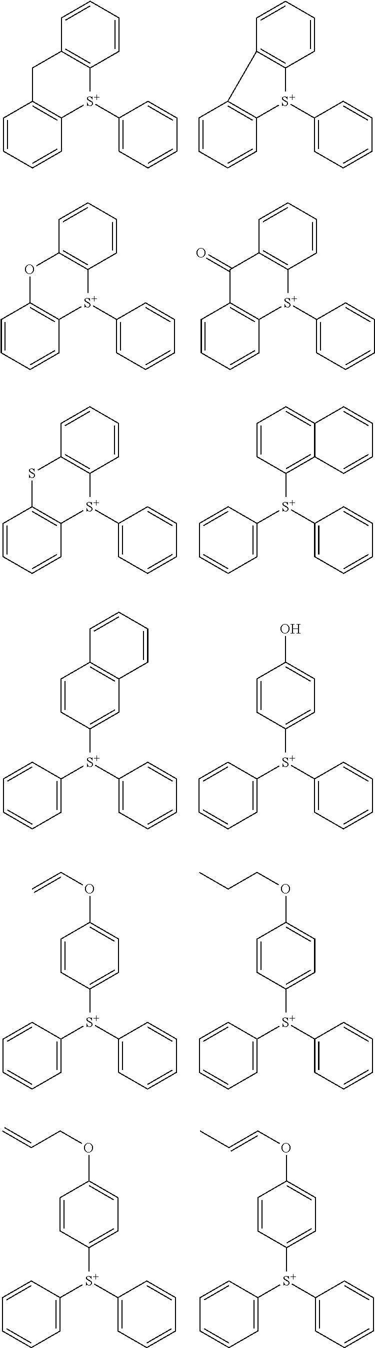

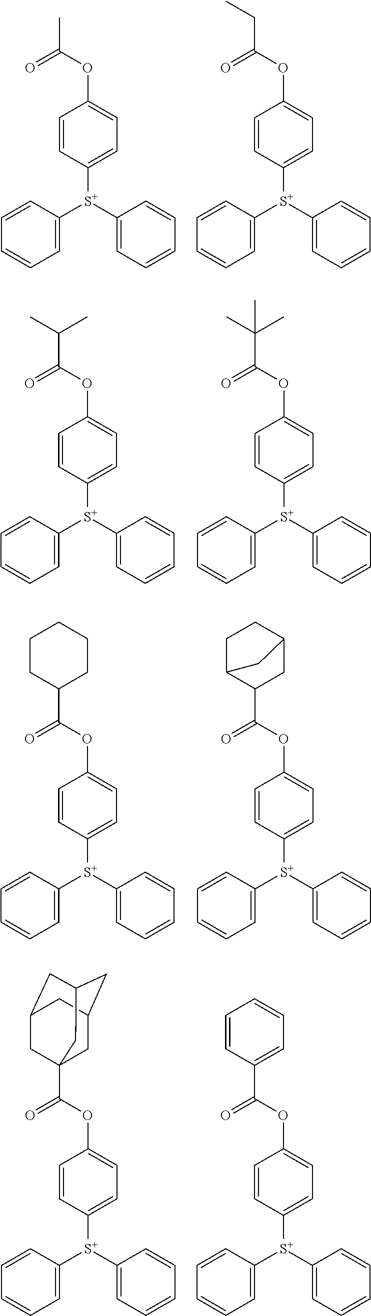

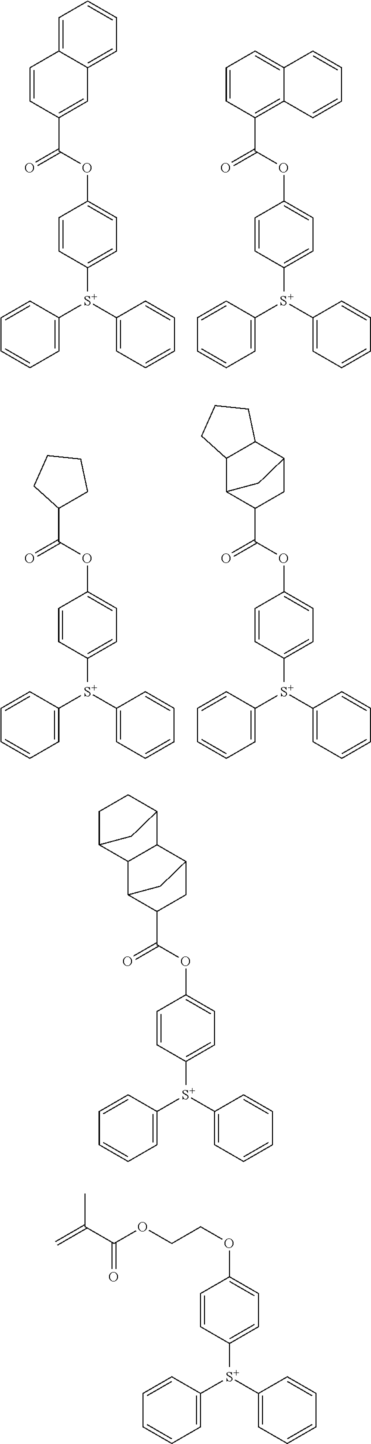 [SMILES: O=C(Oc1ccc([S+](c2ccccc2)c2ccccc2)cc1)c1ccc2ccccc2c1 ; O=C(Oc1ccc([S+](c2ccccc2)c2ccccc2)cc1)c1cccc2ccccc12 ; O=C(Oc1ccc([S+](c2ccccc2)c2ccccc2)cc1)C1CCCC1 ; O=C(Oc1ccc([S+](c2ccccc2)c2ccccc2)cc1)C1CC2CC1C1CCCC21 ; O=C(Oc1ccc([S+](c2ccccc2)c2ccccc2)cc1)C1CC2CC1C1C3CCC(C3)C21 ; C=C(C)C(=O)OCCOc1ccc([S+](c2ccccc2)c2ccccc2)cc1]

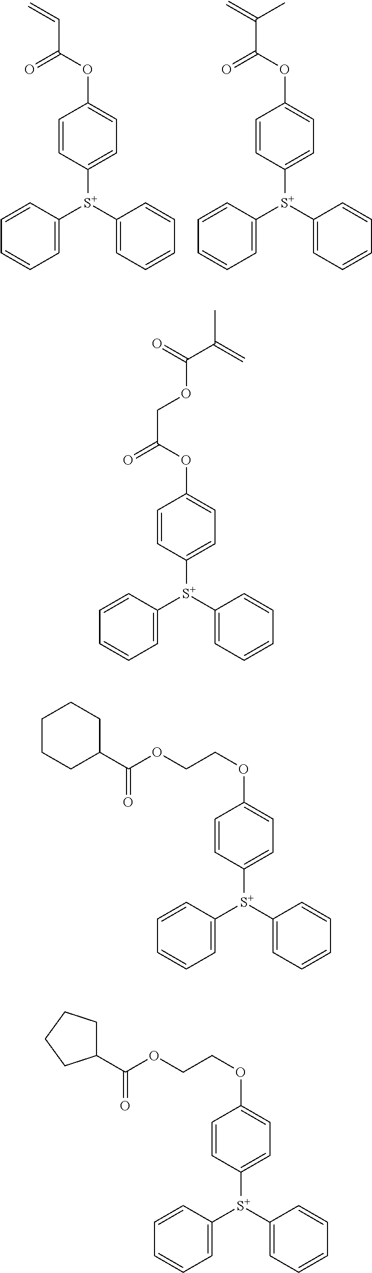 [SMILES: C=CC(=O)Oc1ccc([S+](c2ccccc2)c2ccccc2)cc1 ; C=C(C)C(=O)Oc1ccc([S+](c2ccccc2)c2ccccc2)cc1 ; C=C(C)C(=O)OCC(=O)Oc1ccc([S+](c2ccccc2)c2ccccc2)cc1 ; O=C(OCCOc1ccc([S+](c2ccccc2)c2ccccc2)cc1)C1CCCCC1 ; O=C(OCCOc1ccc([S+](c2ccccc2)c2ccccc2)cc1)C1CCCC1]

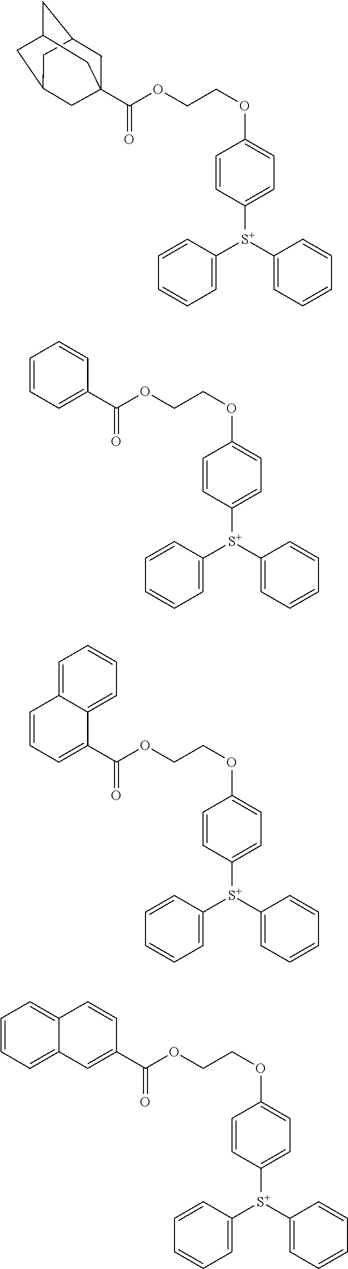 [SMILES: O=C(OCCOc1ccc([S+](c2ccccc2)c2ccccc2)cc1)C12CC3CC(CC(C3)C1)C2 ; O=C(OCCOc1ccc([S+](c2ccccc2)c2ccccc2)cc1)c1ccccc1 ; O=C(OCCOc1ccc([S+](c2ccccc2)c2ccccc2)cc1)c1cccc2ccccc12 ; O=C(OCCOc1ccc([S+](c2ccccc2)c2ccccc2)cc1)c1ccc2ccccc2c1]

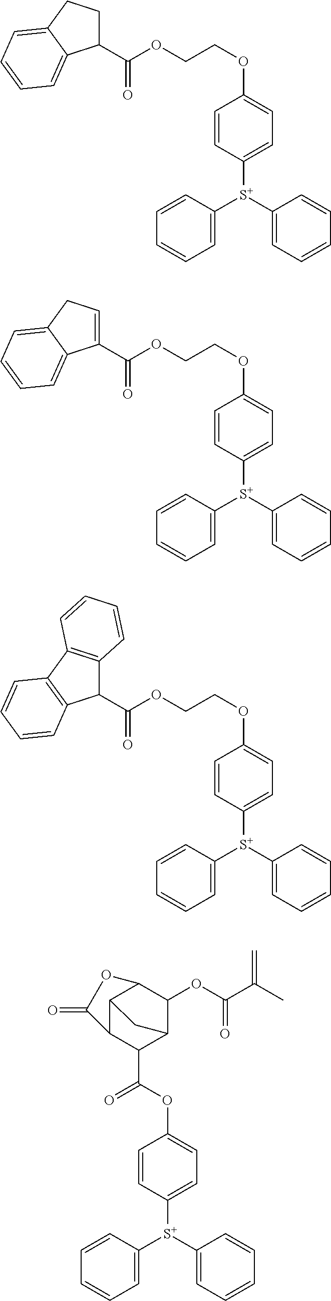 [SMILES: O=C(OCCOc1ccc([S+](c2ccccc2)c2ccccc2)cc1)C1CCc2ccccc21 ; O=C(OCCOc1ccc([S+](c2ccccc2)c2ccccc2)cc1)C1=CCc2ccccc21 ; O=C(OCCOc1ccc([S+](c2ccccc2)c2ccccc2)cc1)C1c2ccccc2-c2ccccc21 ; C=C(C)C(=O)OC1C2CC3C1OC(=O)C3C2C(=O)Oc1ccc([S+](c2ccccc2)c2ccccc2)cc1]

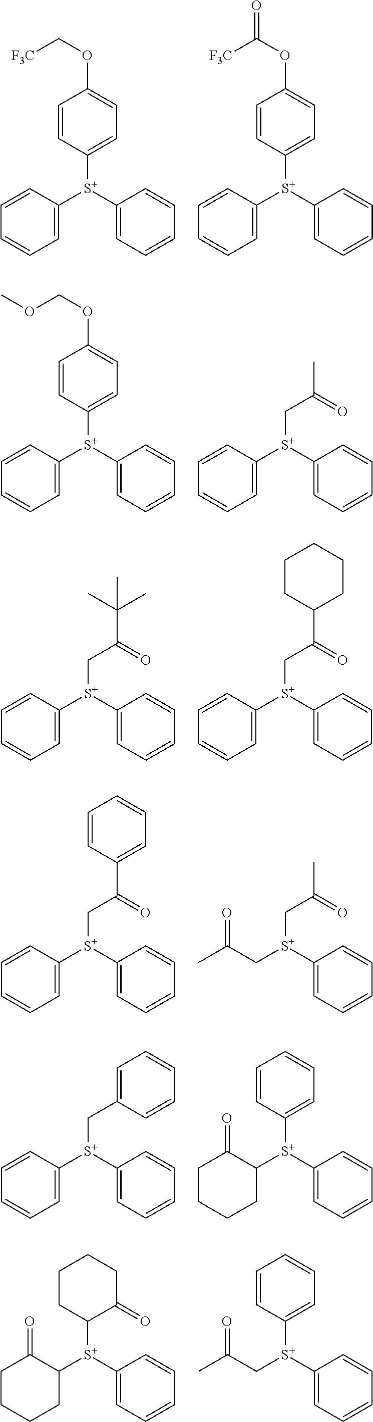

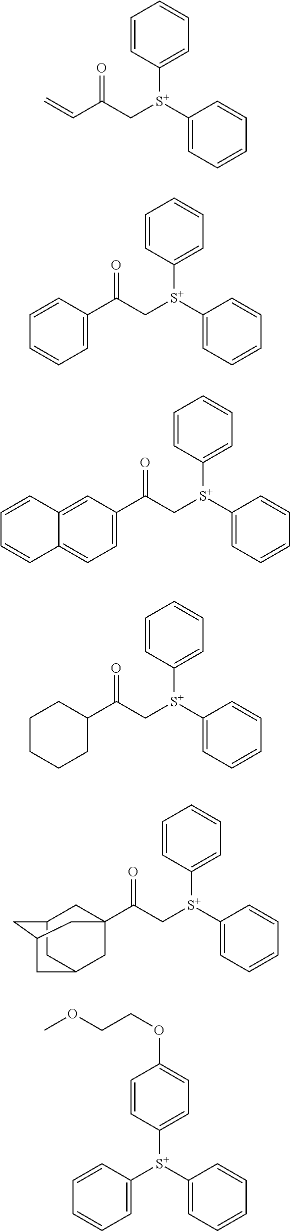 [SMILES: C=CC(=O)C[S+](c1ccccc1)c1ccccc1 ; O=C(C[S+](c1ccccc1)c1ccccc1)c1ccccc1 ; O=C(C[S+](c1ccccc1)c1ccccc1)c1ccc2ccccc2c1 ; O=C(C[S+](c1ccccc1)c1ccccc1)C1CCCCC1 ; O=C(C[S+](c1ccccc1)c1ccccc1)C12CC3CC(CC(C3)C1)C2 ; COCCOc1ccc([S+](c2ccccc2)c2ccccc2)cc1]

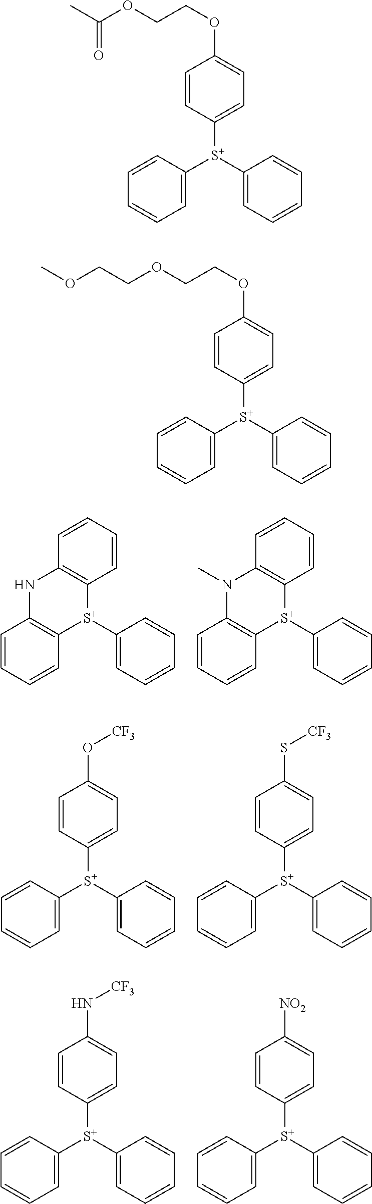

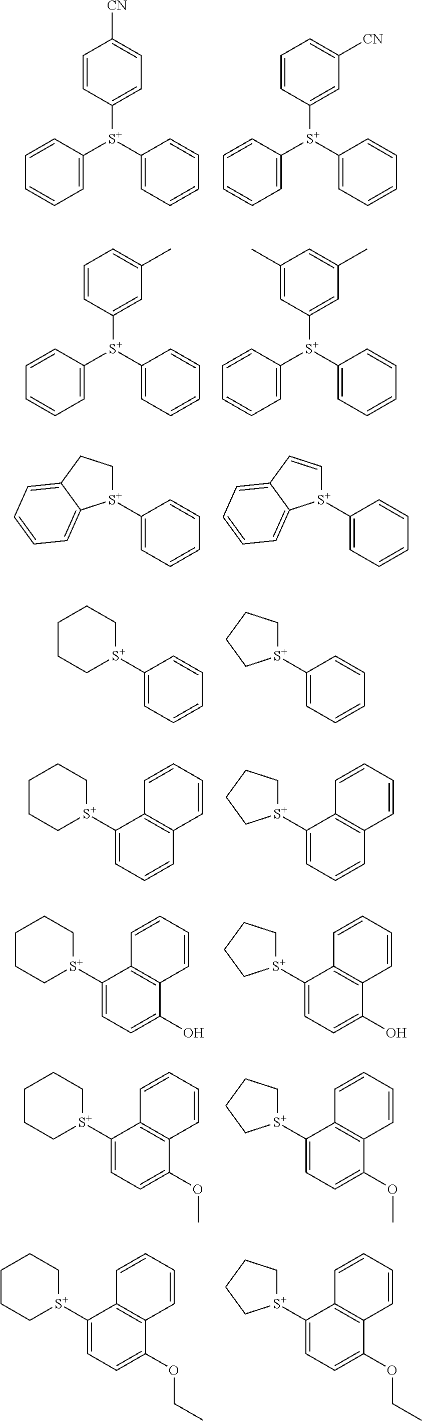

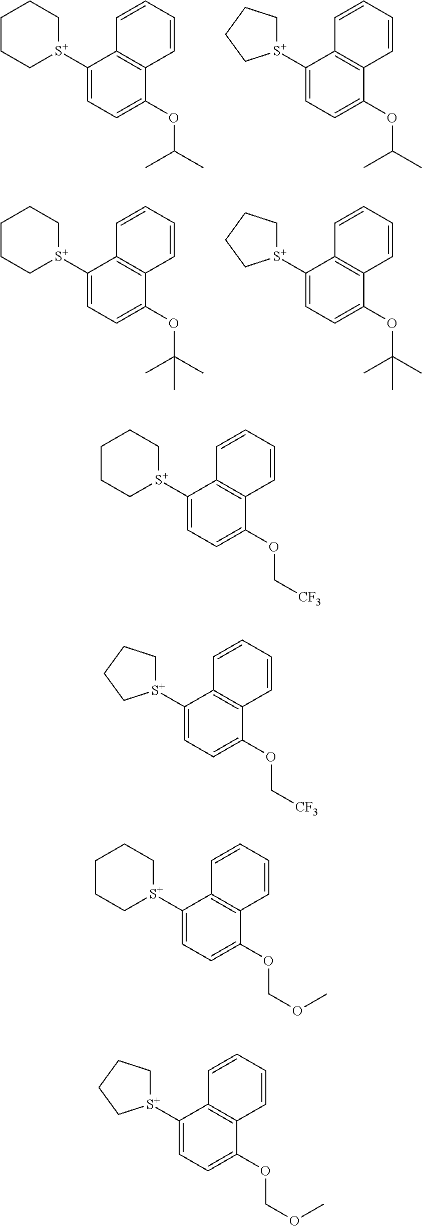

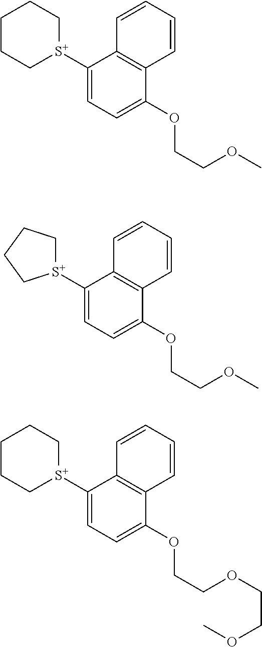 [SMILES: COCCOc1ccc([S+]2CCCCC2)c2ccccc12 ; COCCOc1ccc([S+]2CCCC2)c2ccccc12 ; COCCOCCOc1ccc([S+]2CCCCC2)c2ccccc12]

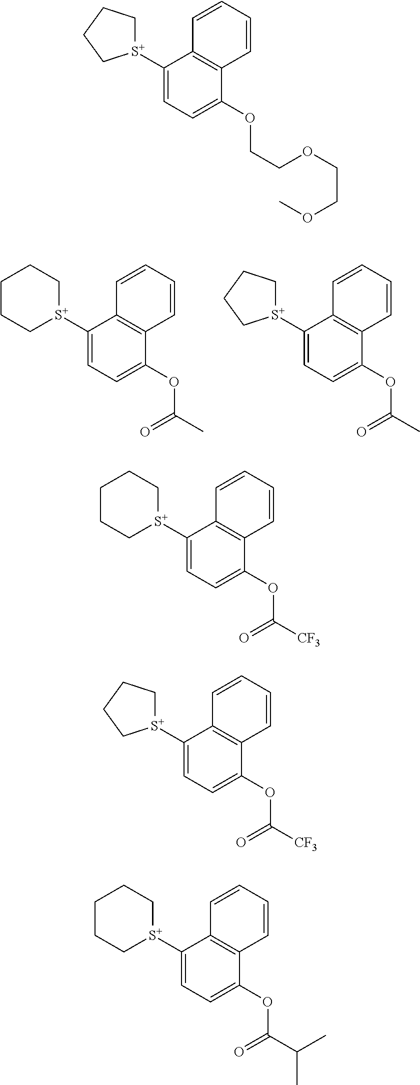 [SMILES: COCCOCCOc1ccc([S+]2CCCC2)c2ccccc12 ; CC(=O)Oc1ccc([S+]2CCCCC2)c2ccccc12 ; CC(=O)Oc1ccc([S+]2CCCC2)c2ccccc12 ; O=C(Oc1ccc([S+]2CCCCC2)c2ccccc12)C(F)(F)F ; O=C(Oc1ccc([S+]2CCCC2)c2ccccc12)C(F)(F)F ; CC(C)C(=O)Oc1ccc([S+]2CCCCC2)c2ccccc12]

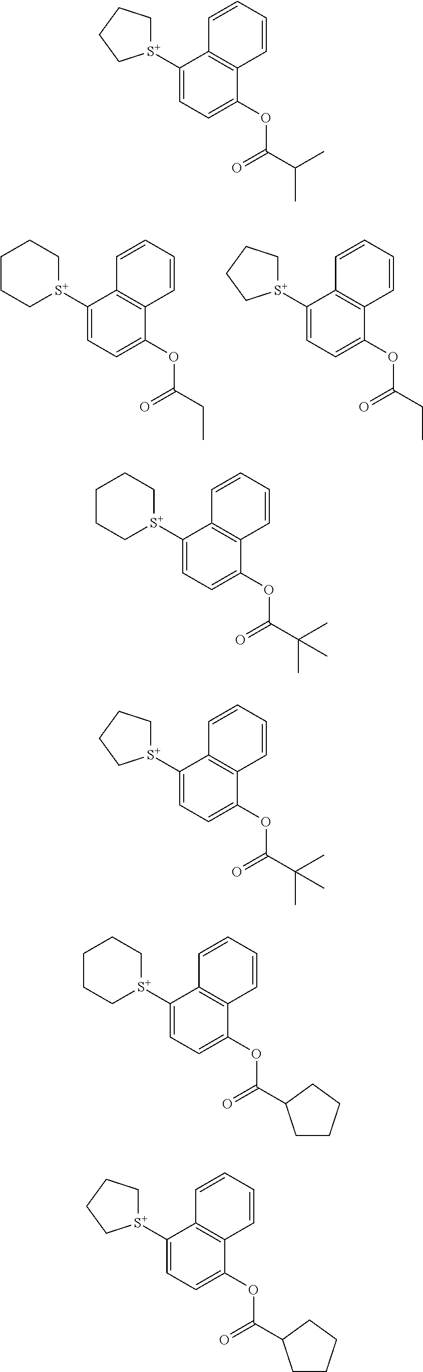

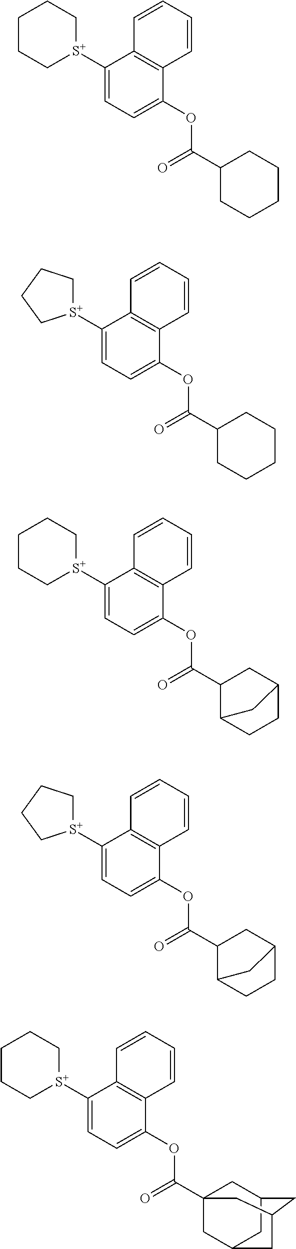 [SMILES: O=C(Oc1ccc([S+]2CCCCC2)c2ccccc12)C1CCCCC1 ; O=C(Oc1ccc([S+]2CCCC2)c2ccccc12)C1CCCCC1 ; O=C(Oc1ccc([S+]2CCCCC2)c2ccccc12)C1CC2CCC1C2 ; O=C(Oc1ccc([S+]2CCCC2)c2ccccc12)C1CC2CCC1C2 ; O=C(Oc1ccc([S+]2CCCCC2)c2ccccc12)C12CC3CC(CC(C3)C1)C2]

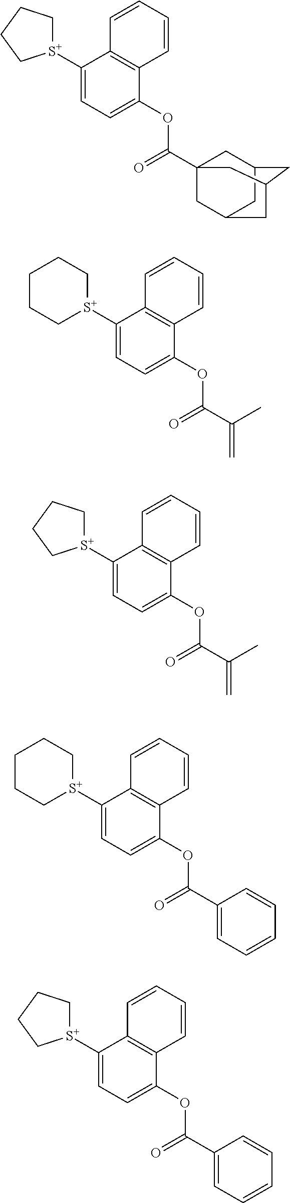 [SMILES: O=C(Oc1ccc([S+]2CCCC2)c2ccccc12)C12CC3CC(CC(C3)C1)C2 ; C=C(C)C(=O)Oc1ccc([S+]2CCCCC2)c2ccccc12 ; C=C(C)C(=O)Oc1ccc([S+]2CCCC2)c2ccccc12 ; O=C(Oc1ccc([S+]2CCCCC2)c2ccccc12)c1ccccc1 ; O=C(Oc1ccc([S+]2CCCC2)c2ccccc12)c1ccccc1]

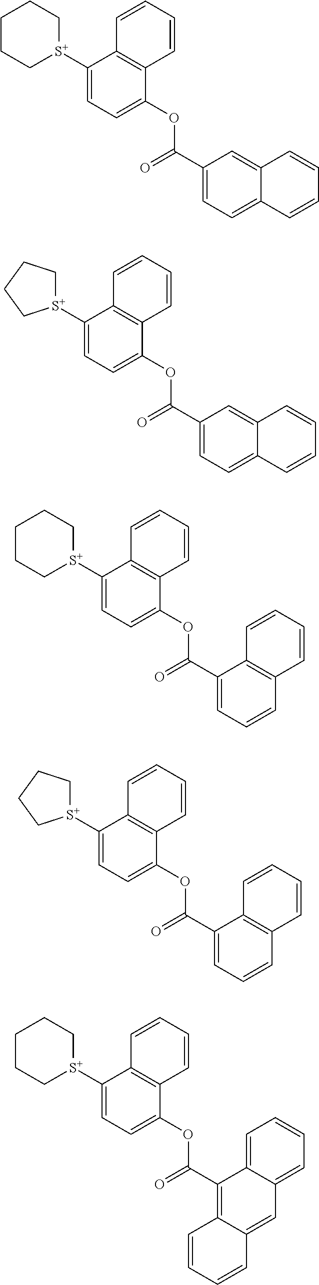 [SMILES: O=C(Oc1ccc([S+]2CCCCC2)c2ccccc12)c1ccc2ccccc2c1 ; O=C(Oc1ccc([S+]2CCCC2)c2ccccc12)c1ccc2ccccc2c1 ; O=C(Oc1ccc([S+]2CCCCC2)c2ccccc12)c1cccc2ccccc12 ; O=C(Oc1ccc([S+]2CCCC2)c2ccccc12)c1cccc2ccccc12 ; O=C(Oc1ccc([S+]2CCCCC2)c2ccccc12)c1c2ccccc2cc2ccccc12]

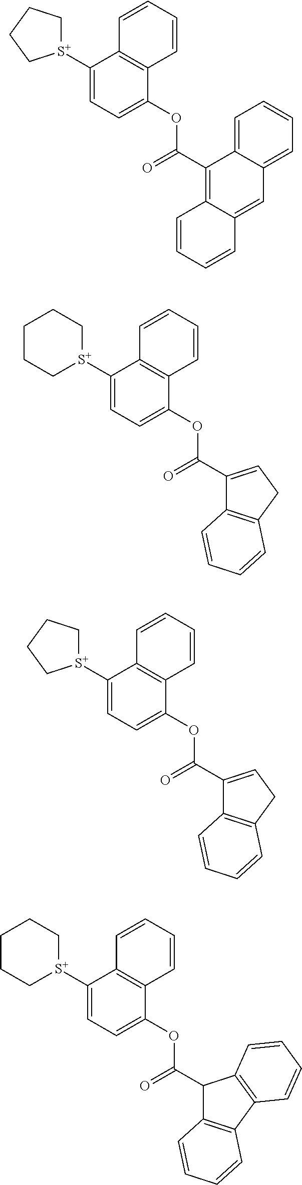 [SMILES: O=C(Oc1ccc([S+]2CCCC2)c2ccccc12)c1c2ccccc2cc2ccccc12 ; O=C(Oc1ccc([S+]2CCCCC2)c2ccccc12)C1=CCc2ccccc21 ; O=C(Oc1ccc([S+]2CCCC2)c2ccccc12)C1=CCc2ccccc21 ; O=C(Oc1ccc([S+]2CCCCC2)c2ccccc12)C1c2ccccc2-c2ccccc21]

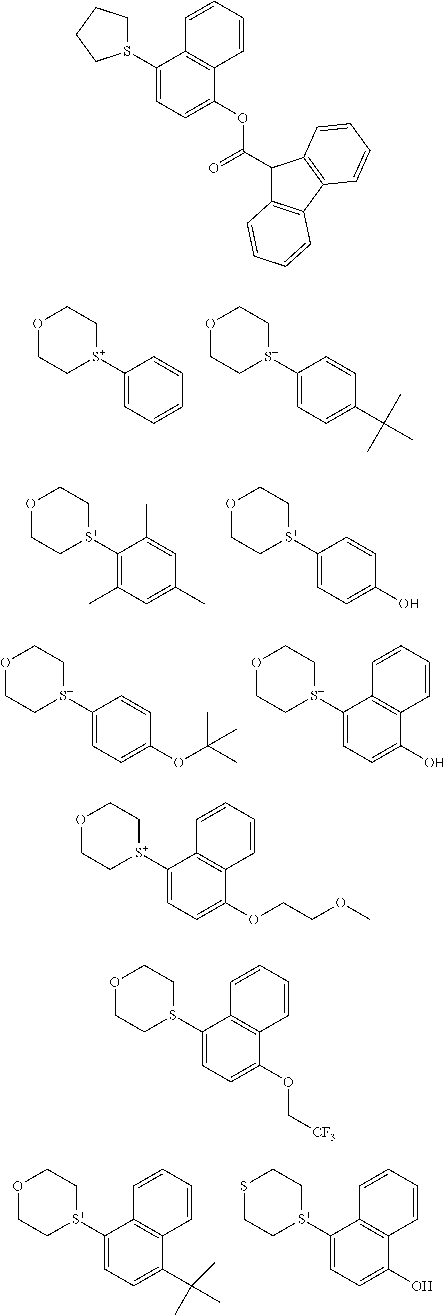

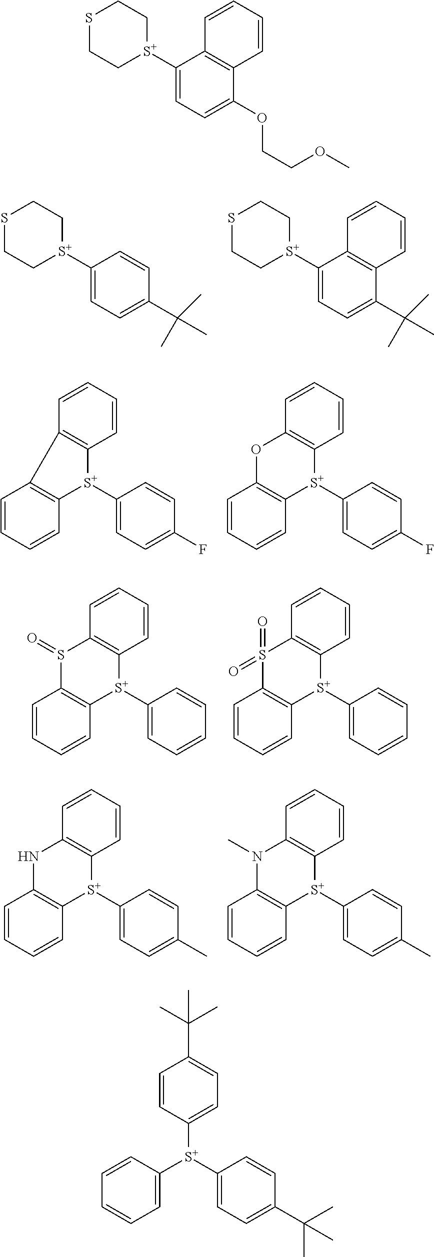

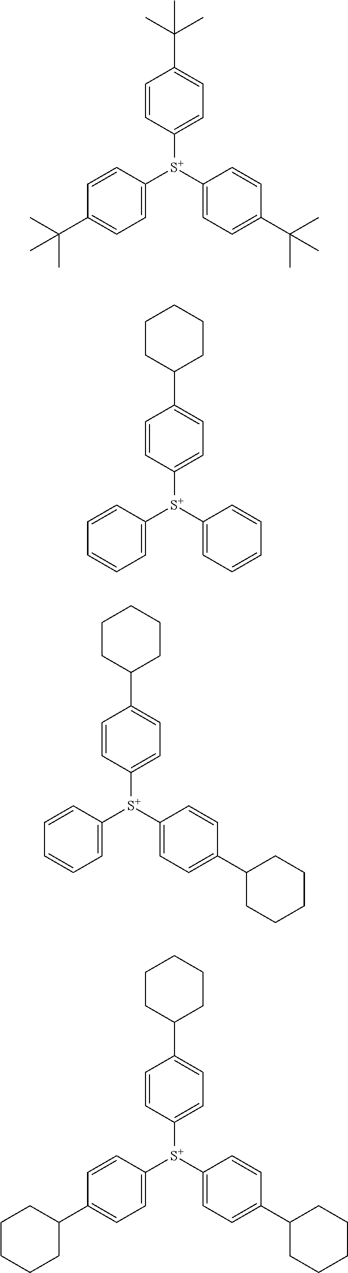 [SMILES: CC(C)(C)c1ccc([S+](c2ccc(C(C)(C)C)cc2)c2ccc(C(C)(C)C)cc2)cc1 ; c1ccc([S+](c2ccccc2)c2ccc(C3CCCCC3)cc2)cc1 ; c1ccc([S+](c2ccc(C3CCCCC3)cc2)c2ccc(C3CCCCC3)cc2)cc1 ; c1cc([S+](c2ccc(C3CCCCC3)cc2)c2ccc(C3CCCCC3)cc2)ccc1C1CCCCC1]

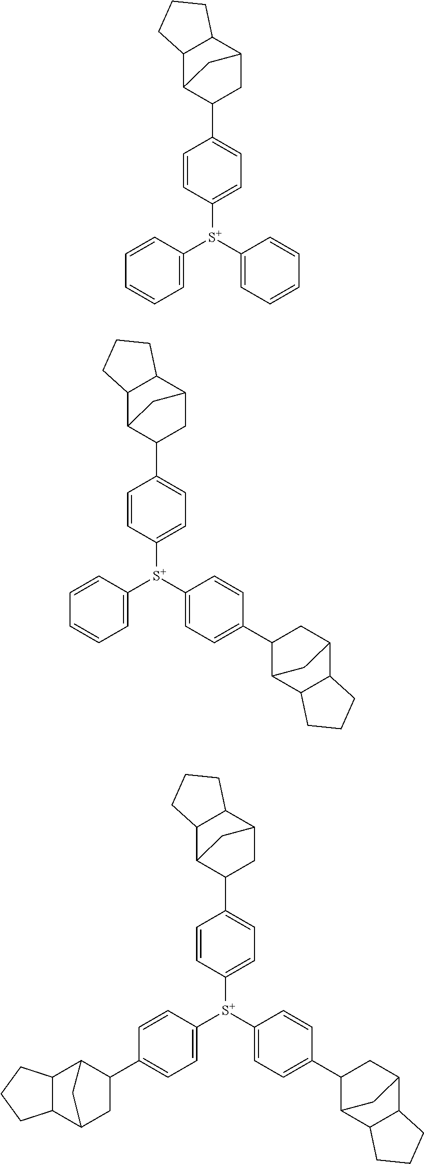 [SMILES: c1ccc([S+](c2ccccc2)c2ccc(C3CC4CC3C3CCCC43)cc2)cc1 ; c1ccc([S+](c2ccc(C3CC4CC3C3CCCC43)cc2)c2ccc(C3CC4CC3C3CCCC43)cc2)cc1 ; c1cc([S+](c2ccc(C3CC4CC3C3CCCC43)cc2)c2ccc(C3CC4CC3C3CCCC43)cc2)ccc1C1CC2CC1C1CCCC21]

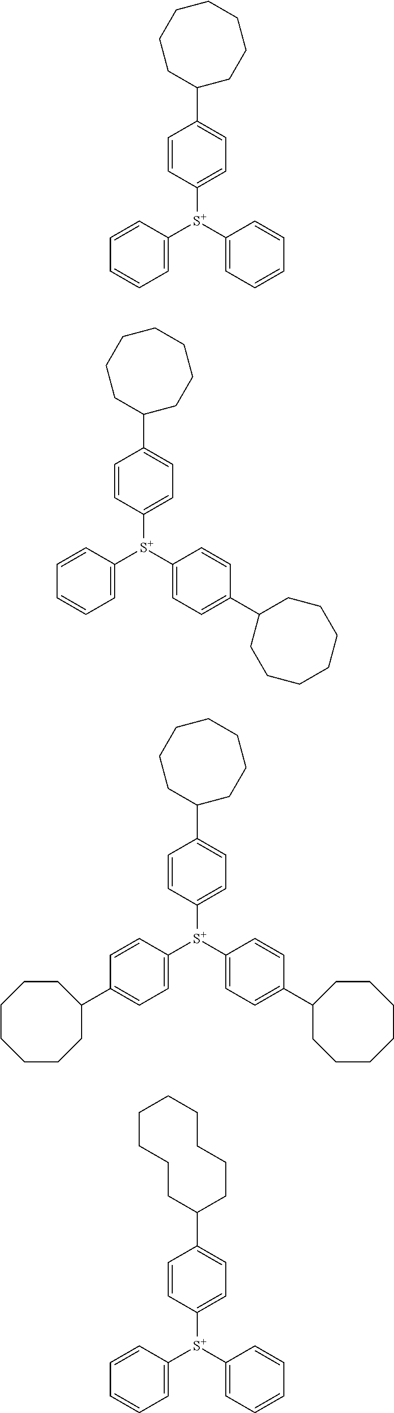 [SMILES: c1ccc([S+](c2ccccc2)c2ccc(C3CCCCCCC3)cc2)cc1 ; c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1 ; c1ccc([S+](c2ccccc2)c2ccc(C3CCCCCCCCC3)cc2)cc1]

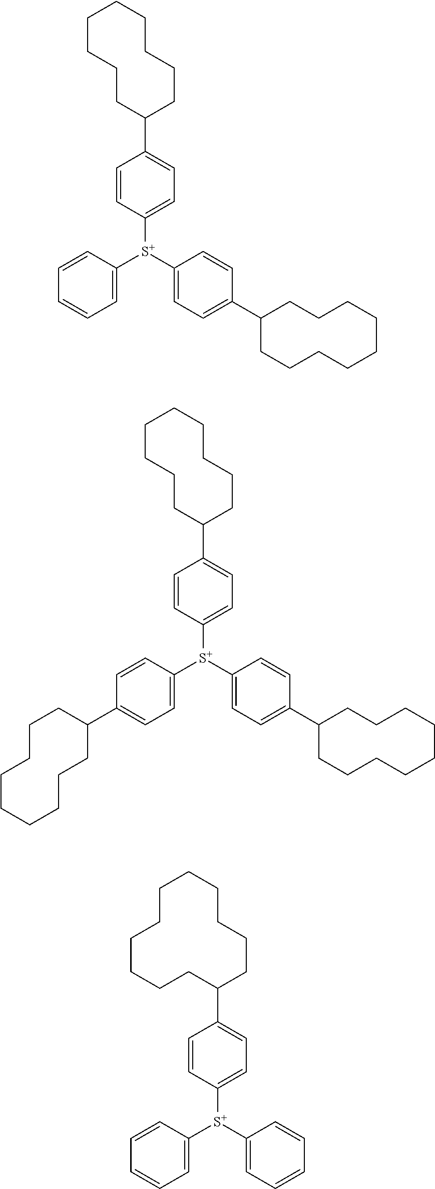 [SMILES: c1ccc([S+](c2ccc(C3CCCCCCCCC3)cc2)c2ccc(C3CCCCCCCCC3)cc2)cc1 ; c1cc([S+](c2ccc(C3CCCCCCCCC3)cc2)c2ccc(C3CCCCCCCCC3)cc2)ccc1C1CCCCCCCCC1 ; c1ccc([S+](c2ccccc2)c2ccc(C3CCCCCCCCCCC3)cc2)cc1]

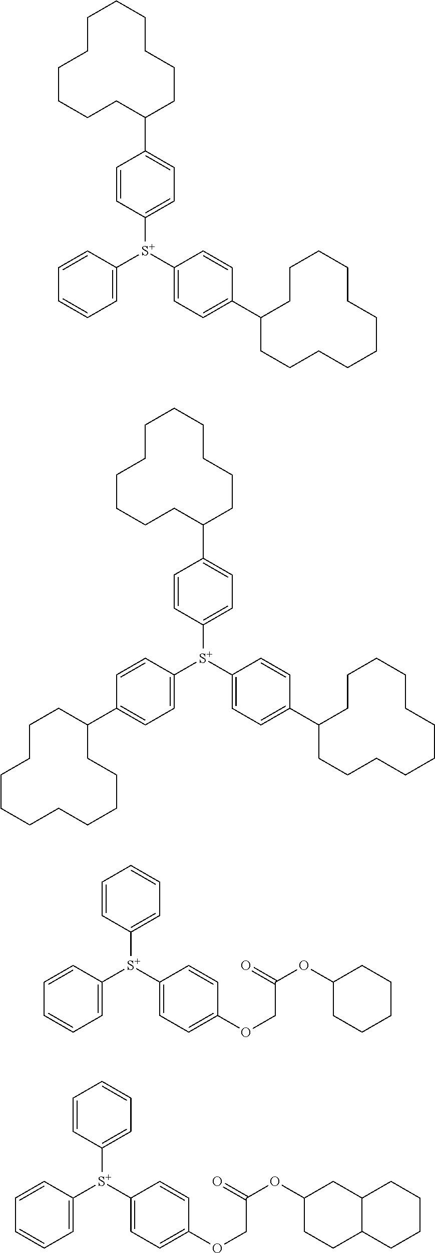 [SMILES: c1ccc([S+](c2ccc(C3CCCCCCCCCCC3)cc2)c2ccc(C3CCCCCCCCCCC3)cc2)cc1 ; c1cc([S+](c2ccc(C3CCCCCCCCCCC3)cc2)c2ccc(C3CCCCCCCCCCC3)cc2)ccc1C1CCCCCCCCCCC1 ; O=C(COc1ccc([S+](c2ccccc2)c2ccccc2)cc1)OC1CCCCC1 ; O=C(COc1ccc([S+](c2ccccc2)c2ccccc2)cc1)OC1CCC2CCCCC2C1]

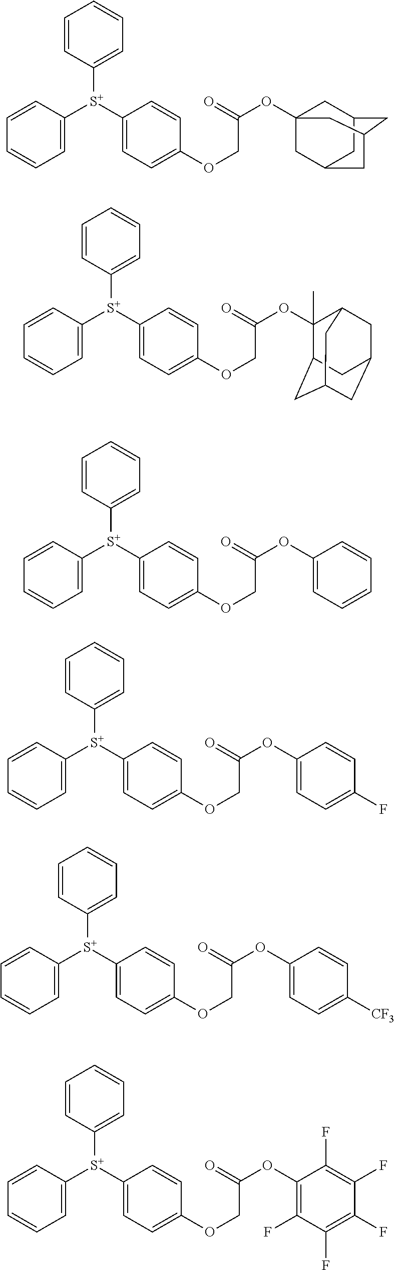 [SMILES: O=C(COc1ccc([S+](c2ccccc2)c2ccccc2)cc1)OC12CC3CC(CC(C3)C1)C2 ; CC1(OC(=O)COc2ccc([S+](c3ccccc3)c3ccccc3)cc2)C2CC3CC(C2)CC1C3 ; O=C(COc1ccc([S+](c2ccccc2)c2ccccc2)cc1)Oc1ccccc1 ; O=C(COc1ccc([S+](c2ccccc2)c2ccccc2)cc1)Oc1ccc(F)cc1 ; O=C(COc1ccc([S+](c2ccccc2)c2ccccc2)cc1)Oc1ccc(C(F)(F)F)cc1 ; O=C(COc1ccc([S+](c2ccccc2)c2ccccc2)cc1)Oc1c(F)c(F)c(F)c(F)c1F]

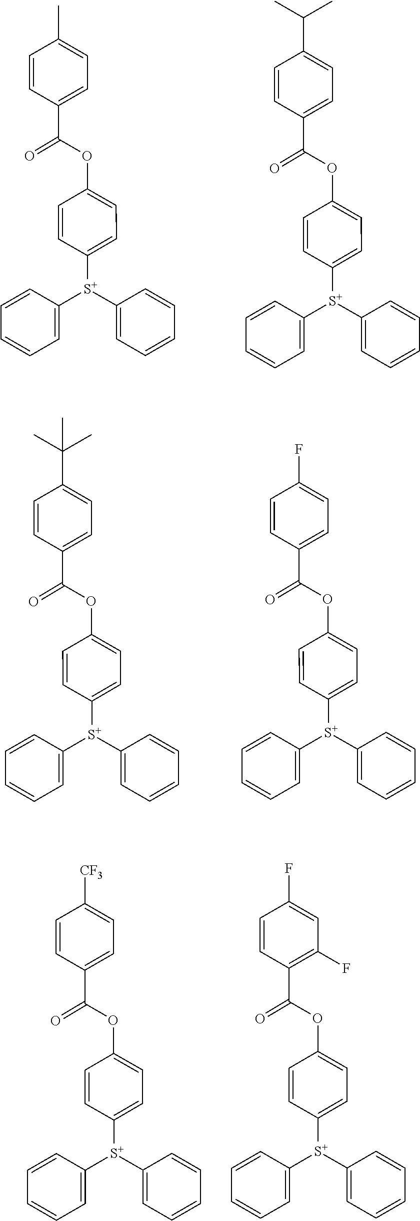 [SMILES: Cc1ccc(C(=O)Oc2ccc([S+](c3ccccc3)c3ccccc3)cc2)cc1 ; CC(C)c1ccc(C(=O)Oc2ccc([S+](c3ccccc3)c3ccccc3)cc2)cc1 ; CC(C)(C)c1ccc(C(=O)Oc2ccc([S+](c3ccccc3)c3ccccc3)cc2)cc1 ; O=C(Oc1ccc([S+](c2ccccc2)c2ccccc2)cc1)c1ccc(F)cc1 ; O=C(Oc1ccc([S+](c2ccccc2)c2ccccc2)cc1)c1ccc(C(F)(F)F)cc1 ; O=C(Oc1ccc([S+](c2ccccc2)c2ccccc2)cc1)c1ccc(F)cc1F]

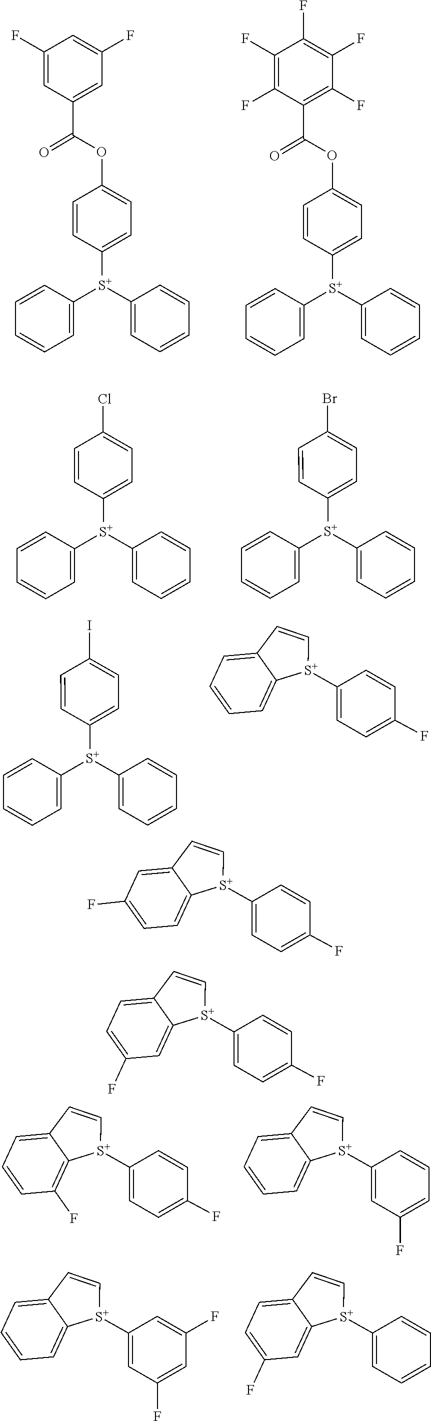

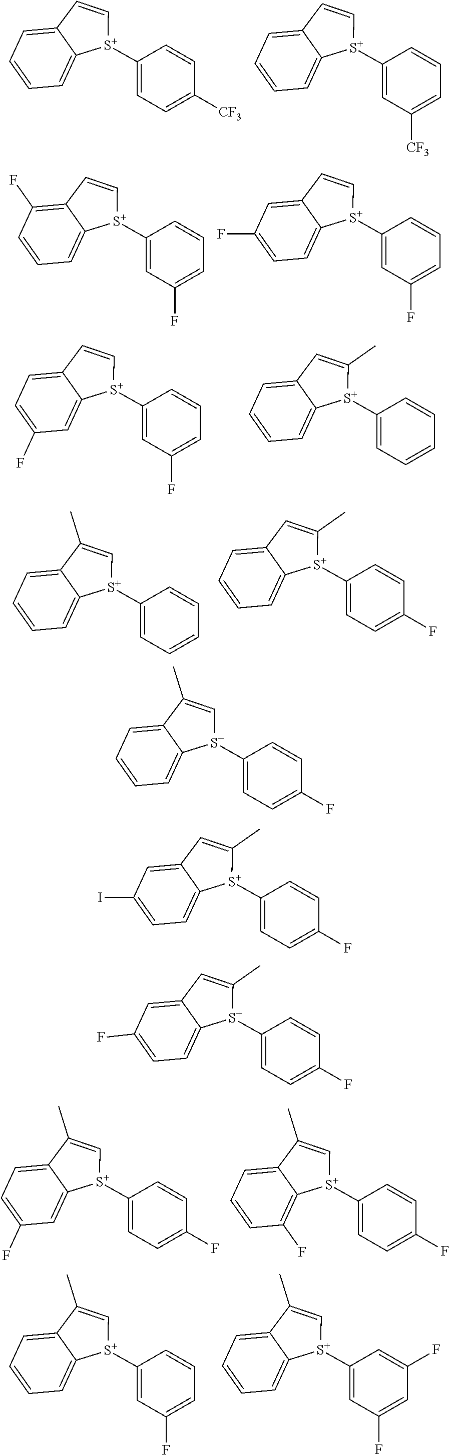

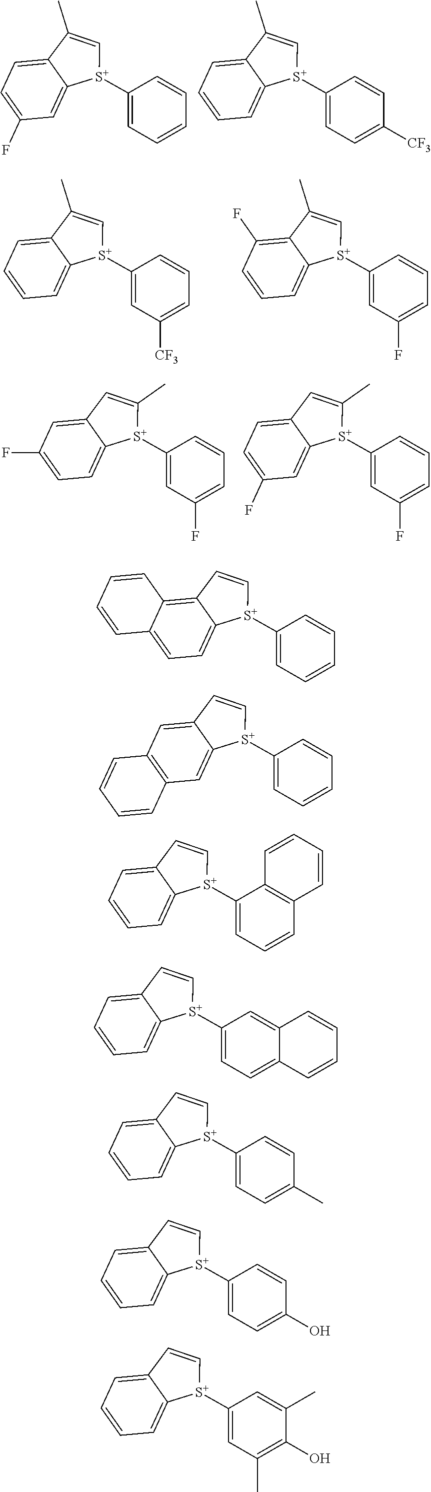

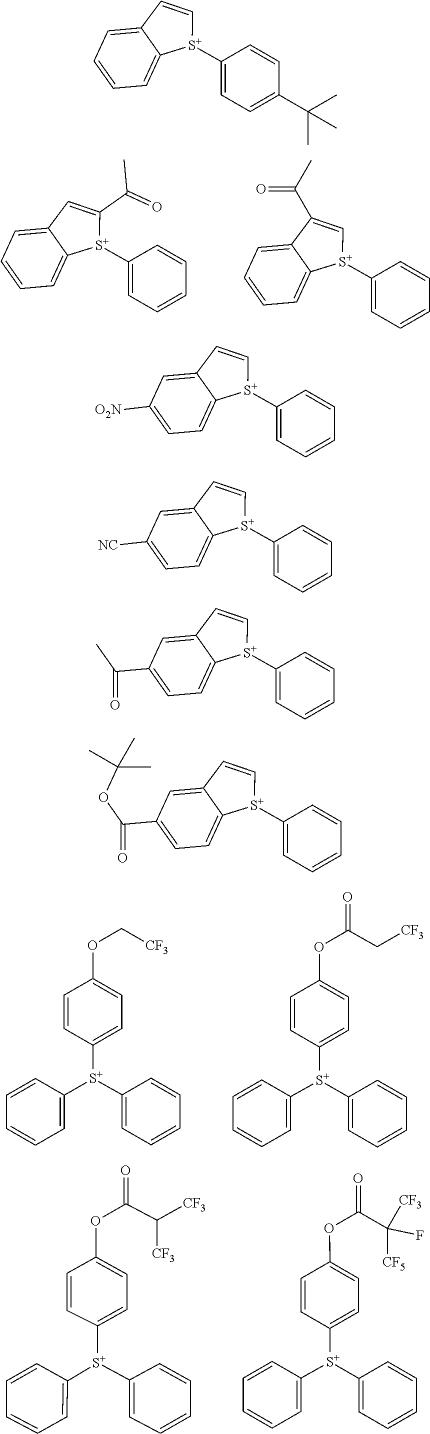

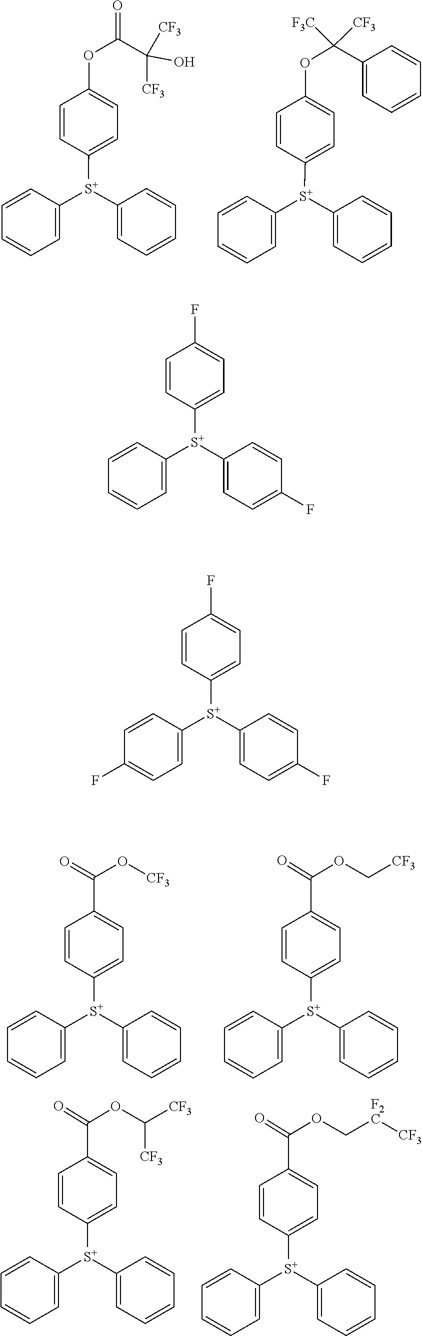

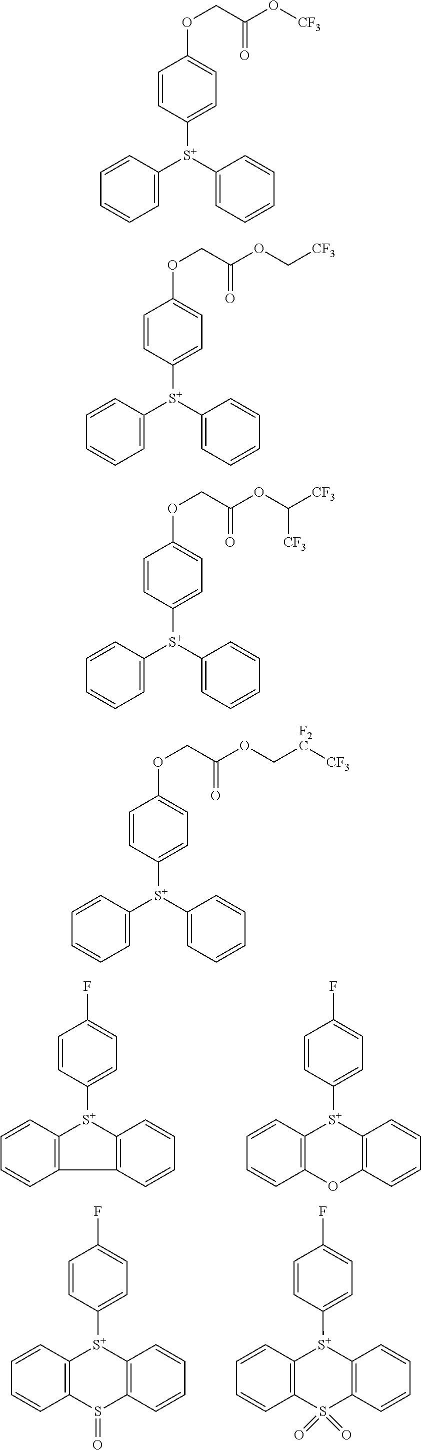

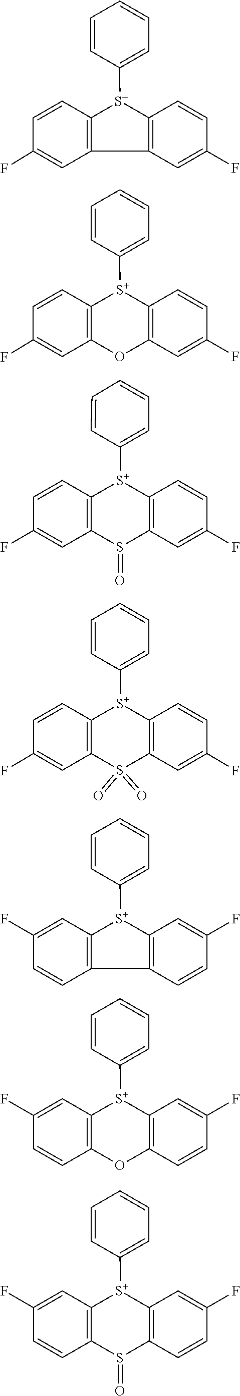

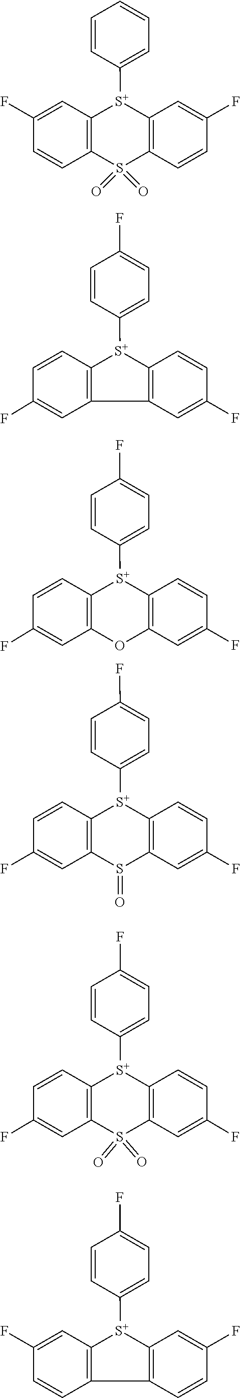 [SMILES: O=S1(=O)c2ccc(F)cc2[S+](c2ccccc2)c2cc(F)ccc21 ; Fc1ccc(-[s+]2c3ccc(F)cc3c3cc(F)ccc32)cc1 ; Fc1ccc([S+]2c3ccc(F)cc3Oc3cc(F)ccc32)cc1 ; O=S1c2cc(F)ccc2[S+](c2ccc(F)cc2)c2ccc(F)cc21 ; O=S1(=O)c2cc(F)ccc2[S+](c2ccc(F)cc2)c2ccc(F)cc21 ; Fc1ccc(-[s+]2c3cc(F)ccc3c3ccc(F)cc32)cc1]

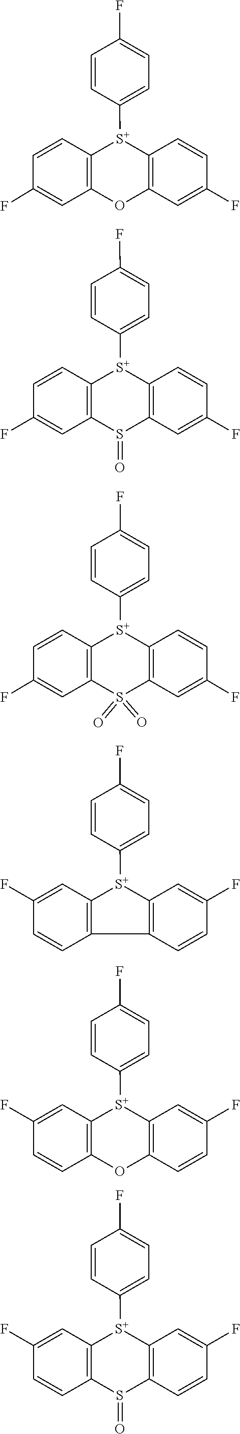 [SMILES: Fc1ccc([S+]2c3ccc(F)cc3Oc3cc(F)ccc32)cc1 ; O=S1c2cc(F)ccc2[S+](c2ccc(F)cc2)c2ccc(F)cc21 ; O=S1(=O)c2cc(F)ccc2[S+](c2ccc(F)cc2)c2ccc(F)cc21 ; Fc1ccc(-[s+]2c3cc(F)ccc3c3ccc(F)cc32)cc1 ; Fc1ccc([S+]2c3cc(F)ccc3Oc3ccc(F)cc32)cc1 ; O=S1c2ccc(F)cc2[S+](c2ccc(F)cc2)c2cc(F)ccc21]

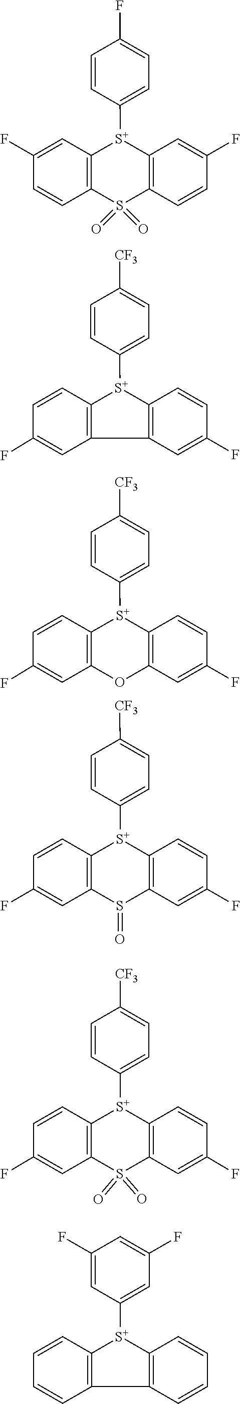 [SMILES: O=S1(=O)c2ccc(F)cc2[S+](c2ccc(F)cc2)c2cc(F)ccc21 ; Fc1ccc2c(c1)c1cc(F)ccc1[s+]2-c1ccc(C(F)(F)F)cc1 ; Fc1ccc2c(c1)Oc1cc(F)ccc1[S+]2c1ccc(C(F)(F)F)cc1 ; O=S1c2cc(F)ccc2[S+](c2ccc(C(F)(F)F)cc2)c2ccc(F)cc21 ; O=S1(=O)c2cc(F)ccc2[S+](c2ccc(C(F)(F)F)cc2)c2ccc(F)cc21 ; Fc1cc(F)cc(-[s+]2c3ccccc3c3ccccc32)c1]

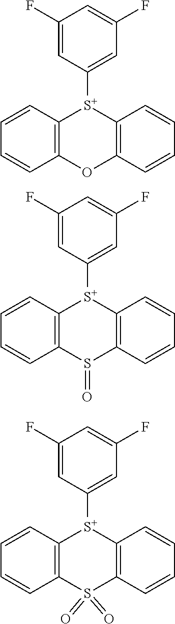 [SMILES: Fc1cc(F)cc([S+]2c3ccccc3Oc3ccccc32)c1 ; O=S1c2ccccc2[S+](c2cc(F)cc(F)c2)c2ccccc21 ; O=S1(=O)c2ccccc2[S+](c2cc(F)cc(F)c2)c2ccccc21]

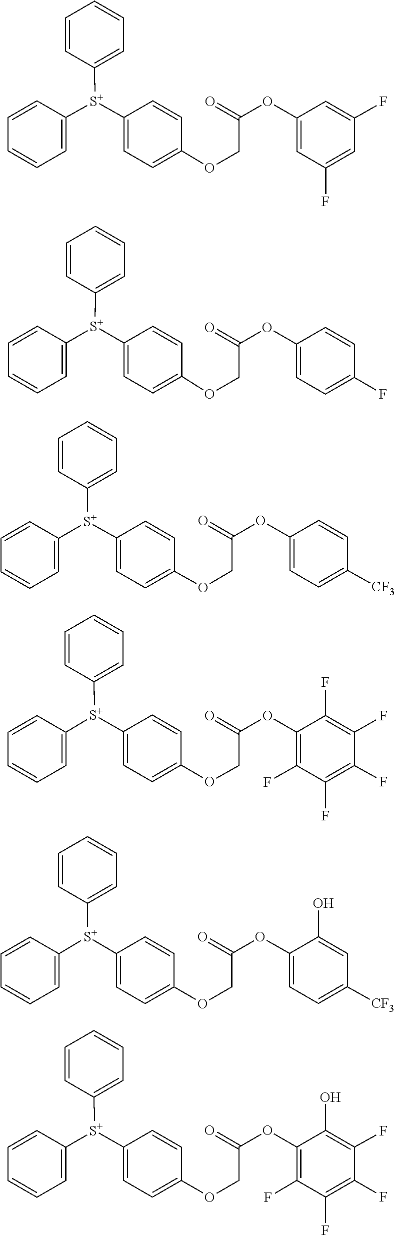 [SMILES: O=C(COc1ccc([S+](c2ccccc2)c2ccccc2)cc1)Oc1cc(F)cc(F)c1 ; O=C(COc1ccc([S+](c2ccccc2)c2ccccc2)cc1)Oc1ccc(F)cc1 ; O=C(COc1ccc([S+](c2ccccc2)c2ccccc2)cc1)Oc1ccc(C(F)(F)F)cc1 ; O=C(COc1ccc([S+](c2ccccc2)c2ccccc2)cc1)Oc1c(F)c(F)c(F)c(F)c1F ; O=C(COc1ccc([S+](c2ccccc2)c2ccccc2)cc1)Oc1ccc(C(F)(F)F)cc1O ; O=C(COc1ccc([S+](c2ccccc2)c2ccccc2)cc1)Oc1c(O)c(F)c(F)c(F)c1F]

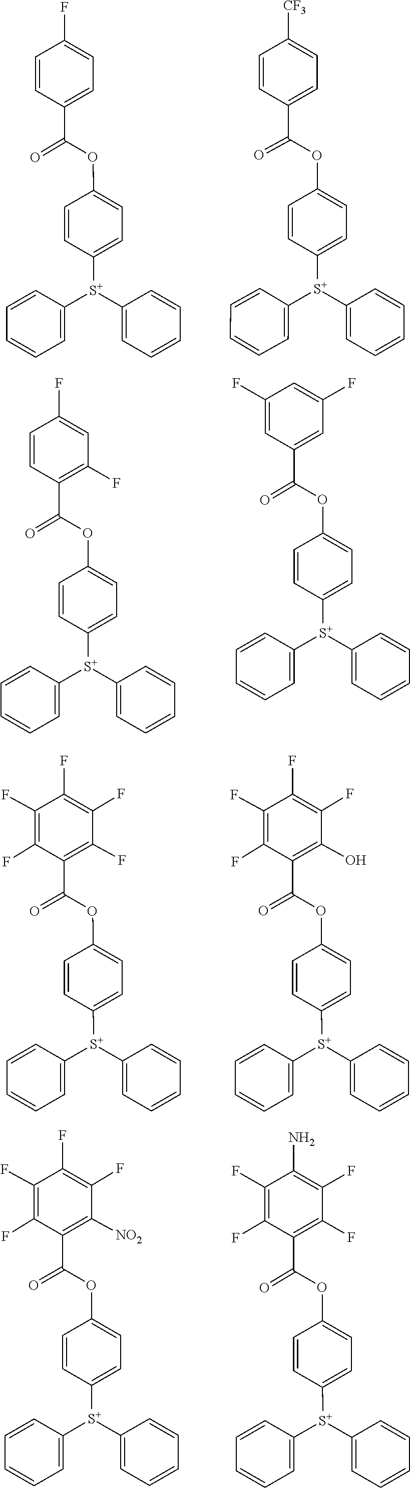

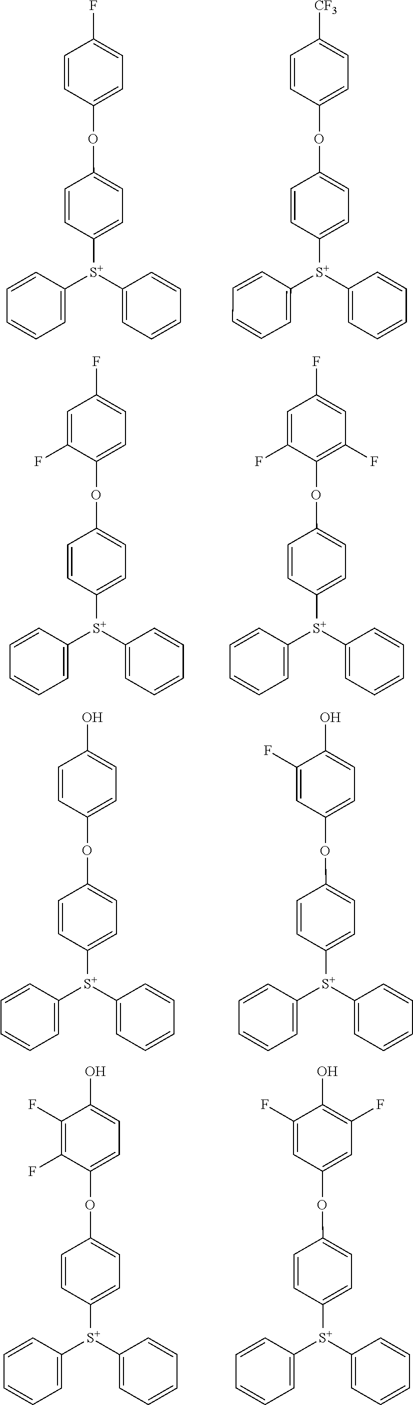

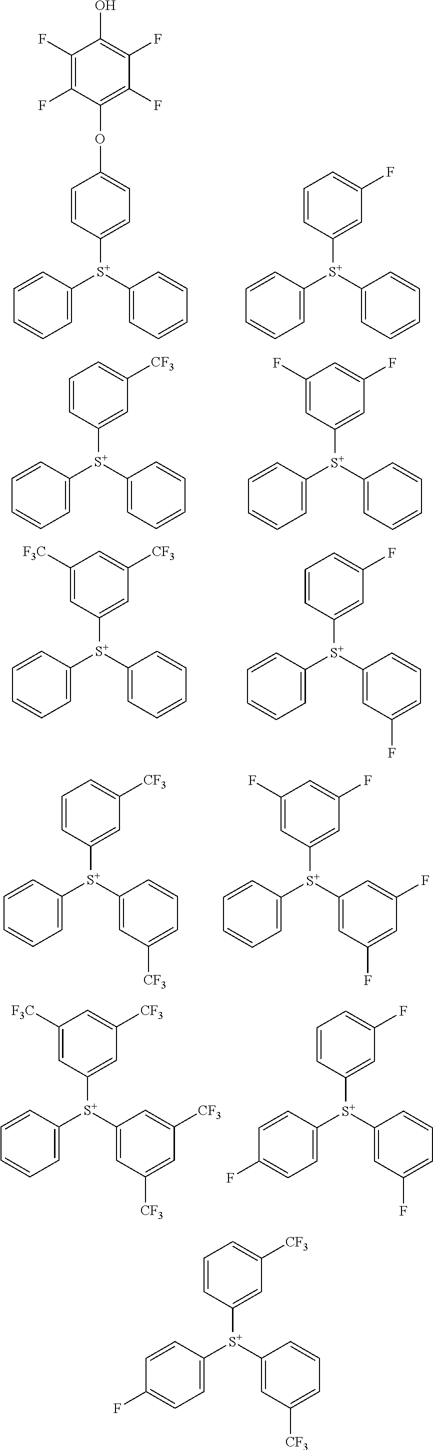

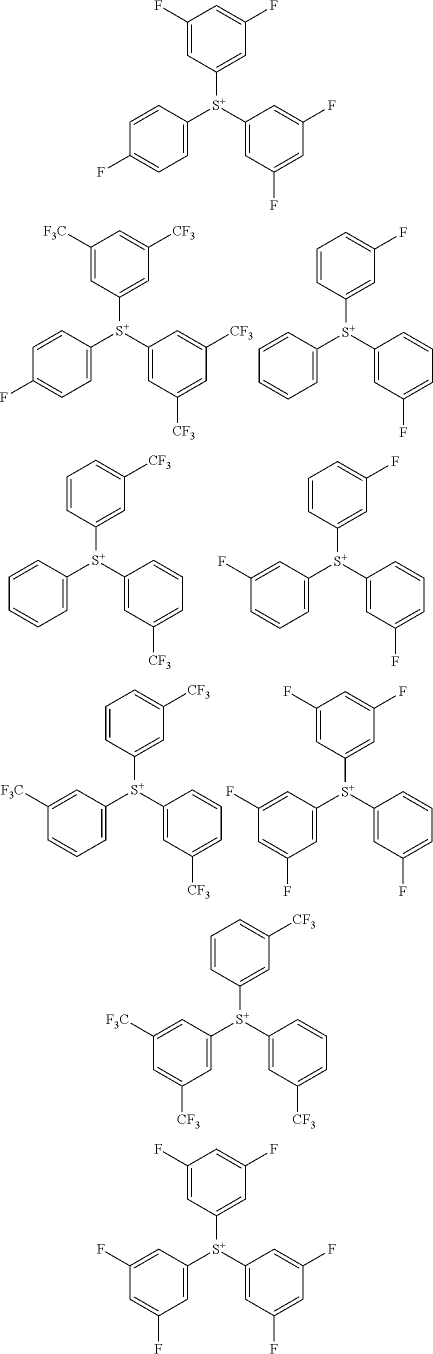

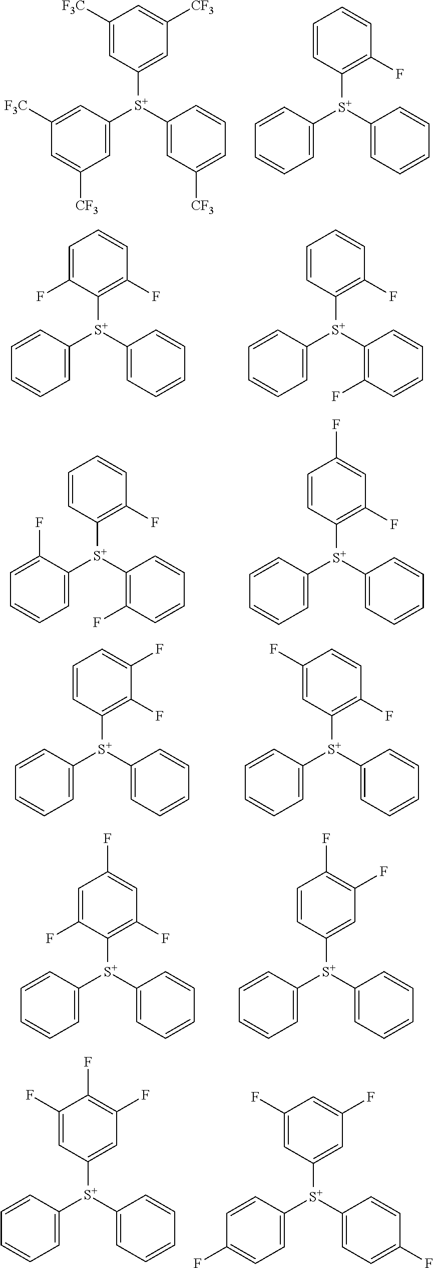

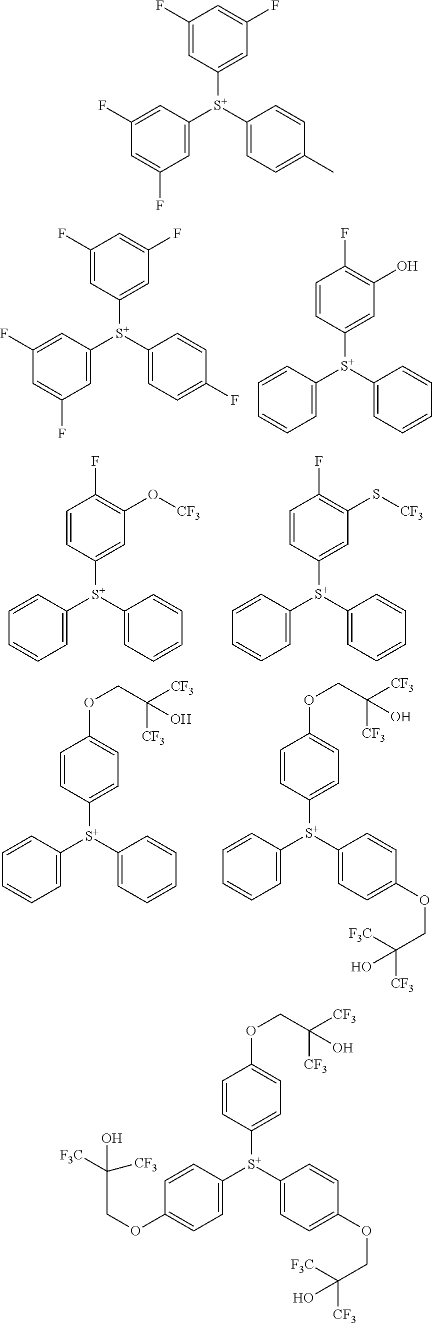

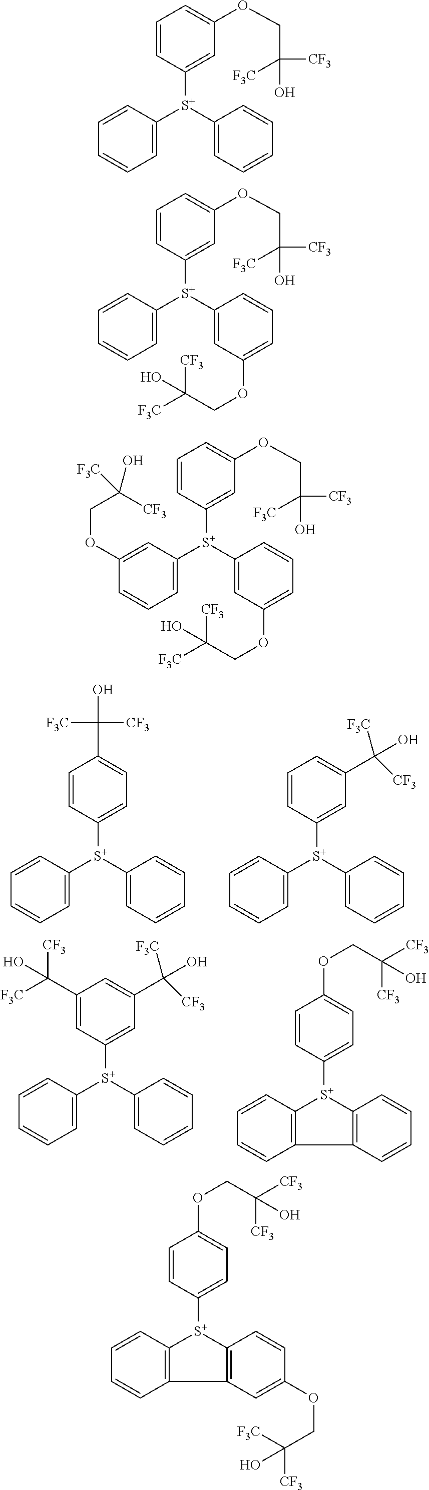

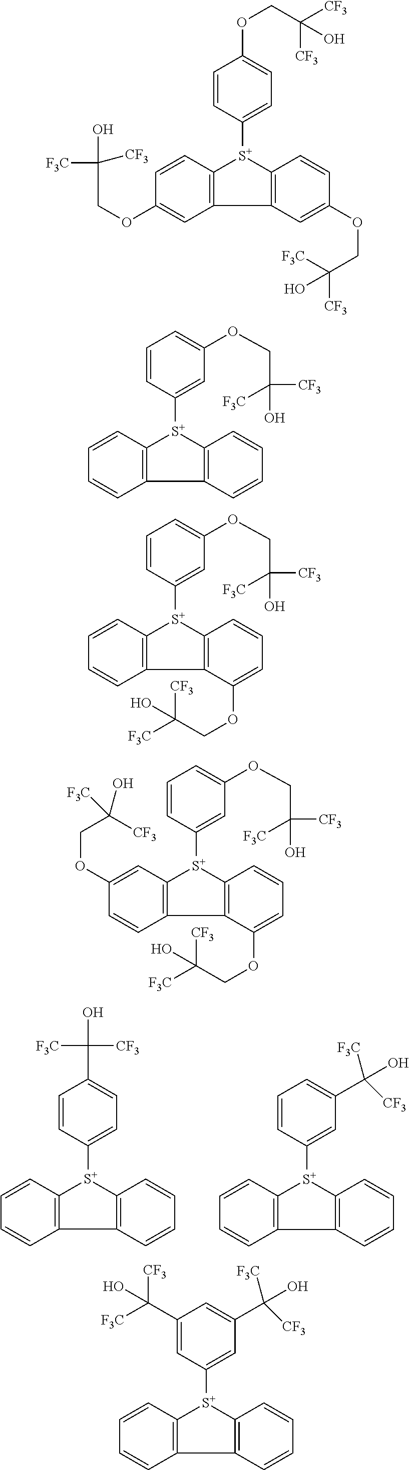

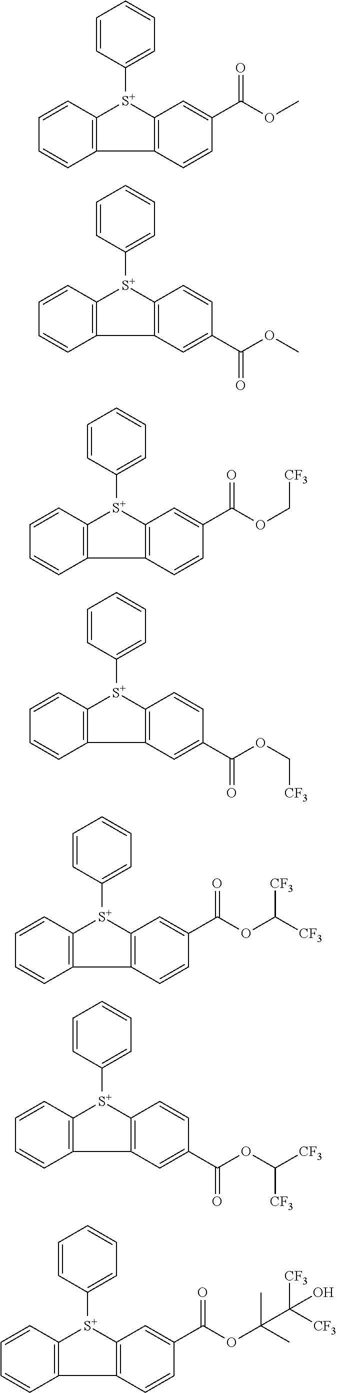

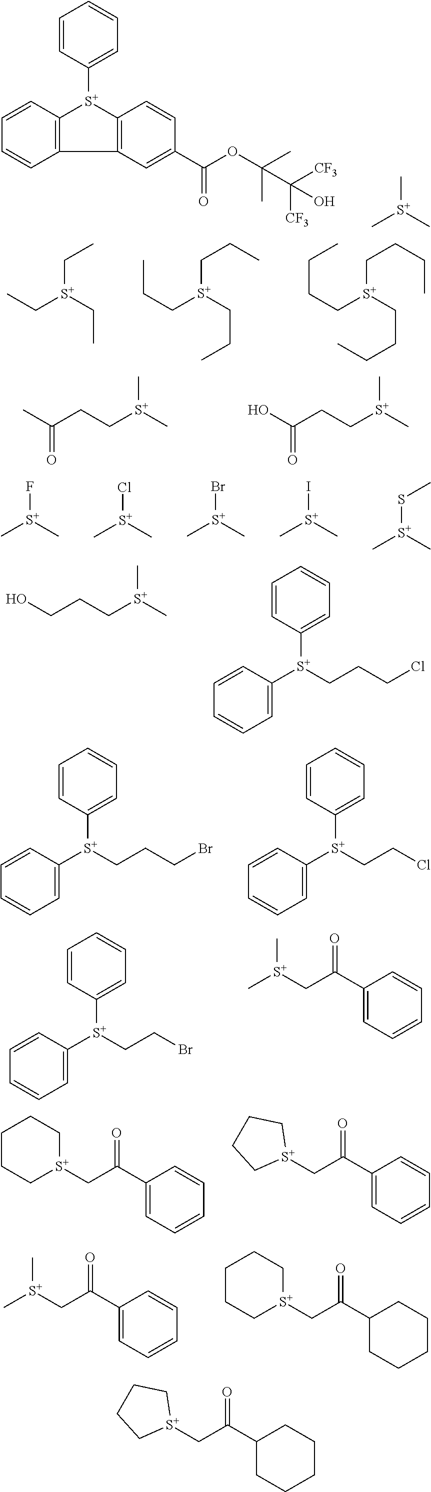

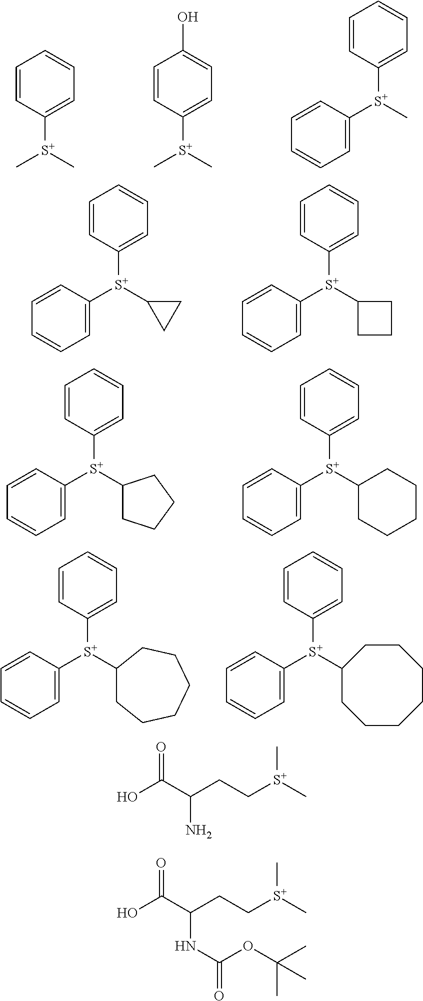

Specific examples of the iodonium cation having formula (cation-2) are shown below, but not limited thereto.

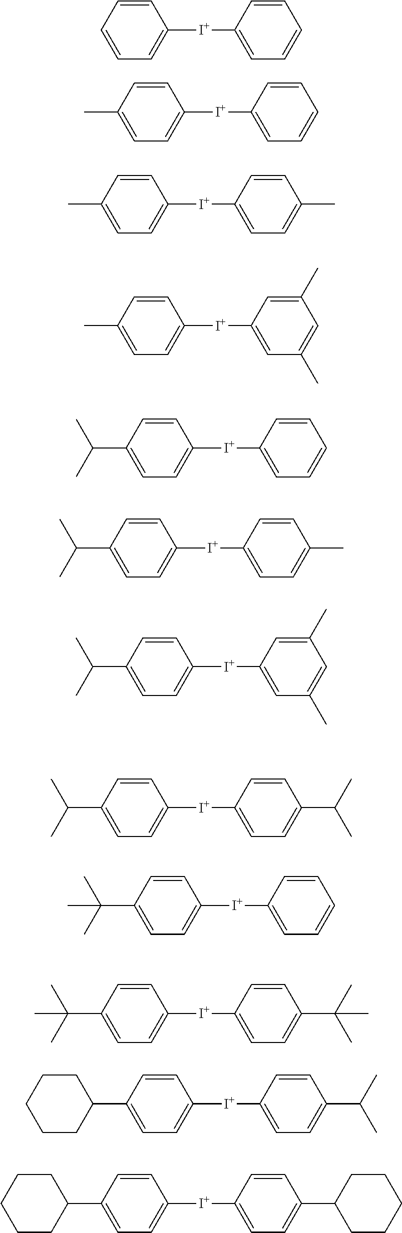

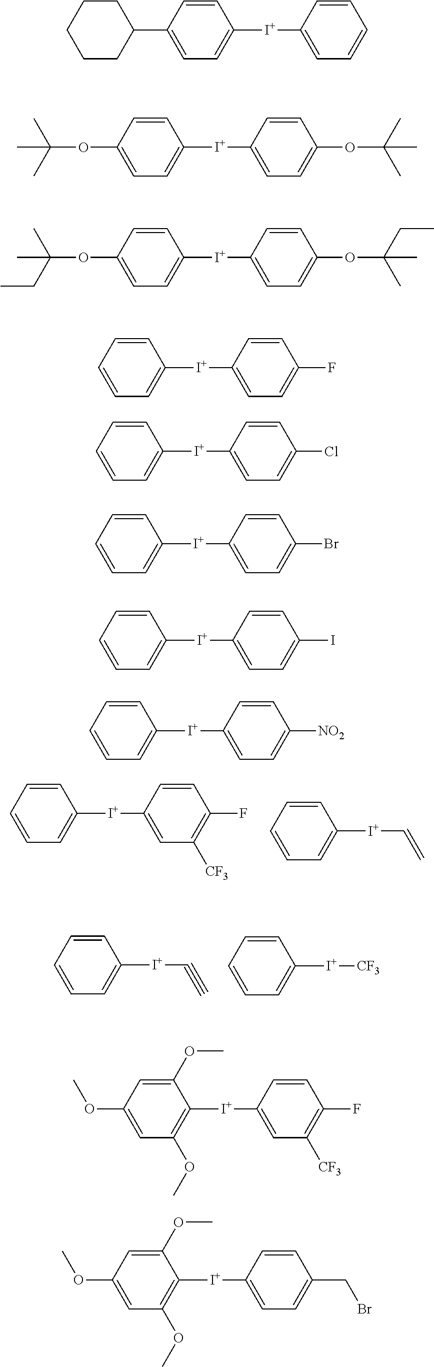

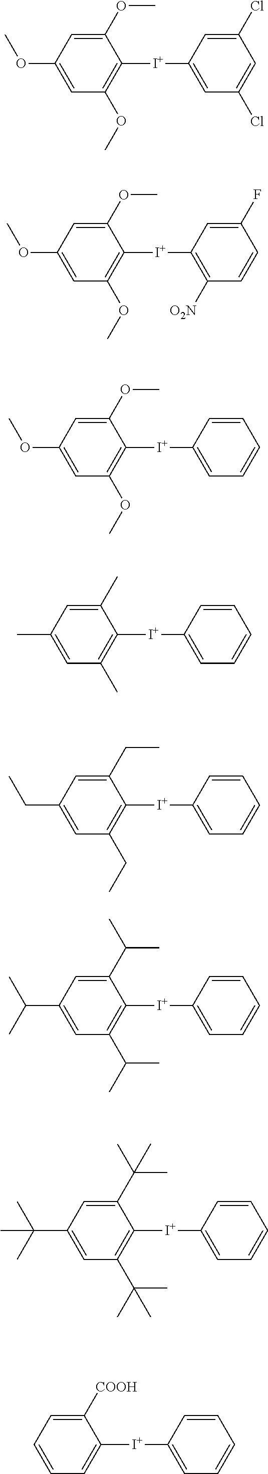

Specific examples of the ammonium cation having formula (cation-3) are shown below, but not limited thereto.

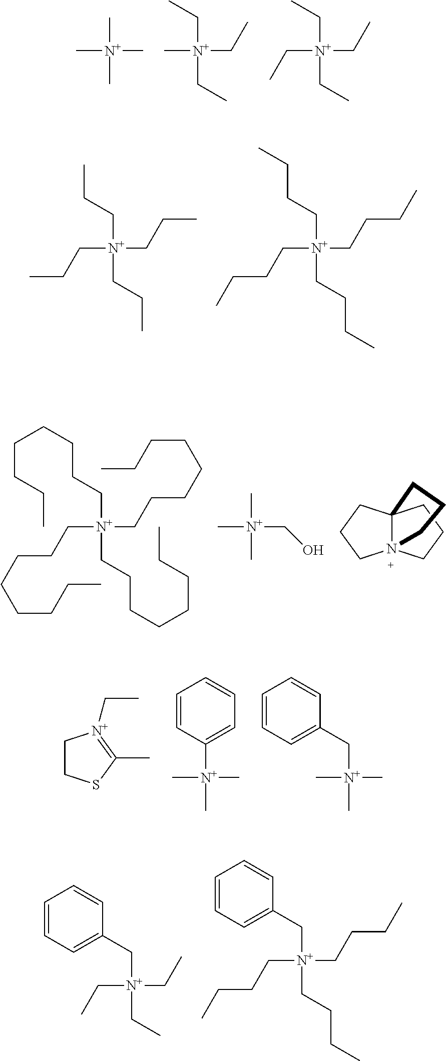

Specific examples of the onium salt of the present invention include arbitrary combinations of anions with cations, both as exemplified above.

The onium salt of the present invention can be synthesized by a known method. As an example, a method for producing an onium salt having the following formula (NSQ-1-ex) is described.

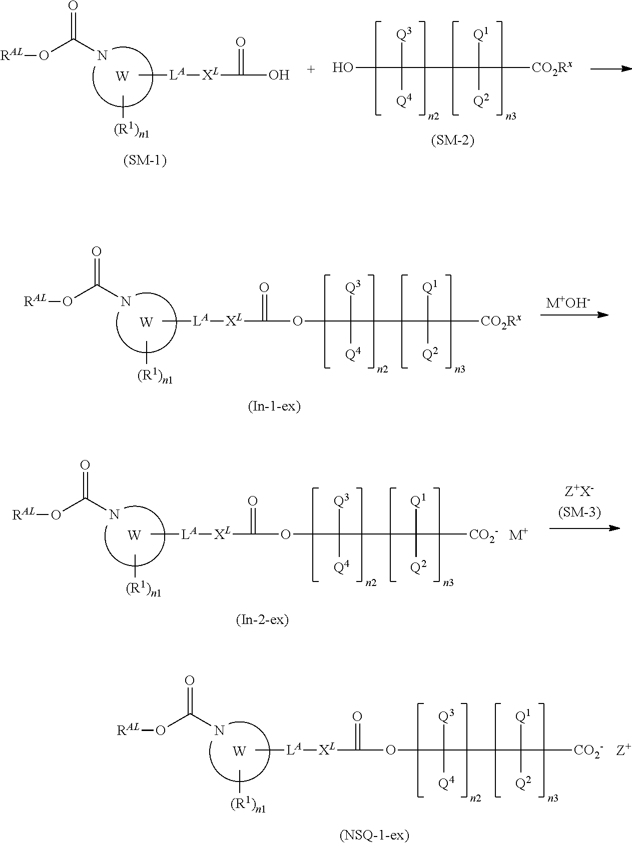

wherein n1 to n3, W, L^{A}, X^{L}, R¹, Q¹ to Q⁴, R^{AL}, and Z⁺ are as defined above, RX is a group forming a primary or secondary ester with the adjacent —CO₂—, M⁺ is a counter cation, and X⁻ is a counter anion.

The first step is to produce Intermediate In-1-ex by reaction between Raw Material SM-1 and Raw Material SM-2, which is commercially available or can be synthesized by a known synthesis method. Various condensing agents can be used when an ester bond is formed directly from the carboxy group in Raw Material SM-1 and the hydroxy group in Raw Material SM-2. Examples of the condensing agent to be used include N,N′-dicyclohexylcarbodiimide, N,N′-diisopropylcarbodiimide, 1-[3-(dimethylamino) propyl]-3-ethylcarbodiimide, and 1-ethyl-3-(3-dimethylaminopropyl) carbodiimide hydrochloride, and from the viewpoint of easy removal of a urea compound formed as the by-product after the reaction, it is preferred to use 1-ethyl-3-(3-dimethylaminopropyl) carbodiimide hydrochloride. The reaction is performed by dissolving Raw Material SM-1 and Raw Material SM-2 in a halogen-based solvent such as methylene chloride and adding a condensing agent. The reaction rate can be improved by adding 4-dimethylaminopyridine as a catalyst. The reaction time is determined as appropriate by monitoring the reaction process by silica gel thin layer chromatography (TLC) because it is desirable from the yield aspect to drive the reaction to completion, but the reaction time is usually about 12 to 24 hours. After stopping the reaction, if necessary, the urea compound produced as a by-product is removed by filtration or water washing, and then the reaction solution is subjected to ordinary aqueous work-up, whereby Intermediate In-1-ex can be obtained. The resulting Intermediate In-1-ex can be purified by a conventional method such as distillation, chromatography, or recrystallization, if necessary.

The second step is to obtain Intermediate In-2-ex by subjecting Intermediate In-1-ex to alkaline hydrolysis. Specifically, this is a step of obtaining Intermediate In-2-ex, which is a carboxylate salt, by subjecting the carboxylic acid ester in Intermediate In-1-ex to alkaline hydrolysis using an alkali metal hydroxide salt or a hydroxide salt of an organic cation. Examples of the alkali metal hydroxide salt to be used include lithium hydroxide, sodium hydroxide, and potassium hydroxide. Examples of the hydroxide salt of an organic cation include tetramethylammonium hydroxide and benzyltrimethylammonium hydroxide. The reaction is performed by dissolving Intermediate In-1-ex in an ether-based solvent such as tetrahydrofuran or 1,4-dioxane, and adding aqueous solutions of various hydroxide salts. The reaction time is determined as appropriate by monitoring the reaction process by silica gel thin layer chromatography (TLC) because it is desirable from the yield aspect to drive the reaction to completion, but the reaction time is usually about 12 to 24 hours. Intermediate In-2-ex can be obtained by subjecting the reaction mixture to ordinary aqueous work-up. The resulting Intermediate In-2-ex can be purified by a conventional method such as chromatography, or recrystallization, if necessary.

The third step is to obtain Onium Salt NSQ-1-ex by subjecting the resulting Intermediate In-2-ex to salt exchange with an onium salt represented by Z⁺X⁻. As X⁻, a chloride ion, a bromide ion, an iodide ion, or a methylsulfate anion is preferable because the exchange reaction easily proceeds in a quantitative manner. It is preferred from the yield aspect to monitor the progress of reaction by silica gel thin layer chromatography (TLC). Onium Salt NSQ-1-ex can be obtained by subjecting the reaction mixture to ordinary aqueous work-up. The resulting Onium Salt NSQ-1-ex can be purified by a conventional method such as chromatography, or recrystallization, if necessary.

In the above scheme, the ion exchange in the third step may be readily performed by a known method, for example, with reference to JP-A 2007-145797.

The producing method is merely exemplary and the method for producing an onium salt of the present invention is not limited thereto.

### [Quencher]

The onium salt of the present invention is useful as a quencher. In the present invention, the quencher is a material capable of trapping the acid generated by the photoacid generator in the resist composition to prevent the acid from diffusing to the unexposed area, for thereby forming a desired pattern.

When the onium salt of the present invention and an onium salt capable of generating strong acid such as α-fluorinated sulfonic acid, imide acid, or methide acid are co-present, a corresponding carboxylic acid and strong acid generate upon light exposure. On the other hand, in the region receiving a reduced exposure dose, much onium salt remains undecomposed. The strong acid functions as a catalyst for inducing deprotection reaction to the acid labile group of the base polymer whereas the onium salt of the present invention induces little deprotection reaction. The strong acid undergoes ion exchange with the residual carboxylic acid sulfonium salt, is converted into a strong acid onium salt and instead, carboxylic acid is released. Differently stated, through ion exchange, the strong acid is neutralized with the carboxylic acid onium salt. That is, the onium salt of the present invention functions as a quencher. This onium salt type quencher tends to form a resist pattern with reduced LWR as compared with the conventional quenchers in the form of amine compounds.

Salt exchange between the strong acid and the carboxylic acid onium salt is infinitely repeated. The site where strong acid is generated at the end of exposure is different from the site where the onium salt of strong acid generation type is initially present. It is believed that since the cycle of photo-acid generation and salt exchange is repeated many times, the points of acid generation are averaged, which leads to a resist pattern with reduced LWR after development.

As the material that exerts a quencher effect by a similar mechanism, for example, Patent Documents 1 to 6 describe a carboxylic acid onium salt, an alkanesulfonic acid onium salt, an arenesulfonic acid onium salt, an α,α-difluorocarboxylic acid onium salt, and the like. With respect to the type of the onium salt, sulfonium salts, iodonium salts, or ammonium salts are included. On use of an alkanesulfonic acid onium salt or arenesulfonic acid onium salt, the generated acid has a certain acid strength so that part thereof may induce deprotection reaction as the acid generator rather than as the quencher, leading to a lowering of resolution performance and an increase of acid diffusion, which invite losses of resist performance factors like exposure latitude (EL) and mask error factor (MEF). Also, the α,α-difluorocarboxylic acid onium salt as described in Patent Document 6, despite a carboxylic acid onium salt, has possibility to provoke deprotection reaction depending on a choice of acid labile group on the base polymer, for the reason that the generated acid has a relatively high acidity like the sulfonic acid onium salt, due to the inclusion of a fluorine atom at α-position of the carboxylate anion. Fluorocarboxylic acid onium salts obtained by simply extending the straight chain similarly allow for substantial acid diffusion and undergo salt exchange with strong acid in the unexposed area, probably leading to losses of resolution, EL, and MEF. The alkanecarboxylic acid onium salt is highly hydrophilic though it functions as a quencher. The fluoroalkanecarboxylic acid onium salt as described in Patent Document 3 has a somewhat controlled level of hydrophilicity as compared with the non-fluorinated type, but the control of hydrophilicity is insufficient when the carbon count is small. Although some onium salts of perfluoroalkanecarboxylic acid having a larger carbon count are known, they are deemed incompatible with resist compositions because the carboxylic acids have surfactant-like physical properties. Incompatibility with resist compositions can cause defect formation. Additionally, perfluoroalkanecarboxylic acids are unfavorable from the biotic and environmental aspects.

Patent Documents 7 to 9 describe a carboxylic acid onium salt having indole or indoline as a nitrogen-containing heterocyclic compound and a piperidinecarboxylic acid structure, Patent Document 10 describes a carboxylic acid onium salt having an aminobenzoic acid structure, and Patent Document 11 describes a carboxylic acid onium salt having an amide bond. Although these also act as a quencher, since aromatic amines and amide bonds are not highly basic, and thus acid diffusion controllability is not sufficient, and piperidinecarboxylic acid has extremely high water solubility, the permeation of the alkaline developer into the unexposed area is promoted, and there is a concern that the resist pattern may collapse or peeling of the resist pattern from the substrate may occur.

The onium salt of the present invention can solve the above problems. An onium salt having a nitrogen atom-containing aliphatic heterocyclic ring and a fluorocarboxylic acid structure in an anion acts as a quencher, effectively traps the strong acid generated from an acid generator in the exposed area at a fluorocarboxylic acid anion site, and causes an acid labile group protecting the nitrogen atom-containing aliphatic heterocyclic site to undergo a deprotection reaction by the strong acid, thereby producing a nitrogen atom-containing aliphatic heterocyclic compound having high basicity. Since the deprotection reaction with an acid proceeds to form a nitrogen atom-containing aliphatic heterocyclic structure having high basicity, a fluorocarboxylic acid is formed in the exposed area, but the deprotection reaction of the base polymer is not caused. The nitrogen atom-containing aliphatic heterocyclic site having high basicity suppresses excessive diffusion of the acid into the unexposed area, and the carboxylic acid anion site continuously repeats proton exchange with the strong acid. It is considered that favorable lithographic performance can be achieved even in fine pattern formation by improving the dissolution contrast between the exposed area and the unexposed area and appropriately controlling the acid diffusion of the strong acid by these synergistic effects. Since the organic solvent solubility is improved by having an appropriate number of carbon atoms due to the fluorocarboxylic acid structure and the acid labile group protecting the nitrogen atom-containing aliphatic heterocyclic site, it is possible to effectively suppress the permeation of the alkaline developer into the unexposed area and the collapse and peeling of the resist pattern accompanying the permeation.

### [Resist Composition]

The resist composition of the present invention comprises
(A) a quencher comprising the onium salt having formula (1) as an essential component.

The content of the quencher (A) in the resist composition of the present invention is preferably 0.1 to 40 parts by weight and more preferably 1 to 20 parts by weight per 80 parts by weight of a base polymer (C) described below. When the content of the quencher (A) is in the above range, the quencher sufficiently functions as a quencher, and there is no possibility of performance deterioration such as deterioration in sensitivity or generation of foreign matter due to insufficient solubility.

### [Organic Solvent (B)]

The resist composition of the present invention may comprise an organic solvent as a component (B). The organic solvent (B) is not particularly limited as long as the component (A) and each component described below can be dissolved. Specific examples of such an organic solvent include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; ketoalcohols such as diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as γ-butyrolactone (GBL), and mixed solvents thereof.

Of the foregoing organic solvents, it is recommended to use PGME, PGMEA, cyclohexanone, GBL, DAA, ethyl lactate, and mixed solvents thereof because the base polymer of the component (C) is most soluble therein.

The content of the organic solvent (B) in the resist composition of the present invention is preferably 200 to 5000 parts by weight and more preferably 400 to 3500 parts by weight per 80 parts by weight of a base polymer (C) described below. The organic solvent (B) may be used alone or in admixture of two or more kinds thereof.

### [Base Polymer (C)]

The resist composition of the present invention may comprise a base polymer as a component (C). The base polymer (C) comprises repeat units having the following formula (a1) (also referred to as repeat units a1, hereinafter).

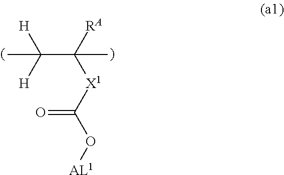

In formula (a1), R^{A} is a hydrogen atom, a fluorine atom, a methyl group, or a trifluoromethyl group. X¹ is a single bond, a phenylene group, a naphthylene group, or *—C(═O)—O—X¹¹—, the phenylene group or the naphthylene group may be substituted with a C₁-C₁₀ alkoxy group which may contain a fluorine atom, or a halogen atom. X¹¹ is a C₁-C₁₀ saturated hydrocarbylene group, a phenylene group, or a naphthylene group, the saturated hydrocarbylene group may contain a hydroxy group, an ether bond, an ester bond, or a lactone ring. * designates a point of attachment to the carbon atom in the backbone.

In formula (a1), AL¹ is an acid labile group. Examples of the acid labile group include those groups described in JP-A 2013-80033 and JP-A 2013-83821.

Typical examples of the acid labile group include groups having the following formulae (AL-3) to (AL-5).

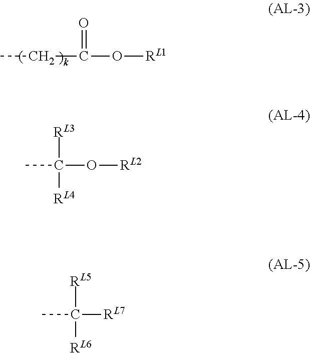

wherein the broken line denotes a point of attachment.

In formulae (AL-3) and (AL-4), R^{L1} and R^{L2} are each independently a C₁-C₄₀ saturated hydrocarbyl group, which may contain a heteroatom such as an oxygen atom, a sulfur atom, a nitrogen atom, or a fluorine atom. The saturated hydrocarbyl group may be straight, branched, or cyclic. The saturated hydrocarbyl group is preferably a C₁-C₂₀ saturated hydrocarbyl group.

In formula (AL-3), k is an integer of 0 to 10 and preferably an integer of 1 to 5.

In formula (AL-4), R^{L3} and R^{L4} are each independently a hydrogen atom or a C₁-C₂₀ saturated hydrocarbyl group, which may contain a heteroatom such as an oxygen atom, a sulfur atom, a nitrogen atom, or a fluorine atom. The hydrocarbylene group may be straight, branched, or cyclic. Any two of R^{L2}, R^{L3}, and R^{L4} may bond together to form a C₃-C₂₀ ring with the carbon atom or carbon and oxygen atoms to which they are attached. The ring is preferably a C₄-C₁₆ ring and particularly preferably an alicyclic ring.

In formula (AL-5), R^{L5}, R^{L6}, and R^{L7} are each independently a C₁-C₂₀ saturated hydrocarbyl group, which may contain a heteroatom such as an oxygen atom, a sulfur atom, a nitrogen atom, or a fluorine atom. The hydrocarbylene group may be straight, branched, or cyclic. Any two of R^{L5}, R^{L6}, and R^{L7} may bond together to form a C₃-C₂₀ ring with the carbon atom to which they are attached. The ring is preferably a C₄-C₁₆ ring and particularly preferably an alicyclic ring.

Specific examples of the repeat units a1 are shown below, but not limited thereto. In the following formula, R^{A} and AL¹ are as defined above.

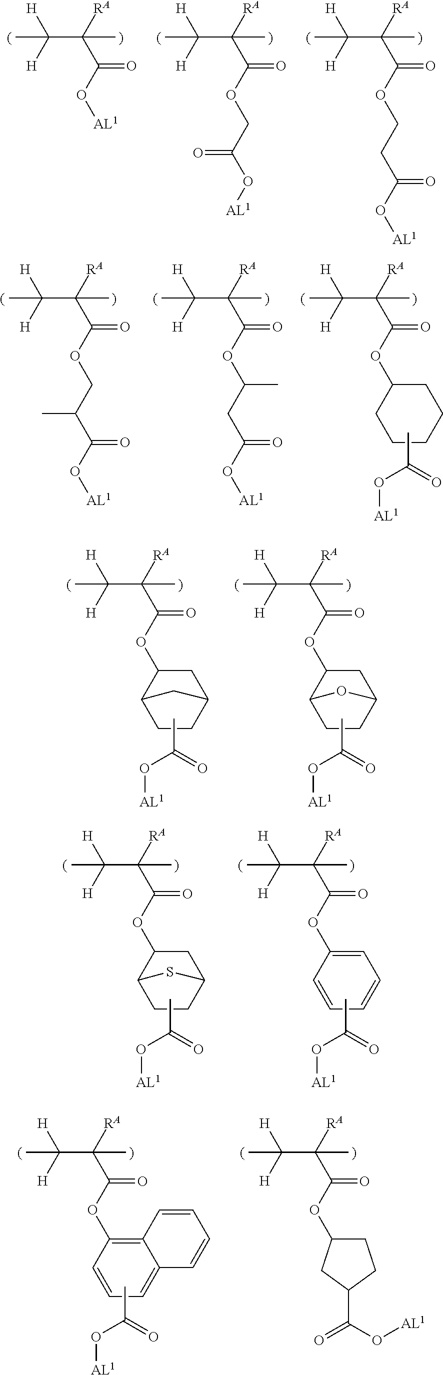

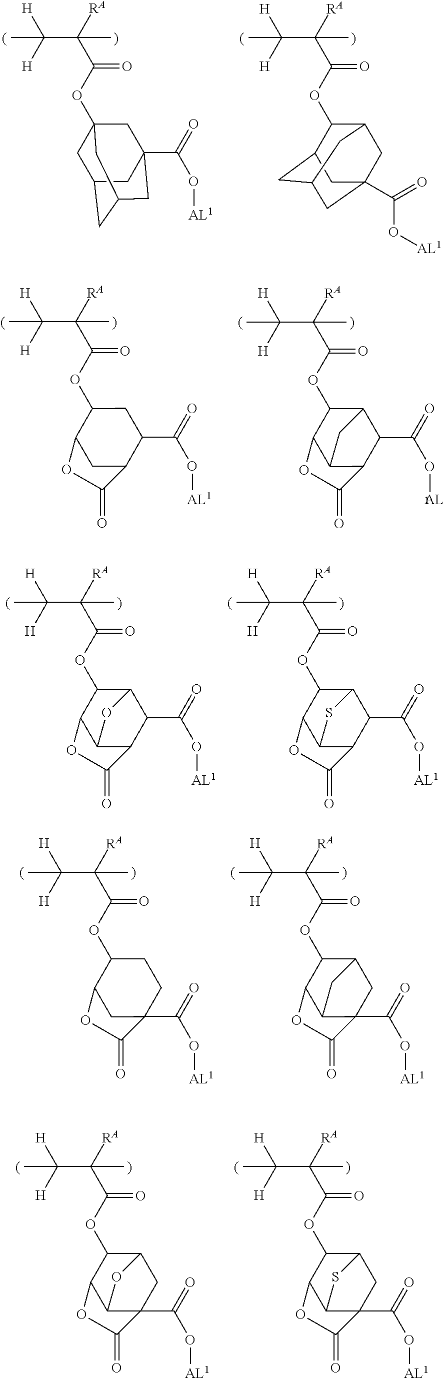

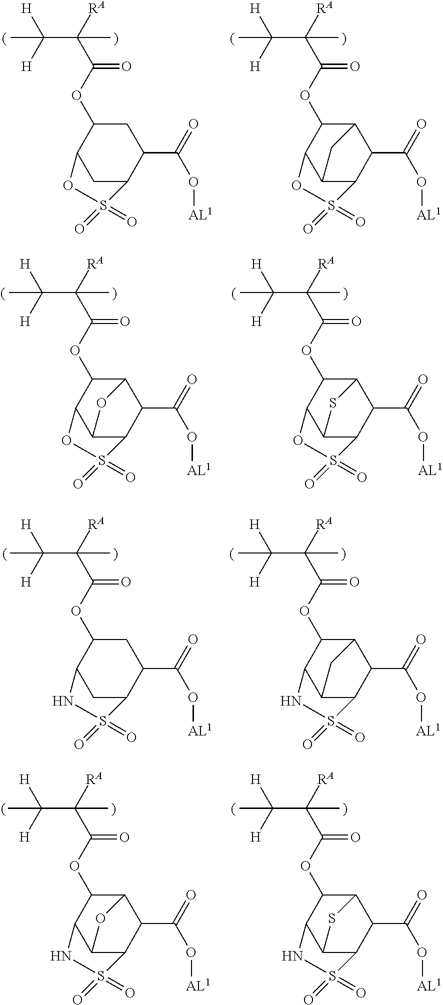

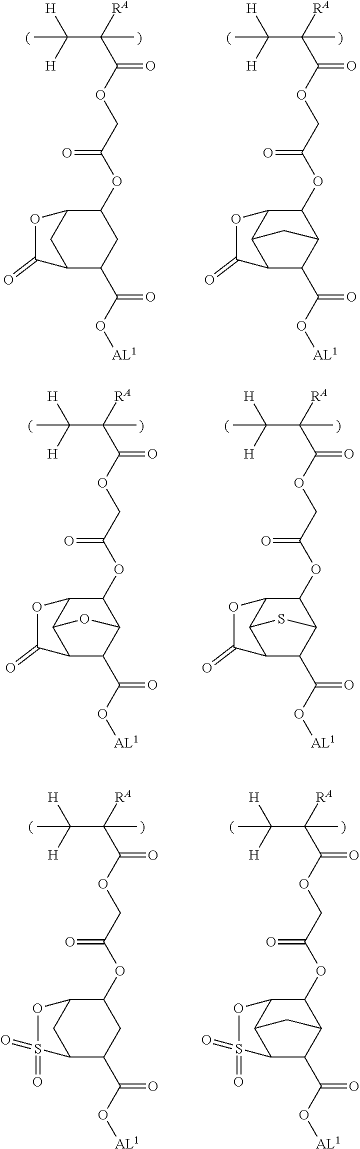

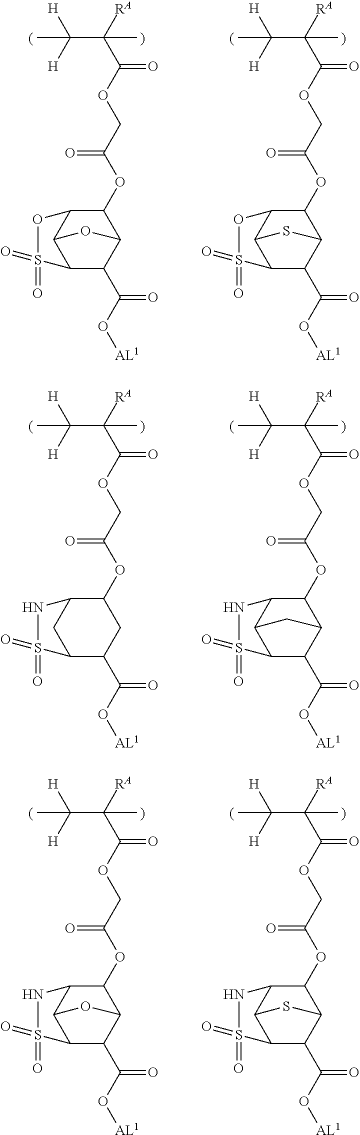

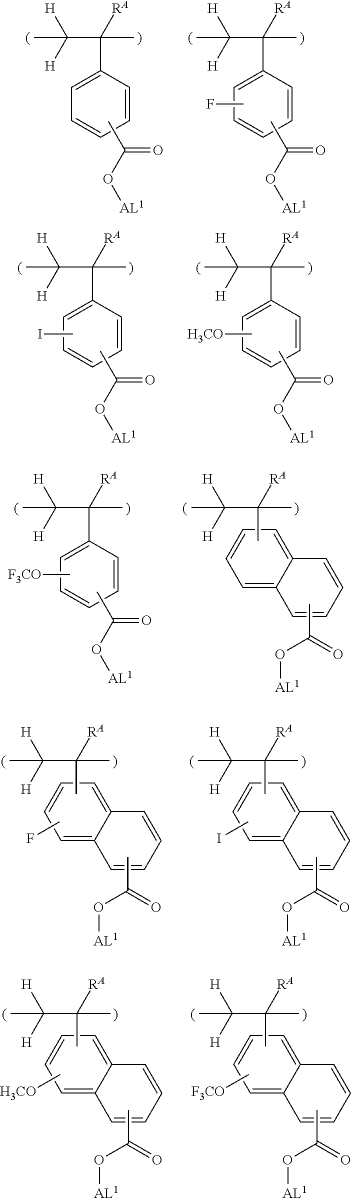

The base polymer may further comprise repeat units having the following formula (a2) (also referred to as repeat units a2, hereinafter).

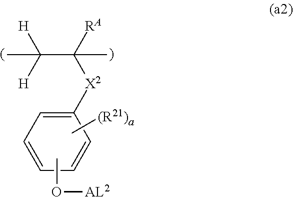

In formula (a2), R^{A} is a hydrogen atom, a fluorine atom, a methyl group, or a trifluoromethyl group. X² is a single bond or *—C(═O)—O—. * designates a point of attachment to the carbon atom in the backbone. R²¹ is a halogen atom, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. AL² is an acid labile group. Examples of the acid labile group are as exemplified above for the acid labile group represented by AL¹. a is an integer of 0 to 4, preferably 0 or 1.

Specific examples of the repeat units a2 are shown below, but not limited thereto. In the following formula, R^{A} and AL² are as defined above.

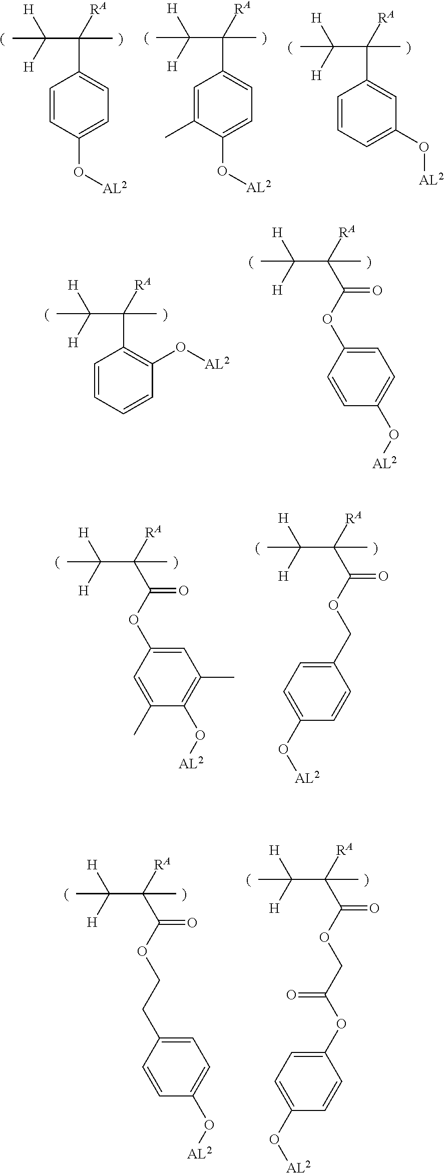

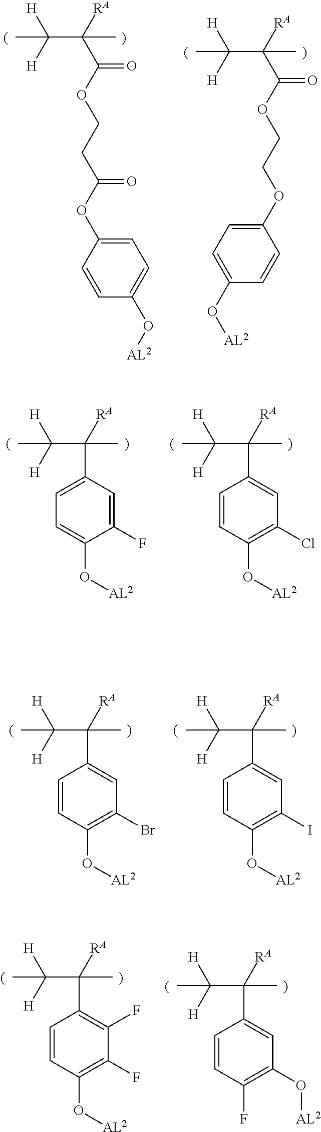

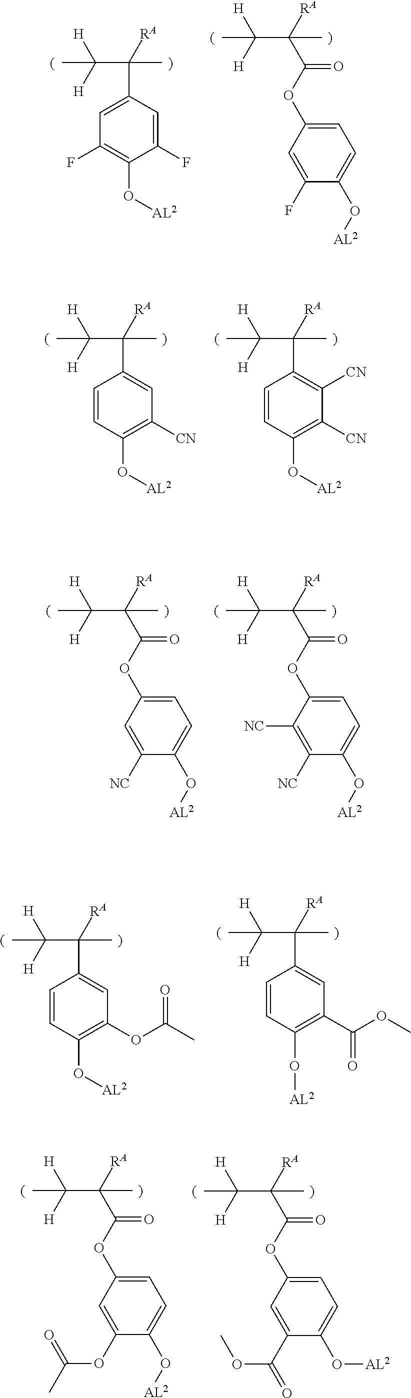

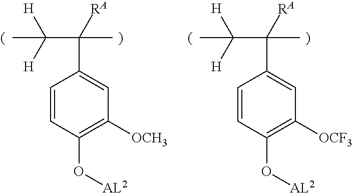

The base polymer preferably further comprises repeat units having the following formula (b1) (also referred to as repeat units b1, hereinafter) or repeat units having the following formula (b2) (also referred to as repeat units b2, hereinafter).

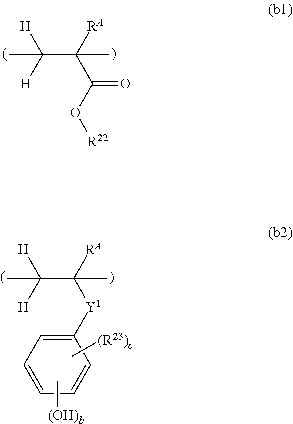

In formulae (b1) and (b2), R^{A} is each independently a hydrogen atom, a fluorine atom, a methyl group, or a trifluoromethyl group. Y¹ is a single bond or *—C(═O)—O—. * designates a point of attachment to the carbon atom in the backbone. R²² is a hydrogen atom or a C₁-C₂₀ group containing at least one or more structures selected from a hydroxy group other than a phenolic hydroxy group, a cyano group, a carbonyl group, a carboxy group, an ether bond, an ester bond, a sulfonic acid ester bond, a carbonate bond, a lactone ring, a sultone ring, and a carboxylic anhydride (—C(═O)—O—C(═O)—). R²³ is a halogen atom, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. b is an integer of 1 to 4. c is an integer of 0 to 4. Provided that b+c is from 1 to 5.

Specific examples of the repeat units b1 are shown below, but not limited thereto. In the following formula, R^{A} is as defined above.

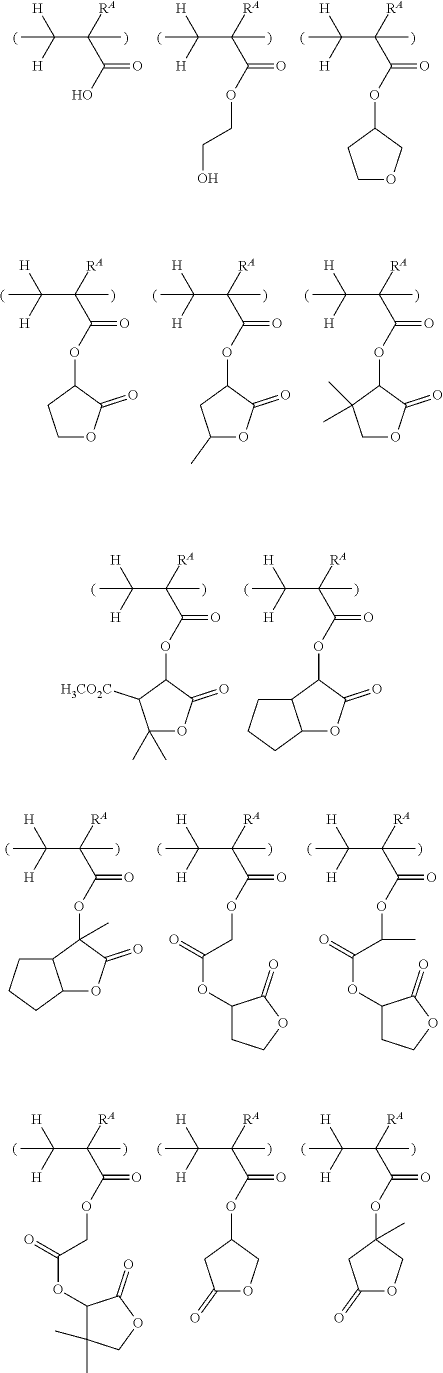

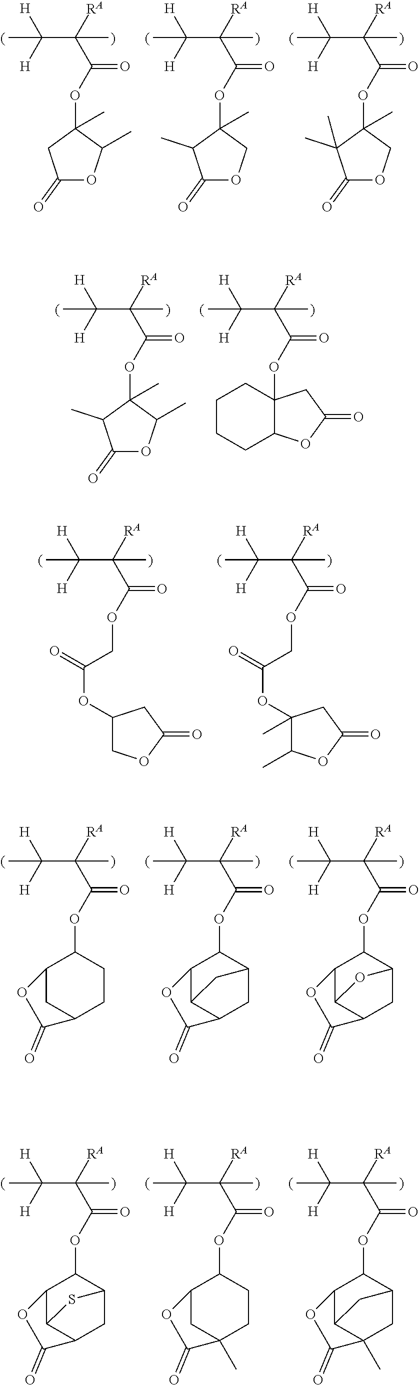

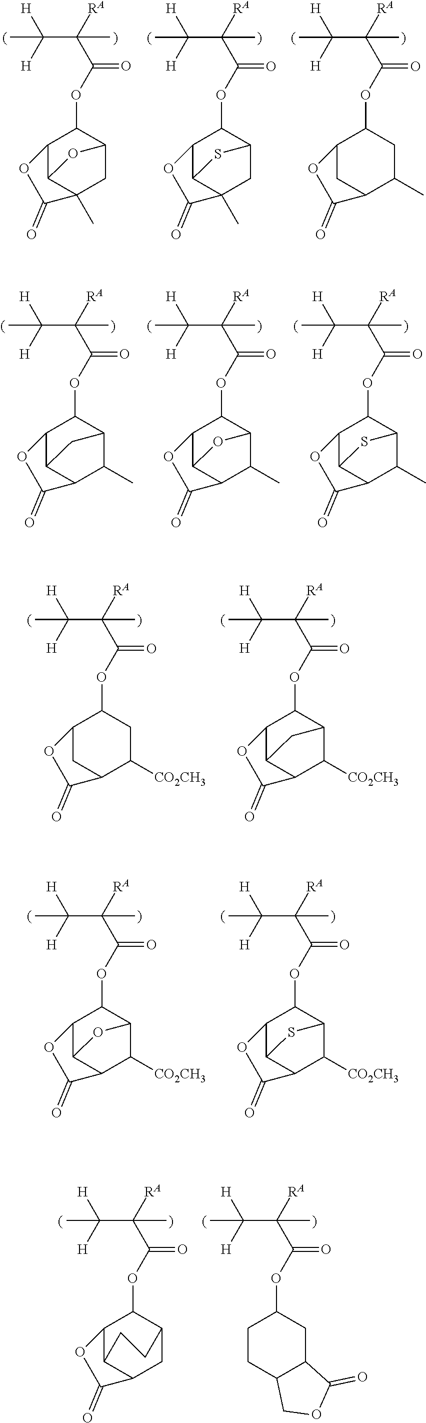

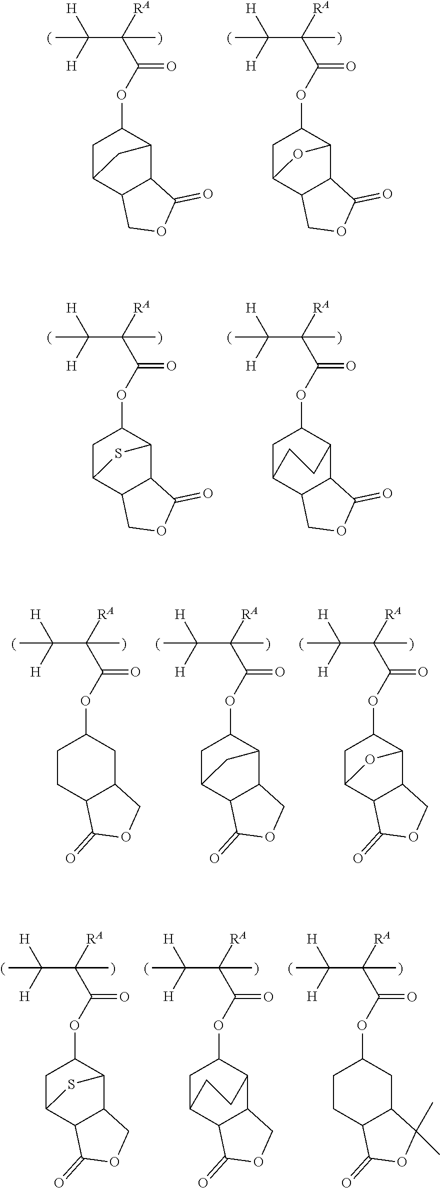

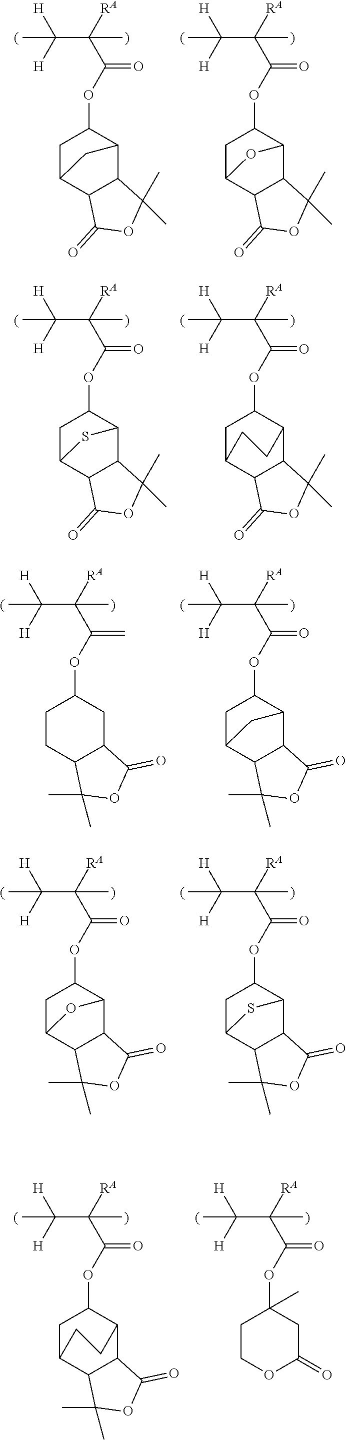

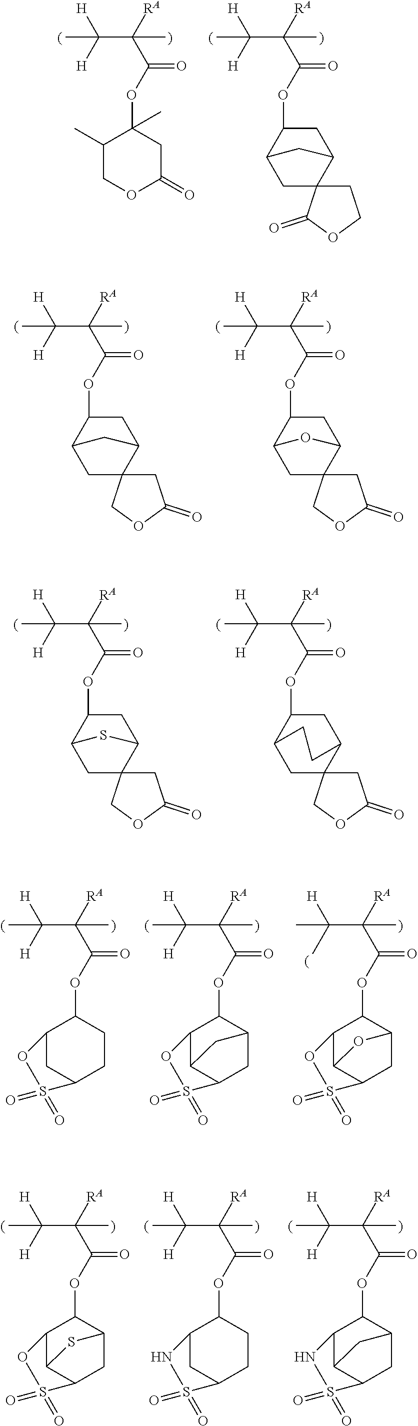

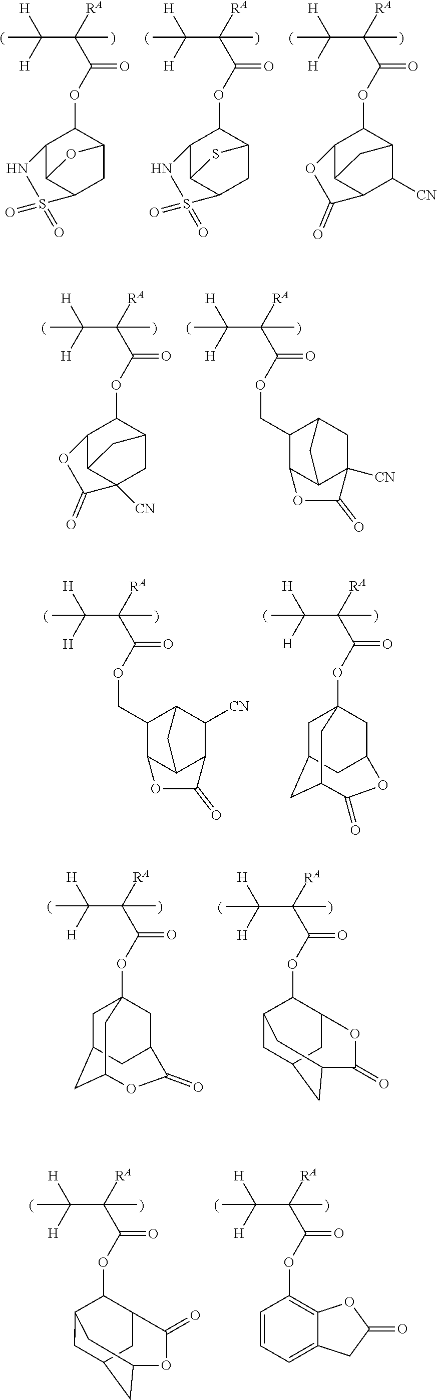

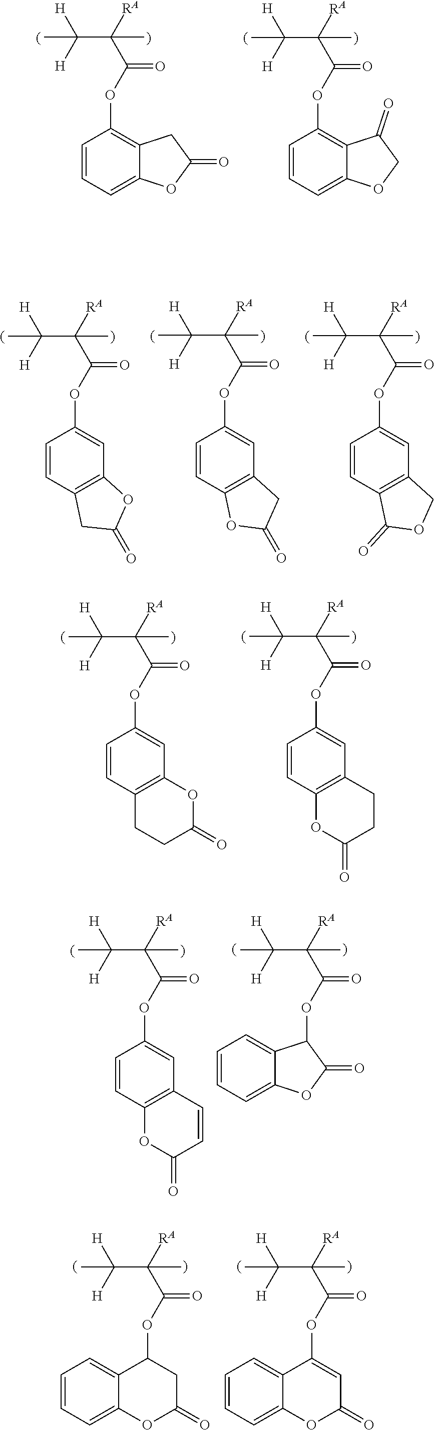

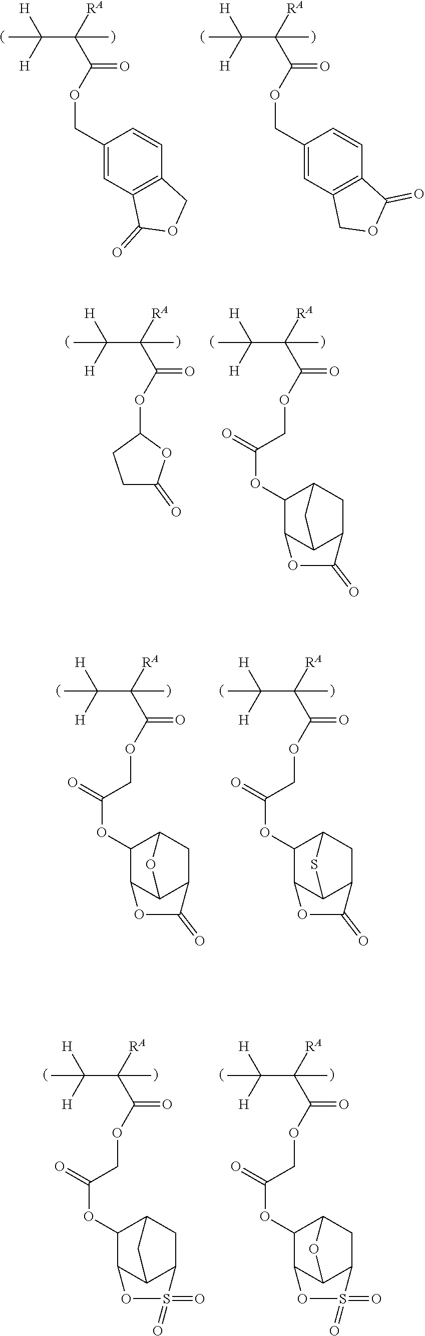

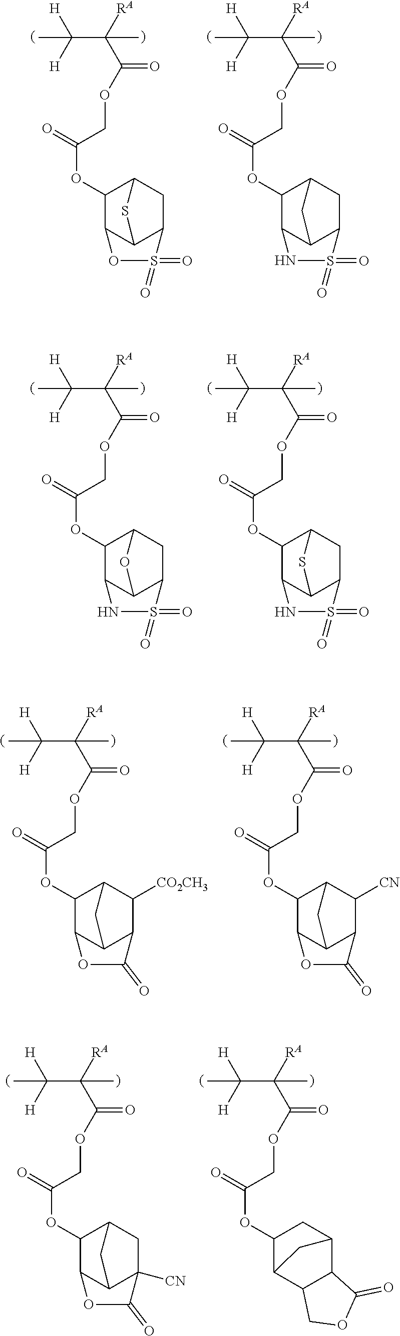

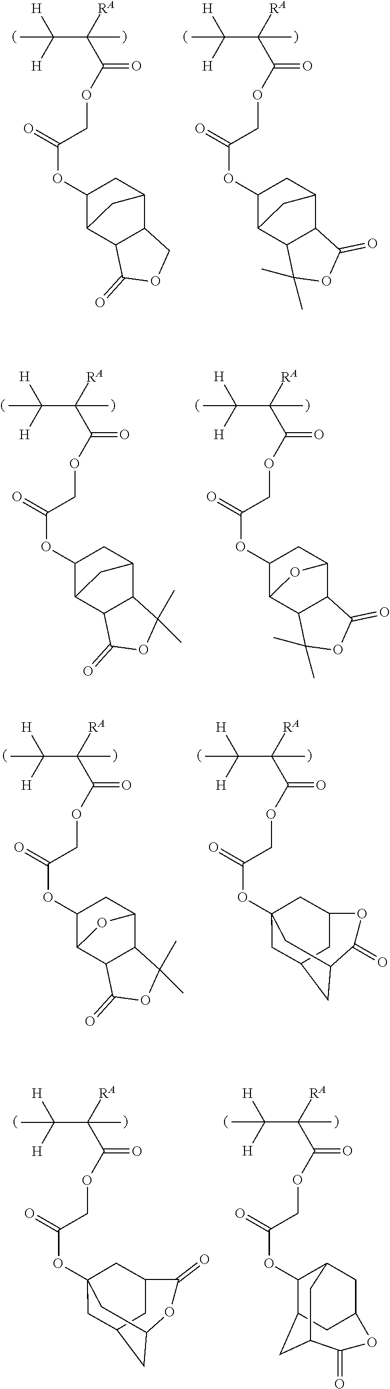

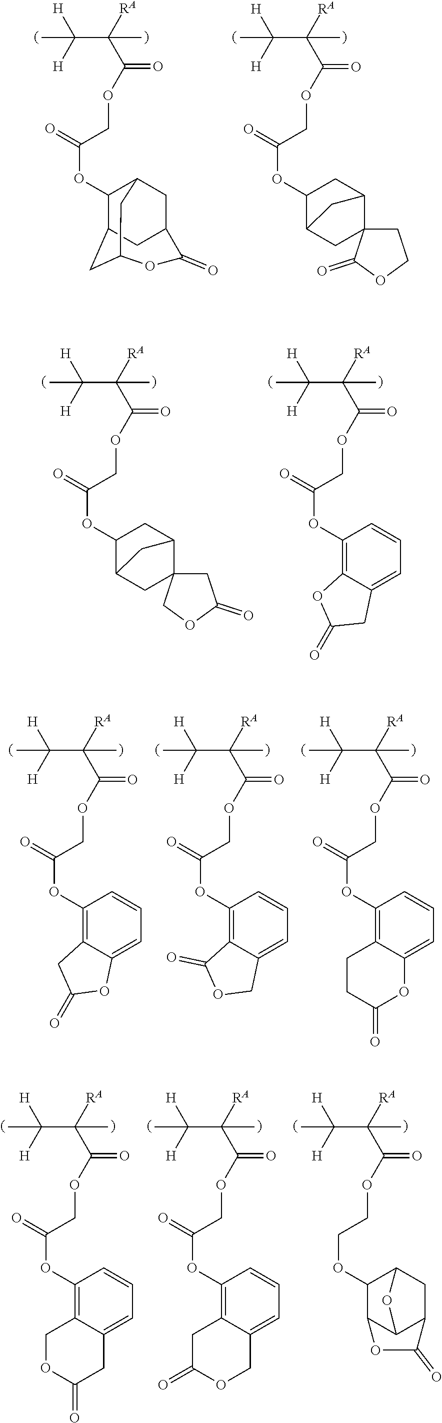

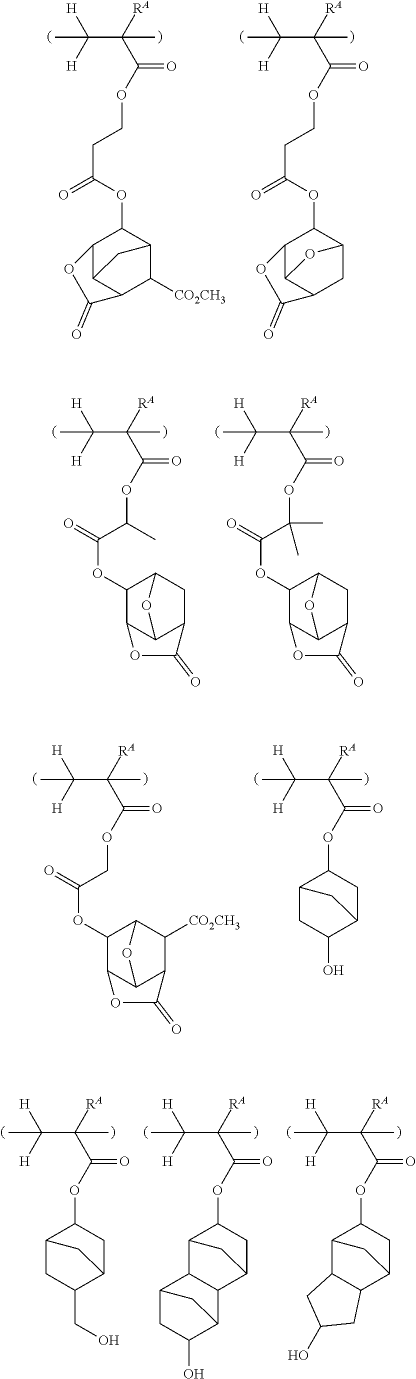

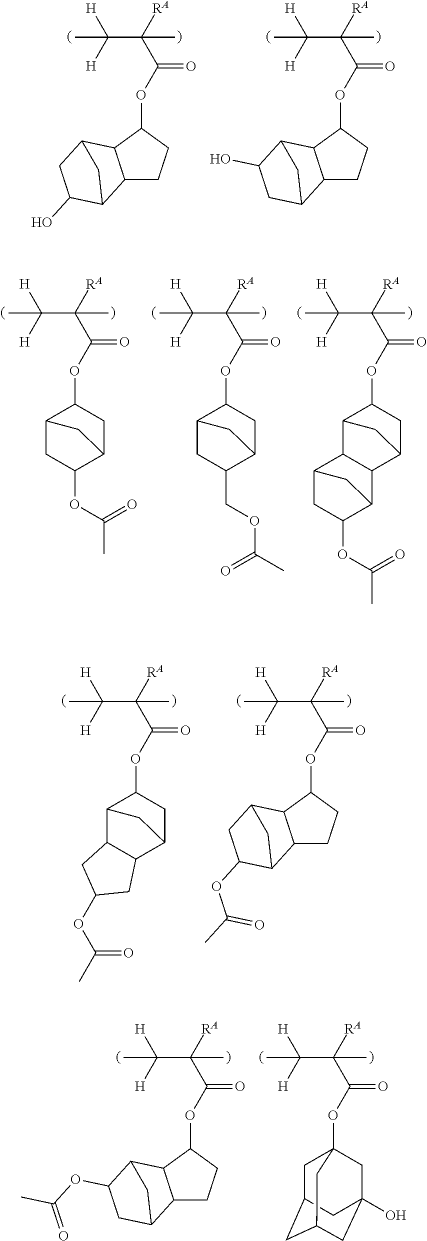

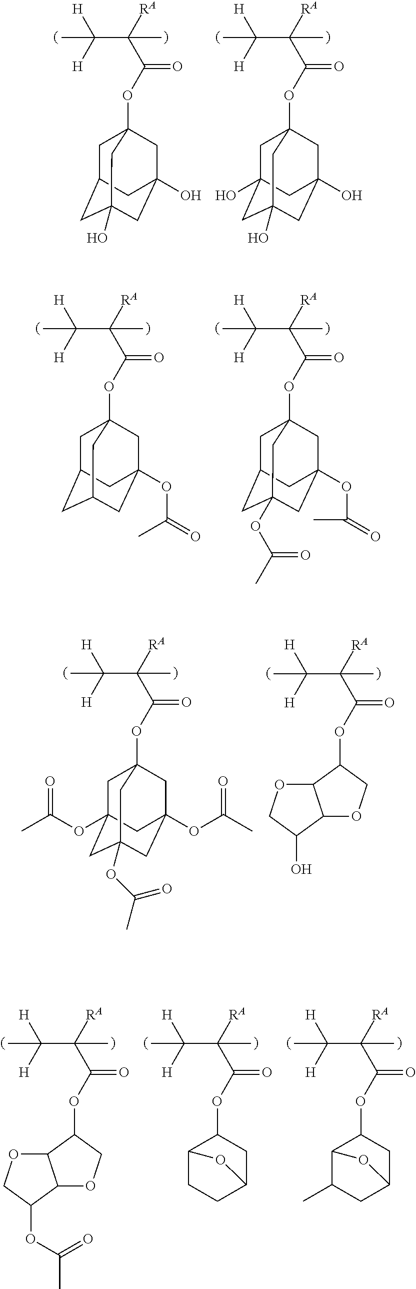

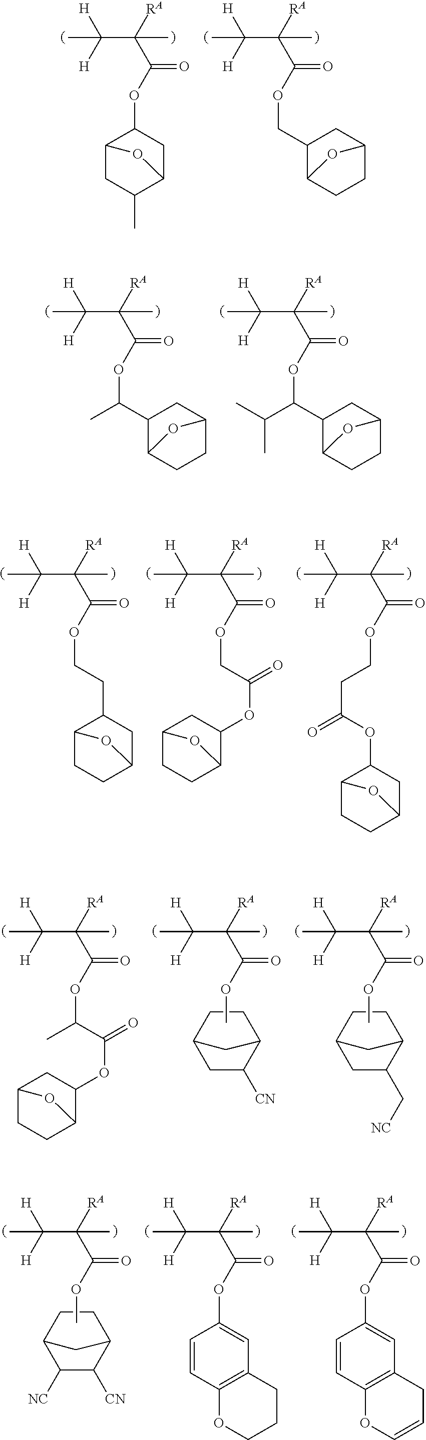

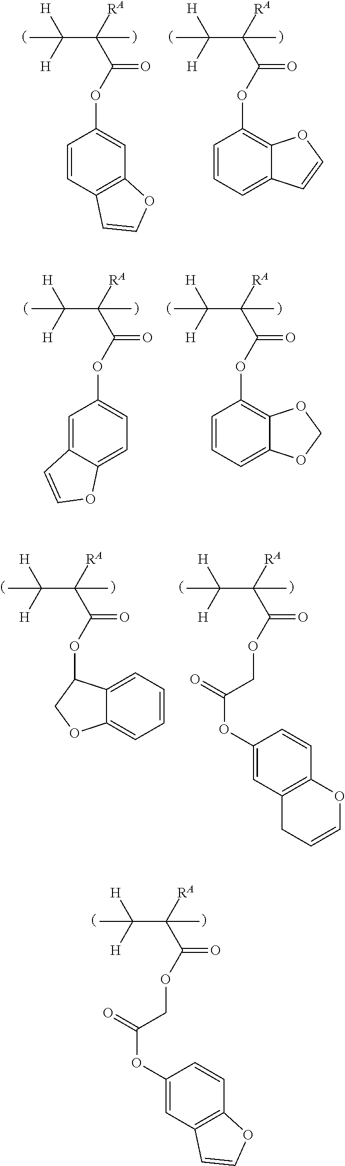

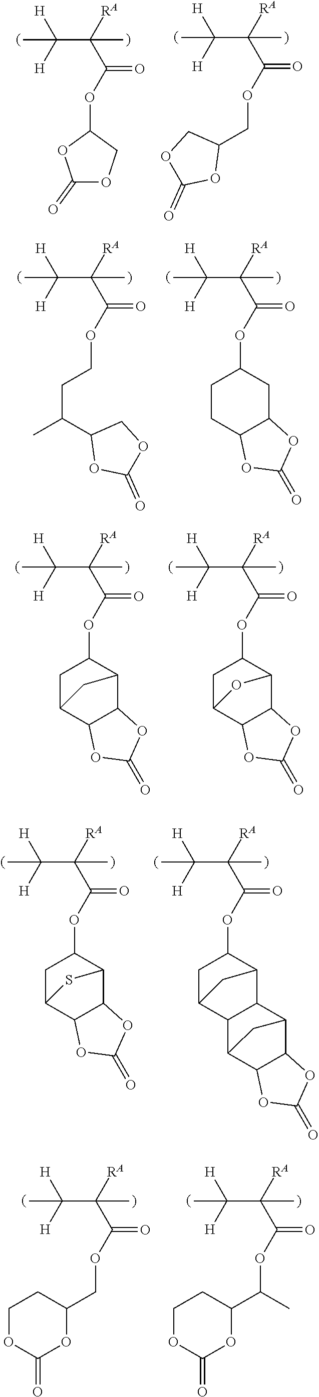

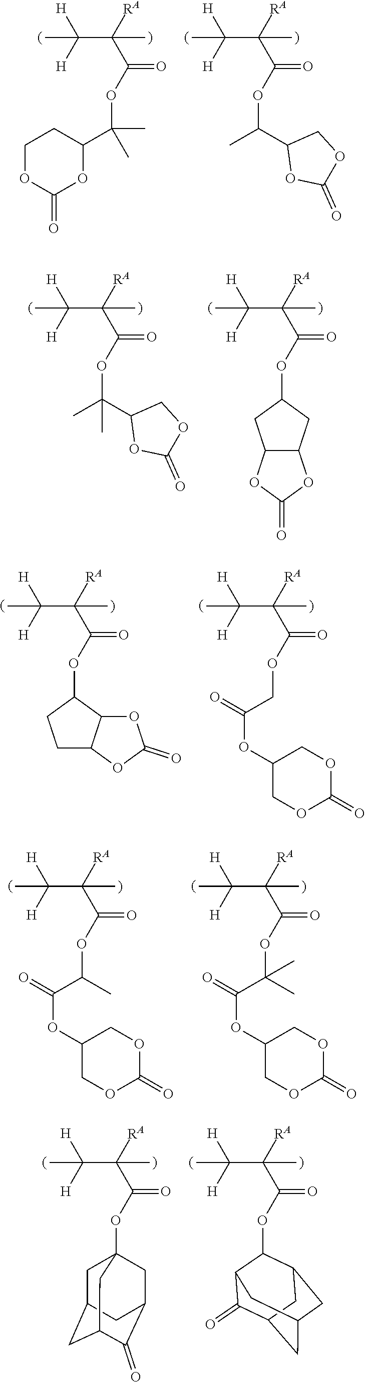

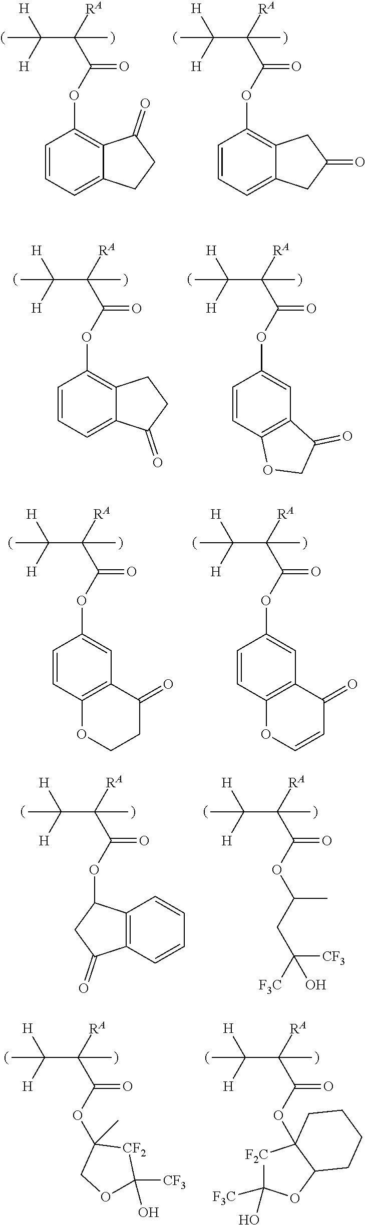

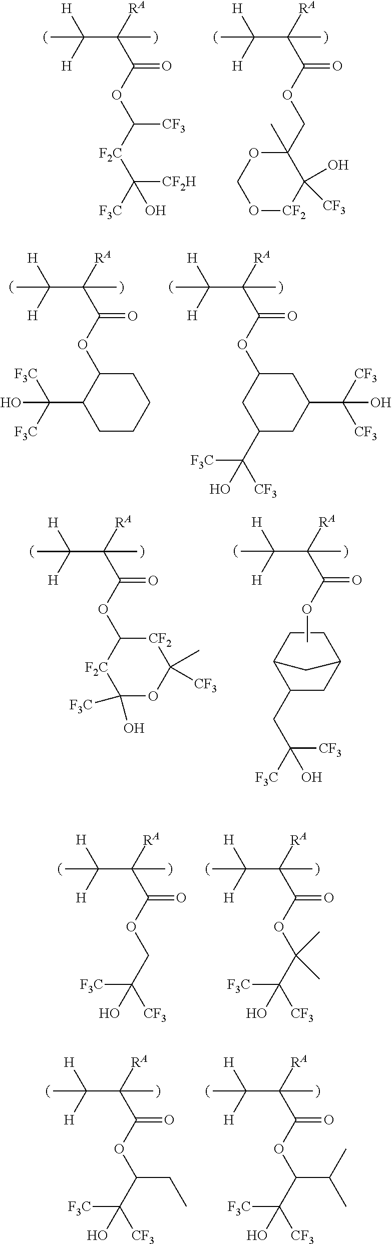

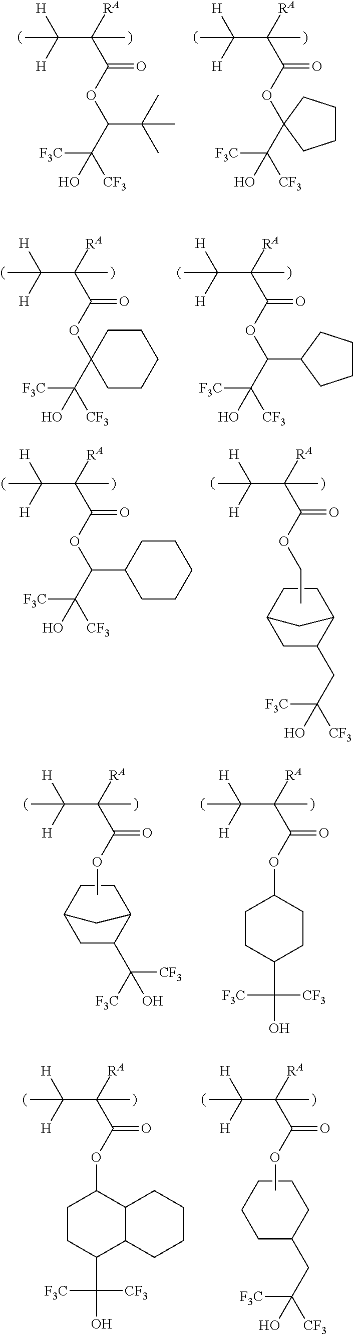

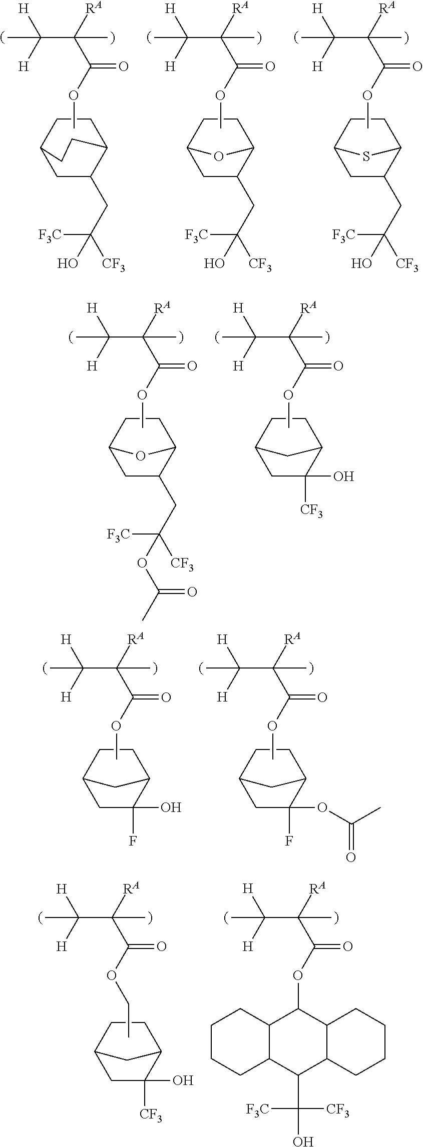

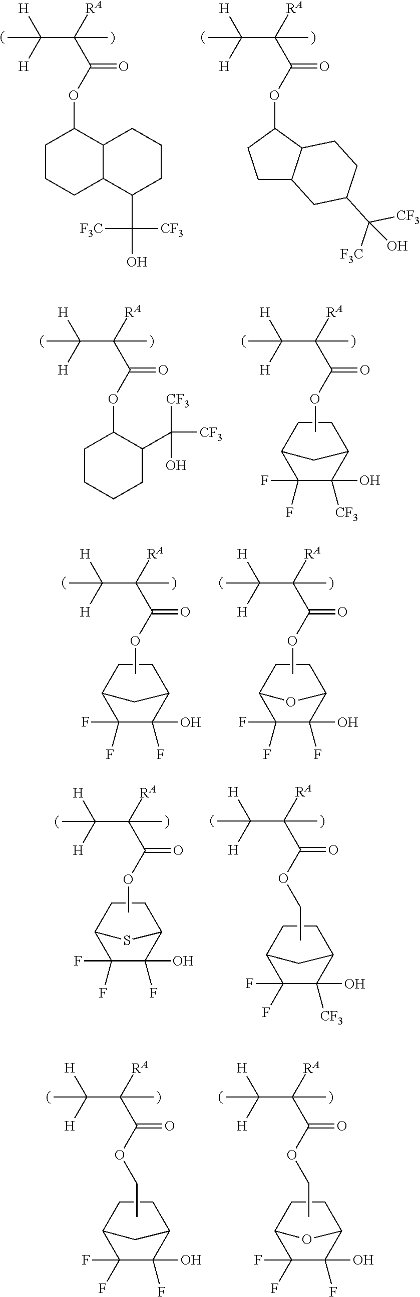

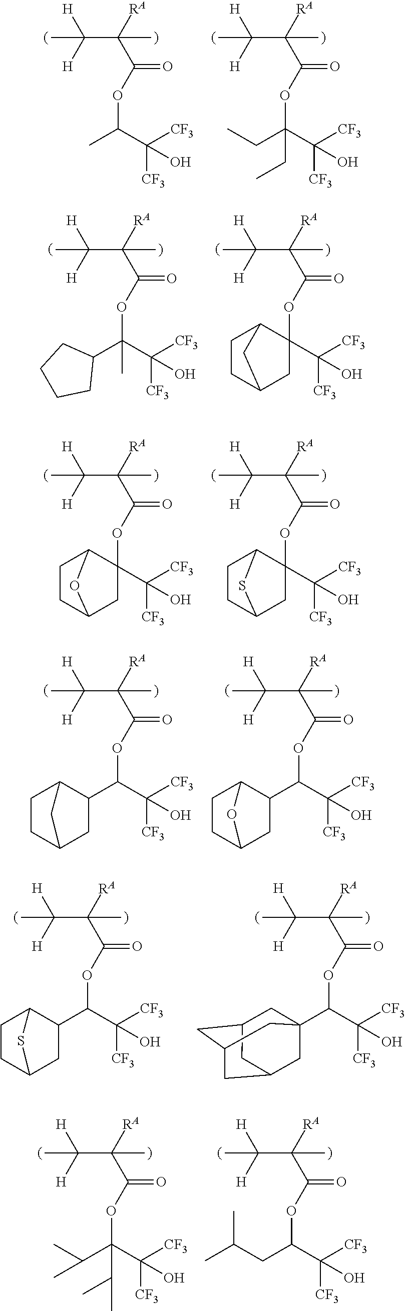

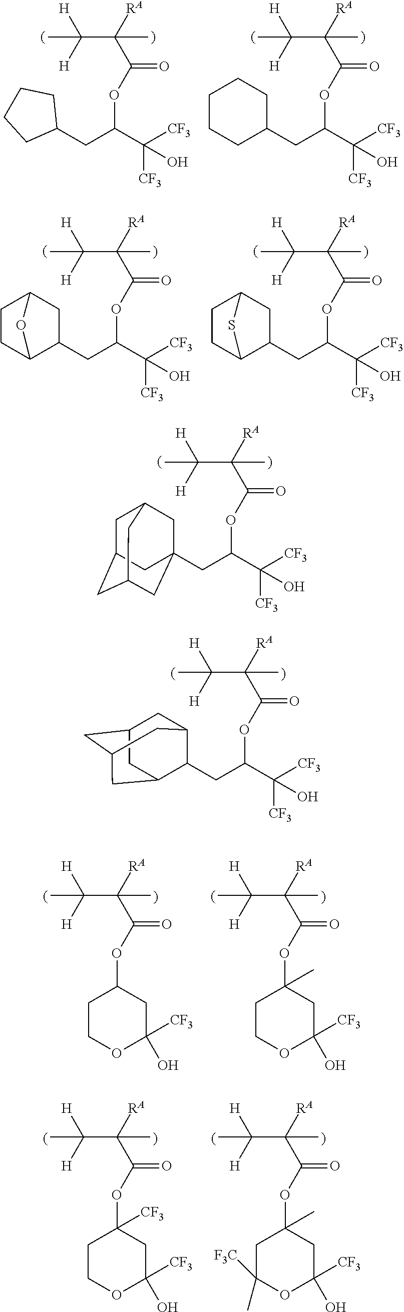

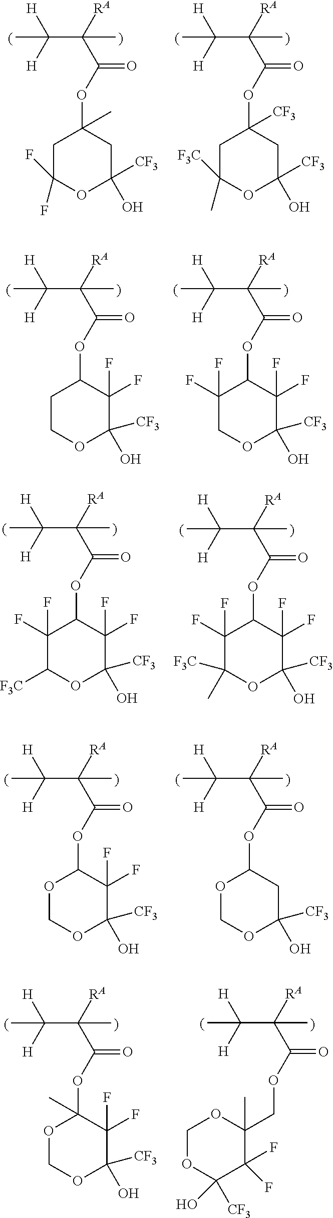

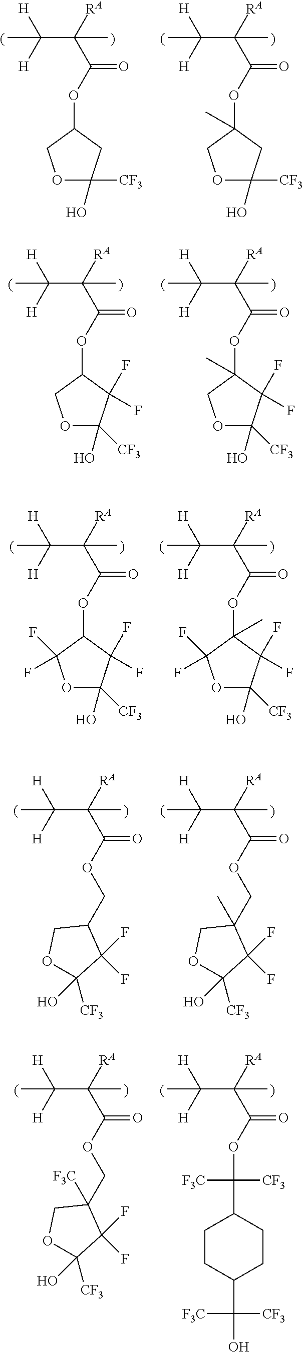

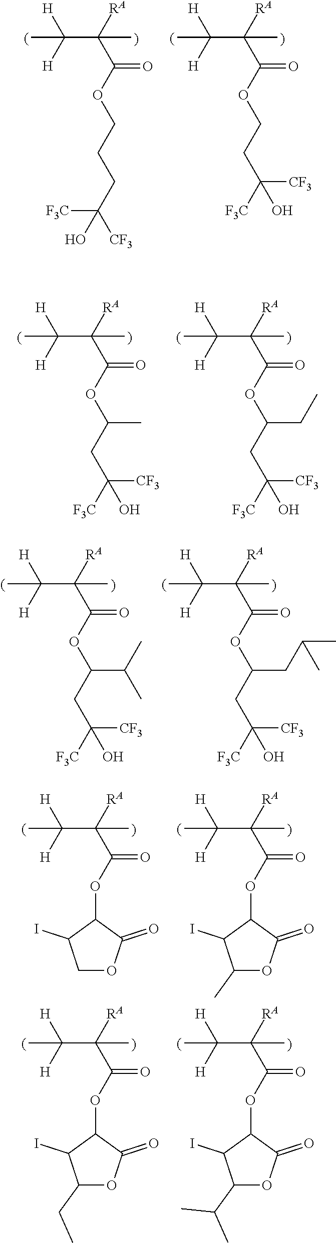

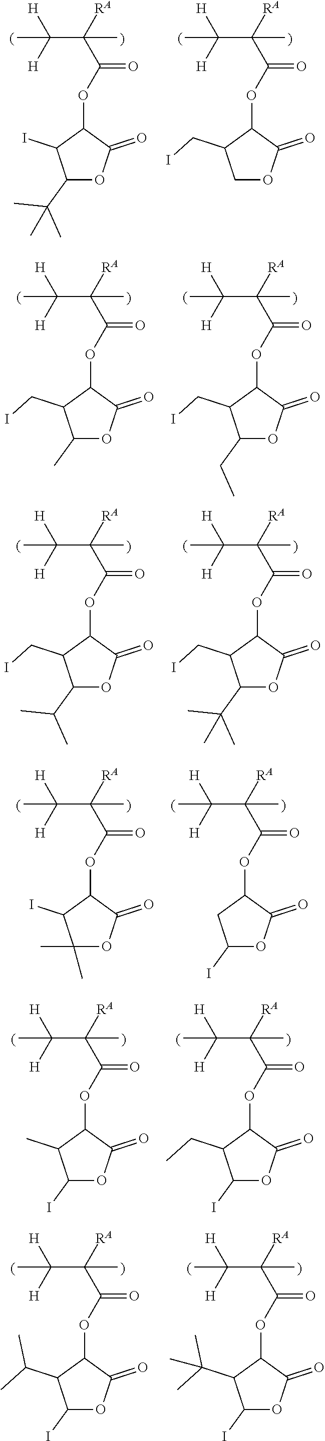

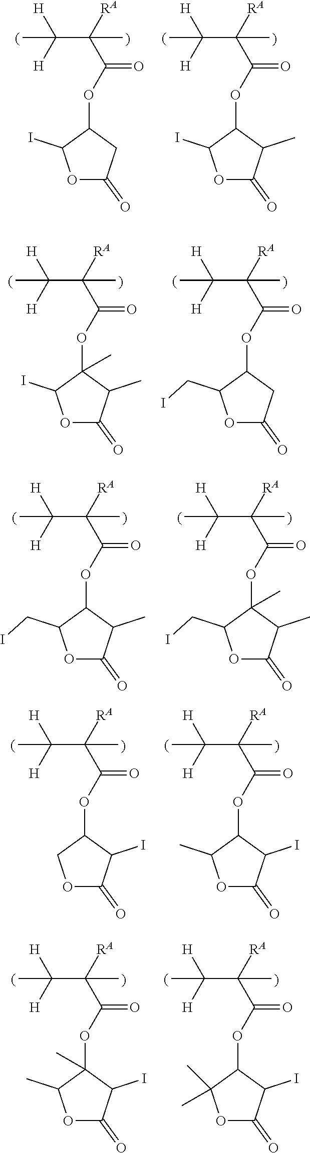

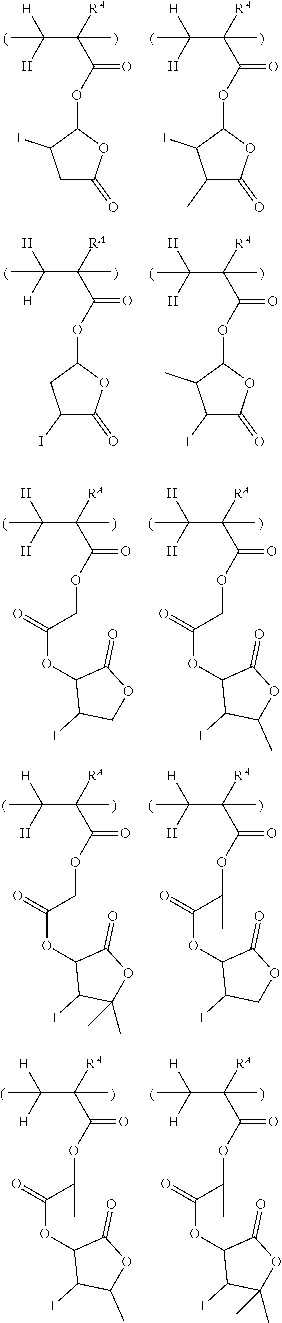

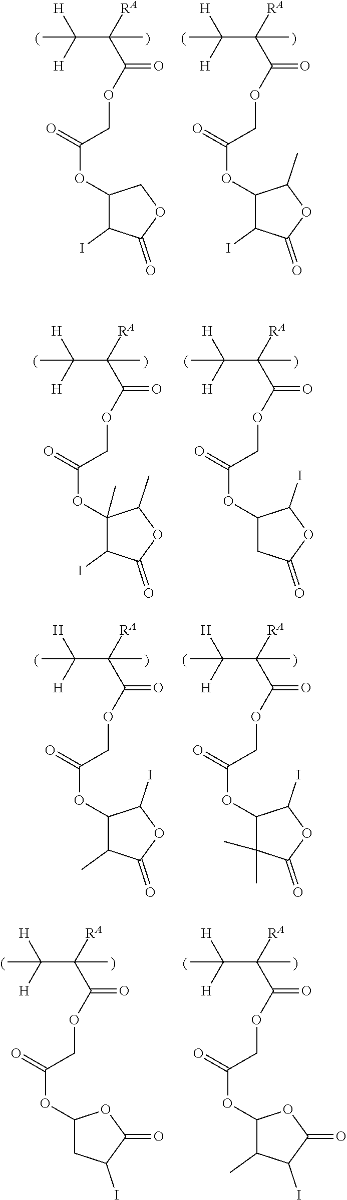

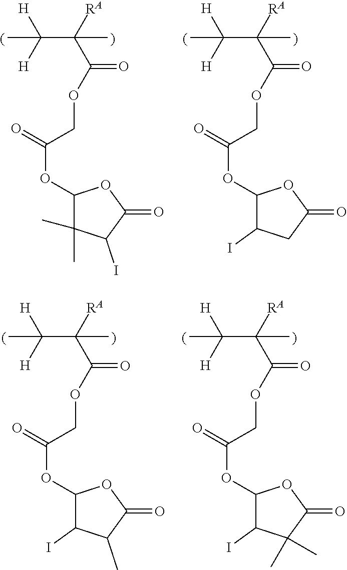

Specific examples of the repeat units b2 are shown below, but not limited thereto. In the following formula. R^{A} is as defined above.

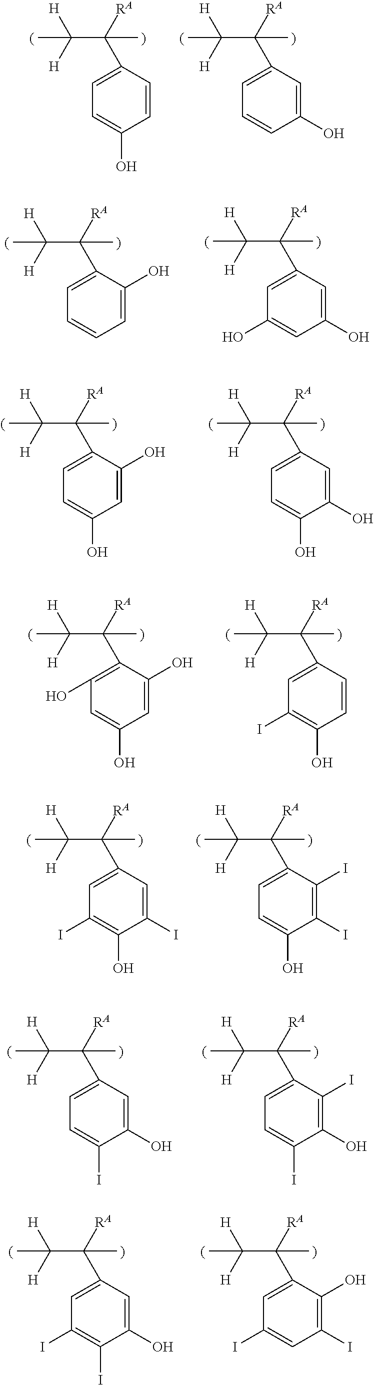

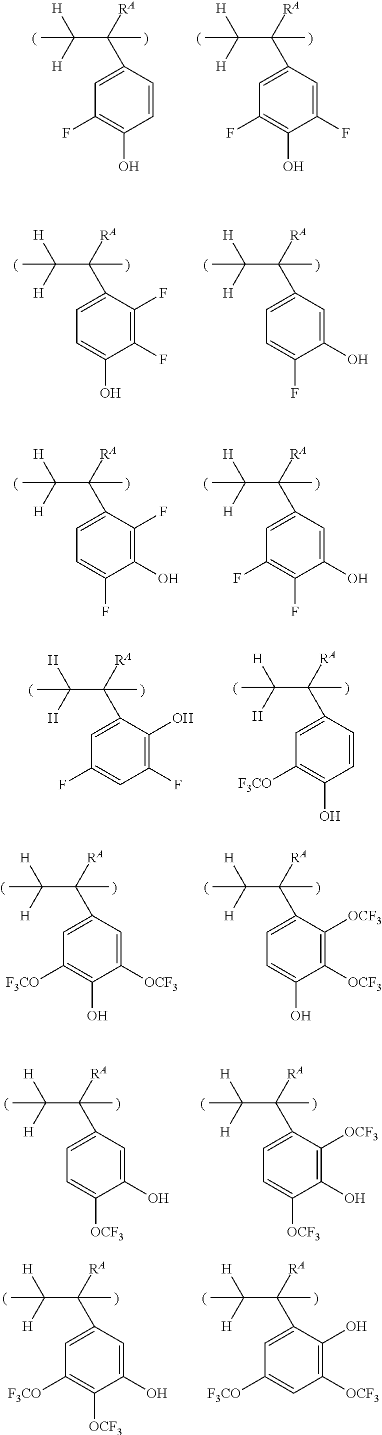

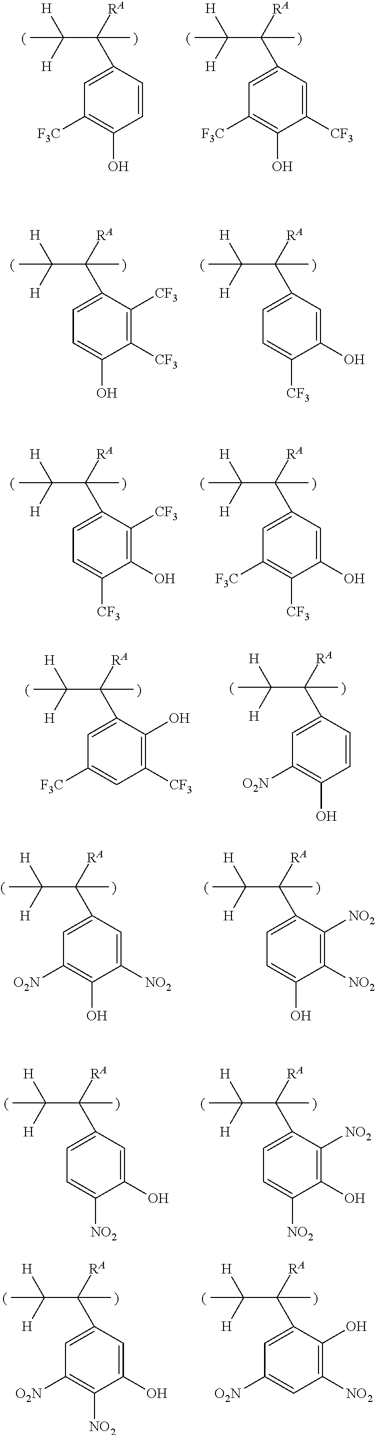

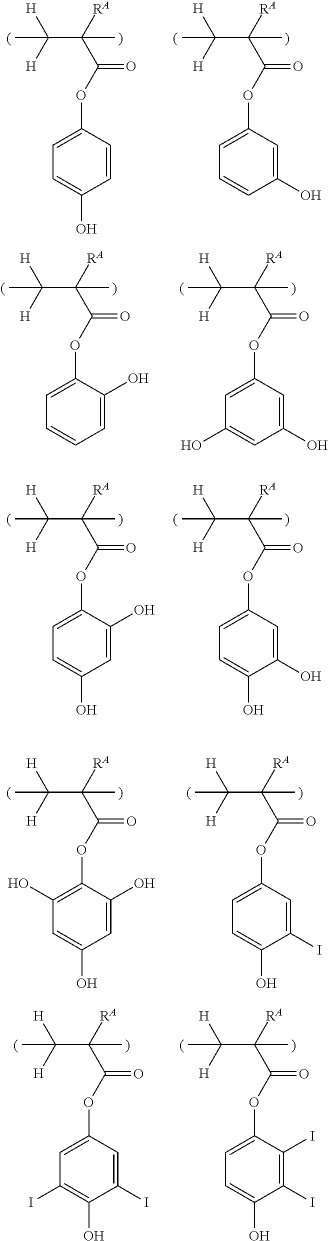

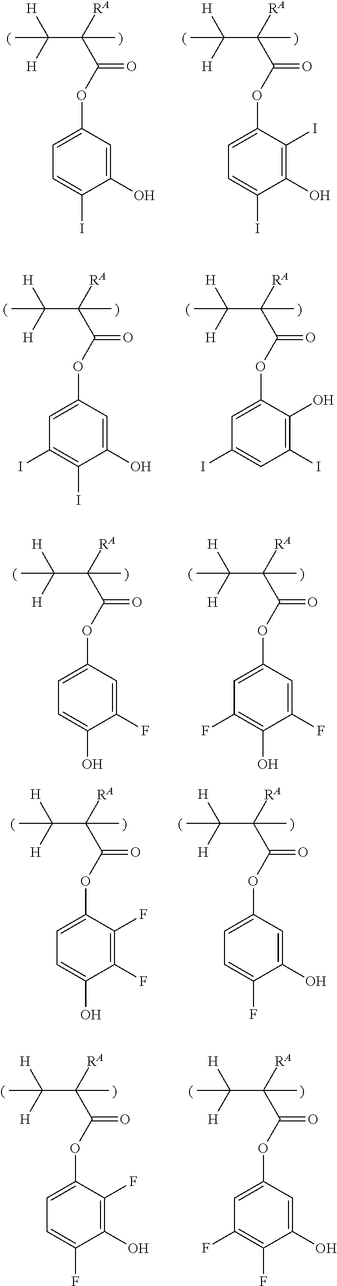

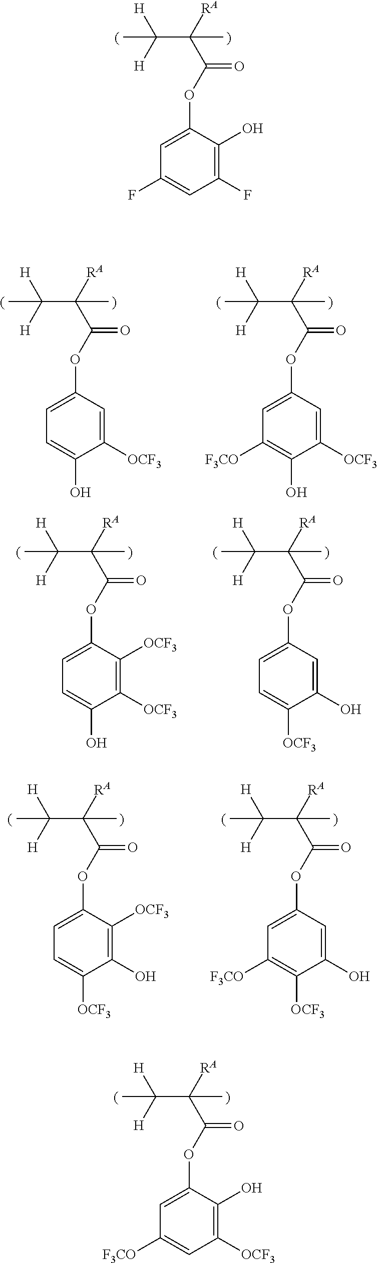

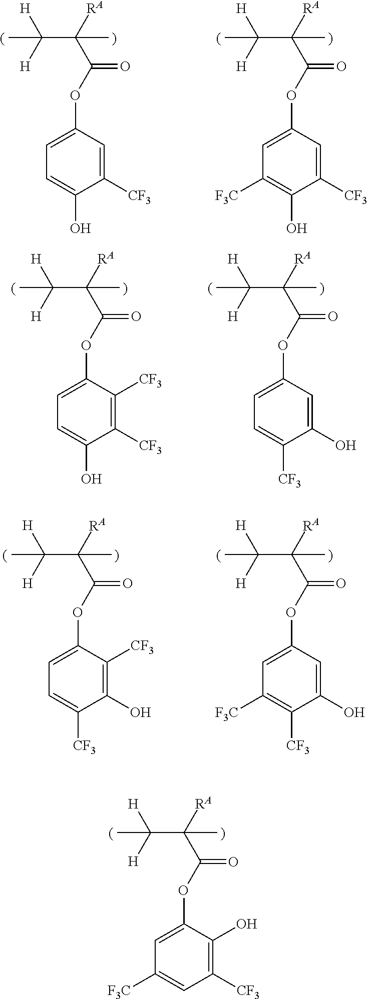

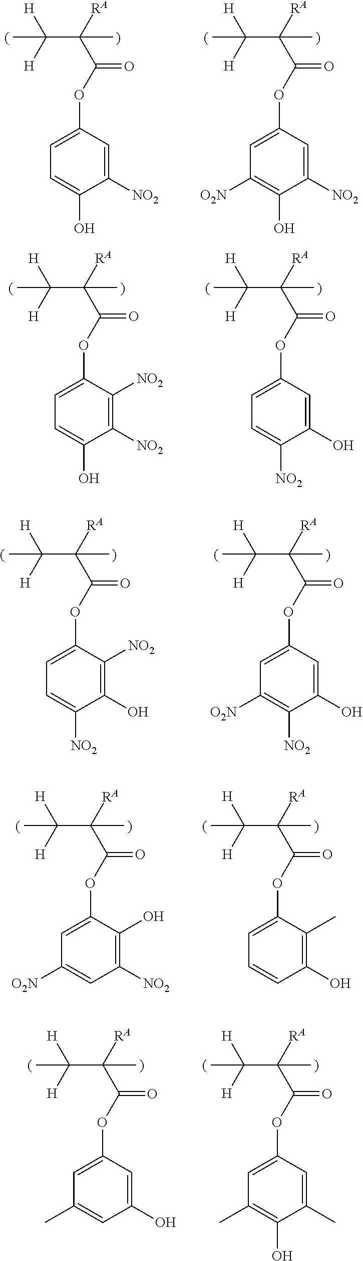

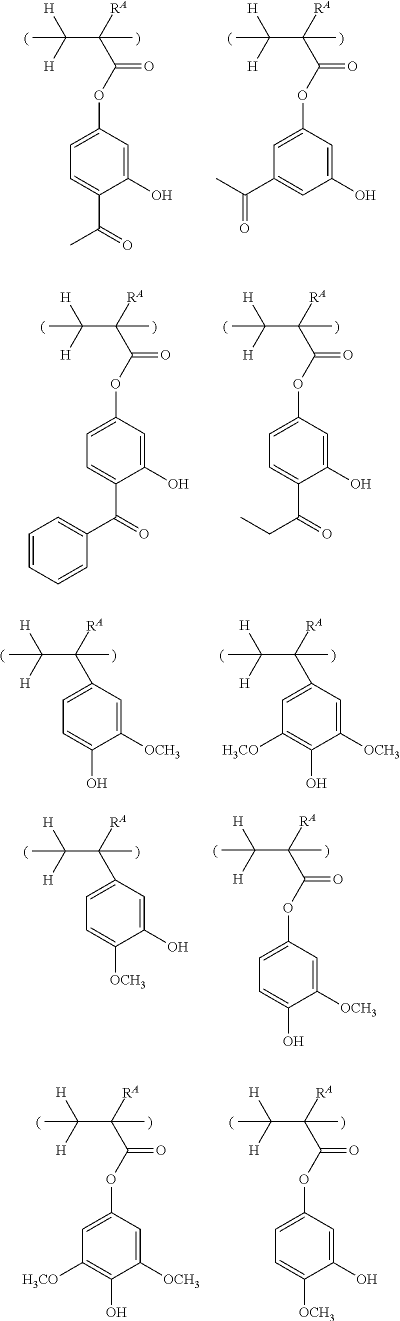

As the repeat units b1 or b2, those units having a lactone ring as a polar group are particularly preferred in the case of ArF lithography, and those units having a phenol site as a polar group are preferred in the case of KrF lithography, EB lithography, and EUV lithography.

The base polymer may further comprise repeat units having any one of the following formulae (c1) to (c4) (also referred to as repeat units c1 to c4, hereinafter).

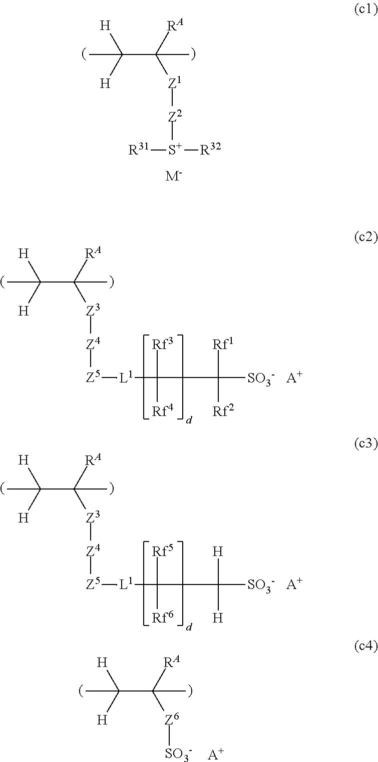

In formulae (c1) to (c4), R^{A} is each independently a hydrogen atom, a fluorine atom, a methyl group, or a trifluoromethyl group. Z¹ is a single bond or a phenylene group. Z² is *—C(═O)—O—Z²¹—, *—C(═O)—NH—Z²¹—, or *—O—Z²¹—. Z²¹ is a C₁-C₆ aliphatic hydrocarbylene group, a phenylene group, or a divalent group obtained by combining the foregoing, which may contain a carbonyl group, an ester bond, an ether bond, or a hydroxy group. Z³ is each independently a single bond, a phenylene group, a naphthylene group, or *—C(═O)—O—Z³¹—. Z³¹ is a C₁-C₁₀ aliphatic hydrocarbylene group, a phenylene group, or a naphthylene group, the aliphatic hydrocarbylene group may contain a hydroxy group, an ether bond, an ester bond, or a lactone ring. Z⁴ is each independently a single bond, *—Z⁴¹—C(═O)—O—, *—C(═O)—NH—Z⁴¹—, or *—O—Z⁴¹—. Z⁴¹ is a C₁-C₂₀ hydrocarbylene group which may contain a heteroatom. Z⁵ is each independently a single bond, *—Z⁵¹—C(═O)—O—, *—C(═O)—NH—Z⁵¹—, or *—O—Z⁵¹—. 751 is a C₁-C₂₀ hydrocarbylene group which may contain a heteroatom. Z⁶ is a single bond, a methylene group, an ethylene group, a phenylene group, a fluorinated phenylene group, a trifluoromethyl group-substituted phenylene group, *—C(═O)—O—Z⁶¹—, * C(═O)—N(H)—Z⁶¹—, or *—O—Z⁶¹—. Z⁶¹ is a C₁-C₆ aliphatic hydrocarbylene group, a phenylene group, a fluorinated phenylene group, or a trifluoromethyl group-substituted phenylene group, which may contain a carbonyl group, an ester bond, an ether bond, or a hydroxy group. * designates a point of attachment to the carbon atom in the backbone.

The aliphatic hydrocarbylene group represented by Z²¹, Z³¹, and Z⁶¹ may be straight, branched, or cyclic, and specific examples thereof include alkanediyl groups such as a methanediyl group, an ethane-1,1-diyl group, an ethane-1,2-diyl group, a propane-1,1-diyl group, a propane-1,2-diyl group, a propane-1,3-diyl group, a propane-2,2-diyl group, a butane-1,1-diyl group, a butane-1,2-diyl group, a butane-1,3-diyl group, a butane-2,3-diyl group, a butane-1,4-diyl group, a 1,1-dimethylethane-1,2-diyl group, a pentane-1,5-diyl group, a 2-methylbutane-1,2-diyl group, and a hexane-1,6-diyl group; cycloalkanediyl groups such as a cyclopropanediyl group, a cyclobutanediyl group, a cyclopentanediyl group, and a cyclohexanediyl group; and groups obtained by combining these.

The hydrocarbylene group represented by Z⁴¹ and Z⁵¹ may be saturated or unsaturated and straight, branched, or cyclic. Specific examples thereof are shown below, but not limited thereto.

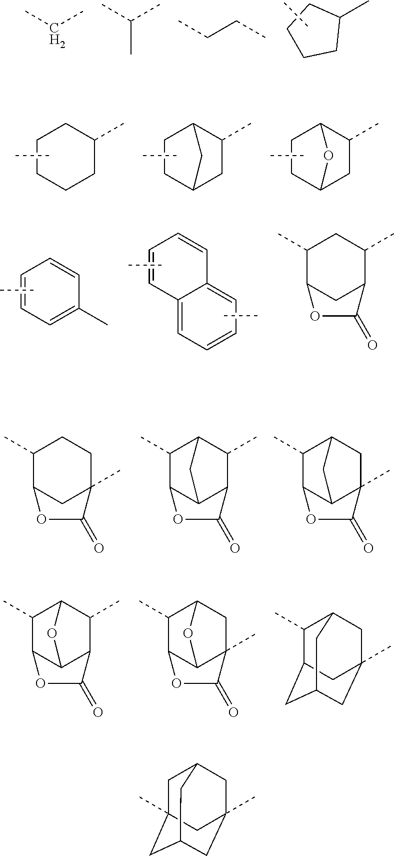

wherein the broken line denotes a point of attachment.

In formula (c1), R³¹ and R³² are each independently a C₁-C₂₀ hydrocarbyl group which may contain a heteroatom. The hydrocarbyl group may be saturated or unsaturated and straight, branched, or cyclic. Specific examples thereof include C₁-C₂₀ alkyl groups such as a methyl group, an ethyl group, a n-propyl group, an isopropyl group, a n-butyl group, and a tert-butyl group; C₃-C₂₀ cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₂₀ alkenyl groups such as a vinyl group, an allyl group, a propenyl group, a butenyl group, and a hexenyl group; C₃-C₂₀ cyclic unsaturated hydrocarbyl groups such as a cyclohexenyl group; C₆-C₂₀ aryl groups such as a phenyl group, a naphthyl group, and a thienyl group; aralkyl groups such as a benzyl group, a 1-phenylethyl group, and a 2-phenylethyl group; and groups obtained by combining these, and an aryl group is preferable. Some or all of the hydrogen atoms in the hydrocarbyl group may be substituted with a group containing a heteroatom such as an oxygen atom, a sulfur atom, a nitrogen atom, or a halogen atom, some of —CH₂— in the hydrocarbyl group may be substituted with a group containing a heteroatom such as an oxygen atom, a sulfur atom, or a nitrogen atom, so that the group may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, or the like.

R³¹ and R³² may bond together to form a ring with the sulfur atom to which they are attached. In this case, examples of the ring are as exemplified above for the ring that R¹¹ and R¹² in the description of formula (cation-1) may bond together to form with the sulfur atom to which they are attached.

Specific examples of the cation of the repeat units c1 are shown below, but not limited thereto. In the following formula, R^{A} is as defined above.

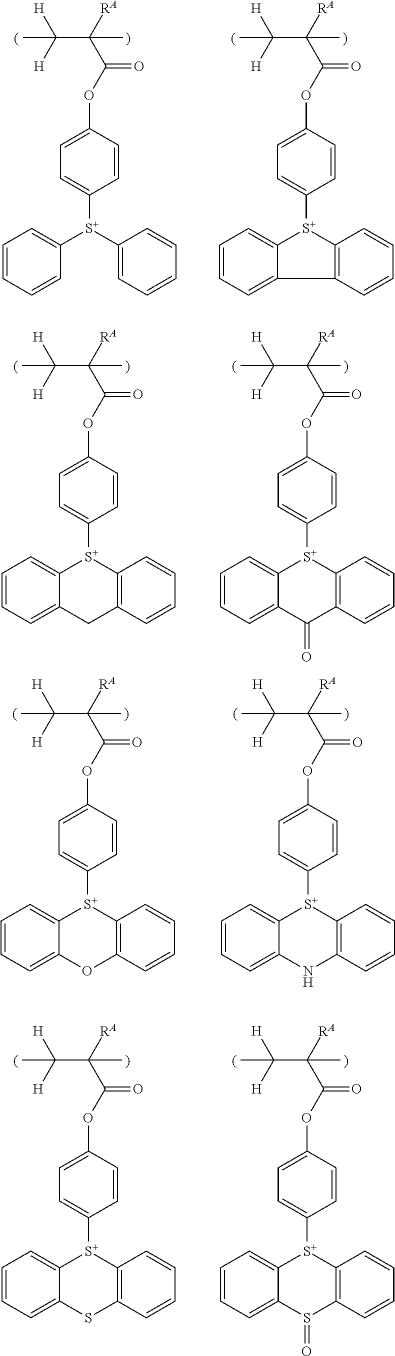

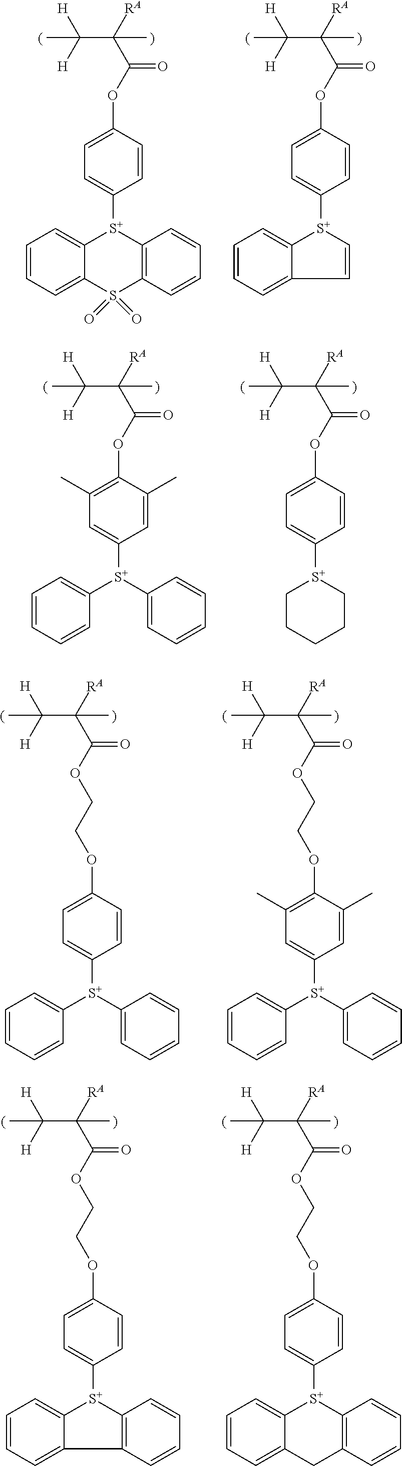

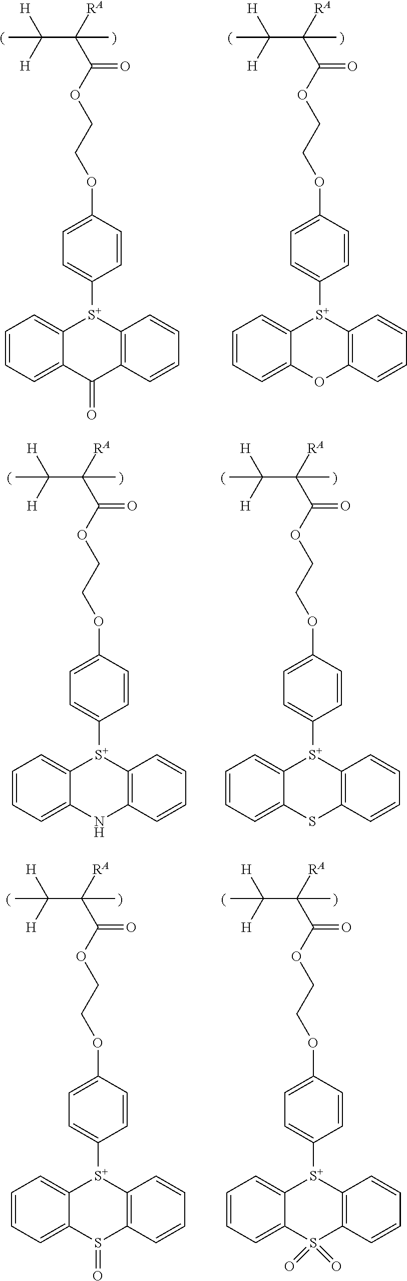

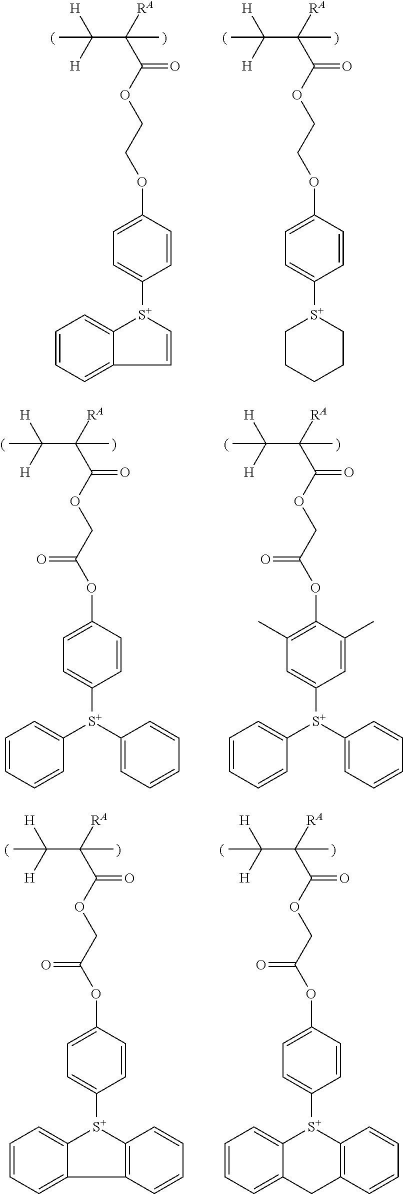

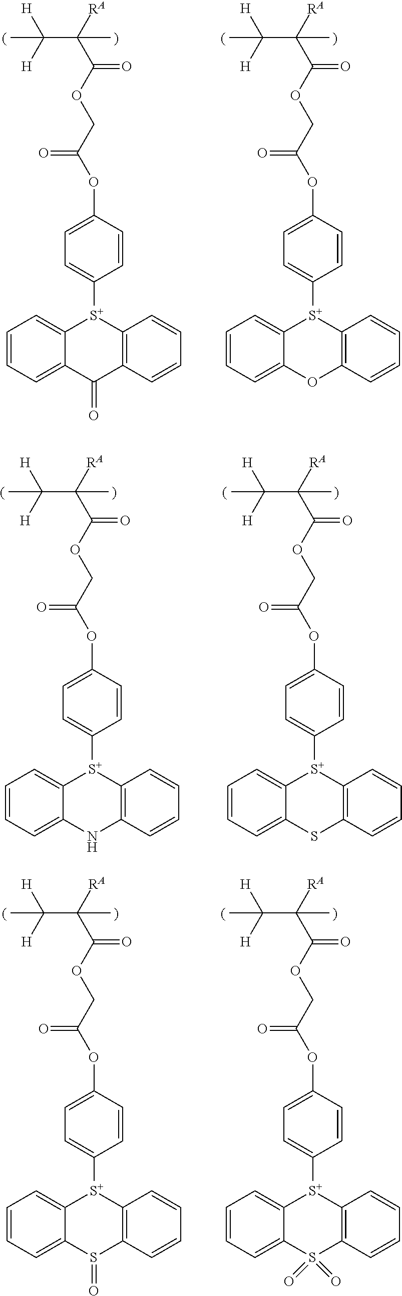

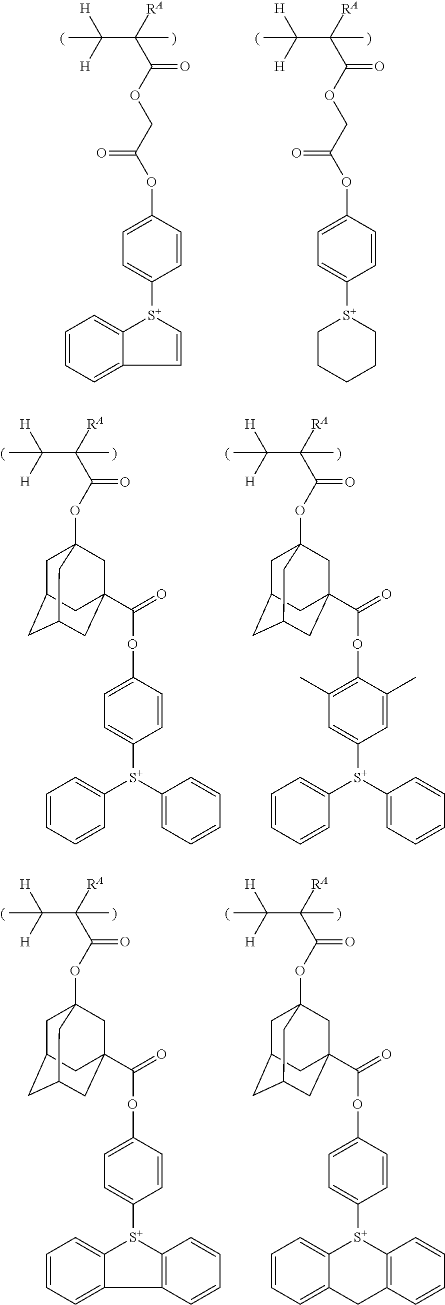

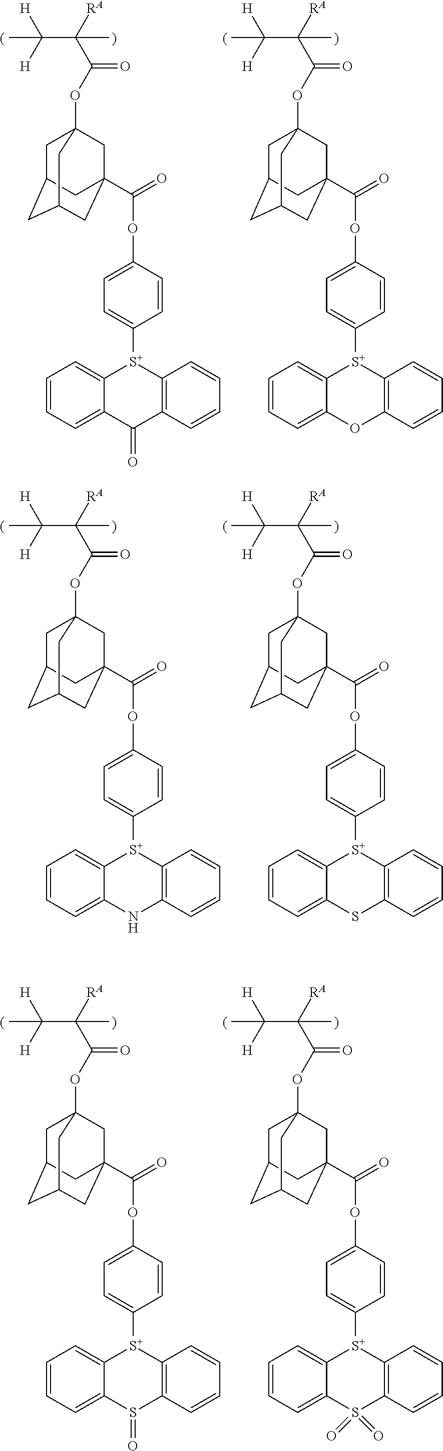

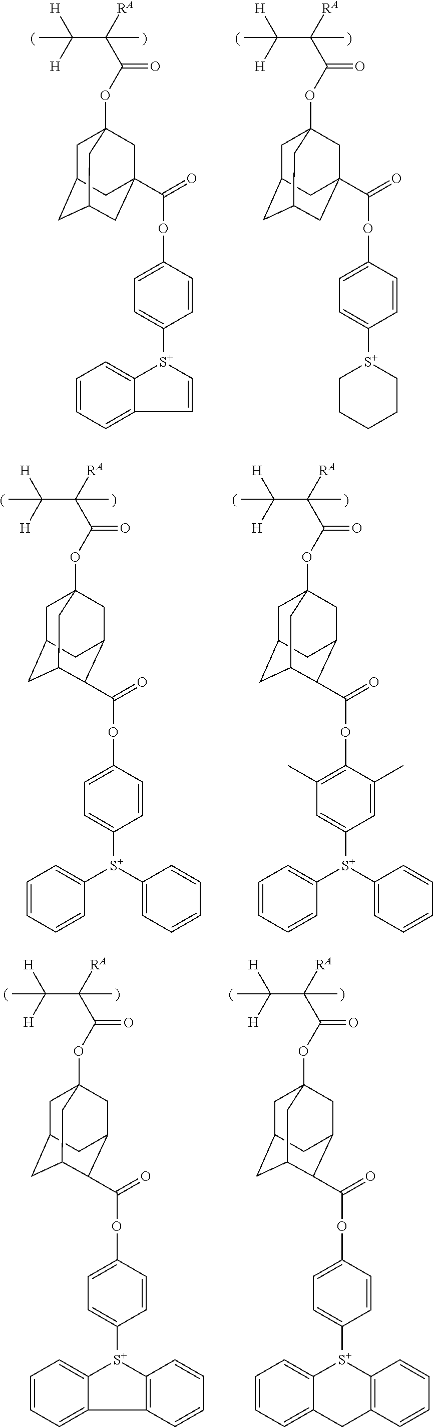

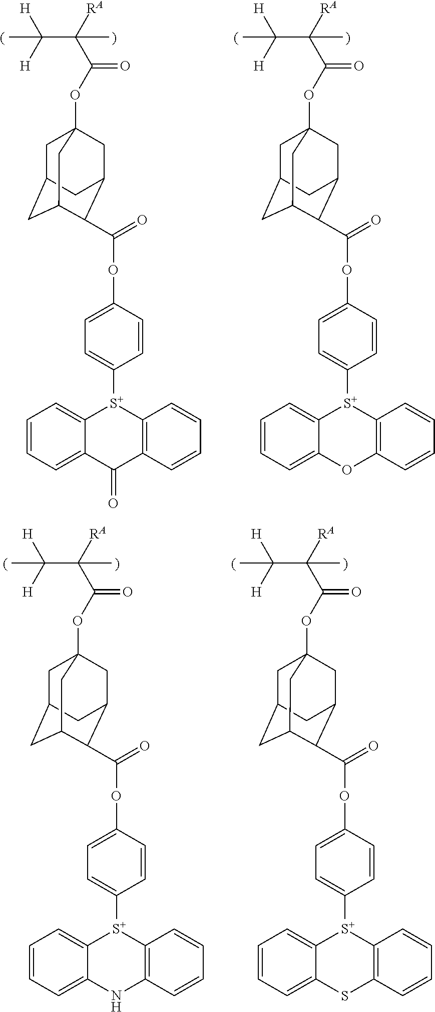

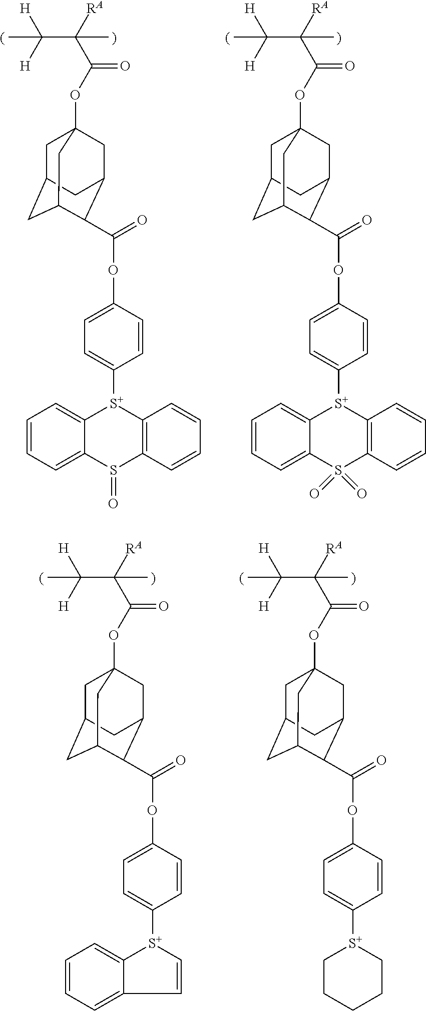

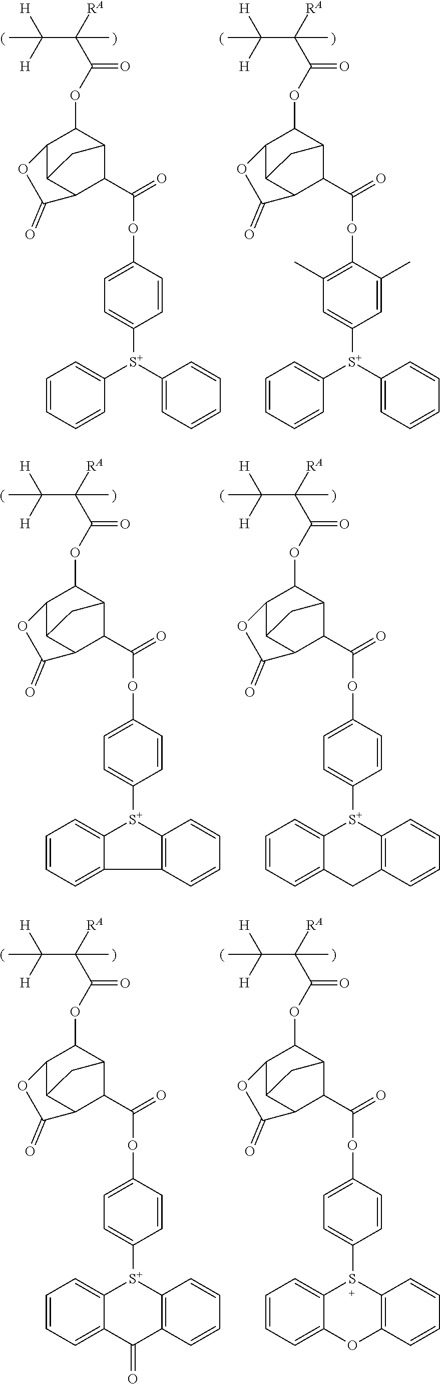

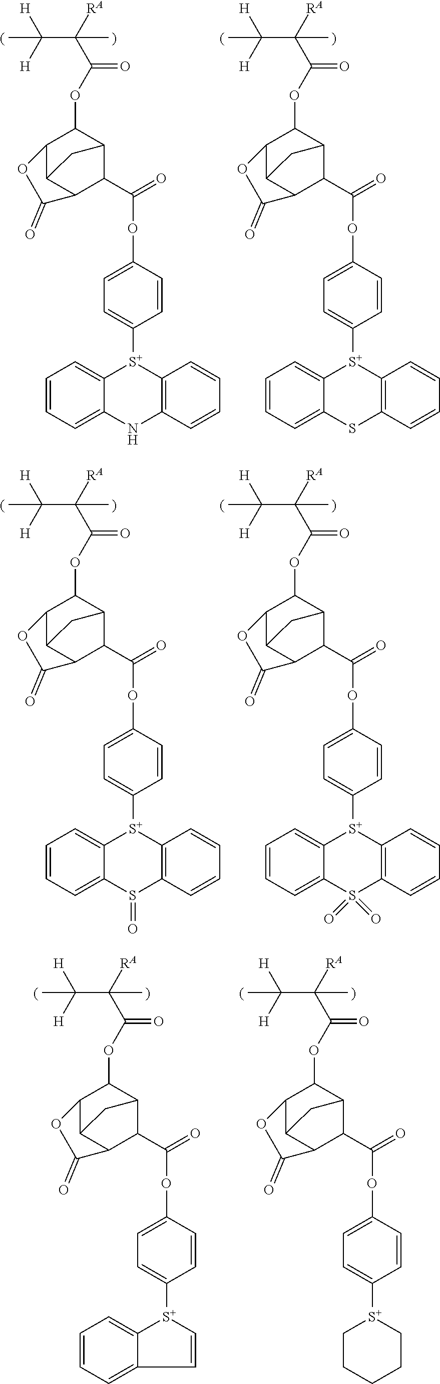

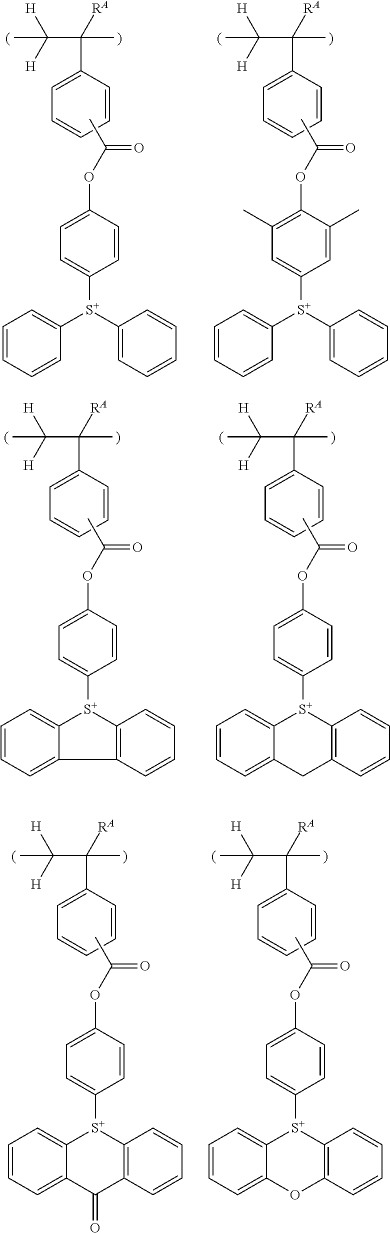

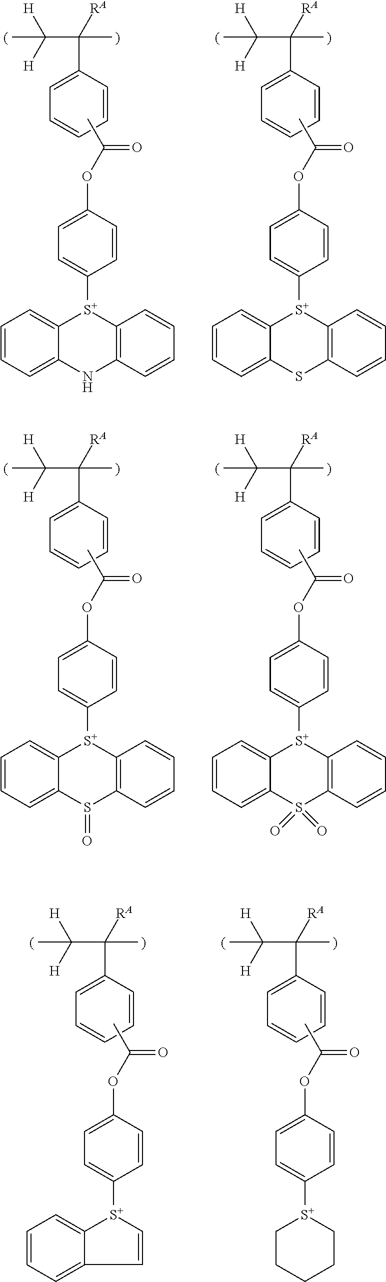

In formula (c1), M⁻ is a non-nucleophilic counter ion. Specific examples of the non-nucleophilic counter ion include halide ions such as chloride ions and bromide ions; fluoroalkylsulfonate ions such as triflate ions, 1,1,1-trifluoroethanesulfonate ions, and nonafluorobutanesulfonate ions; arylsulfonate ions such as tosylate ions, benzenesulfonate ions, 4-fluorobenzenesulfonate ions, and 1,2,3,4,5-pentafluorobenzenesulfonate ions; alkylsulfonate ions such as mesylate ions and butanesulfonate ions; imide ions such as bis(trifluoromethylsulfonyl) imide ions, bis(perfluoroethylsulfonyl) imide ions, and bis(perfluorobutylsulfonyl) imide ions; and methide ions such as tris(trifluoromethylsulfonyl) methide ions and tris(perfluoroethylsulfonyl) methide ions.

Other specific examples of the non-nucleophilic counter ion include sulfonate anions having fluorine substituted at α-position as represented by the following formula (c1-1) and sulfonate anions having fluorine substituted at α-position and trifluoromethyl at β-position as represented by the following formula (c1-2).

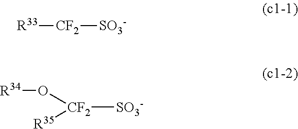

In formula (c1-1), R³³ is a hydrogen atom, a C₁-C₃₀ hydrocarbyl group, a C₂-C₃₀ hydrocarbylcarbonyloxy group, or a C₂-C₃₀ hydrocarbyloxycarbonyl group, and the hydrocarbyl group may contain, a halogen atom, an ether bond, an ester bond, a carbonyl group, or a lactone ring. The hydrocarbyl moiety of the hydrocarbyl group, the hydrocarbylcarbonyloxy group, and the hydrocarbyloxycarbonyl group may be saturated or unsaturated and straight, branched, or cyclic. Specific examples thereof are as exemplified above for the hydrocarbyl group represented by R^{fa1} in formula (2A′) described below.

In formula (c1-2), R³⁴ is a hydrogen atom, a C₁-C₃₀ hydrocarbyl group, or a C₂-C₃₀ hydrocarbylcarbonyl group, and the hydrocarbyl group and hydrocarbylcarbonyl group may contain a halogen atom, an ether bond, an ester bond, a carbonyl group, or a lactone ring. R³⁵ is a hydrogen atom, a fluorine atom, or a C₁-C₆ fluorinated alkyl group. The hydrocarbyl moiety of the hydrocarbyl group and the hydrocarbylcarbonyl group may be saturated or unsaturated and straight, branched, or cyclic. Specific examples thereof are as exemplified above for the hydrocarbyl group represented by R^{fa1} in formula (2A′) described below. R³⁵ is preferably a trifluoromethyl group.

Specific examples of the sulfonate anion having formula (c1-1) or (c1-2) are shown below, but not limited thereto. In the following formula, R³⁵ is as defined above, and Ac is an acetyl group.

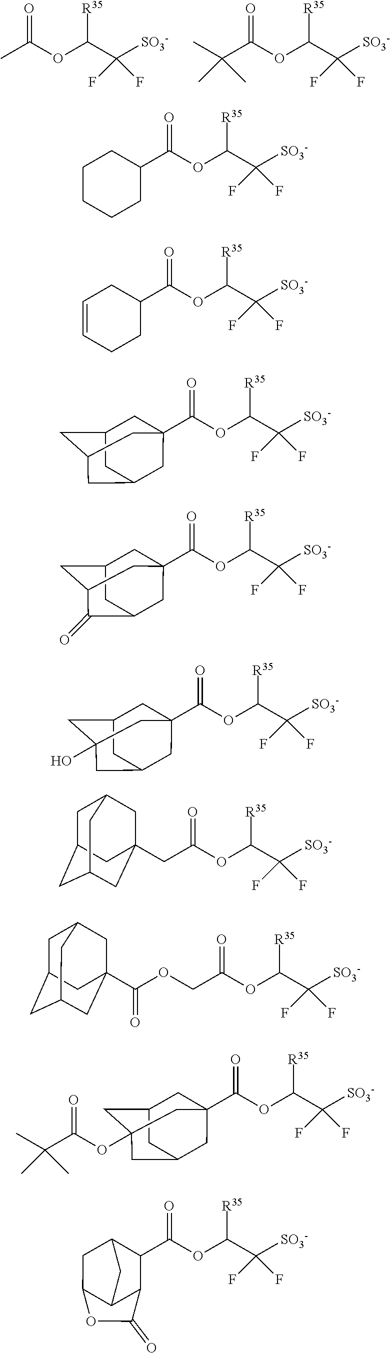

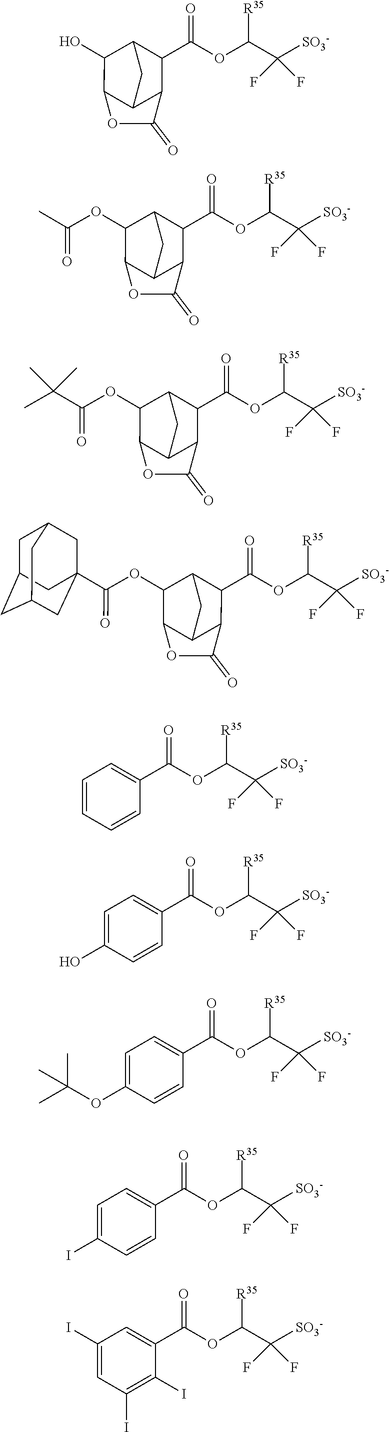

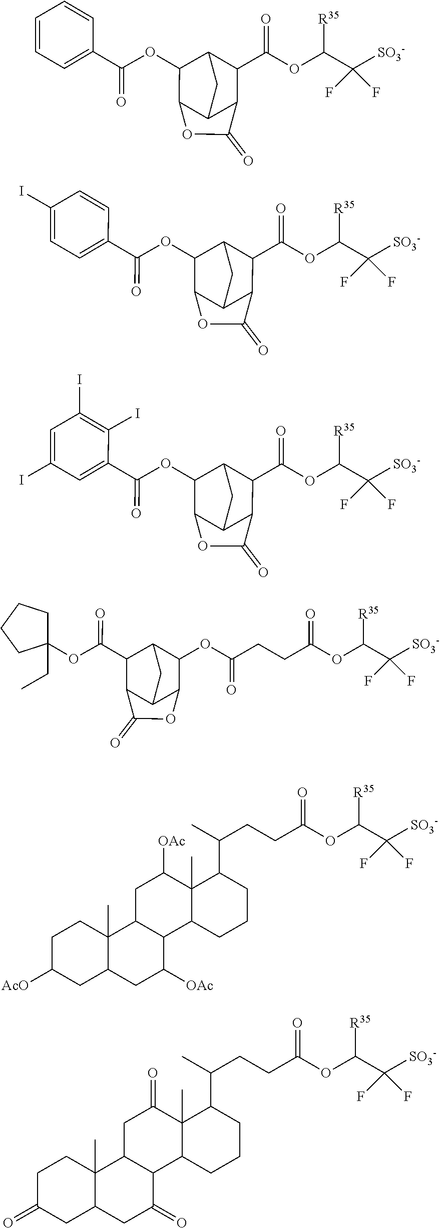

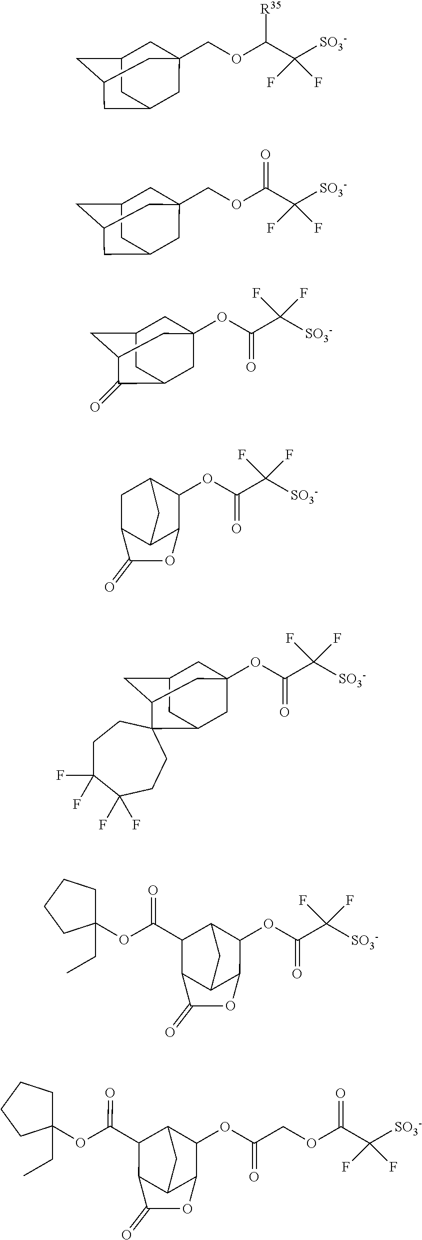

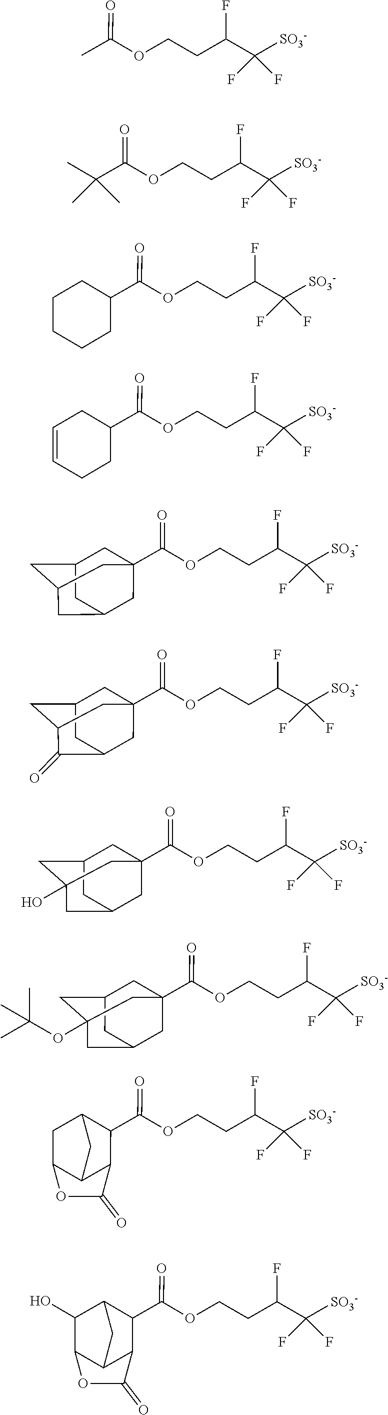

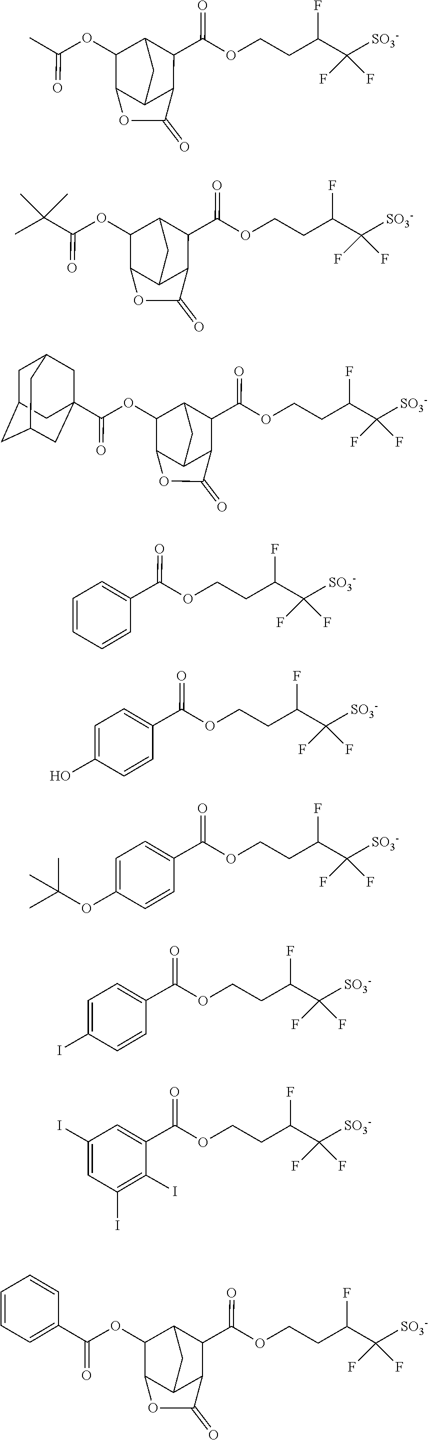

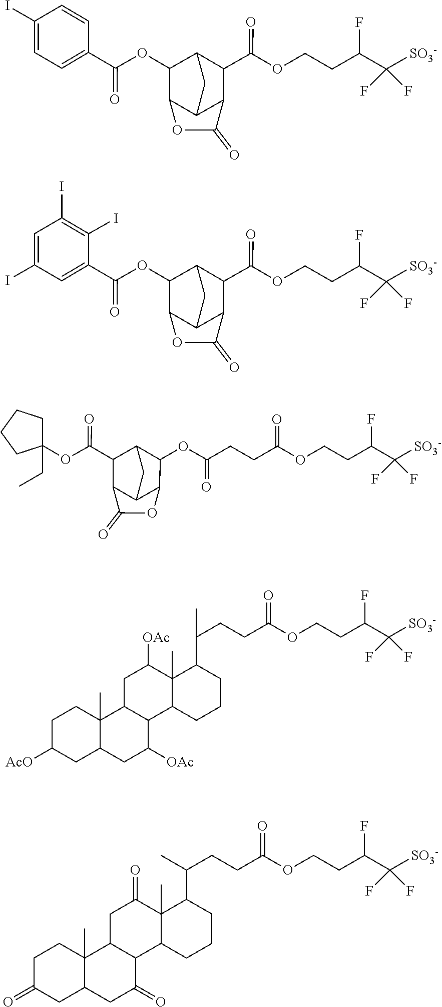

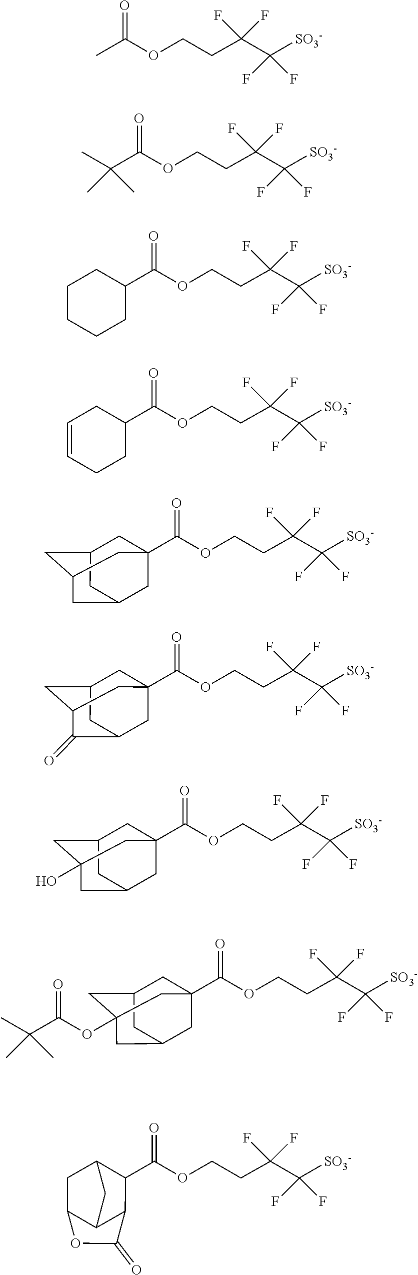

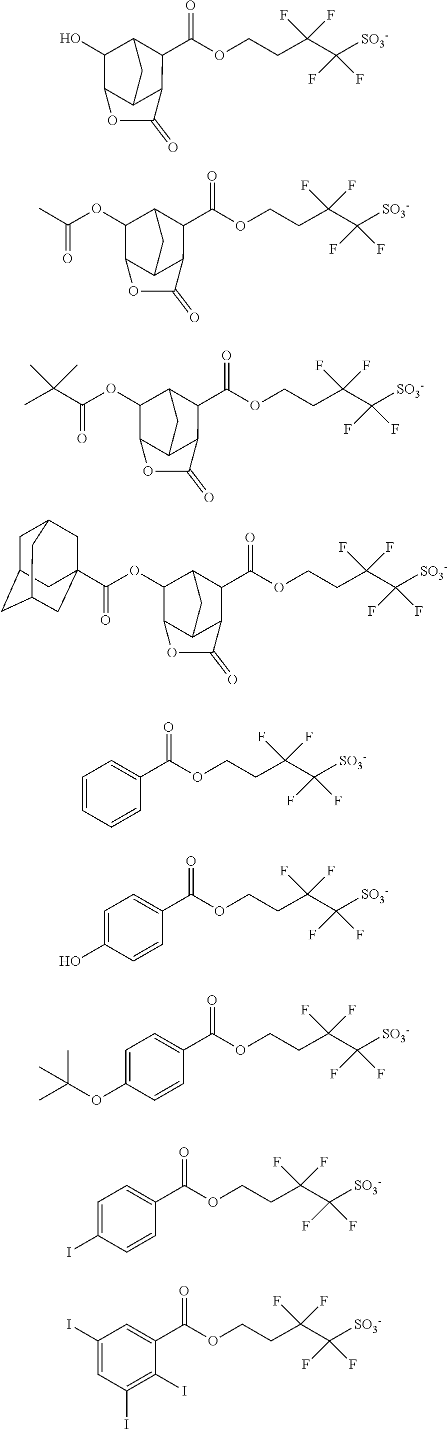

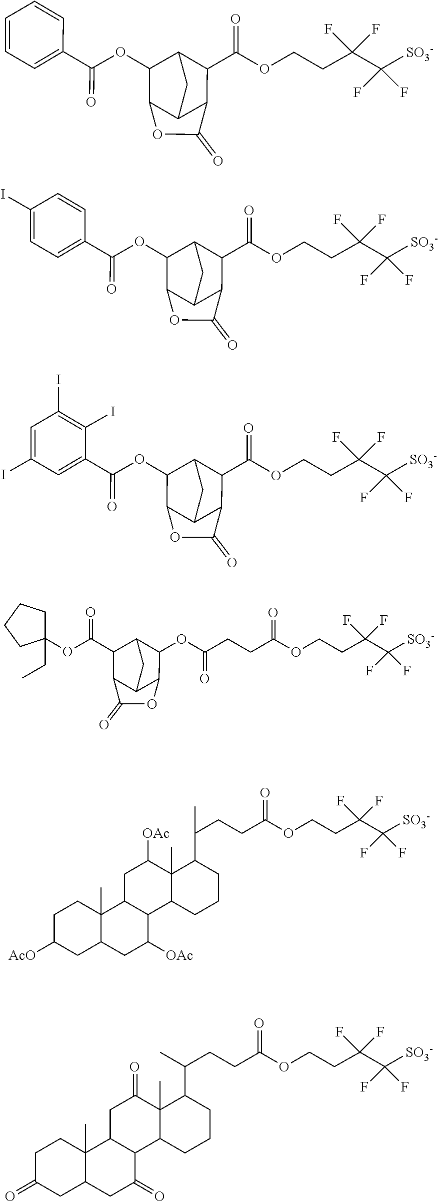

In formulae (C₂) and (C₃). L′ is a single bond, an ether bond, an ester bond, a carbonyl group, a sulfonic acid ester bond, a carbonate bond, or a carbamate bond. Of these, from the viewpoint of synthesis, an ether bond, an ester bond, and a carbonyl group are preferable, and an ester bond and a carbonyl group are more preferable.

In formula (C₂), Rf¹ and Rf² are each independently a fluorine atom or a C₁-C₆ fluorinated saturated hydrocarbyl group. Of these, Rf¹ and Rf² are each preferably a fluorine atom in order to increase the acid strength of the generated acid. Rf³ and Rf⁴ are each independently a hydrogen atom, a fluorine atom, or a C₁-C₆ fluorinated saturated hydrocarbyl group. Of these, at least one of Rf³ and Rf⁴ is preferably a trifluoromethyl group for improving solvent solubility.

In formula (C₃), Rf⁵ and Rf⁶ are each independently a hydrogen atom, a fluorine atom, or a C₁-C₆ fluorinated saturated hydrocarbyl group. Provided that all Rf⁵ and Rf⁶ are not a hydrogen atom at the same time. Of these, at least one of Rf⁵ and Rf⁶ is preferably a trifluoromethyl group for improving solvent solubility.

In formulae (C₂) and (C₃), d is an integer of 0 to 3, preferably 1.

Specific examples of the anion of the repeat units c2 are shown below, but not limited thereto. In the following formula, R^{A} is as defined above.

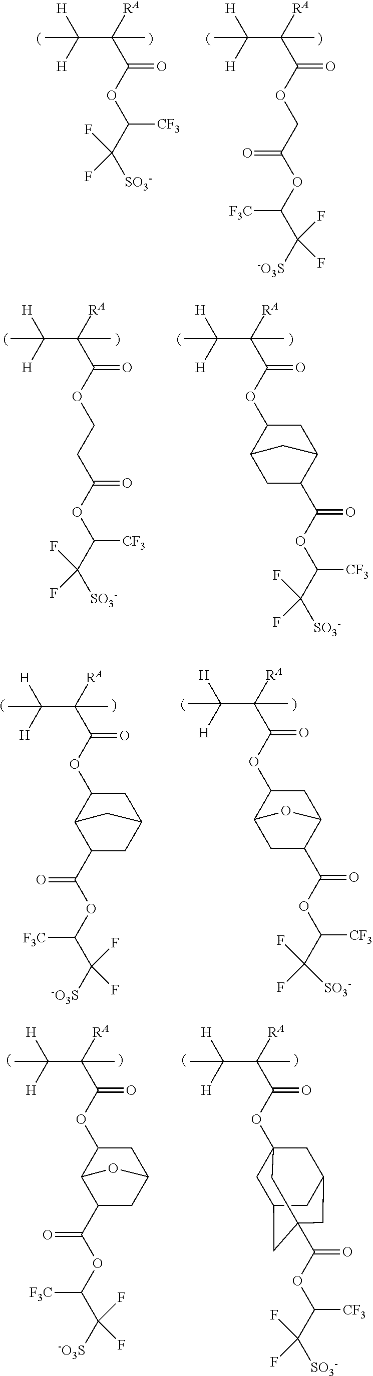

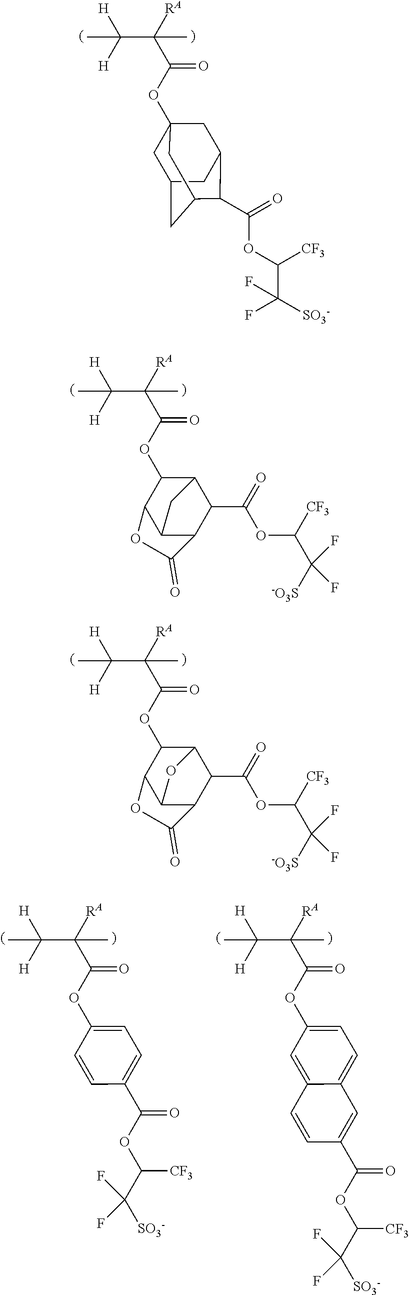

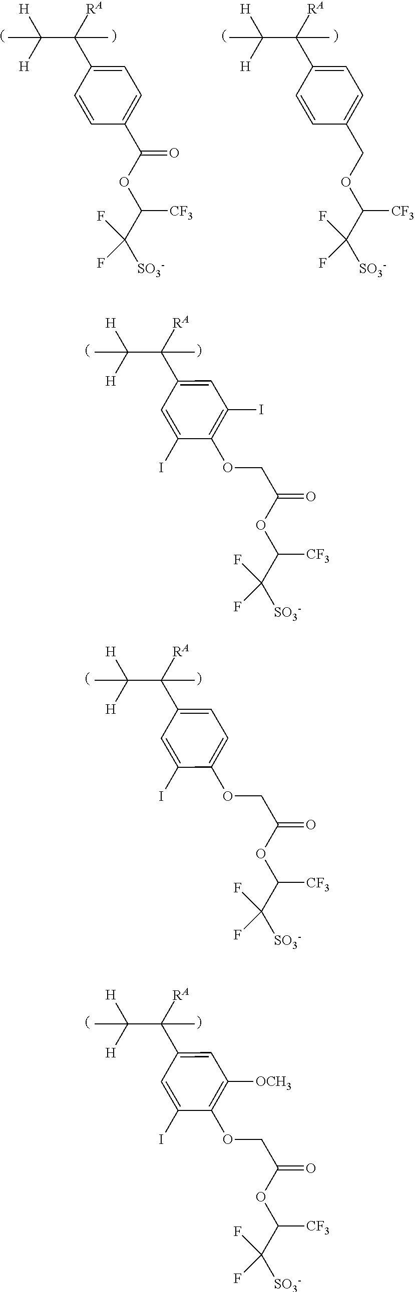

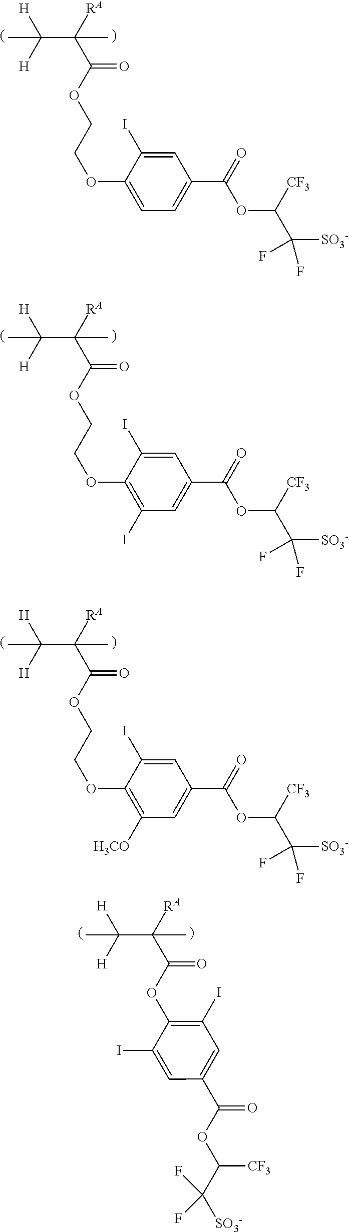

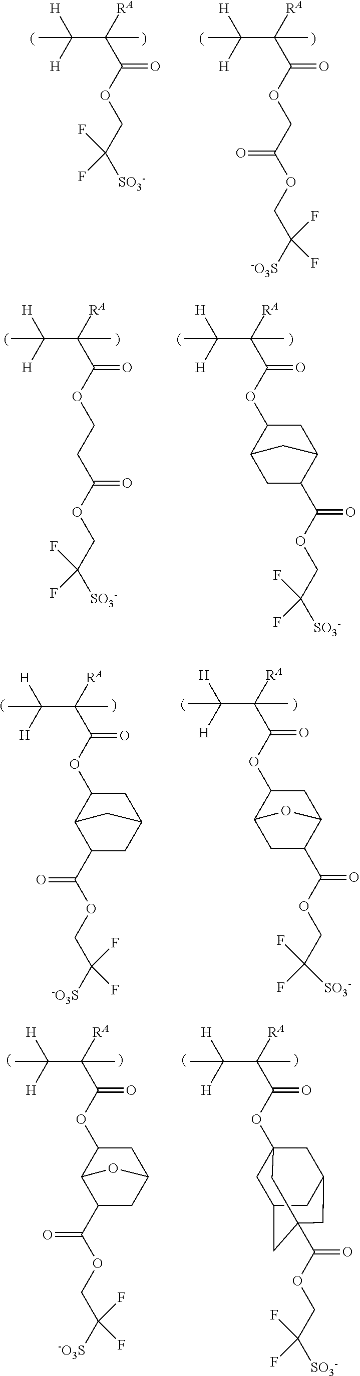

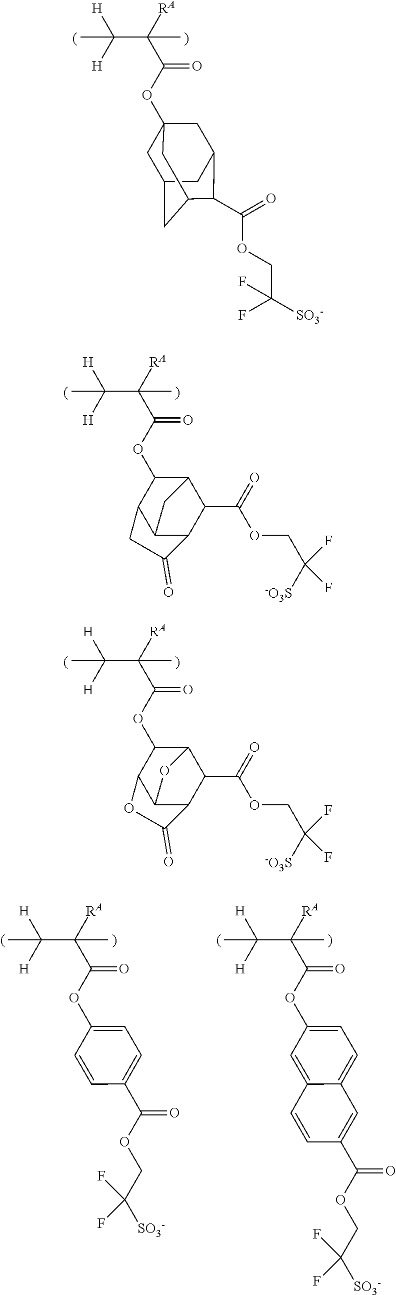

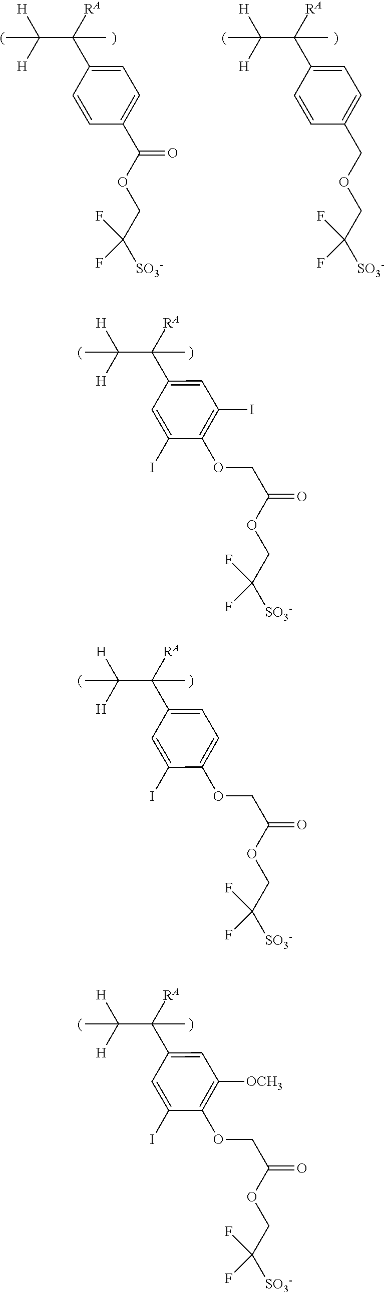

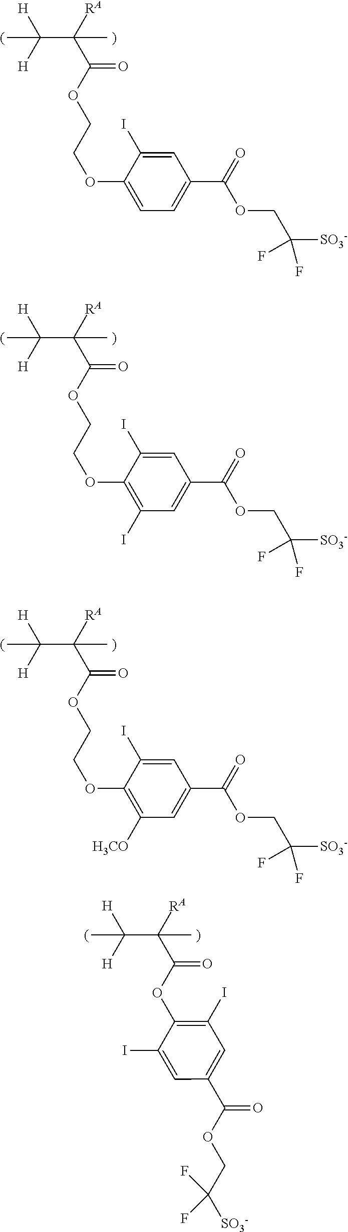

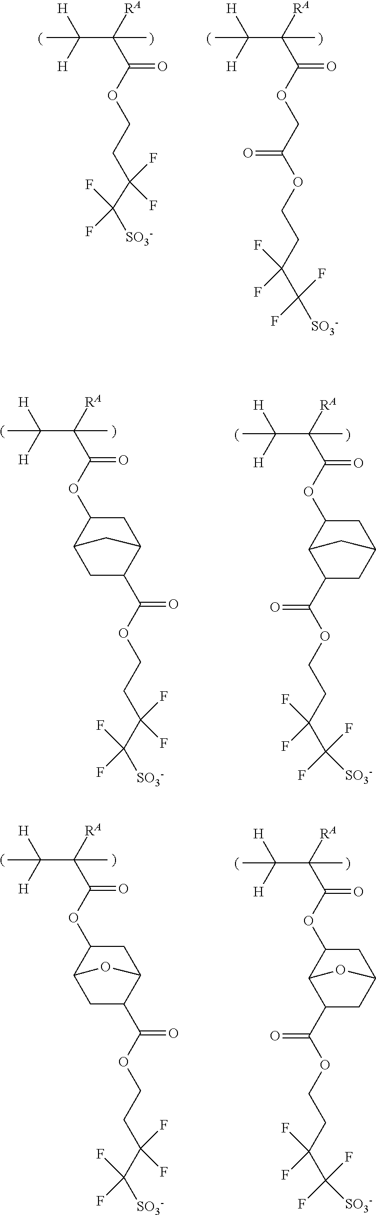

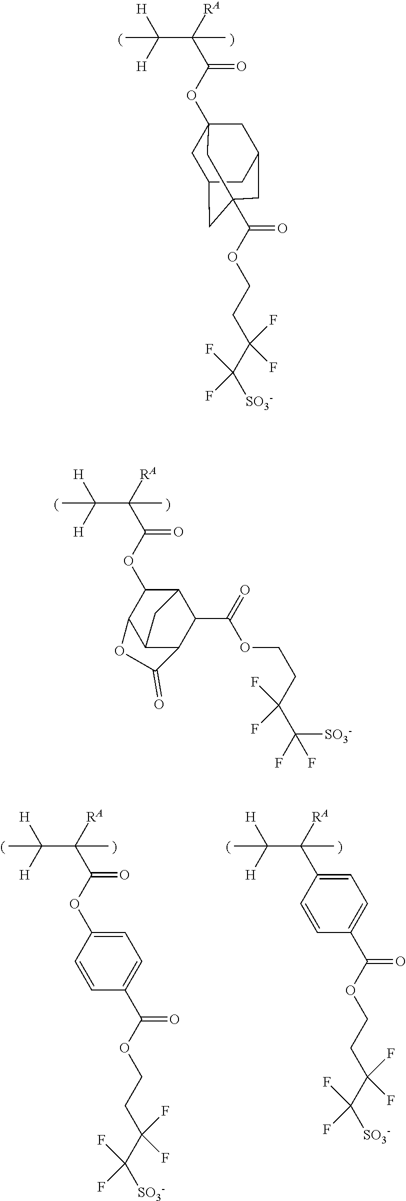

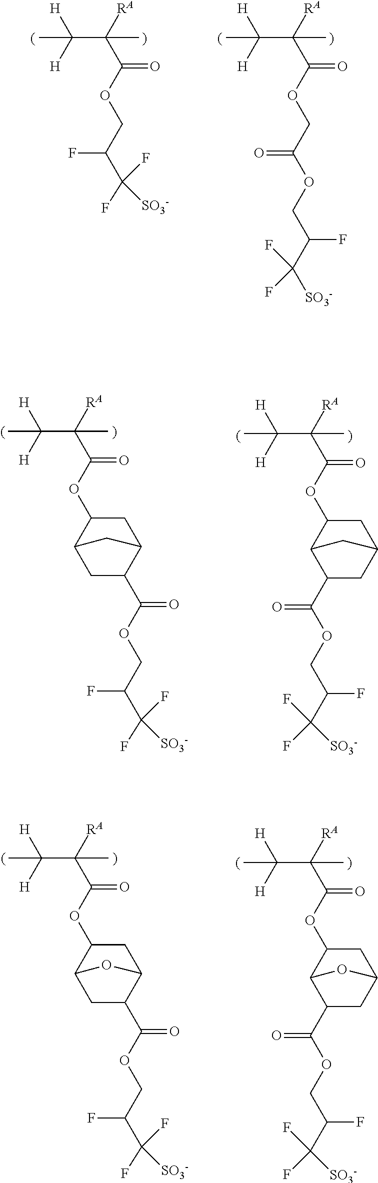

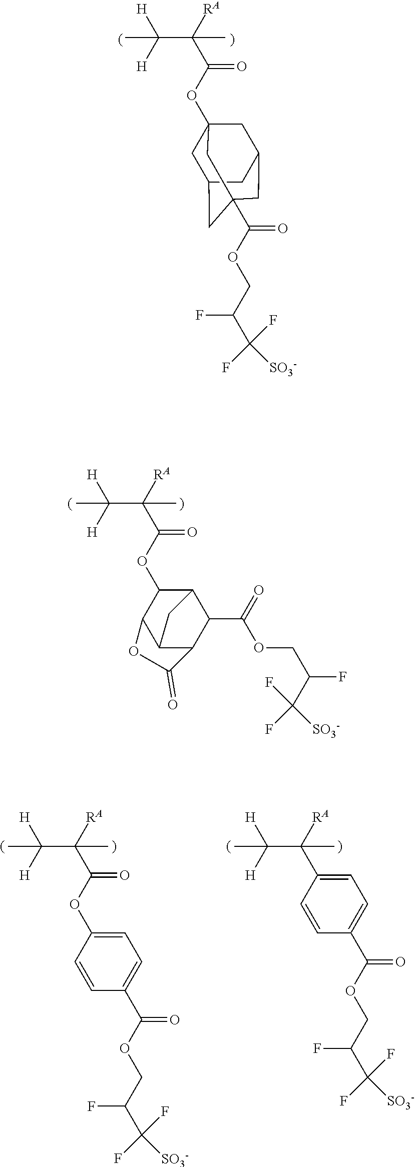

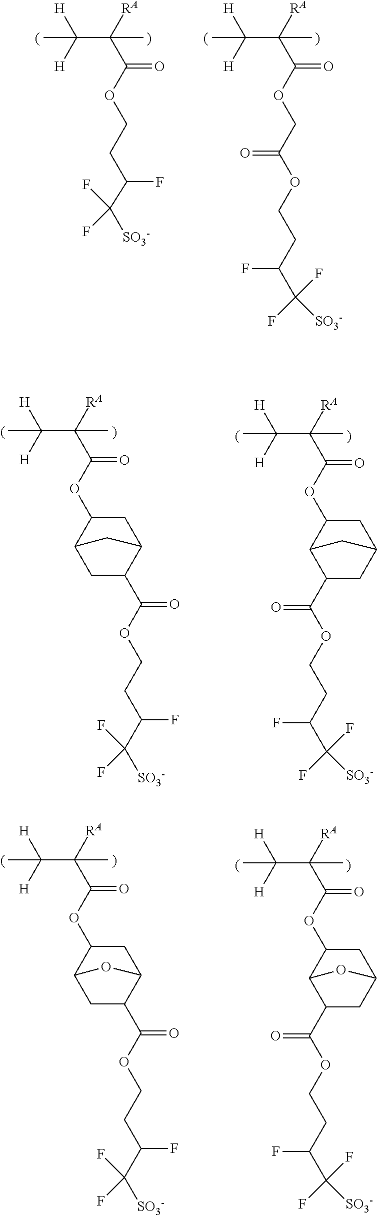

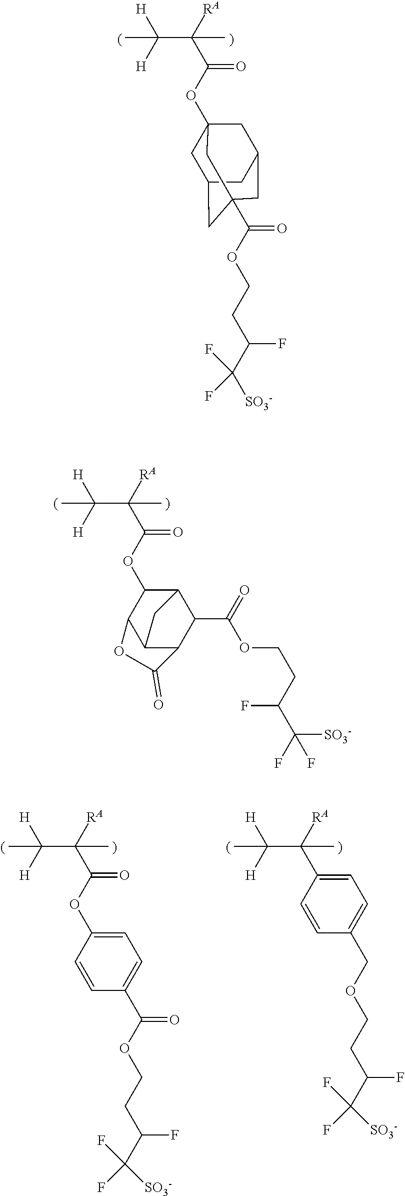

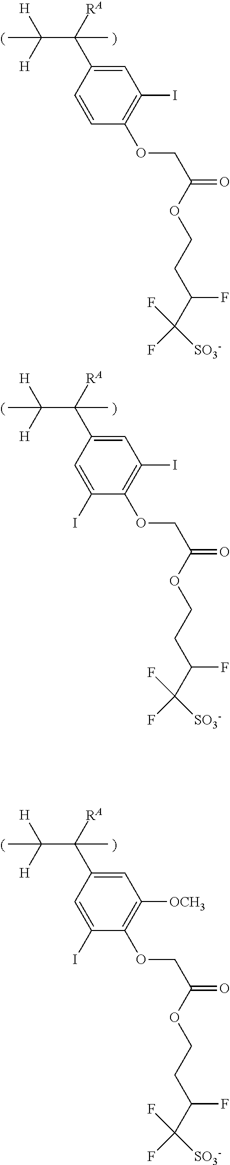

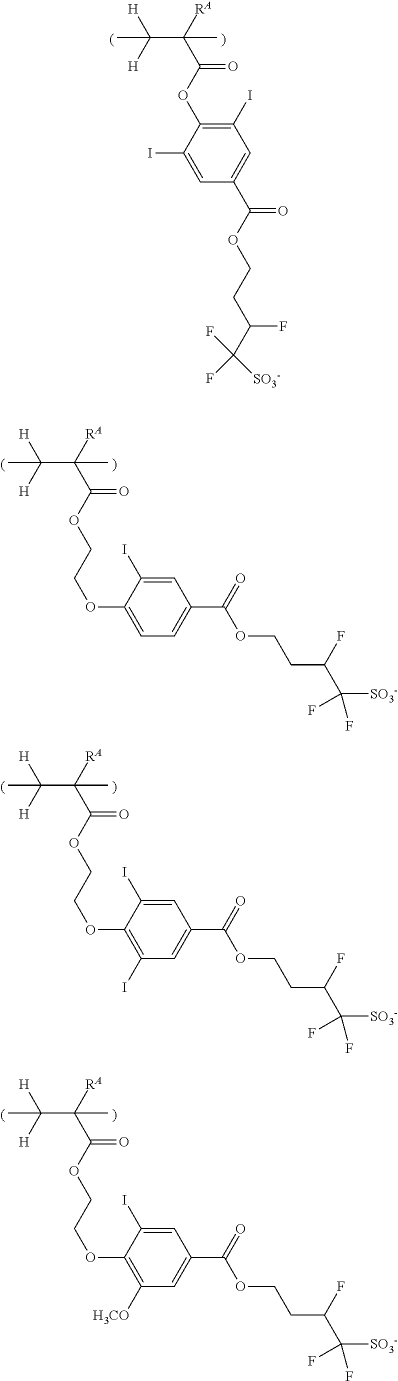

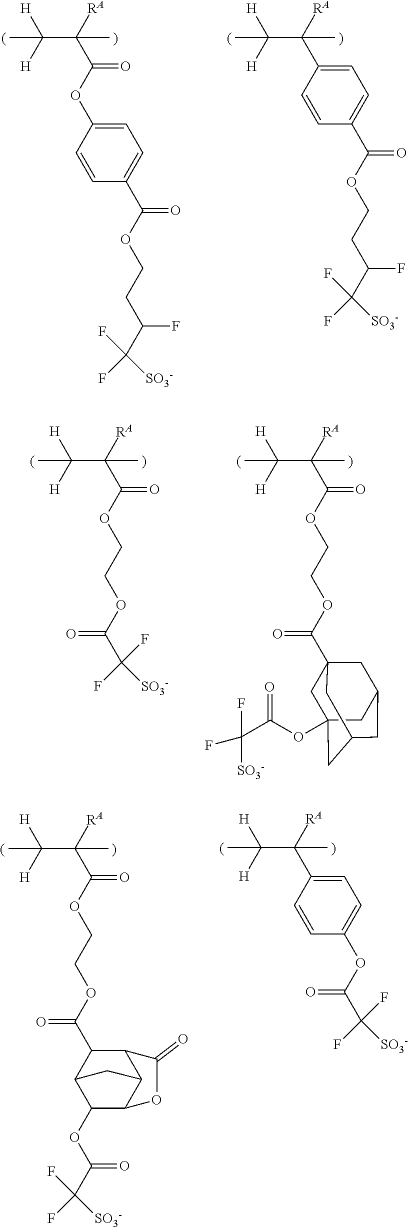

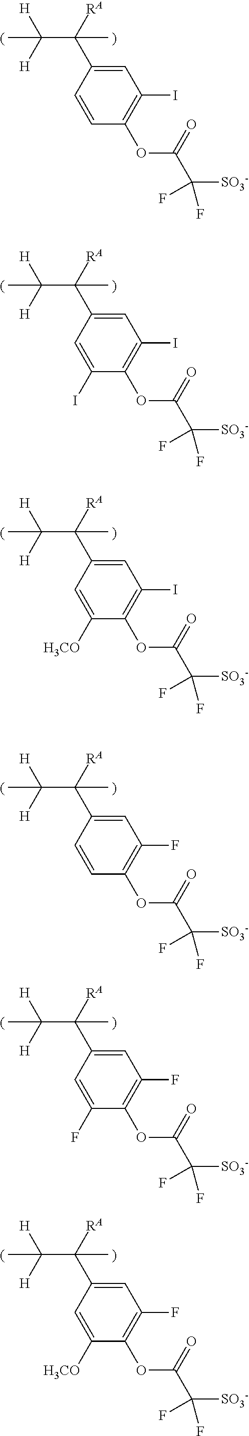

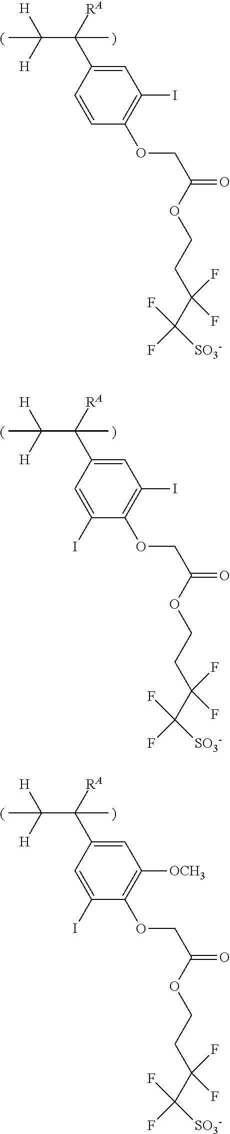

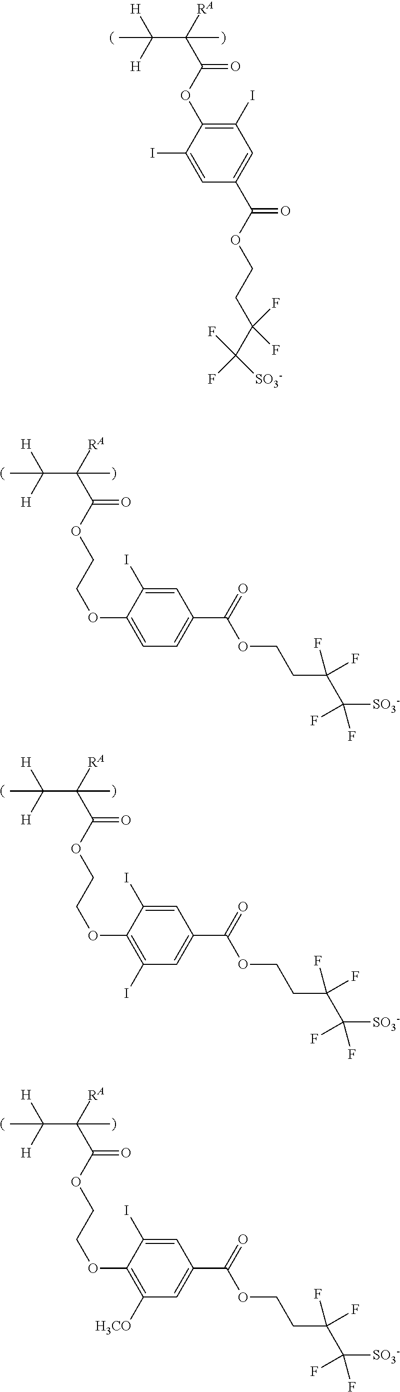

Specific examples of the anion of the repeat units c3 are shown below, but not limited thereto. In the following formula. R^{A} is as defined above.

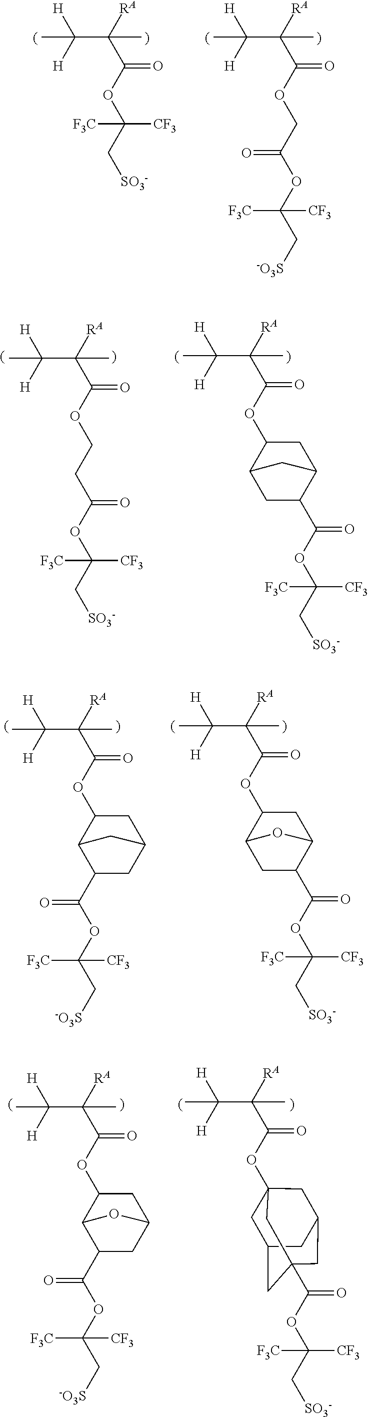

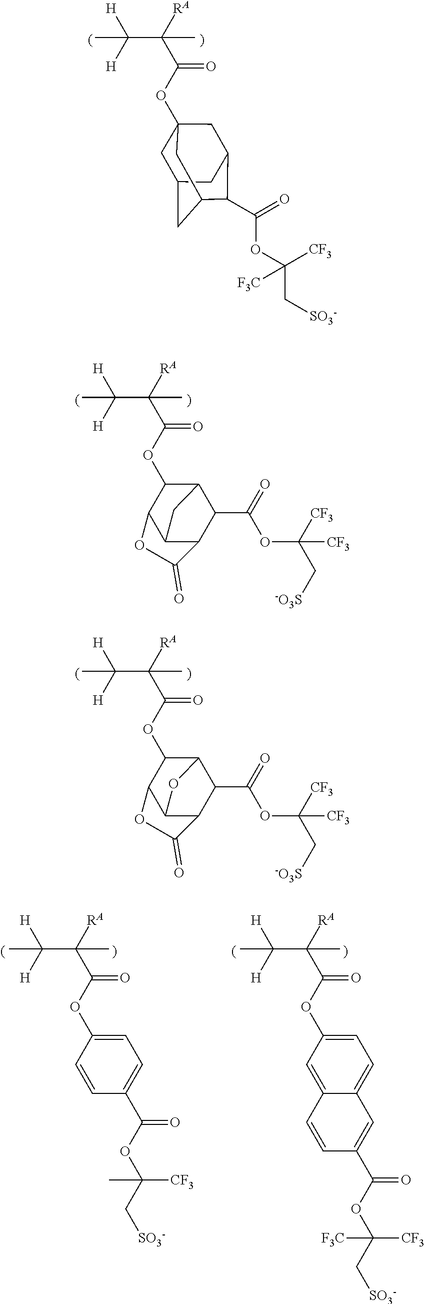

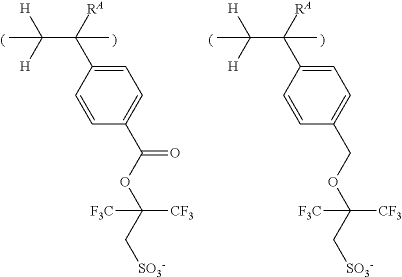

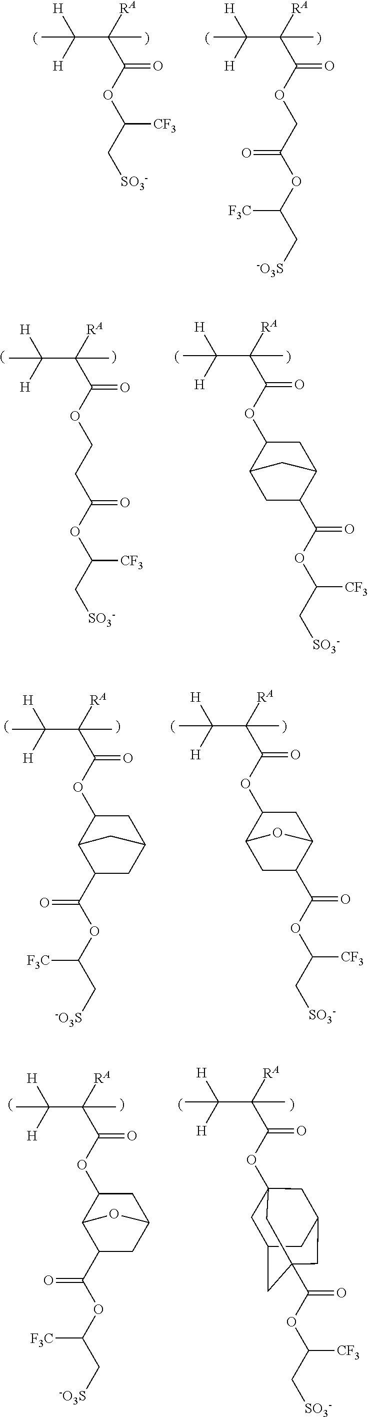

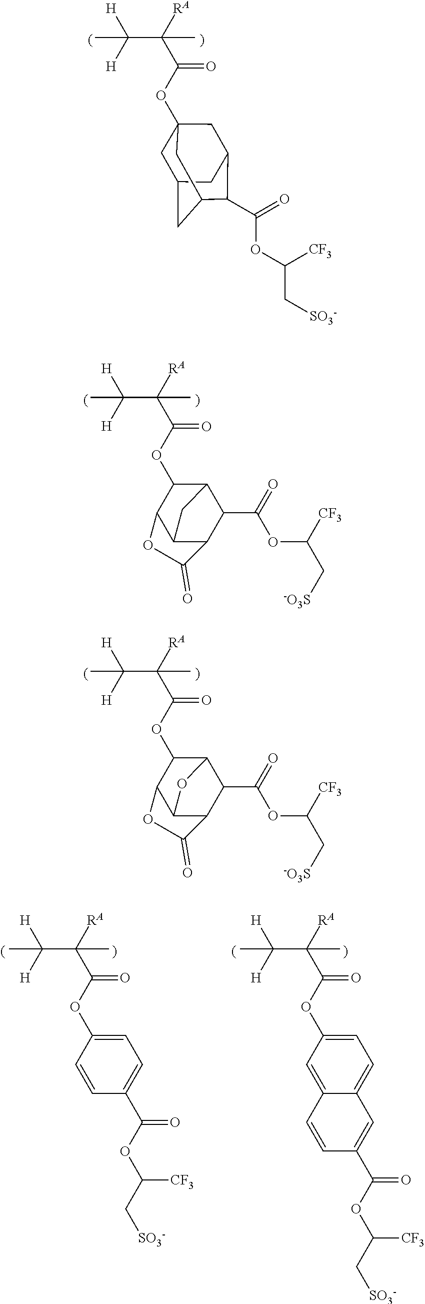

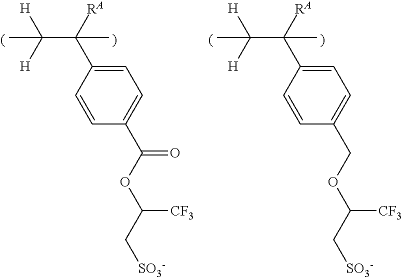

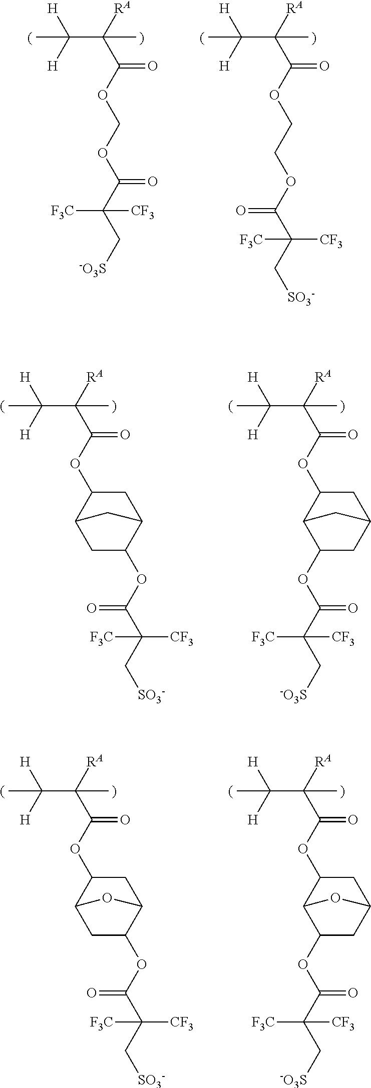

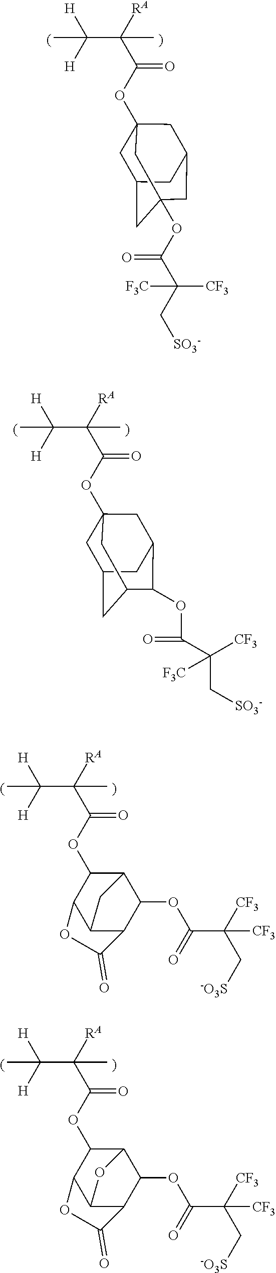

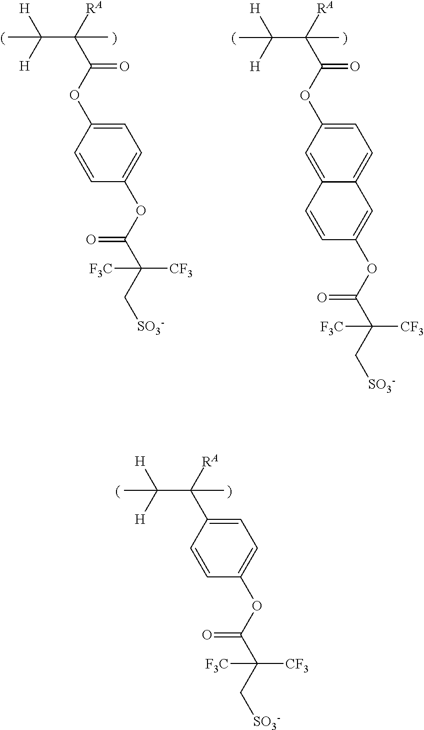

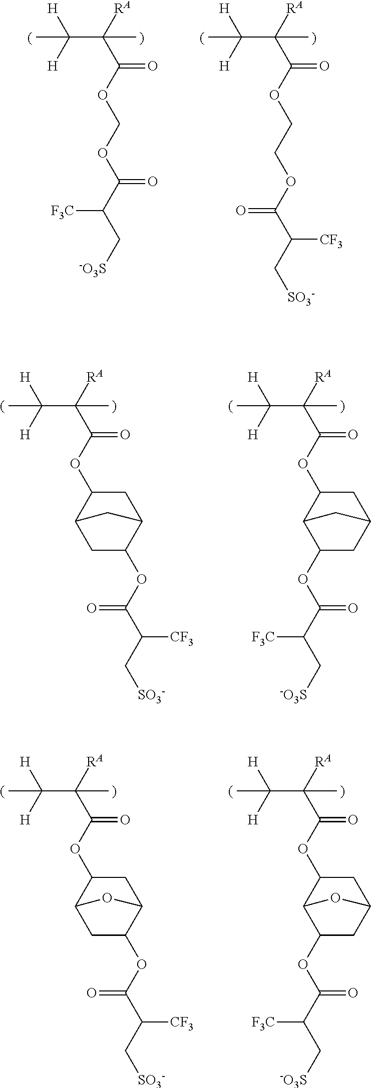

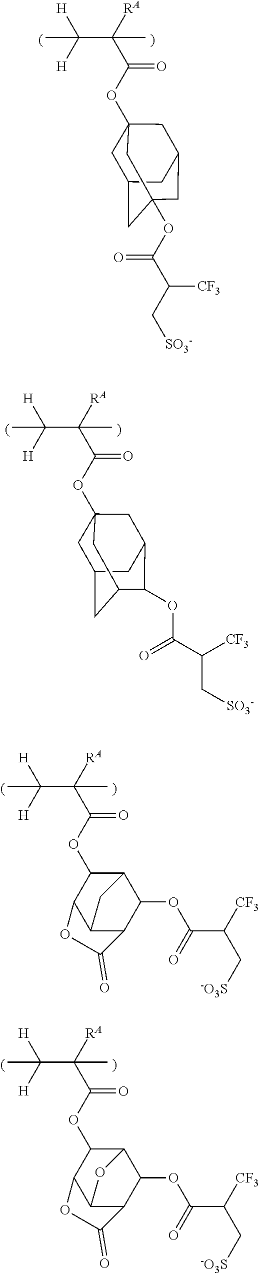

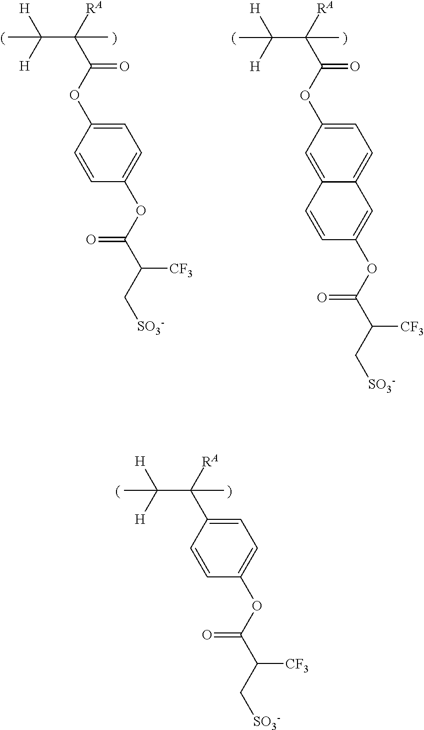

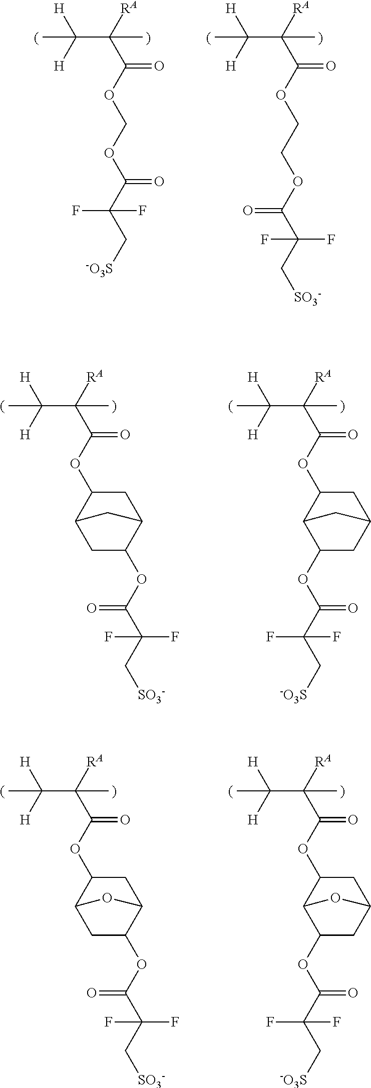

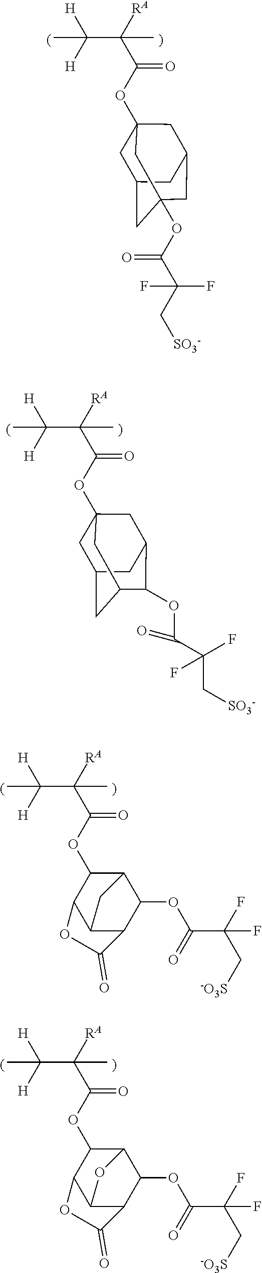

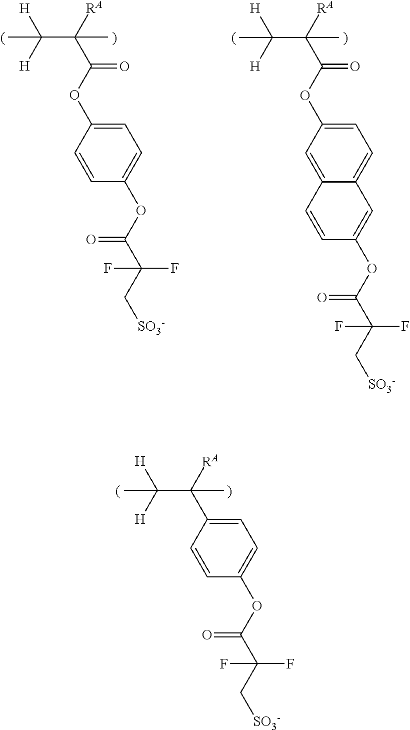

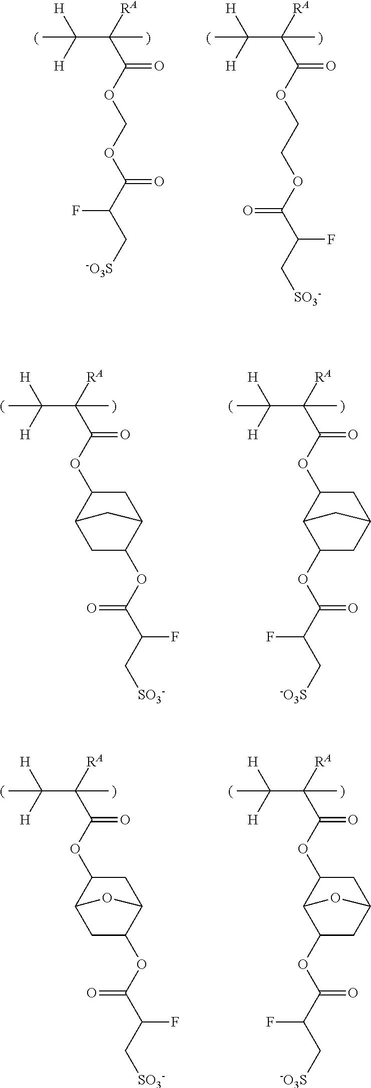

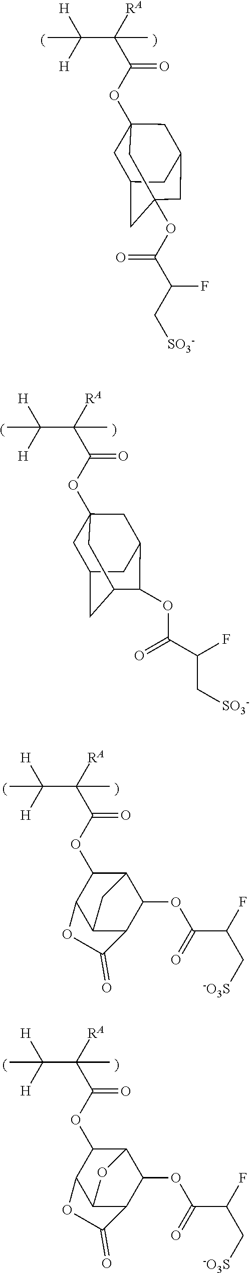

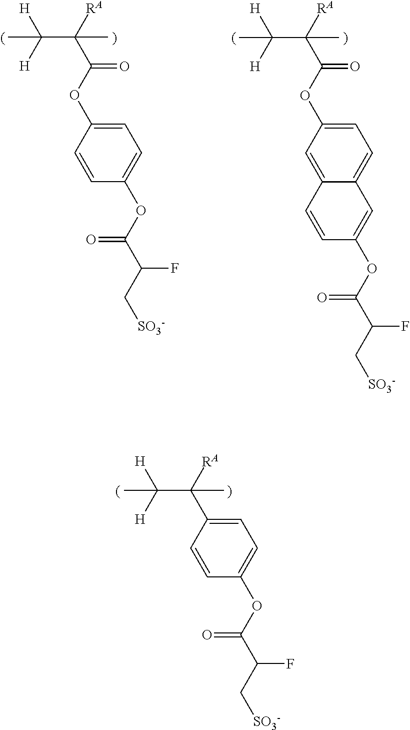

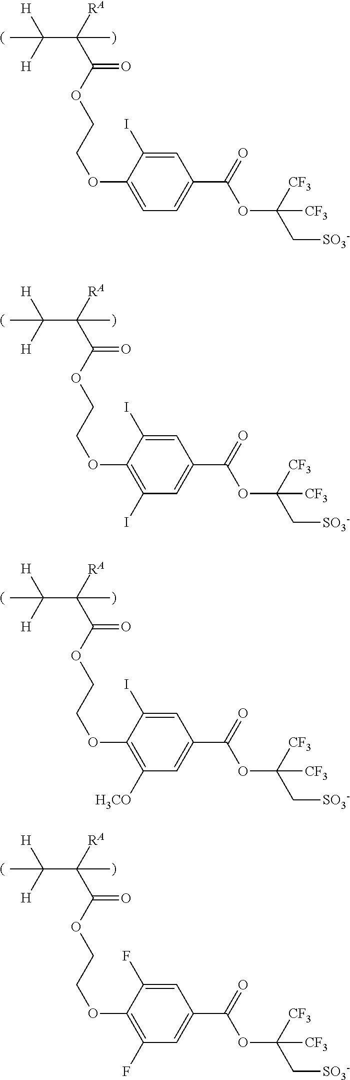

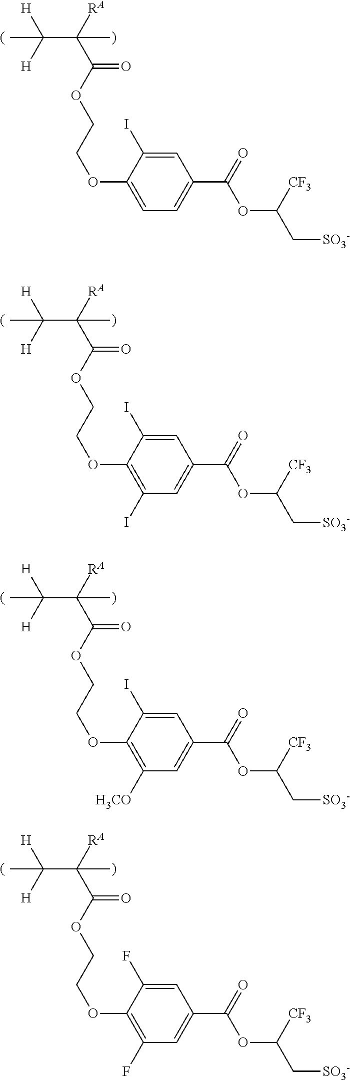

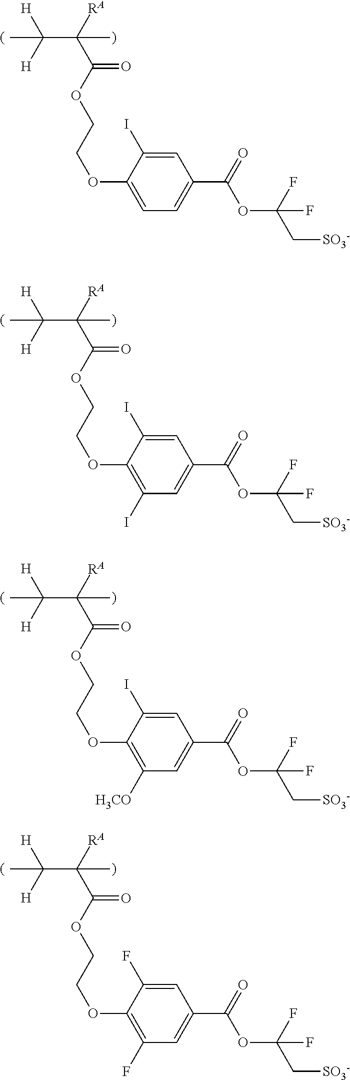

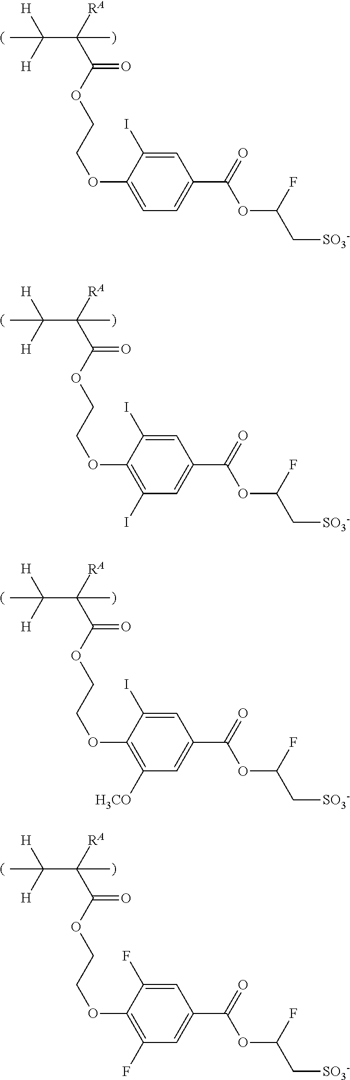

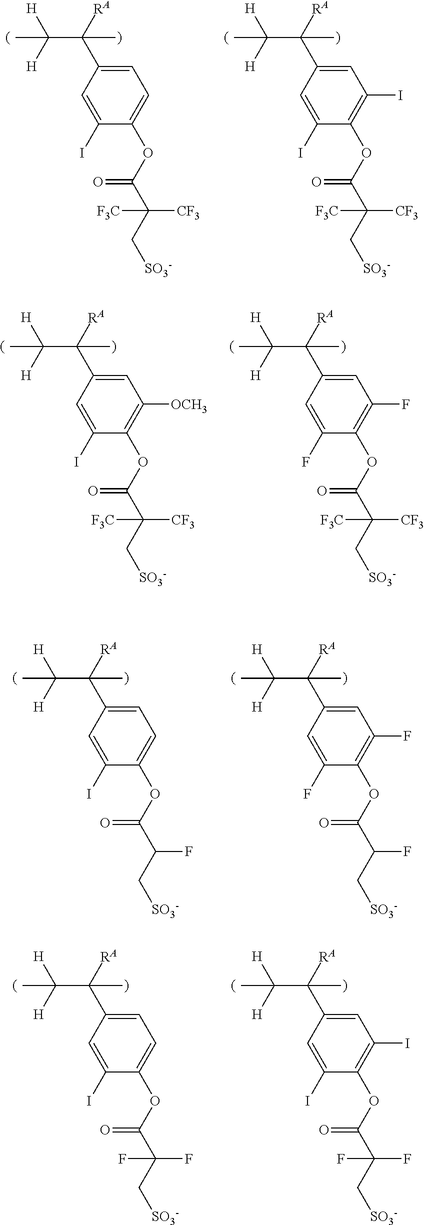

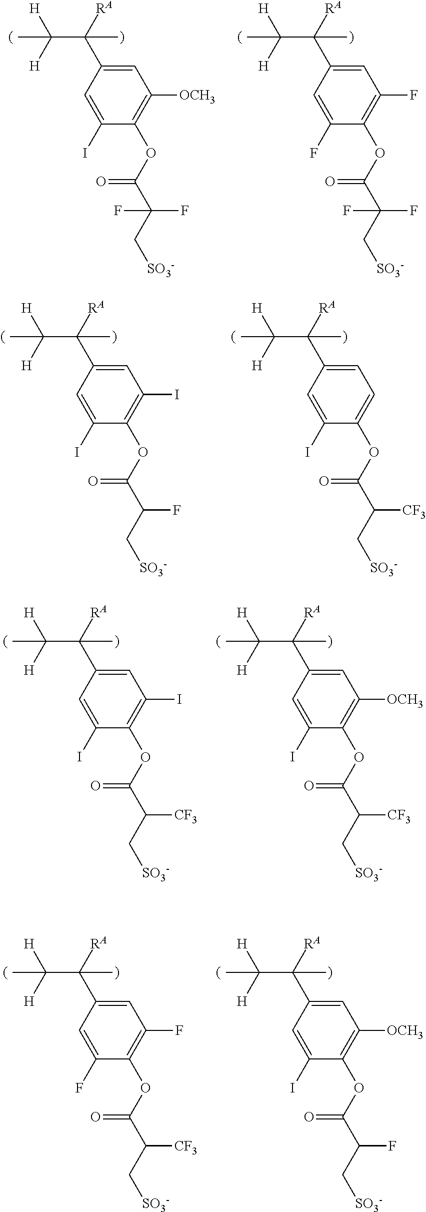

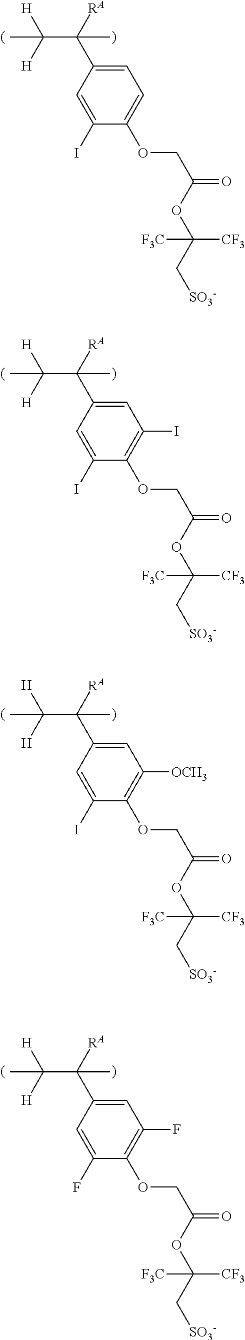

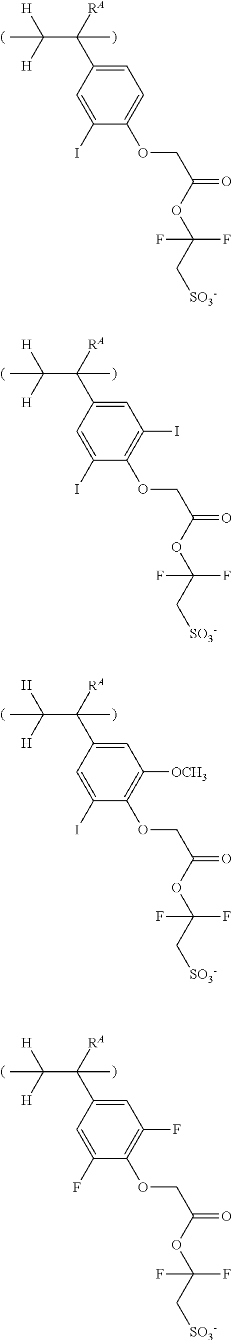

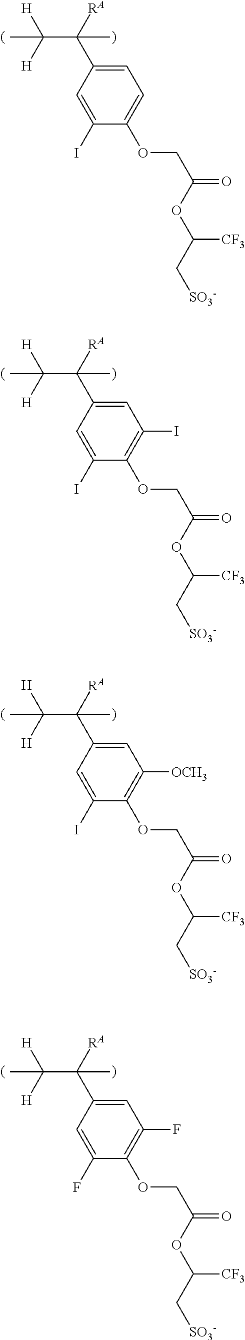

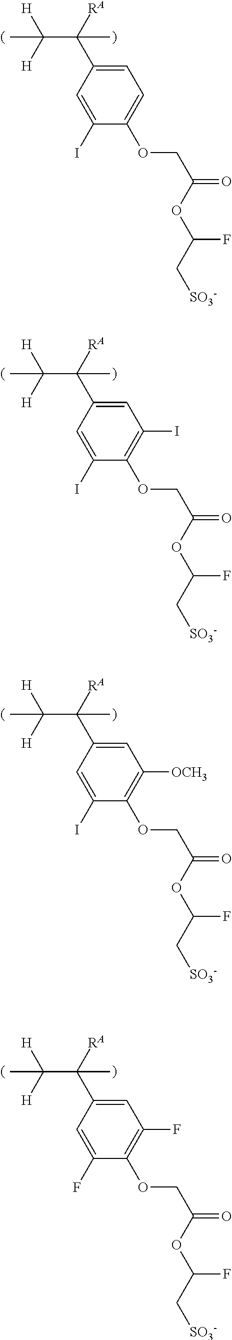

Specific examples of the anion of the repeat units c4 are shown below, but not limited thereto. In the following formula. R^{A} is as defined above.

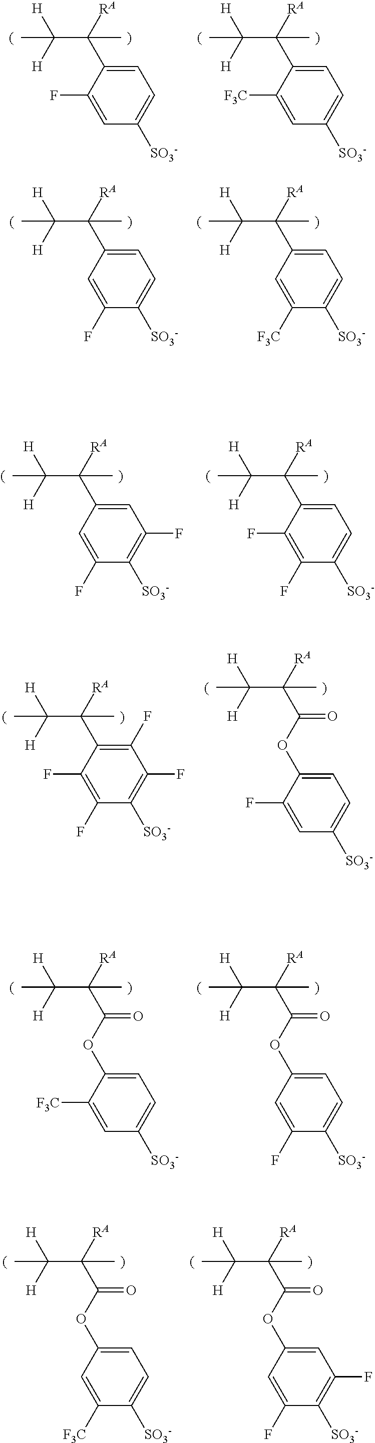

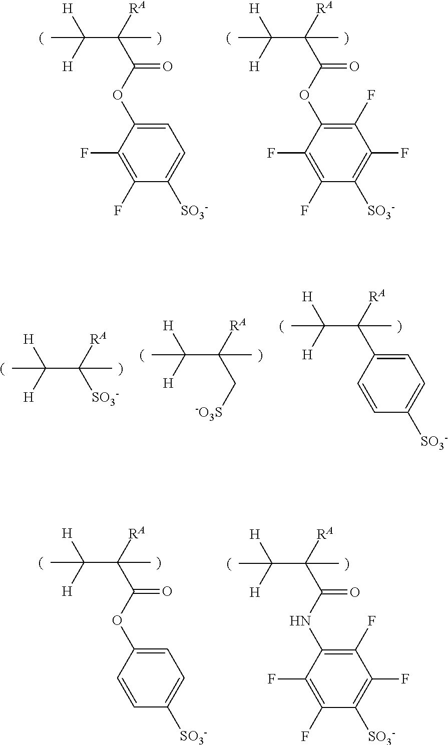

In formulae (C₂) to (C₄), A⁺ is an onium cation. Examples of the onium cation include a sulfonium cation, an iodonium cation, and an ammonium cation, and a sulfonium cation and an iodonium cation are preferable. Specific structures thereof are as exemplified for the cations having formulae (cation-1) to (cation-3).

The repeat units c1 to c4 function as a photoacid generator. In the case of using the base polymer comprising the repeat units c1 to c4 (that is, polymer-bound acid generator), the resist composition of the present invention may or may not comprise a photoacid generator (D) described below.

The base polymer may further comprise repeat units having a structure having a hydroxy group protected with an acid labile group (also referred to as repeat units d, hereinafter). The repeat unit d is not particularly limited as long as the unit includes one or two or more structures having a hydroxy group protected with a protective group such that the protective group is decomposed to generate the hydroxy group under the action of acid, but repeat units having the following formula (d1) are preferable.

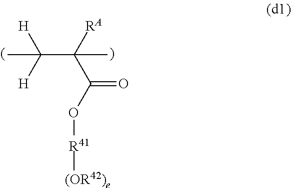

In formula (d1), R^{A} is as defined above. R⁴¹ is a C₁-C₃₀ (e+1)-valent hydrocarbon group which may contain a heteroatom. R⁴² is an acid labile group. e is an integer of 1 to 4.

In formula (d1), the acid labile group represented by R⁴² may be any group that is deprotected under the action of acid so that a hydroxy group is generated. The structure of R⁴² is not particularly limited, an acetal structure, a ketal structure, an alkoxycarbonyl group, an alkoxymethyl group having the following formula (d2), and the like are preferable, and an alkoxymethyl group having the following formula (d2) is particularly preferable.

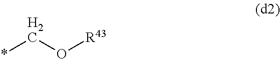

wherein * designates a point of attachment, and R⁴³ is a C₁-C₁₅ hydrocarbyl group.

Specific examples of the acid labile group represented by R⁴², the alkoxymethyl group having formula (d2), and the repeat units d are as exemplified for the repeat units d described in JP-A 2020-111564.

The base polymer may further include repeat units e derived from indene, benzofuran, benzothiophene, acenaphthylene, chromone, coumarin, norbornadiene, or derivatives thereof. Specific examples of the monomer from which repeat units e are derived are shown below, but not limited thereto.

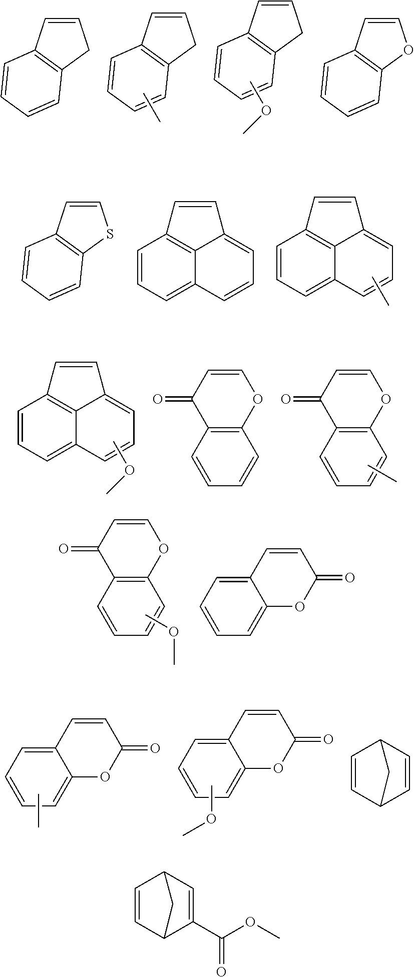

The base polymer may further comprise repeat units f derived from indane, vinylpyridine, or vinylcarbazole.

In the polymer of the present invention, the repeat units a1, a2, b1, b2, c1 to c4, d, e, and f are incorporated in a ratio of preferably 0<a1≤0.8, 0≤a2≤0.8, 0<a1+a2≤0.8, 0≤b1≤0.6, 0≤b2≤0.6, 0≤b1+b2≤0.6, 0≤c1≤0.4, 0≤c2≤0.4, 0≤c3≤0.4, 0≤c4≤0.4, 0≤c1+c2+c3+c4≤0.4, 0≤d≤0.5, 0≤e≤0.3, and 0≤f≤0.3, more preferably 0<a1≤0.7, 0≤a2≤0.7, 0<a1+a2≤0.7, 0≤b1≤0.5, 0≤b2≤0.5, 0≤b1+b2≤0.5, 0≤c1≤0.3, 0≤c2≤0.3, 0≤c3≤0.3, 0≤c4≤0.3, 0≤c1+c2+c3+c4≤0.3, 0≤d≤0.3, 0≤c≤0.3, and 0≤f≤0.3. Provided that a1+a2+b1+b2+c1+c2+c3+c4+d+c+f=1.0.

The weight average molecular weight (Mw) of the polymer is preferably 1000 to 500000 and more preferably 3000 to 100000. When Mw is in this range, sufficient etching resistance is obtained, and there is no possibility of degradation of resolution due to a failure to acquire a difference in dissolution rate before and after exposure. In the present invention, Mw is a value measured by gel permeation chromatography (GPC) with tetrahydrofuran (THF) or N,N-dimethylformamide (DMF) as a solvent, and calculated as polystyrene.

Since the influence of the molecular weight distribution (Mw/Mn) becomes stronger as the pattern rule becomes finer, the Mw/Mn of the polymer preferably has narrow dispersity of 1.0 to 2.0 in order to obtain a resist composition suitable for micropatterning to a small feature size. Within the above range, there is little polymer having a low molecular weight or a high molecular weight, and there is no possibility that foreign matter is observed on the pattern or the shape of the pattern is deteriorated after exposure.

In order to synthesize the polymer, for example, a monomer from which the foregoing repeat units are derived may be heated in an organic solvent with a radical polymerization initiator added thereto to perform polymerization.

Examples of the organic solvent used during polymerization include toluene, benzene, THF, diethyl ether, dioxane, cyclohexane, cyclopentane, methyl ethyl ketone (MEK), PGMEA, and GBL. Examples of the polymerization initiator include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The initiator is preferably added in an amount of 0.01 to 25 mol % based on the total of monomers to be polymerized. The reaction temperature is preferably 50 to 150° C., more preferably 60 to 100° C. The reaction time is preferably 2 to 24 hours, more preferably 2 to 12 hours in viewpoint of production efficiency.

The polymerization initiator may be fed to the reactor either by adding the initiator to the monomer solution and feeding the solution to the reactor, or by dissolving the initiator in a solvent to form an initiator solution and feeding the initiator solution and the monomer solution independently to the reactor. Because of a possibility that in the standby duration, the initiator generates a radical which triggers polymerization reaction to form an ultra high-molecular-weight polymer, it is preferred from the viewpoint of quality control to prepare the monomer solution and the initiator solution separately and add them dropwise. The acid labile group that has been incorporated in the monomer may be kept as such, or polymerization may be followed by protection or partial protection. Any known chain transfer agent such as dodecyl mercaptan or 2-mercaptoethanol may be used in combination for molecular weight control purpose. In this case, these chain transfer agents are preferably added in an amount of 0.01 to 20 mol % based on the total of monomers to be polymerized.

In the case of a monomer containing a hydroxy group, the hydroxy group may be substituted with an acetal group susceptible to deprotection with an acid such as an ethoxyethoxy group during polymerization, and then deprotected by a weak acid and water, or may be substituted with an acetyl group, a formyl group, a pivaloyl group, or the like, and then alkaline hydrolysis may be performed after polymerization.

When hydroxystyrene or hydroxyvinylnaphthalene is copolymerized, hydroxystyrene or hydroxyvinylnaphthalene and another monomer may be heated and polymerized by adding a radical polymerization initiator in an organic solvent, but acetoxystyrene or acetoxyvinylnaphthalene may be used, and the acetoxy group may be deprotected by alkaline hydrolysis after polymerization to obtain polyhydroxystyrene or hydroxypolyvinylnaphthalenc.

As a base during the alkaline hydrolysis, aqueous ammonia, triethylamine, or the like can be used. The reaction temperature is preferably-20 to 100° C. and more preferably 0 to 60° C. The reaction time is preferably 0.2 to 100 hours and more preferably 0.5 to 20 hours.

The amount of each monomer in the monomer solution may be appropriately set, for example, so as to have a preferred content ratio of the repeat units.

Regarding the polymer obtained by the production method, a reaction solution resulting from polymerization reaction may be used as a final product, or a powder obtained through a purifying step such as re-precipitation method in which a polymerization liquid is added to a poor solvent to obtain a powder may be used as a final product, but from the viewpoints of operation efficiency and consistent quality, it is preferable to use a polymer solution obtained by dissolving the powder resulting from the purifying step in a solvent as a final product.

Specific examples of the solvent used at that time include ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; alcohols such as DAA; high-boiling-point alcohol-based solvents such as diethylene glycol, propylene glycol, glycerin, 1,4-butanediol, and 1,3-butanediol; and a mixed solvent thereof, which are described in JP-A 2008-111103, paragraphs [0144] to [0145].

The polymer solution preferably has a polymer concentration of 0.01 to 30 wt %, more preferably 0.1 to 20 wt %.

The reaction solution or polymer solution is preferably filtered through a filter. Filtration is effective in terms of consistent quality because foreign matter and gel which may cause defects can be removed.

Examples of the material for the filter used for the filter filtration include fluorocarbon-based, cellulose-based, nylon-based, polyester-based, and hydrocarbon-based materials, and in the filtration step of the resist composition, a filter formed of a fluorocarbon-based material called Teflon®, a hydrocarbon-based material such as polyethylene and polypropylene, or nylon is preferable. While the pore size of the filter may be selected appropriate to comply with the desired cleanness, the filter preferably has a pore size of 100 nm or less, more preferably 20 nm or less. A single filter may be used or a plurality of filters may be used in combination. Although the filtering method may be single pass of the solution, preferably the filtering step is repeated by flowing the solution in a circulating manner. In the polymer production step, the filtration step may be carried out any times, in any order and in any stage, but the reaction solution after the polymerization reaction or the polymer solution may be filtered, preferably both are filtered.

The base polymer (C) may be used alone or in combination of two or more polymers which are different in compositional ratio, Mw and/or Mw/Mn. The base polymer (C) may contain a hydrogenated ring-opened metathesis polymer in addition to the polymer, and as the hydrogenated ring-opened metathesis polymer, a polymer described in JP-A 2003-66612 can be used.

### [Photoacid Generator (D)]

The resist composition of the present invention may comprise a photoacid generator as a component (D). The photoacid generator of the component (D) is not particularly limited as long as it is a compound capable of generating strong acid by irradiation with high-energy radiation. Specific examples of suitable photoacid generators include those having the following formula (2-1) or (2-2):

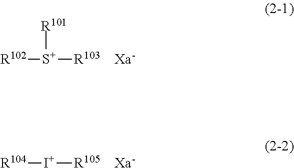

In formulae (2-1) and (2-2), R¹⁰¹ to R¹⁰⁵ are each independently a C₁-C₃₀ hydrocarbyl group which may contain a heteroatom. Any two of R¹⁰¹, R¹⁰², and R¹⁰³ may bond together to form a ring with the sulfur atom to which they are attached.

Specific examples of the sulfonium cation having formula (2-1) are exemplified above for the sulfonium cation having formula (cation-1), but not limited thereto. Specific examples of the iodonium cation having formula (2-2) are exemplified above for the iodonium cation having formula (cation-2), but not limited thereto.

In formulae (2-1) and (2-2), Xa⁻ is an anion selected from the following formulae (2A) to (2D).

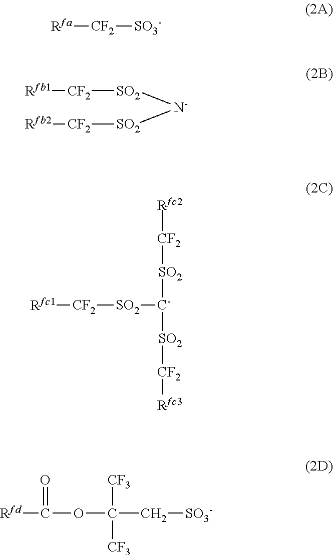

In formula (2A), R^{fa1} is a fluorine atom, or a C₁-C₄₀ hydrocarbyl group which may contain a heteroatom. The hydrocarbyl group may be saturated or unsaturated and straight, branched, or cyclic. Specific examples thereof are as exemplified above for the hydrocarbyl group represented by R^{fa1} in formula (2A′) described below.

The anion having formula (2A) preferably has the following formula (2A′).

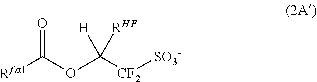

In formula (2A′), R^{HF} represents a hydrogen atom or a trifluoromethyl group, is a hydrogen atom or a trifluoromethyl group, and preferably a trifluoromethyl group.

R^{fa1} is a C₁-C₃₈ hydrocarbyl group which may contain a heteroatom. The heteroatom is preferably an oxygen atom, a nitrogen atom, a sulfur atom, a halogen atom, or the like, and more preferably an oxygen atom. The hydrocarbyl group is particularly preferably a C₆-C₃₀ hydrocarbyl group from the viewpoint of obtaining a high resolution in fine pattern formation.

The C₁-C₃₈ hydrocarbyl group represented by R^{fa1} may be saturated or unsaturated and straight, branched, or cyclic. Specific examples thereof include C₁-C₃₈ alkyl groups such as a methyl group, an ethyl group, a propyl group, an isopropyl group, a butyl group, an isobutyl group, a sec-butyl group, a tert-butyl group, a pentyl group, a neopentyl group, a hexyl group, a heptyl group, an octyl group, a 2-ethylhexyl group, a nonyl group, a decyl group, an undecyl group, a dodecyl group, a tridecyl group, a pentadecyl group, a heptadecyl group, and an icosyl group; C₃-C₃₈ cyclic saturated hydrocarbyl groups such as a cyclopentyl group, a cyclohexyl group, a 1-adamantyl group, a 2-adamantyl group, a 1-adamantylmethyl group, a norbornyl group, a norbornylmethyl group, a tricyclodecyl group, a tetracyclododecyl group, a tetracyclododecylmethyl group, and a dicyclohexylmethyl group; C₂-C₃₈ unsaturated aliphatic hydrocarbyl groups such as an allyl group and a 3-cyclohexenyl group; C₆-C₃₈ aryl groups such as a phenyl group, a 1-naphthyl group, and a 2-naphthyl group; C₇-C₃₈ aralkyl groups such as a benzyl group and a diphenylmethyl group; and groups obtained by combining these.

Some or all of the hydrogen atoms in the hydrocarbyl group may be substituted with a group containing a heteroatom such as an oxygen atom, a sulfur atom, a nitrogen atom, or a halogen atom, some of —CH₂— in the hydrocarbyl group may be substituted with a group containing a heteroatom such as an oxygen atom, a sulfur atom, or a nitrogen atom, so that the group may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, or the like. Specific examples of the heteroatom-containing hydrocarbyl group include a tetrahydrofuryl group, a methoxymethyl group, an ethoxymethyl group, a methylthiomethyl group, an acetamidomethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, a 5-hydroxy-1-adamantyl group, a 5-tert-butylcarbonyloxy-1-adamantyl group, a 4-oxatricyclo[4.2.1.03.7]nonane-5-one-2-yl group, and a 3-oxocyclohexyl group.

With respect to the synthesis of the sulfonium salt having an anion of formula (2A′), reference is made to JP-A 2007-145797, JP-A 2008-106045, JP-A 2009-7327, JP-A 2009-258695, and the like. Also useful are the sulfonium salts described in JP-A 2010-215608, JP-A 2012-41320, JP-A 2012-106986, JP-A 2012-153644, and the like.

Specific examples of the anion having formula (2A) are as exemplified for the anions having formulae (c1-1) and (c1-2).

In formula (2B), R^{fb1} and R^{fb2} are each independently a fluorine atom, or a C₁-C₄₀ hydrocarbyl group which may contain a heteroatom. The hydrocarbyl group may be saturated or unsaturated and straight, branched, or cyclic. Specific examples thereof are as exemplified above for the hydrocarbyl group represented by R^{fa1} in formula (2A′). Preferably, R^{fb1} and R^{fb2} each are a fluorine atom or a straight C₁-C₄ fluorinated alkyl group. R^{fb1} and R^{fb2} may bond together to form a ring with the group (—CF₂—SO₂—N⁻—SO₂—CF₂—) to which they are attached, and in this case, the group obtained by bonding R^{fb1} and R^{fb2} to each other is preferably a fluorinated ethylene group or a fluorinated propylene group.

In formula (2C), R^{fc1}, R^{fc2}, and R^{fc3} are each independently a fluorine atom, or a C₁-C₄₀ hydrocarbyl group which may contain a heteroatom. The hydrocarbyl group may be saturated or unsaturated and straight, branched, or cyclic. Specific examples thereof are as exemplified above for the hydrocarbyl group represented by R^{fa1} in formula (2A′). Preferably, R^{fc1}, R^{fc2}, and R^{fc3} each are a fluorine atom or a straight C₁-C₄ fluorinated alkyl group. R^{fc1} and R^{fc2} may bond together to form a ring with the group (—CF₂—SO₂—C⁻—SO₂—CF₂—) to which they are attached, and in this case, the group obtained by bonding R^{fc1} and R^{fc2} to each other is preferably a fluorinated ethylene group or a fluorinated propylene group.

In formula (2D), R^{fd} is a C₁-C₄₀ hydrocarbyl group which may contain a heteroatom. The hydrocarbyl group may be saturated or unsaturated and straight, branched, or cyclic. Specific examples thereof are as exemplified above for the hydrocarbyl group represented by R^{fa1} in formula (2A′).

With respect to the synthesis of the sulfonium salt having an anion of formula (2D), reference is made to JP-A 2010-215608 and JP-A 2014-133723.

Specific examples of the anion having formula (2D) are shown below, but not limited thereto.

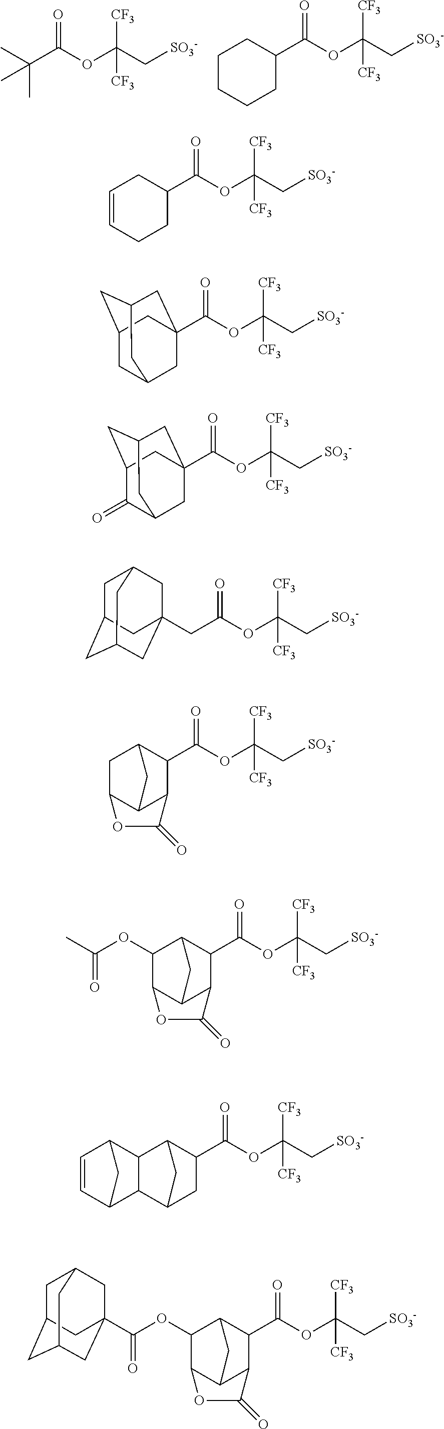

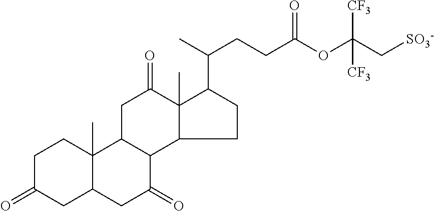

Examples of the non-nucleophilic counter ion further include an anion having an aromatic ring substituted with an iodine atom or a bromine atom. Specific examples of such an anion include those having the following formula (2E).

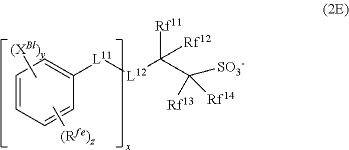

In formula (2E), x is an integer satisfying 1≤x≤3. y and z are integers satisfying 1≤y≤5, 0≤z≤3, and 1≤y+z≤5. y is preferably an integer satisfying 1≤y≤3 and more preferably 2 or 3. z is preferably an integer satisfying 0≤z≤2.

In formula (2E), X^{BI} is an iodine atom or a bromine atom, and may be the same or different when x and/or y is 2 or more.

In formula (2E), L¹¹ is a single bond, an ether bond, an ester bond, or a C₁-C₆ saturated hydrocarbylene group which may contain an ether bond or an ester bond. The saturated hydrocarbylene group may be straight, branched, or cyclic.

In formula (2E), L¹² is a single bond or a C₁-C₂₀ divalent linking group when x is 1, and a C₁-C₂₀ (x+1)-valent linking group which may contain an oxygen atom, a sulfur atom, or a nitrogen atom when x is 2 or 3.

In formula (2E), R^{fc} is a hydroxy group, a carboxy group, a fluorine atom, a chlorine atom, a bromine atom, an amino group, a C₁-C₂₀ hydrocarbyl group, C₁-C₂₀ hydrocarbyloxy group, C₂-C₂₀ hydrocarbylcarbonyl group, C₂-C₁₀ hydrocarbyloxycarbonyl group, C₂-C₂₀ hydrocarbylcarbonyloxy group, or C₁-C₂₀ hydrocarbylsulfonyloxy group, which may contain a fluorine atom, a chlorine atom, a bromine atom, a hydroxy group, an amino group, or an ether bond, or —N(R^{fcA}) (R^{fcB}), —N(R^{fcC})—C(═O)—R^{fcD}, or —N(R^{fcC})—C(═O)—O—R^{fcD}. R^{fcA} and R^{fcB} are each independently a hydrogen atom or a C₁-C₆ saturated hydrocarbyl group. R^{fcC} is a hydrogen atom or a C₁-C₆ saturated hydrocarbyl group, which may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. R^{fcD} is a C₁-C₁₆ aliphatic hydrocarbyl group, a C₆-C₁₂ aryl group, or a C₇-C₁₅ aralkyl group, which may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. The aliphatic hydrocarbyl group may be saturated or unsaturated and straight, branched, or cyclic. The hydrocarbyl group, the hydrocarbyloxy group, the hydrocarbylcarbonyl group, the hydrocarbyloxycarbonyl group, the hydrocarbylcarbonyloxy group, and the hydrocarbylsulfonyloxy group may be straight, branched, or cyclic. When x and/or z is 2 or more, respective R⁸ may be the same as or different from each other.

Of these, R^{fc} is preferably a hydroxy group, —N(R^{fcC})—C(═O)—R^{fcD}, —N(R^{fcC})—C(═O)—O—R^{fcD}, a fluorine atom, a chlorine atom, a bromine atom, a methyl group, a methoxy group, or the like.

In formula (2E), Rf¹¹ to Rf¹⁴ are each independently a hydrogen atom, a fluorine atom, or a trifluoromethyl group, and of these, at least one of Rf¹¹ to Rf¹⁴ is a fluorine atom or a trifluoromethyl group. Rf¹¹ and Rf¹², taken together, may form a carbonyl group. Particularly, both Rf¹³ and Rf¹⁴ are a fluorine atom.

Specific examples of the anion of the onium salt having formula (2E) are shown below, but not limited thereto. In the following formula, X^{BI} is as defined above.

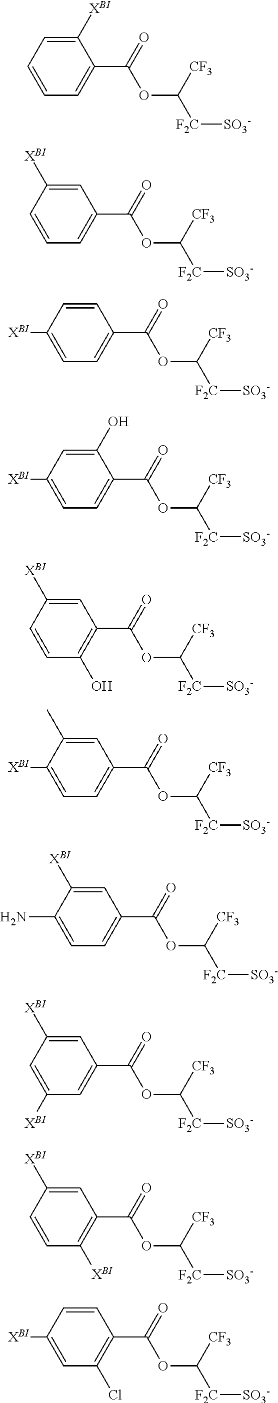

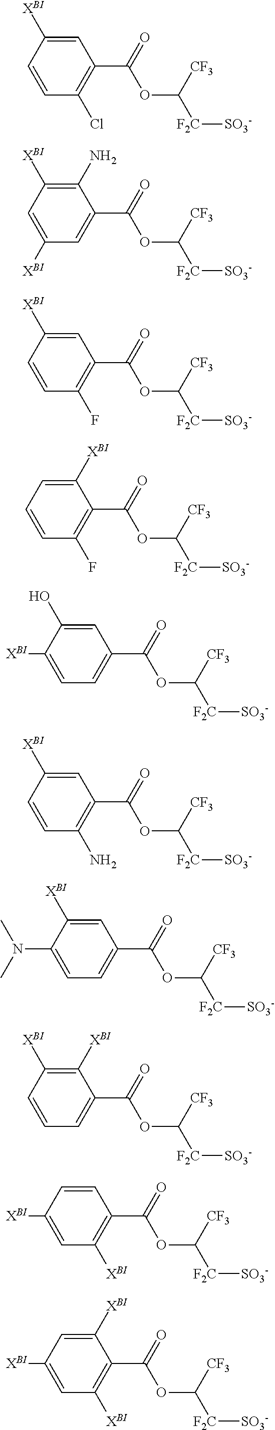

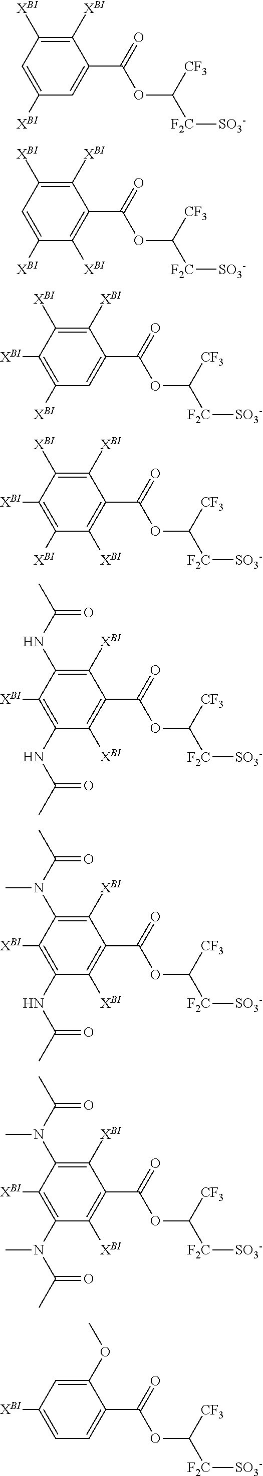

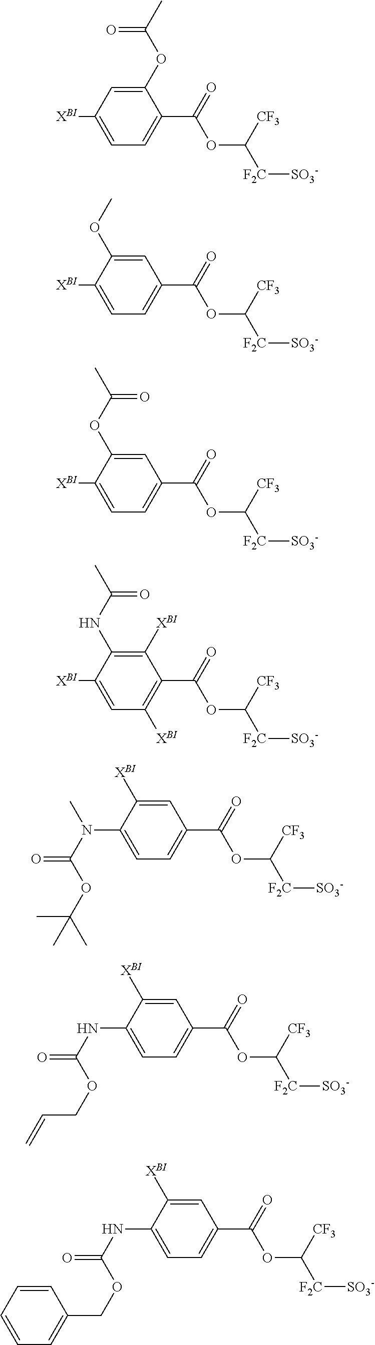

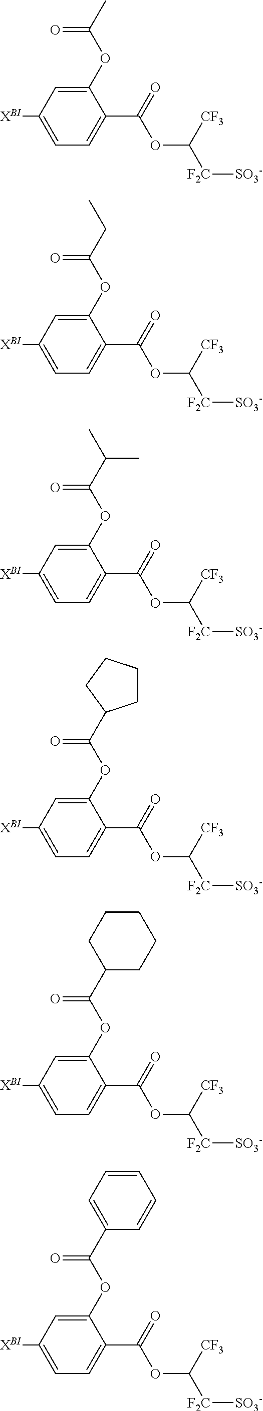

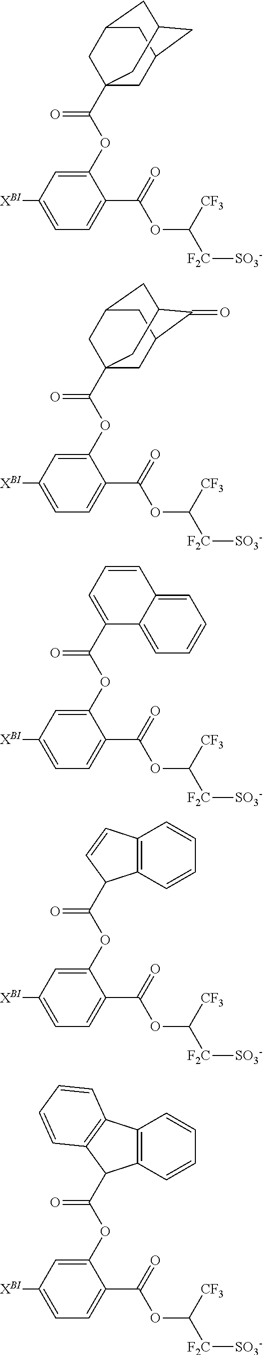

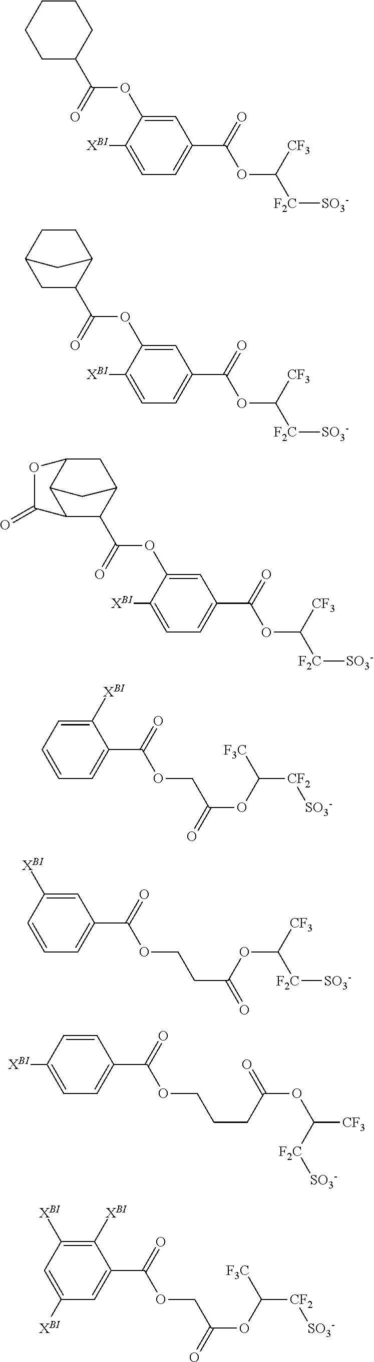

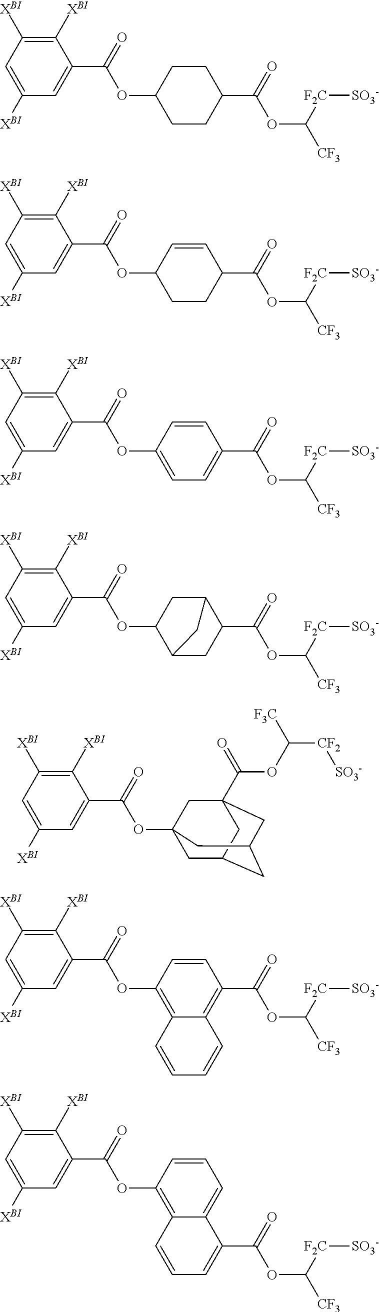

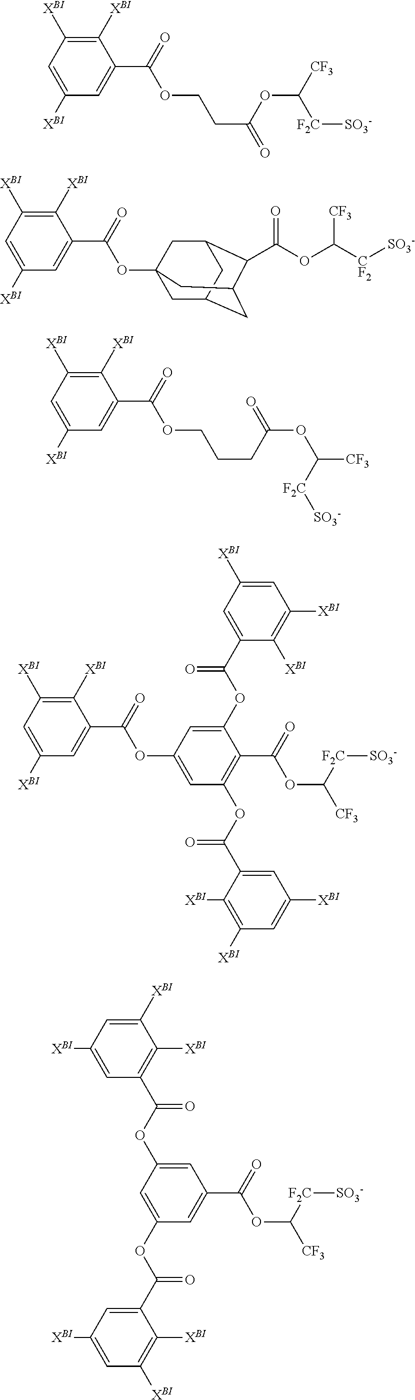

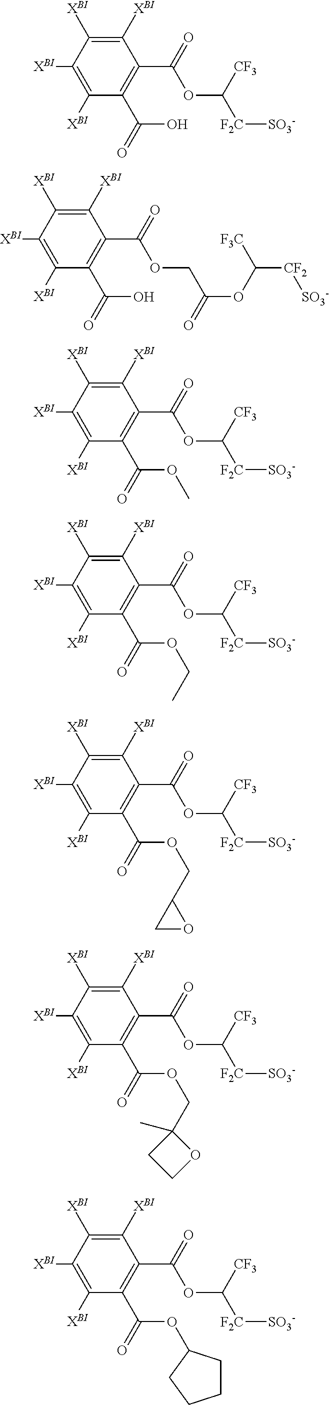

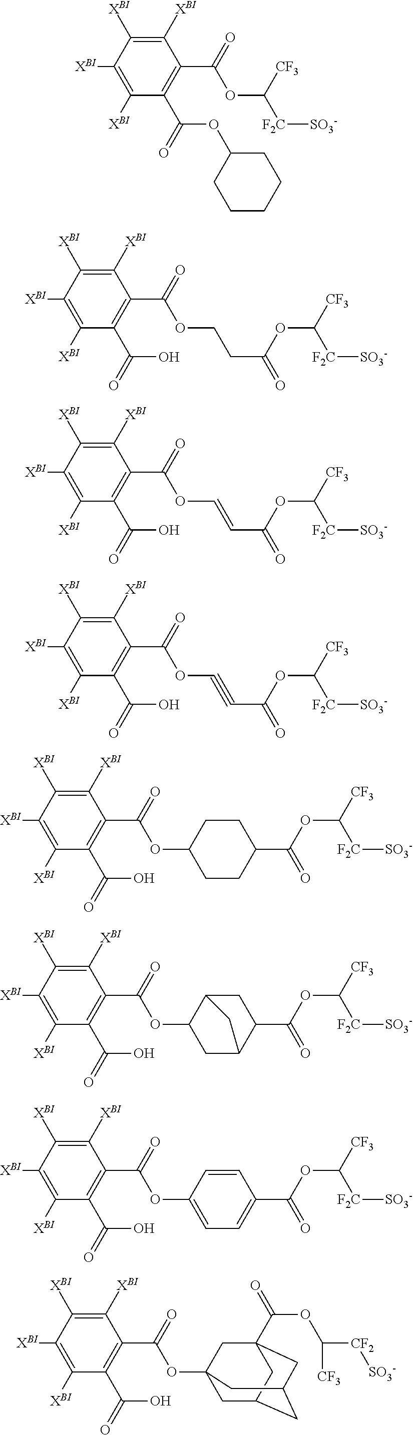

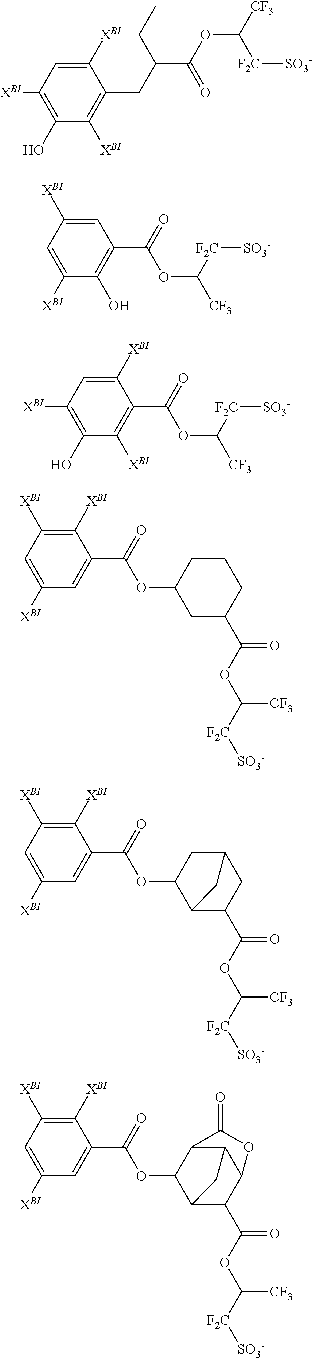

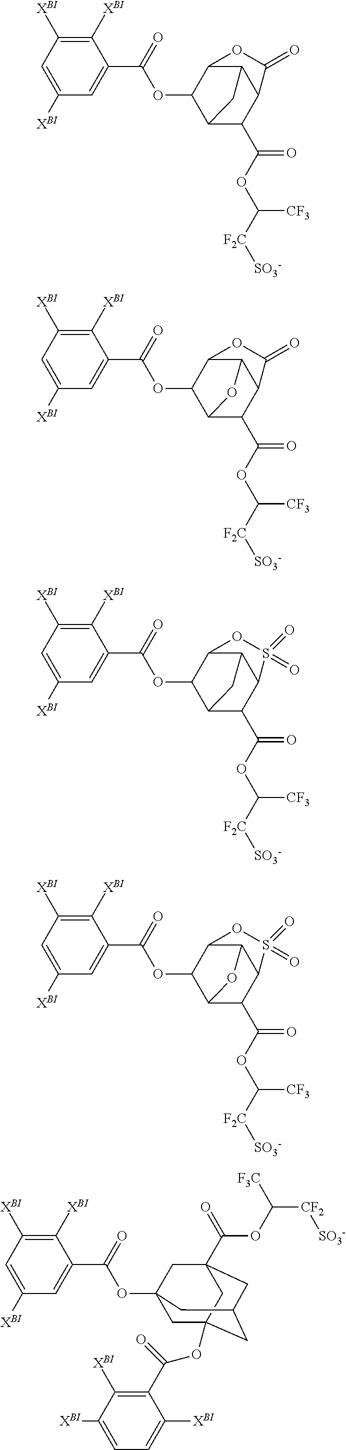

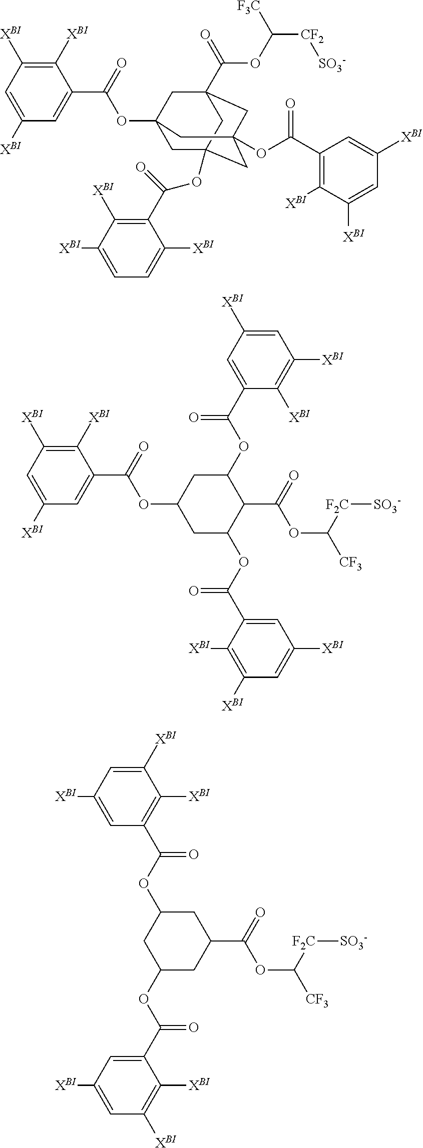

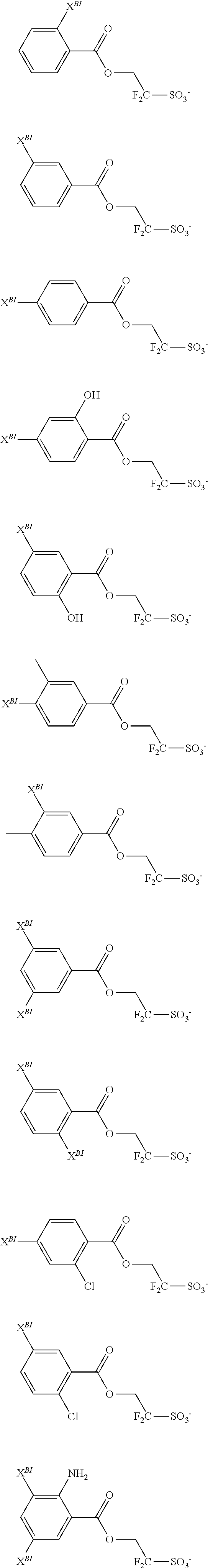

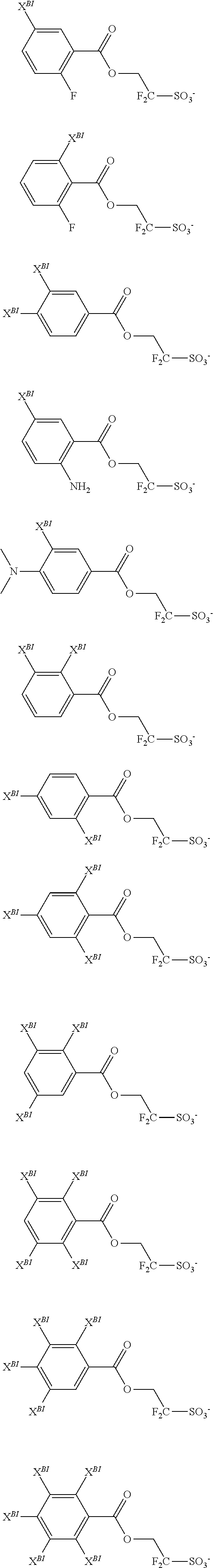

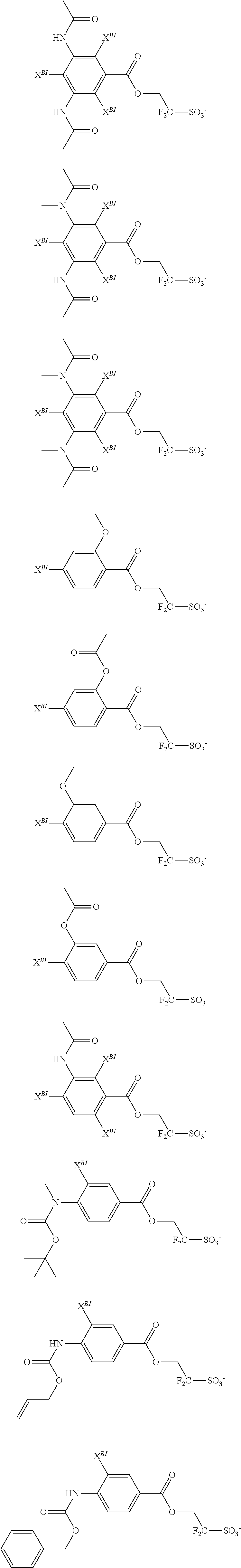

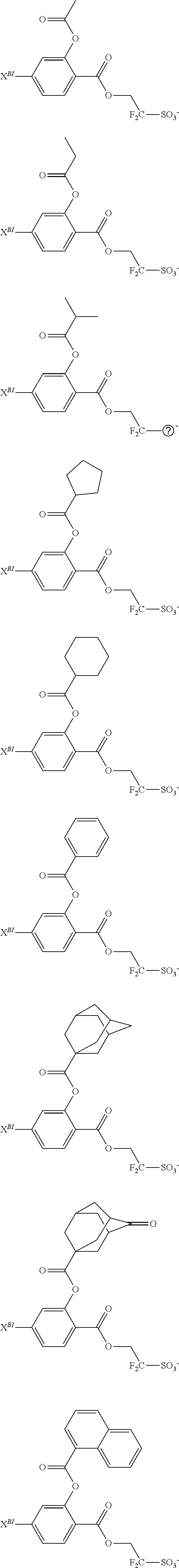

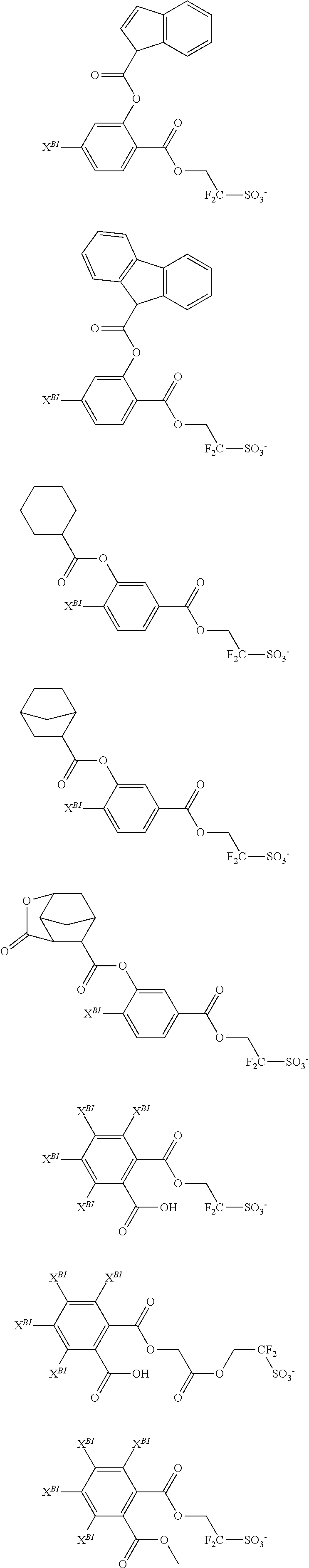

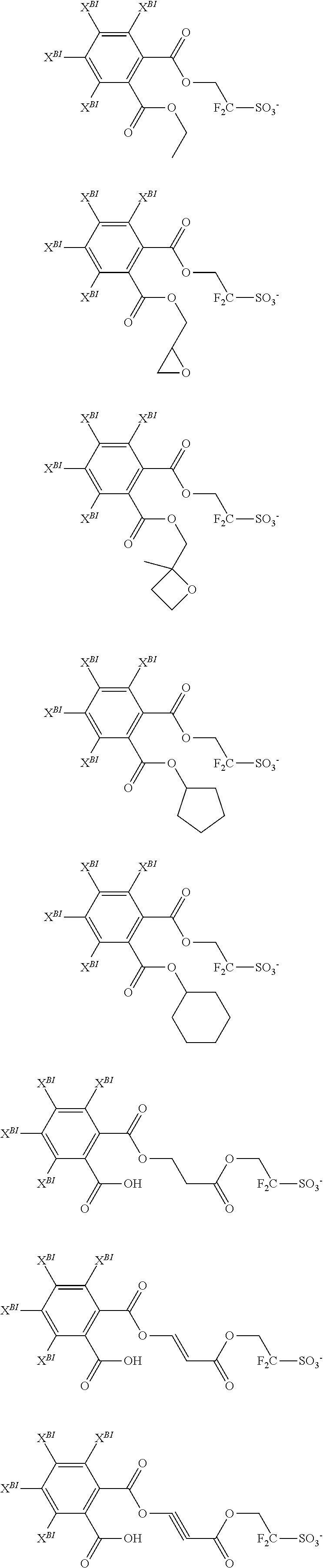

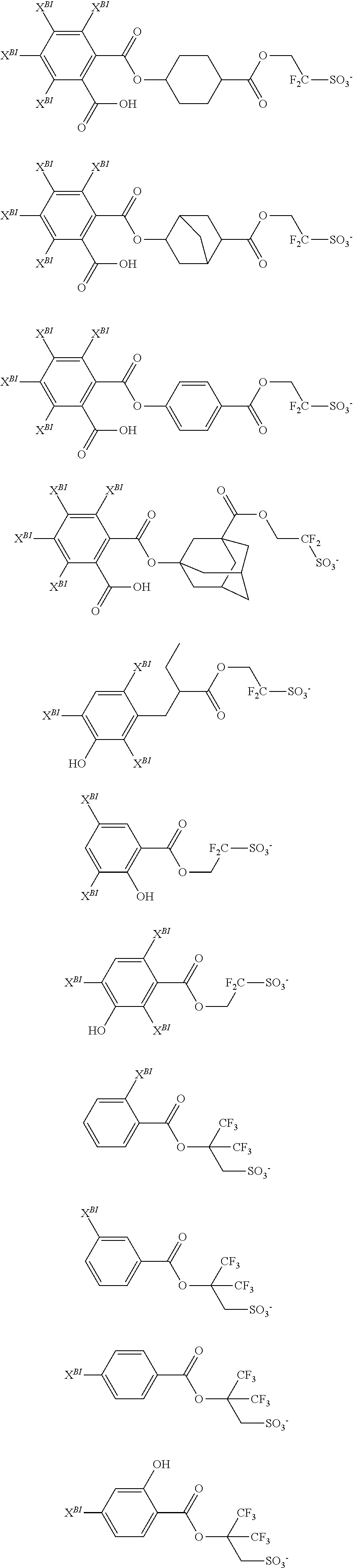

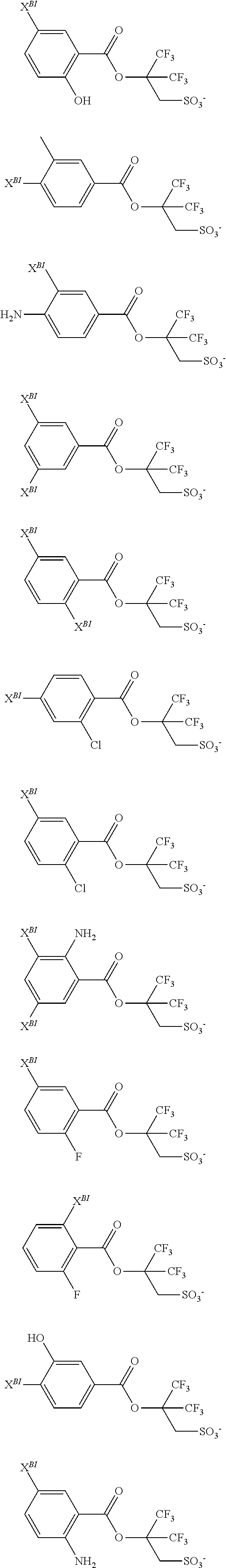

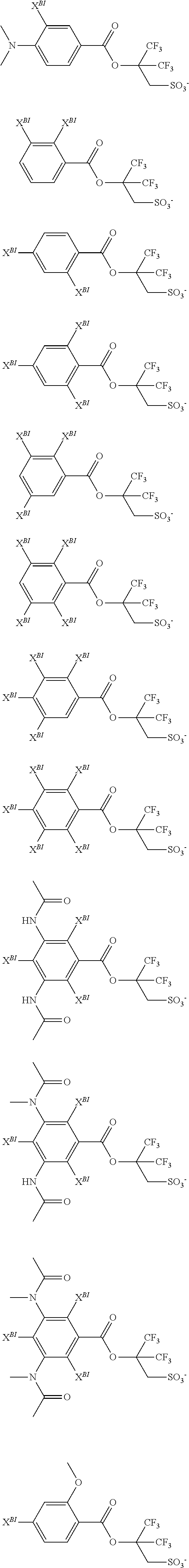

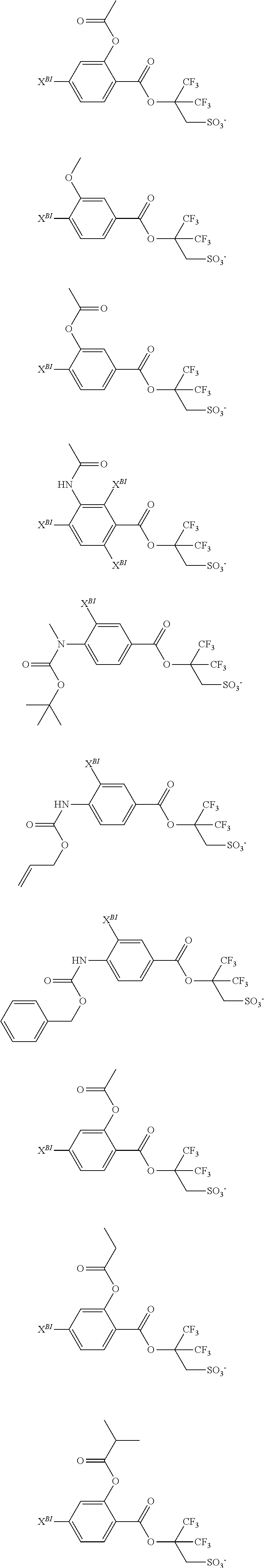

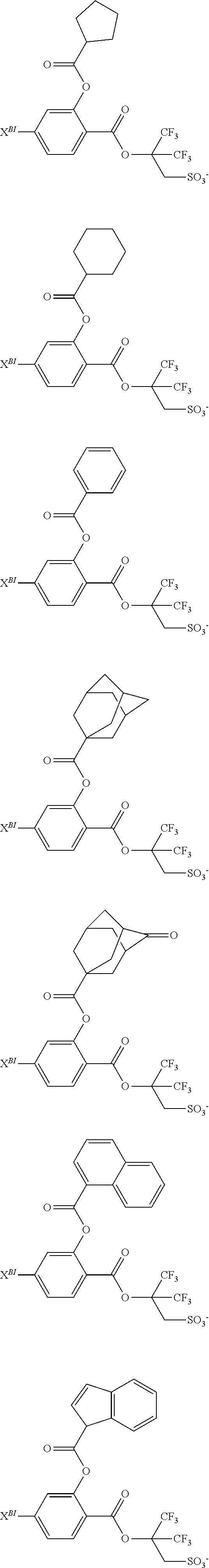

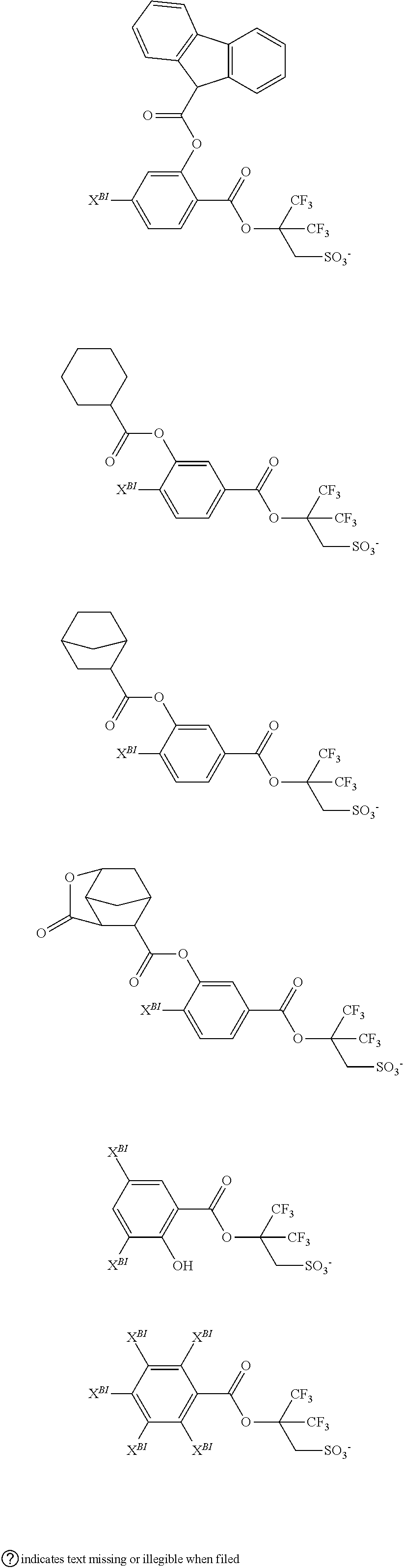

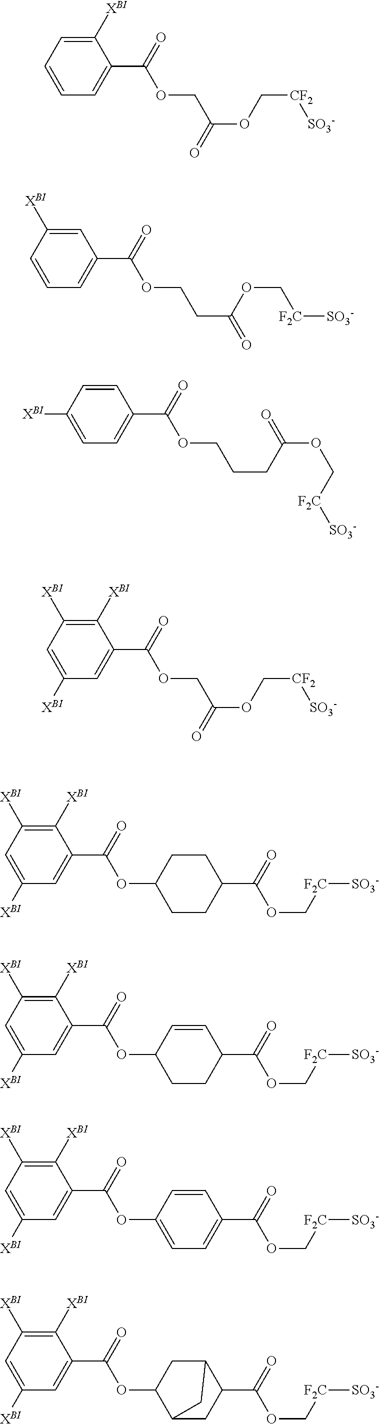

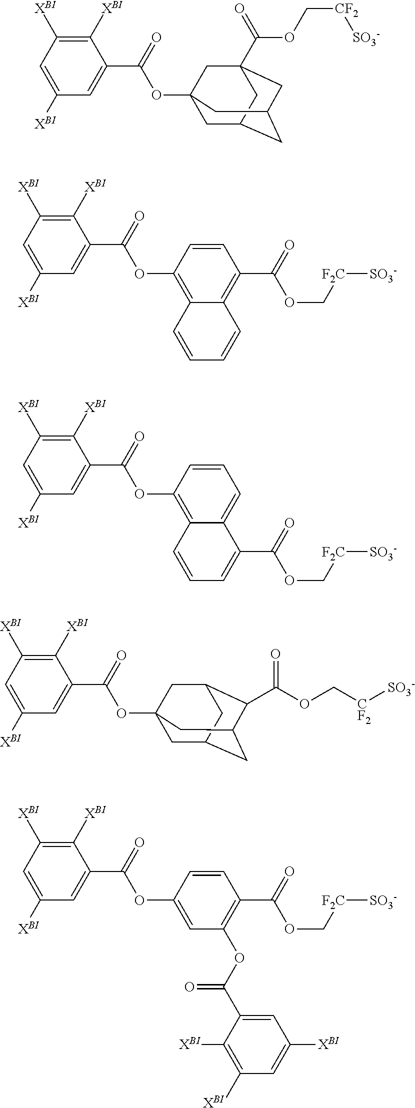

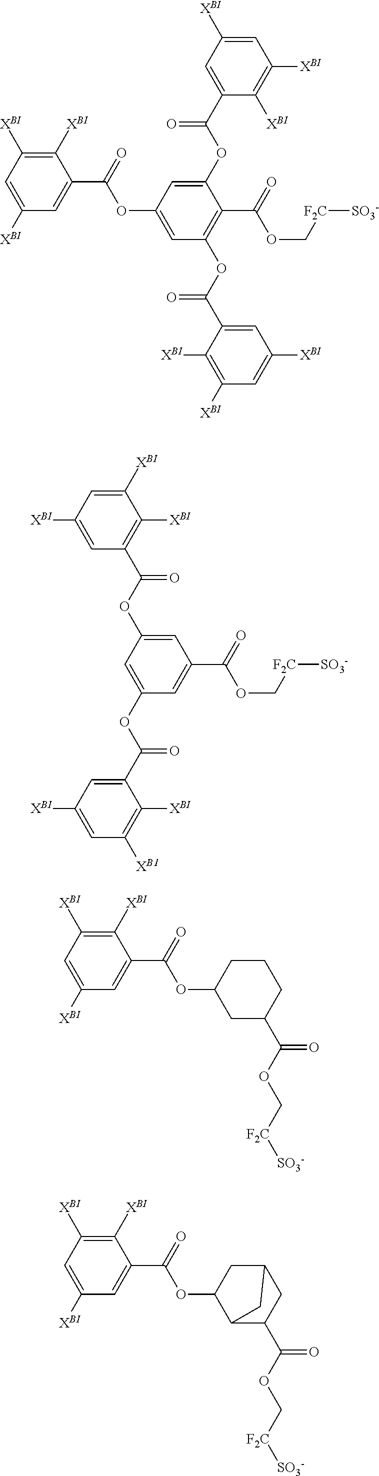

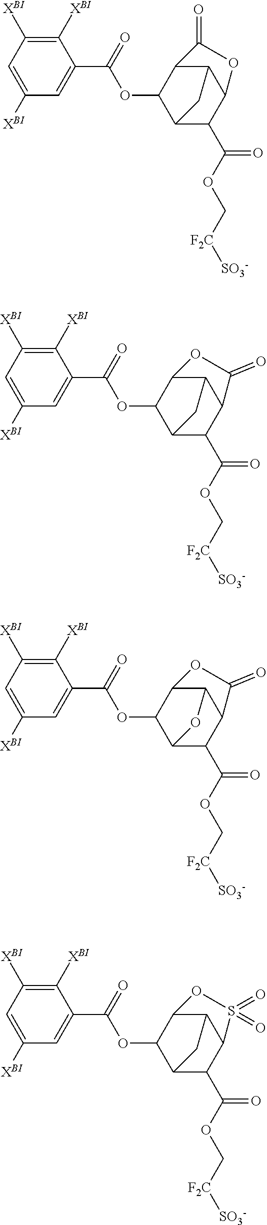

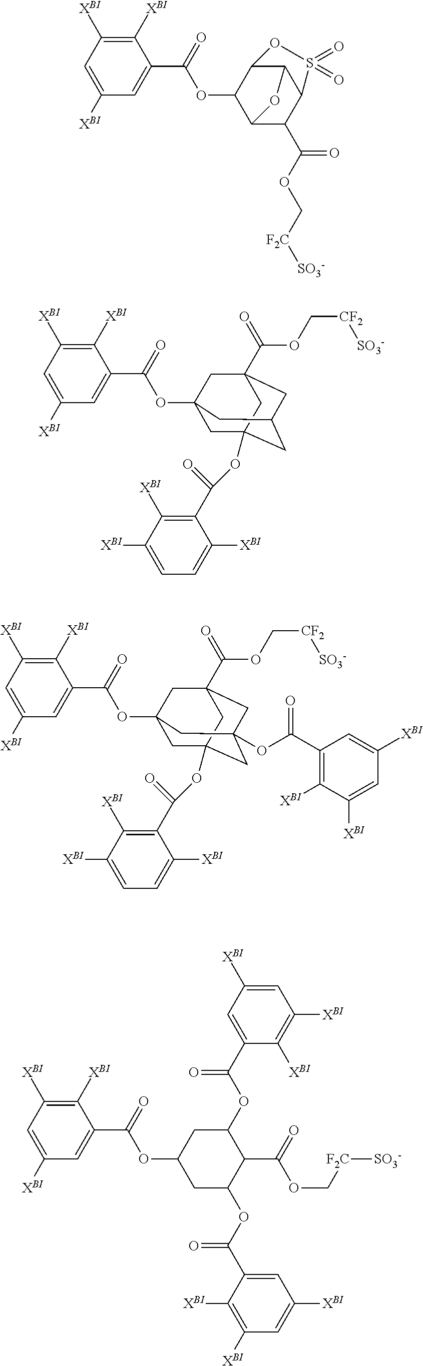

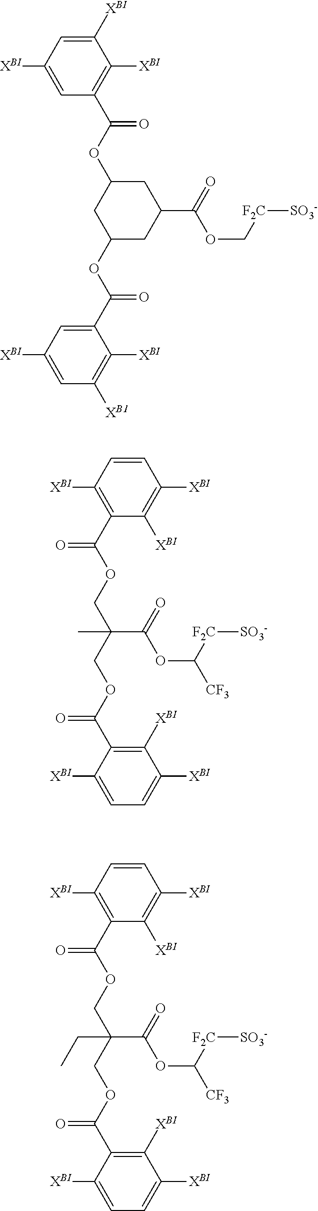

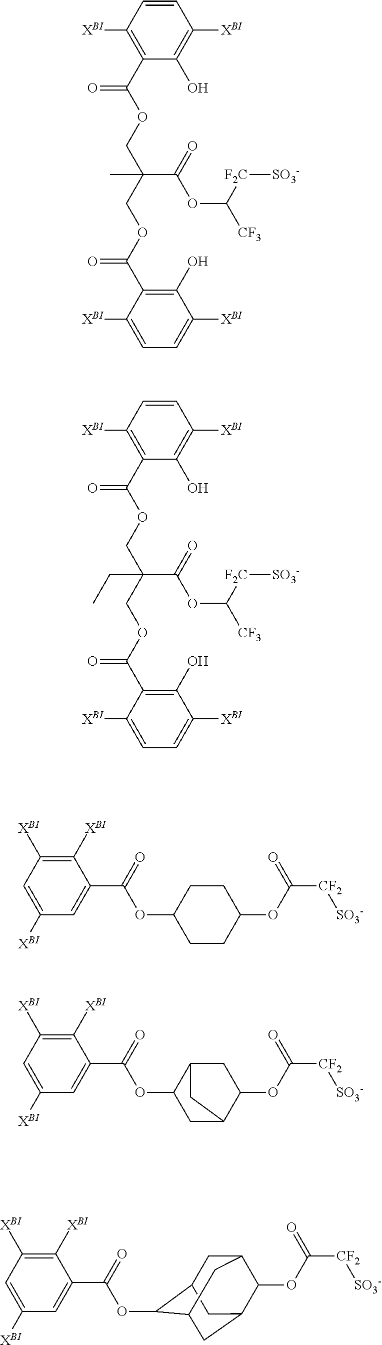

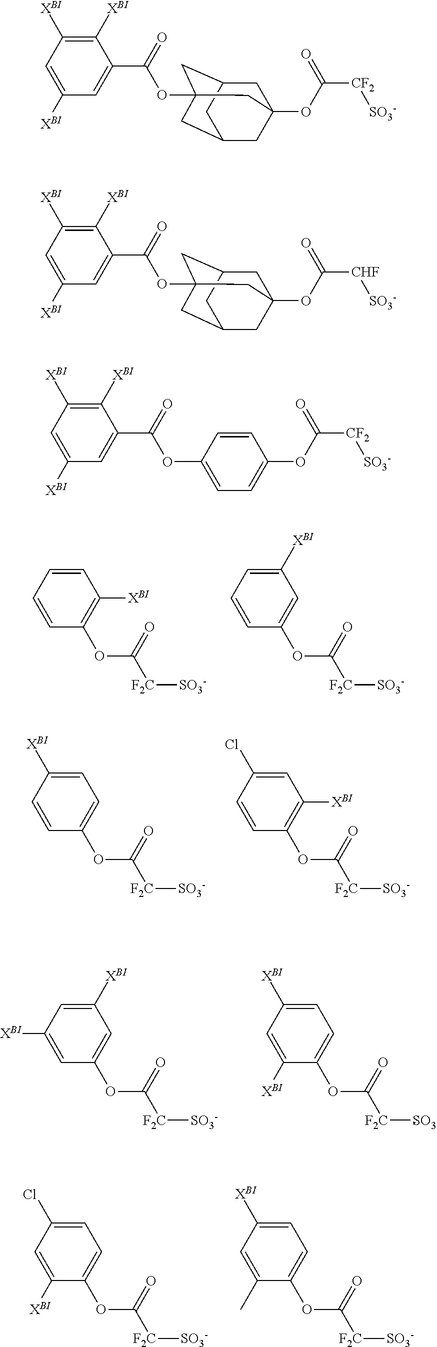

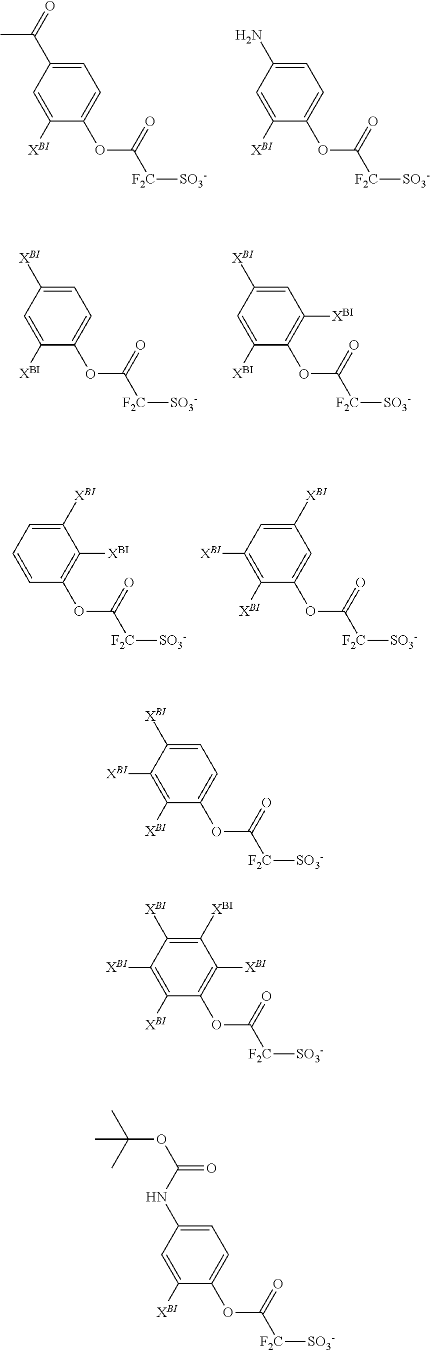

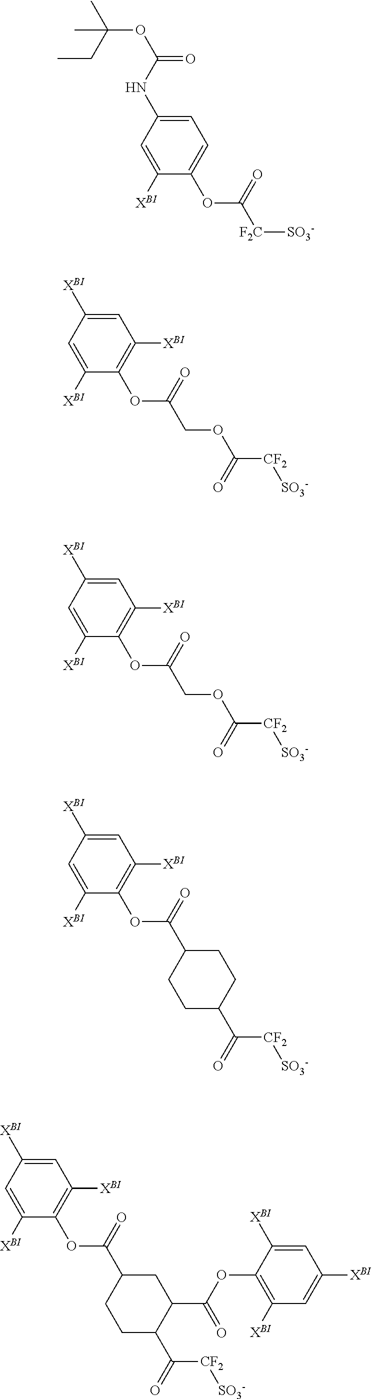

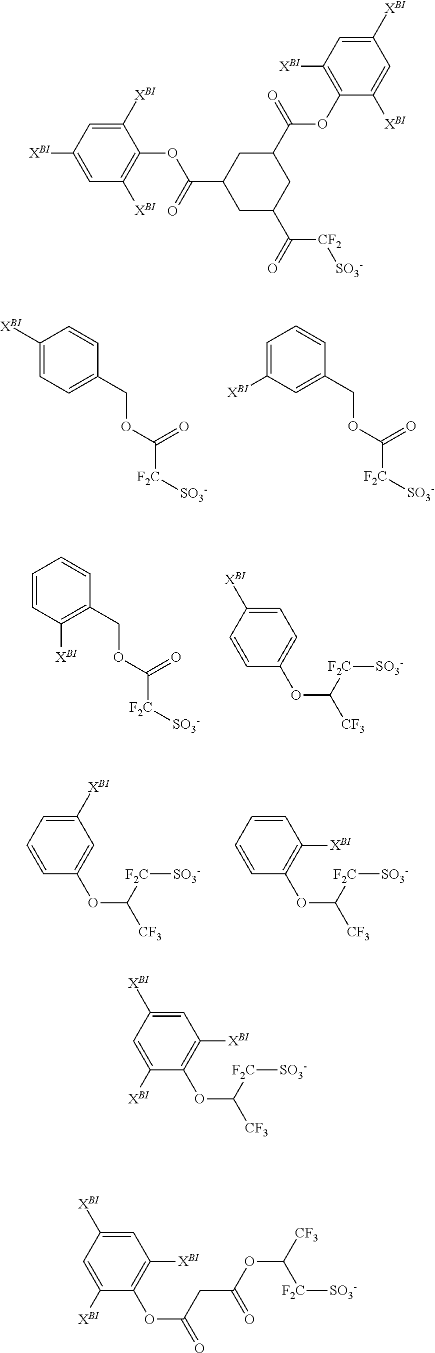

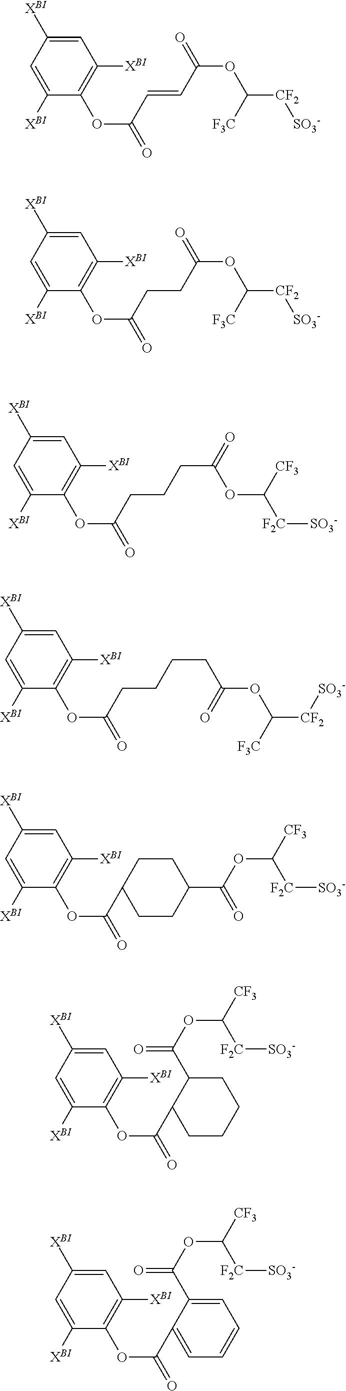

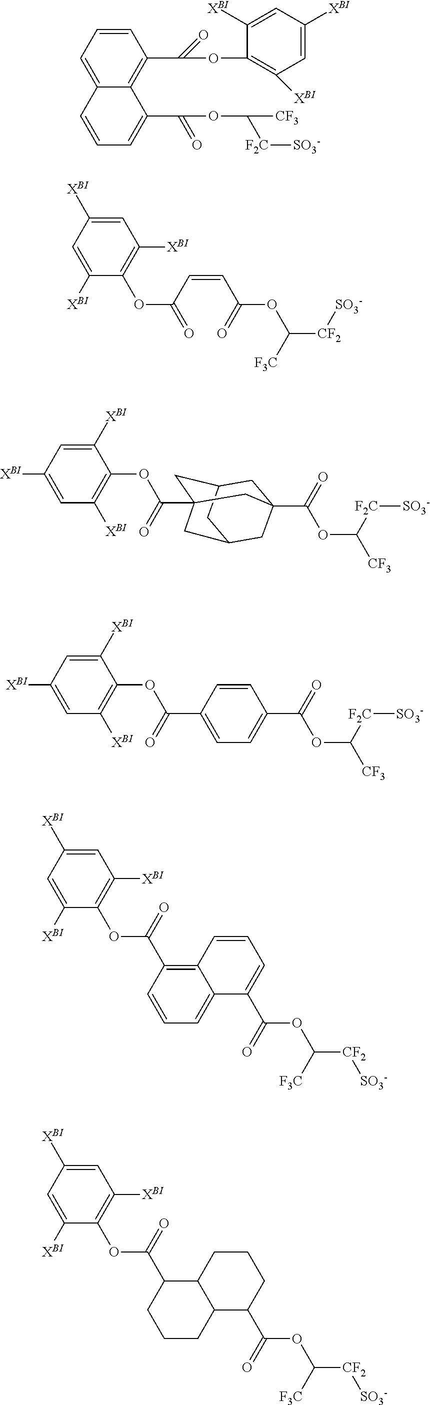

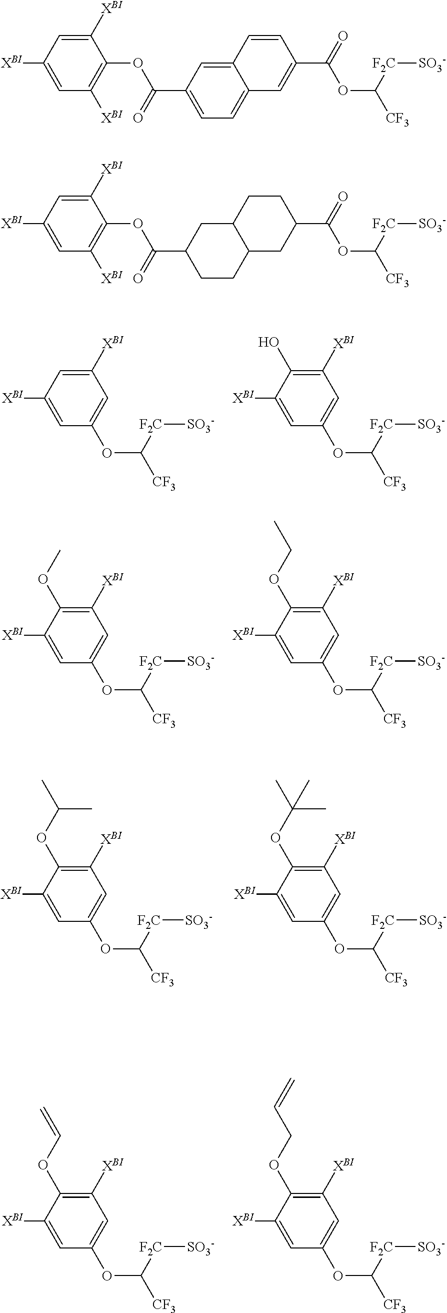

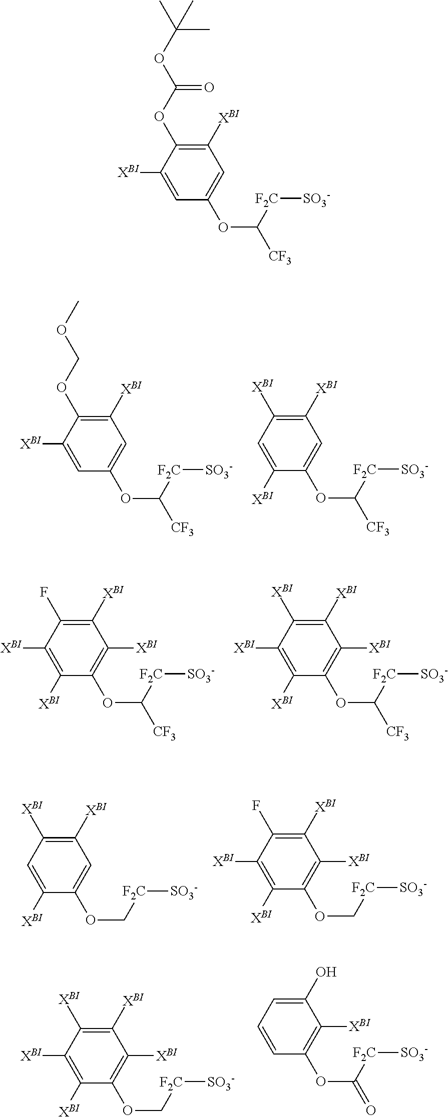

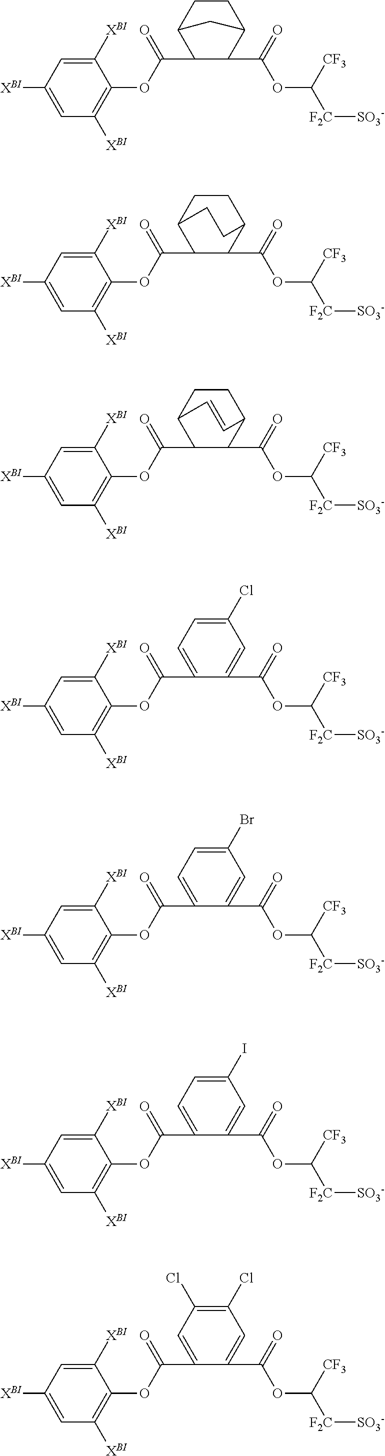

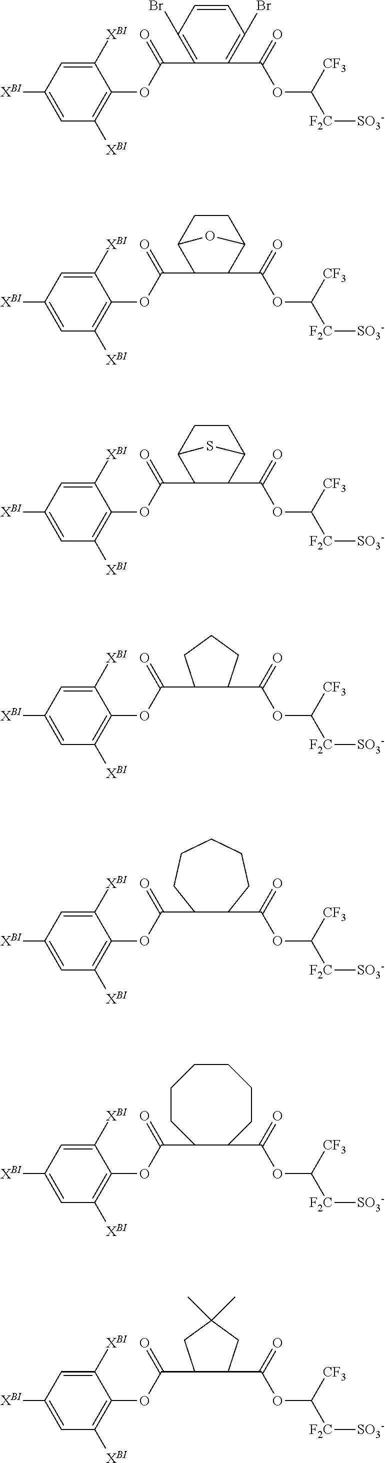

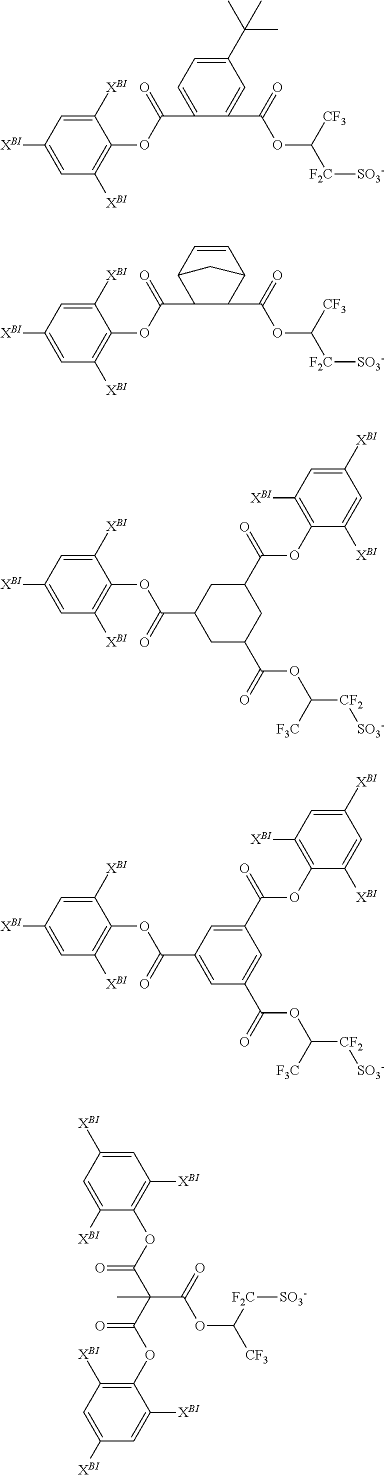

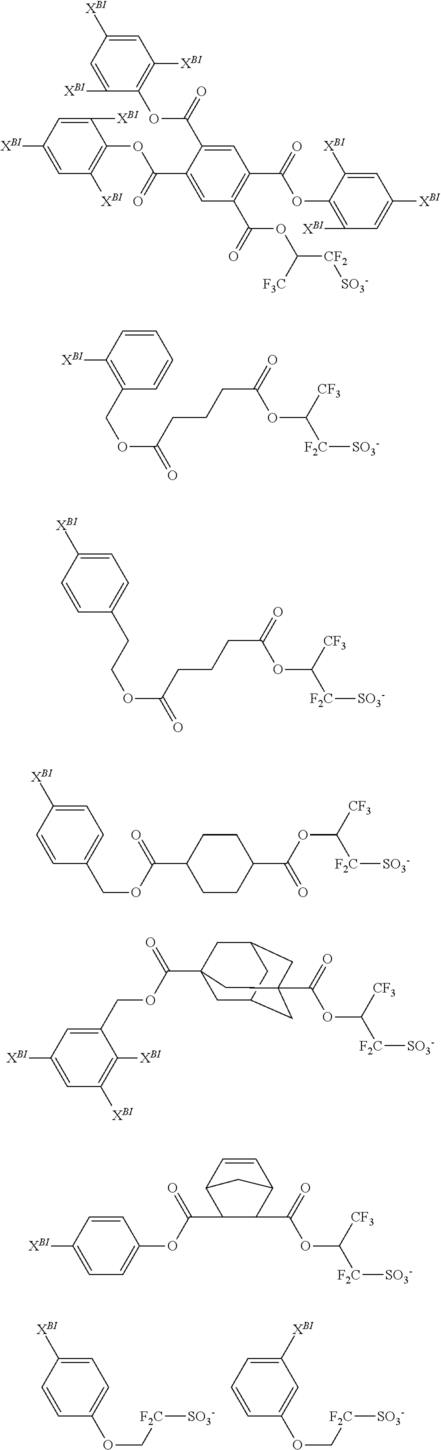

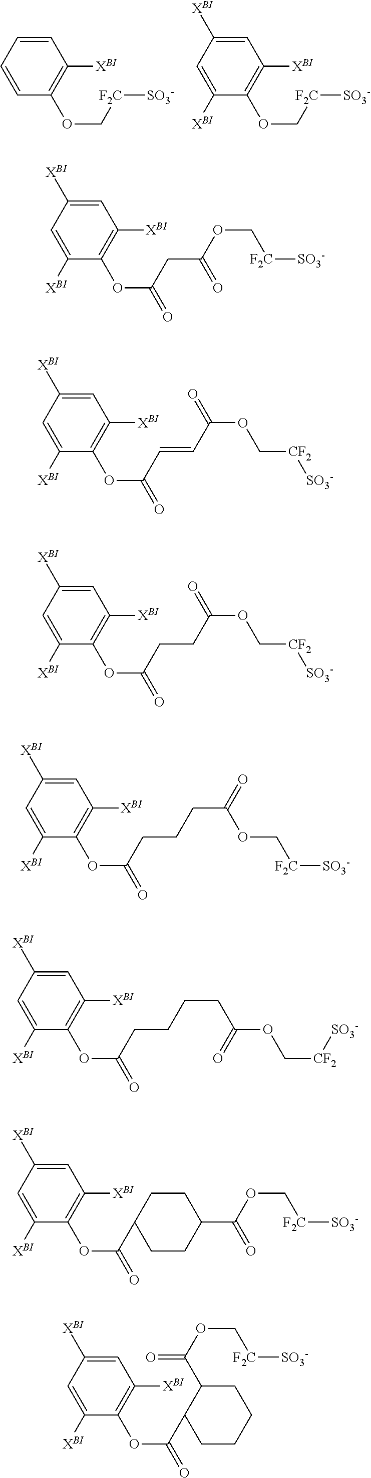

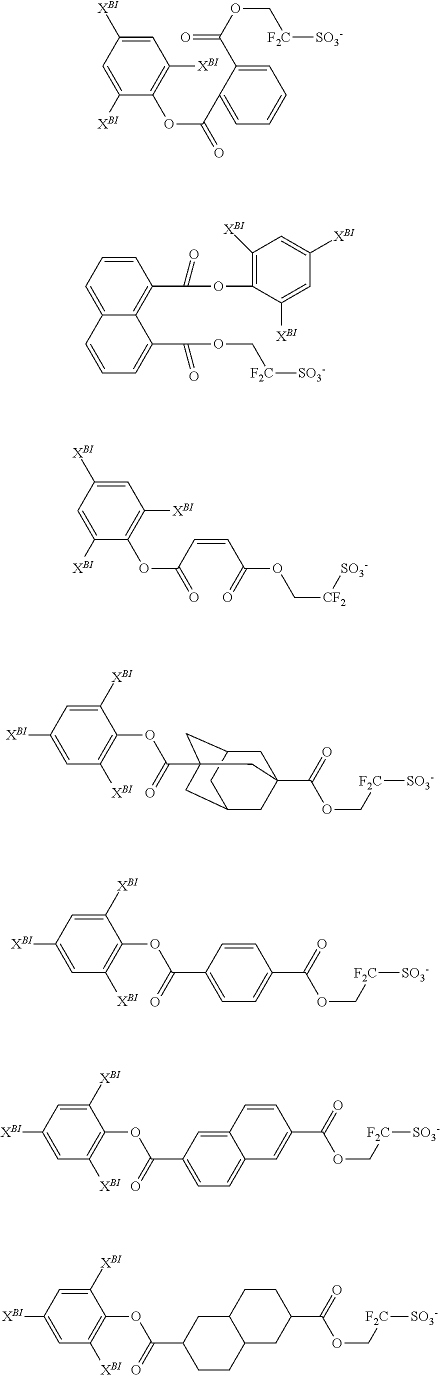

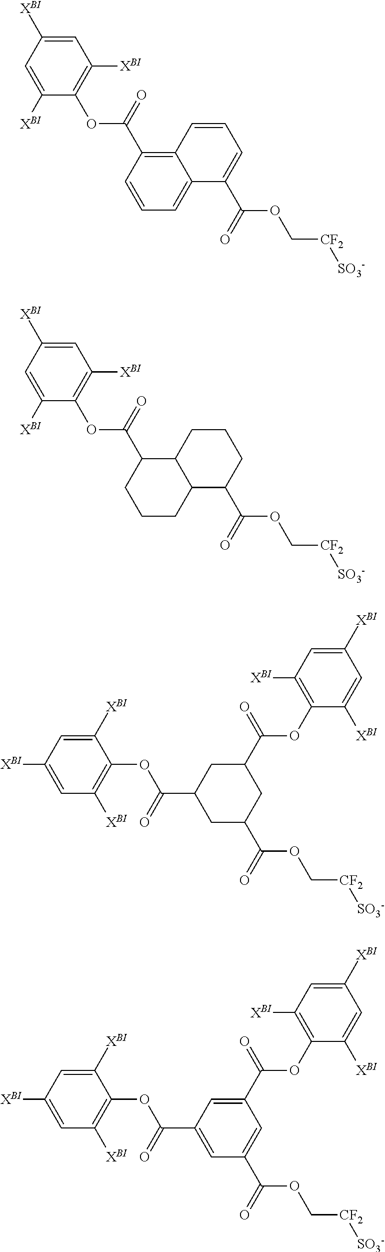

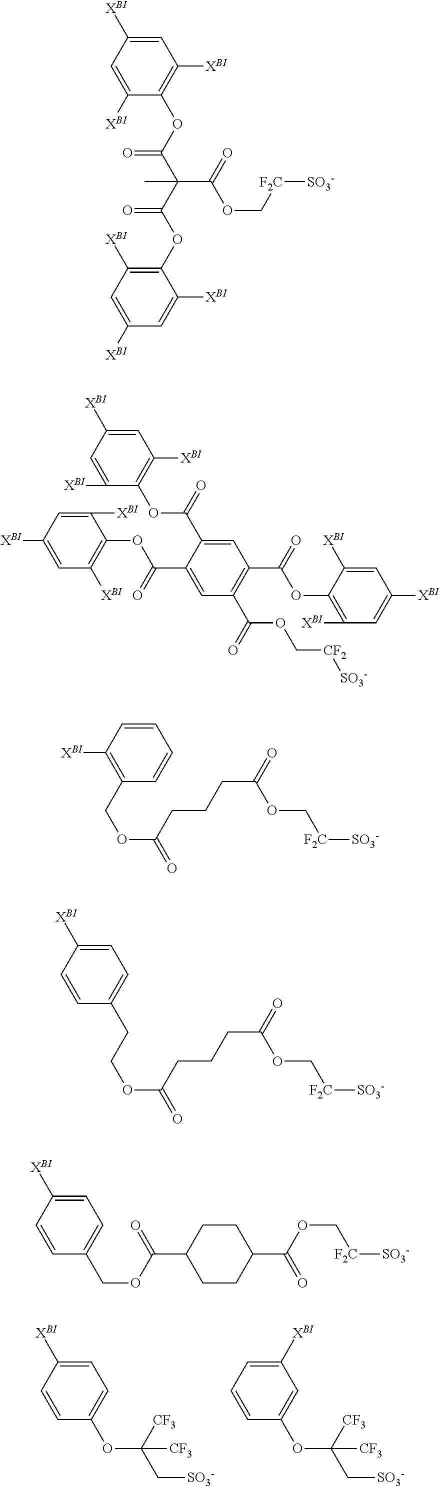

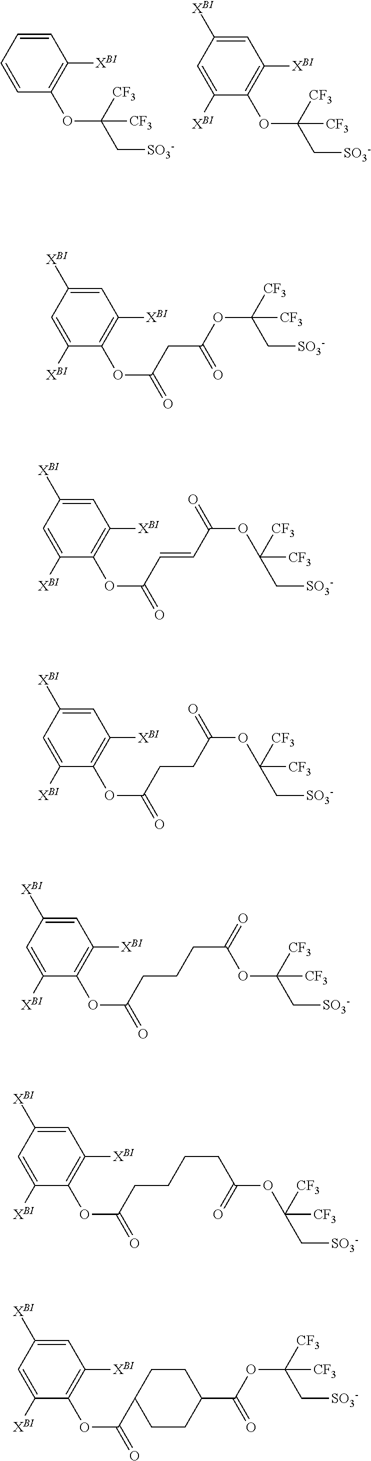

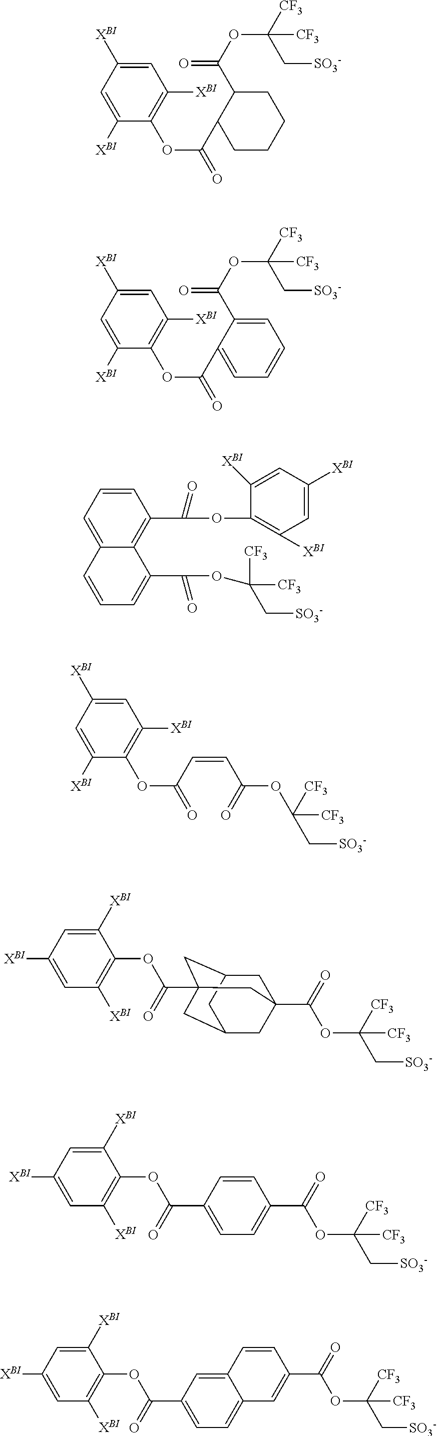

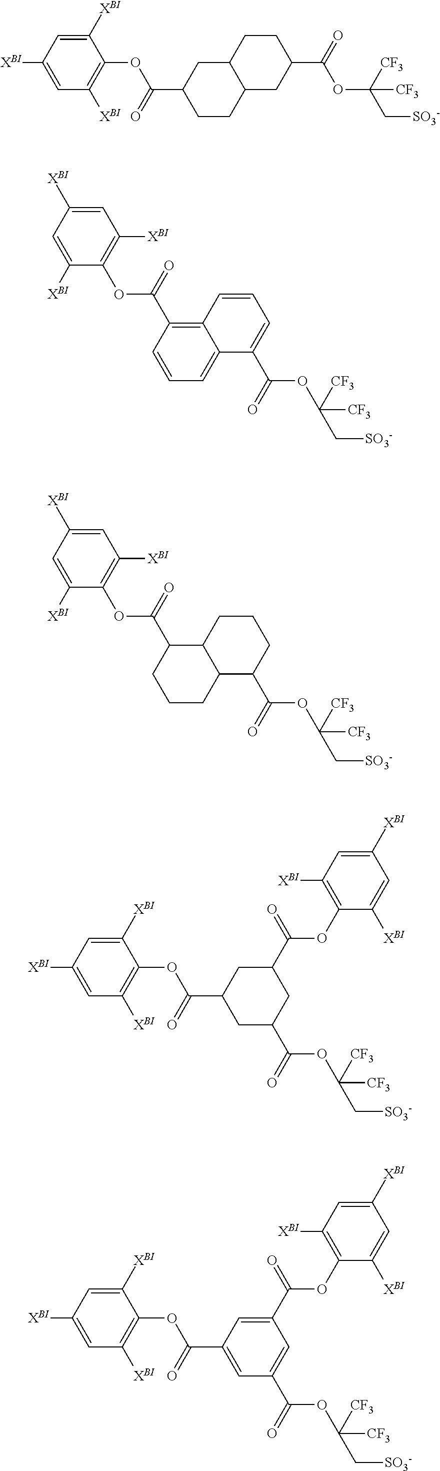

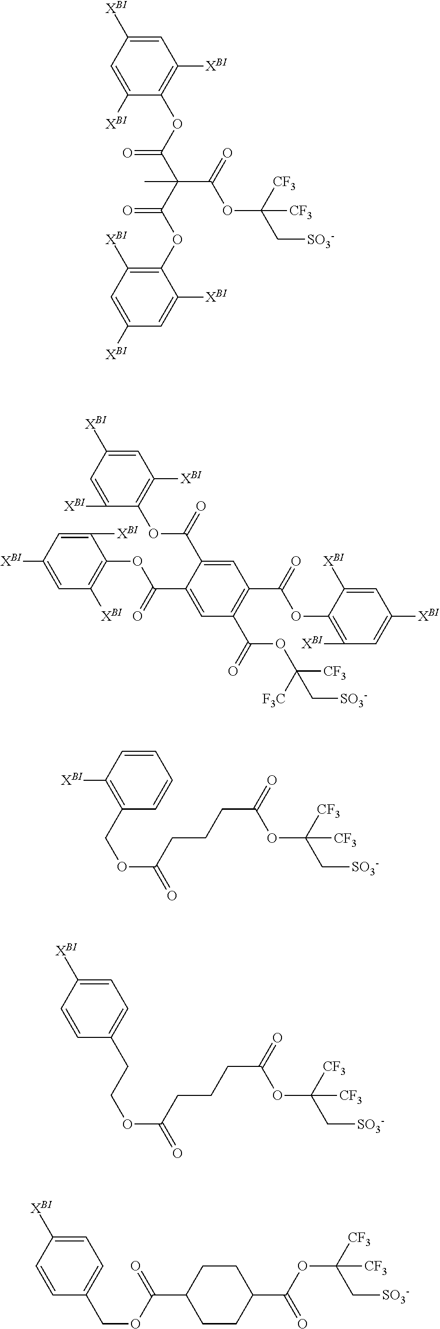

Other useful examples of the non-nucleophilic counter ion include fluorobenzenesulfonic acid anions having an iodized aromatic ring bonded thereto as described in JP 6648726, anions having an acid-catalyzed decomposition mechanism as described in WO 2021/200056 and JP-A 2021-070692, anions having a cyclic ether group as described in JP-A 2018-180525 and JP-A 2021-35935, and anions as described in JP-A 2018-092159.

Further useful examples of the non-nucleophilic counter ion include bulky fluorine-free benzenesulfonic acid anions as described in JP-A 2006-276759, JP-A 2015-117200, JP-A 2016-65016, and JP-A 2019-202974, and fluorine-free benzenesulfonic acid or alkylsulfonic acid anions having an iodized aromatic group bonded thereto as described in JP 6645464.

Also useful examples of the non-nucleophilic counter ion include bissulfonic acid anions as described in JP-A 2015-206932, sulfonamide or sulfonimide anions having sulfonic acid side and different side as described in WO 2020/158366, and anions having a sulfonic acid side and a carboxylic acid side as described in JP-A 2015-024989.

The photoacid generator of the component (D) preferably has the following formula (3).

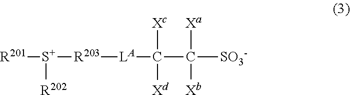

In formula (3), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰², and R²⁰³ may bond together to form a ring with the sulfur atom to which they are attached.

The C₁-C₃₀ hydrocarbyl group represented by R²⁰¹ to R²⁰² may be saturated or unsaturated and straight, branched, or cyclic. Specific examples thereof include C₁-C₃₀ alkyl groups such as a methyl group, an ethyl group, a n-propyl group, an isopropyl group, a n-butyl group, a sec-butyl group, a tert-butyl group, a tert-pentyl group, a n-pentyl group, a n-hexyl group, a n-octyl group, a 2-ethylhexyl group, a n-nonyl group, and a n-decyl group; C₃-C₃₀ cyclic saturated hydrocarbyl groups such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, an oxanorbornyl group, a tricyclo[5.2.1.0^{2.6}]decyl group, and an adamantyl group; C₆-C₃₀ aryl groups such as a phenyl group, a methylphenyl group, an ethylphenyl group, a n-propylphenyl group, an isopropylphenyl group, a n-butylphenyl group, an isobutylphenyl group, a sec-butylphenyl group, a tert-butylphenyl group, a naphthyl group, a methylnaphthyl group, an ethylnaphthyl group, a n-propylnaphthyl group, an isopropylnaphthyl group, a n-butylnaphthyl group, an isobutylnaphthyl group, a sec-butylnaphthyl group, a tert-butylnaphthyl group, and an anthracenyl group; and groups obtained by combining these. Some or all of the hydrogen atoms in the hydrocarbyl group may be substituted with a group containing a heteroatom such as an oxygen atom, a sulfur atom, a nitrogen atom, or a halogen atom, some of —CH₂—in the hydrocarbyl group may be substituted with a group containing a heteroatom such as an oxygen atom, a sulfur atom, or a nitrogen atom, so that the group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, or the like.

The C₁-C₃₀ hydrocarbylene group represented by R²⁰³ may be saturated or unsaturated and straight, branched, or cyclic. Specific examples thereof include C₁-C₃₀ alkanediyl groups such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; C₃-C₃₀ cyclic saturated hydrocarbylene groups such as a cyclopentanediyl group, a cyclohexanediyl group, a norbornanediyl group, and an adamantanediyl group; and C₆-C₃₀ arylene groups such as a phenylene group, a methylphenylene group, an ethylphenylene group, a n-propylphenylene group, an isopropylphenylene group, a n-butylphenylene group, an isobutylphenylene group, a sec-butylphenylene group, a tert-butylphenylene group, a naphthylene group, a methylnaphthylene group, an ethylnaphthylene group, a n-propylnaphthylene group, an isopropylnaphthylene group, a n-butylnaphthylene group, an isobutylnaphthylene group, a sec-butylnaphthylene group, and a tert-butylnaphthylene group. Some or all of the hydrogen atoms in the hydrocarbylene group may be substituted with a group containing a heteroatom such as an oxygen atom, a sulfur atom, a nitrogen atom, or a halogen atom, some of —CH₂—in the hydrocarbylene group may be substituted with a group containing a heteroatom such as an oxygen atom, a sulfur atom, or a nitrogen atom, so that the group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, or the like. The heteroatom is preferably an oxygen atom.

In formula (3), L^{A} is a single bond, an ether bond, or a C₁-C₂₀ hydrocarbylene group which may contain a heteroatom. The hydrocarbylene group may be saturated or unsaturated and straight, branched, or cyclic. Specific examples thereof are as exemplified above for the hydrocarbylene group represented by R²⁰³.

In formula (3), X^{a}, X^{b}, X^{c}, and X^{d} are each independently a hydrogen atom, a fluorine atom, or a trifluoromethyl group. Provided that at least one of X^{a}, X^{b}, X^{c}, and X^{d} is a fluorine atom or a trifluoromethyl group.

The photoacid generator having formula (3) preferably has the following formula (3′).

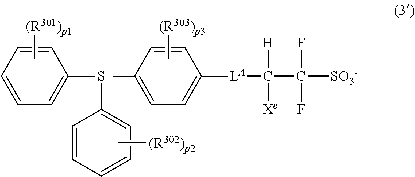

In formula (3′), L^{A} is as defined above. X^{c} is a hydrogen atom or a trifluoromethyl group and preferably a trifluoromethyl group. R³⁰¹, R³⁰², and R³⁰³ are each independently a hydrogen atom, or a C₁-C₂₀ hydrocarbyl group which may contain a heteroatom. The hydrocarbyl group may be saturated or unsaturated and straight, branched, or cyclic. Specific examples thereof are as exemplified above for the hydrocarbyl group represented by R^{fa1} in formula (2A′). p1 and p2 are each independently an integer of 0 to 5, and p3 is an integer of 0 to 4.

Examples of the photoacid generator having formula (3) are as exemplified for the photoacid generator having formula (2) in JP-A 2017-026980.

Of the foregoing other photoacid generators, those having an anion of formula (2A′) or (2D) are particularly preferred because of reduced acid diffusion and high solubility in the solvent. Those having formula (3′) are particularly preferred because of extremely reduced acid diffusion.

When the resist composition of the present invention comprises the photoacid generator (D), the content thereof is preferably 0.1 to 40 parts by weight and more preferably 0.5 to 20 parts by weight per 80 parts by weight of the base polymer (C). When the added amount of the photoacid generator of the component (D) is in the above range, the resolution is favorable, and there is no possibility that a problem of foreign matter occurs after development or peeling of the resist film, which is preferable. The photoacid generator of the component (D) may be used alone or in combination of two or more kinds thereof. The resist composition of the present invention can function as a chemically amplified resist composition when the base polymer comprises any of the repeat units c1 to c4 and/or the photoacid generator (D) is contained.

### [Nitrogen-Containing Compound (E)]

In the resist composition of the present invention, the quencher of the component (A) is an essential component, but in addition to this, a nitrogen-containing compound may be contained as another quencher. Examples of such a nitrogen-containing compound include primary, secondary, and tertiary amine compounds, specifically amine compounds having a hydroxy group, an ether bond, an ester bond, a lactone ring, a cyano group, or a sulfonic acid ester bond as described in JP-A 2008-111103, paragraphs to [0164]. as in the compound described in JP 3790649, a compound in which a primary or secondary amine is protected with a carbamate group can also be mentioned.

A sulfonium sulfonate having a nitrogen-containing substituent may be used as the nitrogen-containing compound. Such a compound functions as a quencher in the unexposed area, and the exposed area functions as a so-called photodegradable base that loses quencher capability by neutralization with its own generated acid. By using the photodegradable base, the contrast between the exposed area and the unexposed area can be further enhanced. As the photodegradable base, for example, JP-A 2009-109595, JP-A 2012-46501, and the like can be referred to.

When the resist composition of the present invention comprises the nitrogen-containing compound (E), the content thereof is preferably 0.001 to 12 parts by weight and more preferably 0.01 to 8 parts by weight per 80 parts by weight of the base polymer (C). The nitrogen-containing compound (E) may be used alone or in combination of two or more kinds thereof.

### [Surfactant (F)]

The resist composition of the present invention may further comprise a surfactant (F). The surfactant of the component (F) is preferably a surfactant insoluble or sparingly soluble in water and soluble in an alkaline developer, or a surfactant insoluble or sparingly soluble in water and an alkaline developer. As such a surfactant, those described in JP-A 2010-215608 and JP-A 2011-16746 can be referred to.

As the surfactant insoluble or sparingly soluble in water and an alkaline developer, among the surfactants described in the above publication, FC-4430 (manufactured by 3M), SURFLON® S-381 (manufactured by AGC Seimi Chemical Co., Ltd.), OLFINE® E1004 (manufactured by Nissin Chemical Industry Co., Ltd.), KH-20, KH-30 (manufactured by AGC Seimi Chemical Co., Ltd.), an oxetane ring-opening polymer having the following formula (surf-1), and the like are preferable.

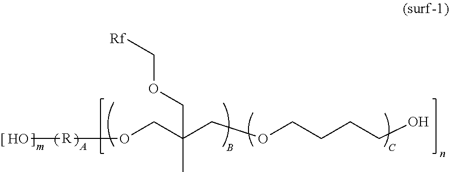

R, Rf, A, B, C, m, and n apply only to formula (surf-1), regardless of the foregoing description. R is a di- to tetra-valent C₂-C₅ aliphatic group. Examples of the divalent aliphatic group include an ethylene group, a 1,4-butylene group, a 1,2-propylene group, a 2,2-dimethyl-1,3-propylene group, and a 1,5-pentylene group, and examples of the tri- and tetra-valent aliphatic group are shown below.

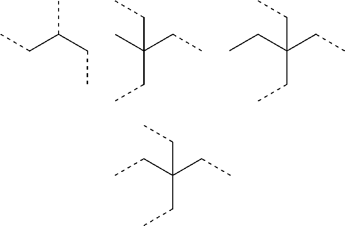

wherein the broken line denotes a point of attachment, and these formulae are partial structures derived from glycerol, trimethylolethane, trimethylolpropane, and pentaerythritol.

Of these, a 1,4-butylene group, a 2,2-dimethyl-1,3-propylene group, and the like are preferable.

Rf is a trifluoromethyl group or a pentafluoroethyl group, preferably a trifluoromethyl group. m is an integer of 0 to 3, n is an integer of 1 to 4, and the sum of n and m, which represents the valence of R, is an integer of 2 to 4. A is 1. B is an integer of 2 to 25, preferably an integer of 4 to 20. C is an integer of 0 to 10, preferably 0 or 1. Each constituent unit in formula (surf-1) does not prescribe the arrangement thereof, and may be arranged either blockwise or randomly. For the preparation of surfactants in the form of partially fluorinated oxetane ring-opened polymers, reference should be made to U.S. Pat. No. 5,650,483 and the like.

When a resist protective film is not used in ArF immersion lithography, the surfactant insoluble or sparingly soluble in water and soluble in an alkaline developer has a function of minimizing water penetration or leaching by being oriented on the surface of the resist film. Therefore, the surfactant is useful for preventing water-soluble components from being leached out of the resist film for minimizing any damage to the exposure tool, and is also useful because it becomes solubilized during alkaline aqueous solution development after exposure or after PEB, and thus forms few or no foreign matter which become defects. Such a surfactant has a property of being insoluble or sparingly soluble in water and being soluble in an alkaline developer, is also called a polymeric surfactant, and is particularly preferably a surfactant having high water repellency and improving lubricity.

Specific examples of such a polymeric surfactant include those containing at least one selected from repeat units having the following formulae (4A) to (4E).

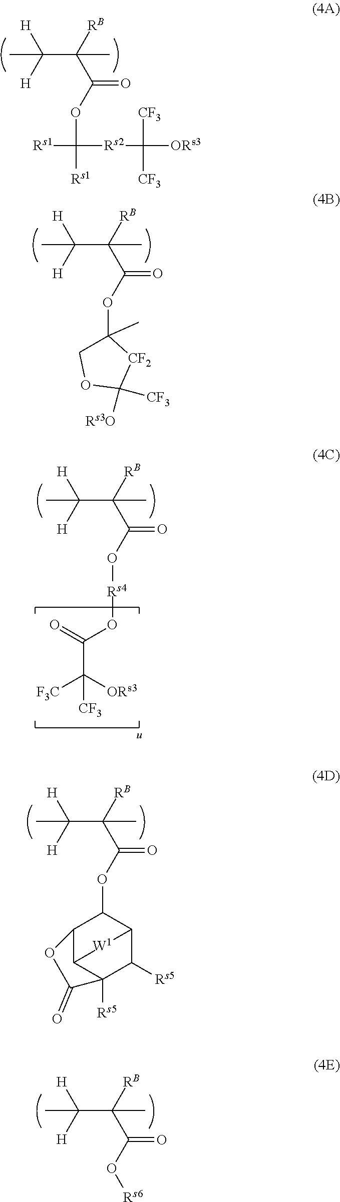

In formulae (4A) to (4E), RB is a hydrogen atom, a fluorine atom, a methyl group, or a trifluoromethyl group. W¹ is-CH₂—, —CH₂CH₂—, —O—, or two separate —H. R^{s1} is each independently a hydrogen atom or a C₁-C₁₀ hydrocarbyl group. R^{s2} is a single bond or a C₁-C₅ straight or branched hydrocarbylene group. R^{s3} is each independently a hydrogen atom, a C₁-Cis hydrocarbyl group or fluorinated hydrocarbyl group, or an acid labile group. When R^{s3} is a hydrocarbyl group or fluorinated hydrocarbyl group, an ether bond or a carbonyl group may intervene in a carbon-carbon bond. R^{s4} is a C₁-C₂₀ (u+1)-valent hydrocarbon group or fluorinated hydrocarbon group. u is an integer of 1 to 3. R^{s5} is each independently a hydrogen atom or a group having formula-C(═O)—O—R^{sa}, and R^{sa} is a C₁-C₂₀ fluorinated hydrocarbyl group. R^{s6} is a C₁-C₁₅ hydrocarbyl group or fluorinated hydrocarbyl group, and an ether bond or a carbonyl group may intervene in a carbon-carbon bond thereof.

The hydrocarbyl group represented by R^{s1} is preferably a saturated hydrocarbyl group and may be straight, branched, or cyclic. Specific examples thereof include alkyl groups such as a methyl group, an ethyl group, a n-propyl group, an isopropyl group, a n-butyl group, an isobutyl group, a sec-butyl group, a tert-butyl group, a n-pentyl group, a n-hexyl group, a n-heptyl group, a n-octyl group, a n-nonyl group, and a n-decyl group; and cyclic saturated hydrocarbyl groups such as a cyclopropyl group, a cyclobutyl group, a cyclopentyl group, a cyclohexyl group, an adamantyl group, and a norbornyl group. Of these, C₁-C₆ groups are preferable.

The hydrocarbylene group represented by R^{s2} is preferably a saturated hydrocarbylene group and may be straight, branched, or cyclic. Specific examples thereof include a methylene group, an ethylene group, a propylene group, a butylene group, and a pentylene group.

The hydrocarbyl group represented by R^{s3} or R^{s6} may be saturated or unsaturated and straight, branched, or cyclic. Specific examples thereof include aliphatic unsaturated hydrocarbyl groups such as a saturated hydrocarbyl group, an alkenyl group, and an alkynyl group, and a saturated hydrocarbyl group is preferable. Examples of the saturated hydrocarbyl group include a n-undecyl group, a n-dodecyl group, a tridecyl group, a tetradecyl group, and a pentadecyl group in addition to those exemplified as the hydrocarbyl group represented by R^{s1}. Examples of the fluorinated hydrocarbyl group represented by R $3 or R^{s6} include groups in which some or all hydrogen atoms bonded to carbon atoms of the foregoing hydrocarbyl group are substituted by fluorine atoms. As described above, an ether bond or a carbonyl group may be interposed between these carbon-carbon bonds.

Specific examples of the acid labile group represented by R^{s3} include the groups having formulae (AL-3) to (AL-5), trialkylsilyl groups in which each alkyl group is a C₁-C₆ alkyl group, and C₄-C₂₀ oxo group-containing alkyl groups.

The (u+1)-valent hydrocarbon or fluorinated hydrocarbon group represented by R $4 may be straight, branched, or cyclic, and specific examples thereof include the foregoing hydrocarbyl or fluorinated hydrocarbyl groups from which “u” number of hydrogen atoms are eliminated.

The fluorinated hydrocarbyl group represented by R^{sa} is preferably saturated and may be straight, branched, or cyclic. Specific examples thereof include the foregoing hydrocarbyl groups in which some or all hydrogen atoms are substituted by fluorine atoms, and examples thereof include a trifluoromethyl group, a 2,2,2-trifluoroethyl group, a 3,3,3-trifluoro-1-propyl group, a 3,3,3-trifluoro-2-propyl group, a 2,2,3,3-tetrafluoropropyl group, a 1,1,1,3,3,3-hexafluoroisopropyl group, a 2,2,3,3,4,4,4-heptafluorobutyl group, a 2,2,3,3,4,4,5,5-octafluoropentyl group, a 2,2,3,3,4,4,5,5,6,6,7,7-dodecafluoroheptyl group, a 2-(perfluorobutyl)ethyl group, a 2-(perfluorohexyl)ethyl group, a 2-(perfluorooctyl)ethyl group, and a 2-(perfluorodecyl)ethyl group.

Specific examples of the repeat units having formulae (4A) to (4E) are shown below, but not limited thereto. In the following formula, RB is as defined above.

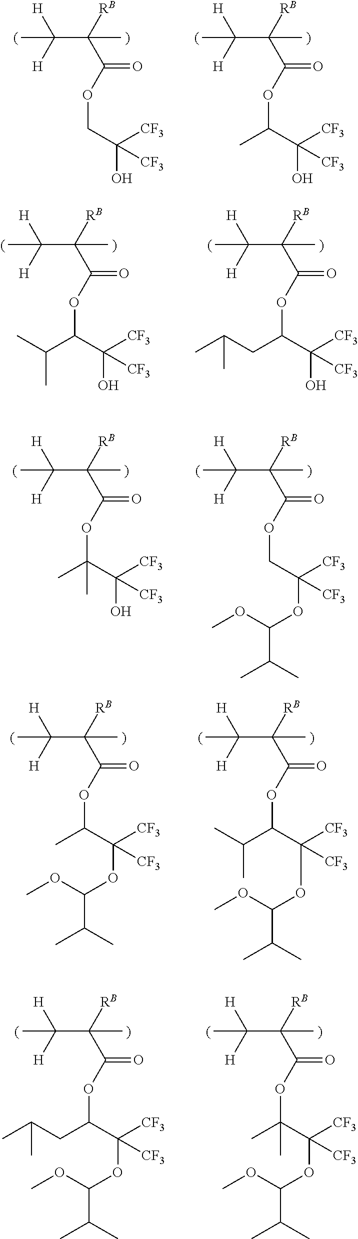

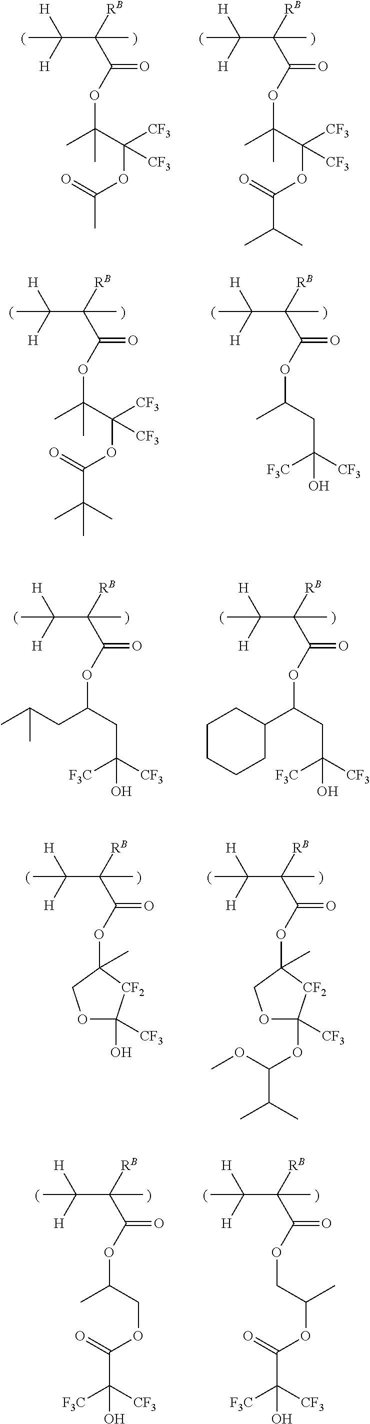

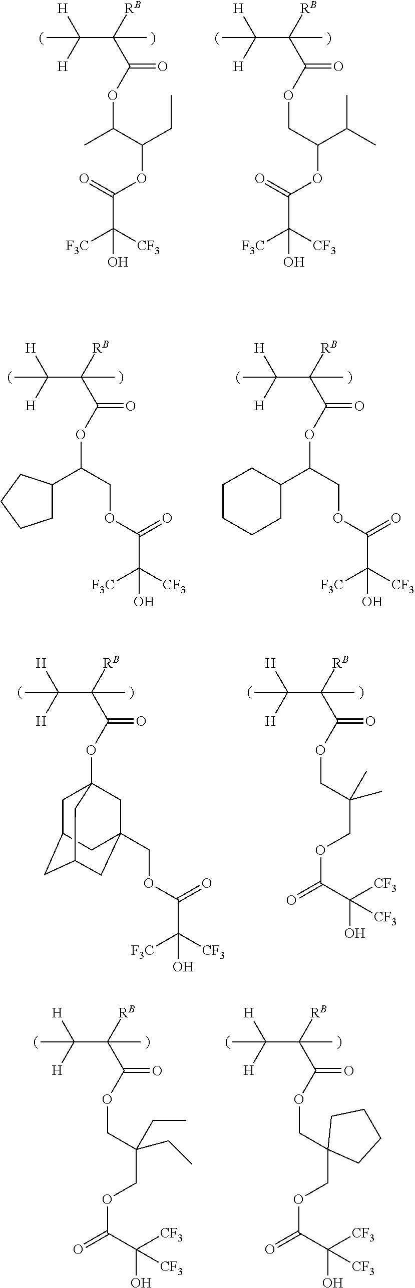

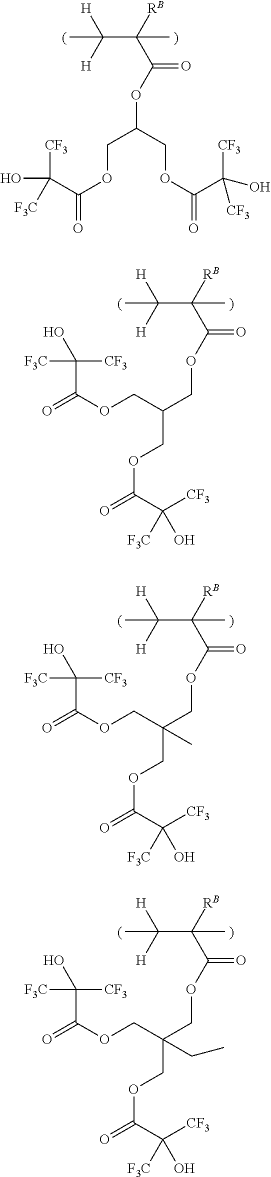

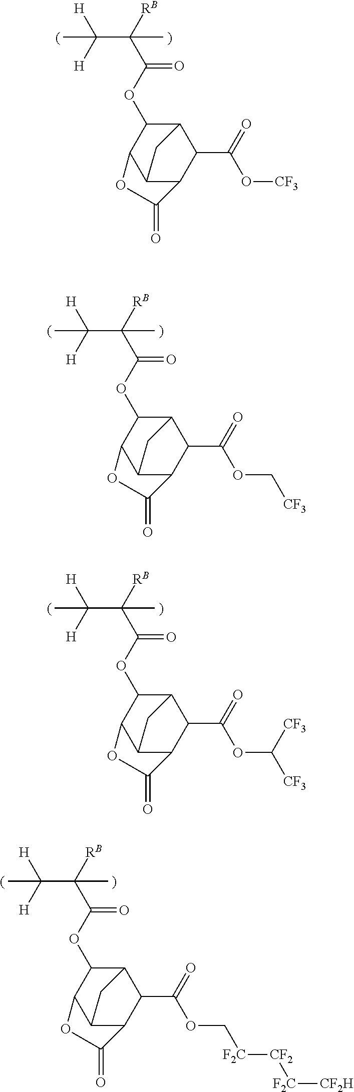

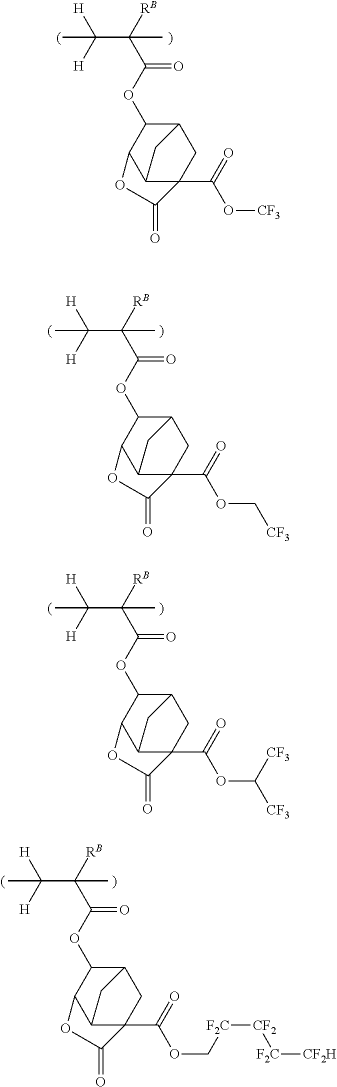

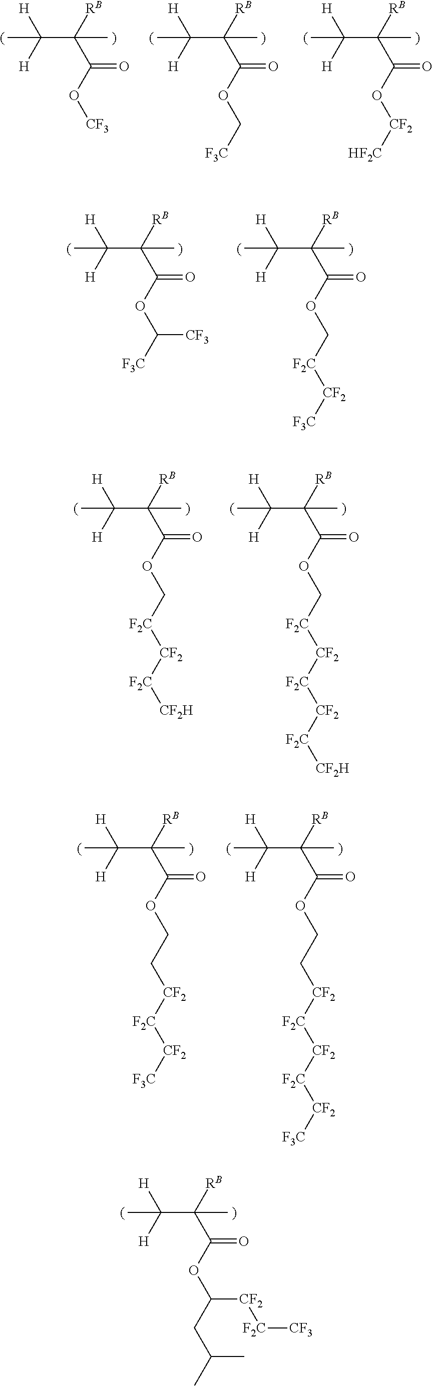

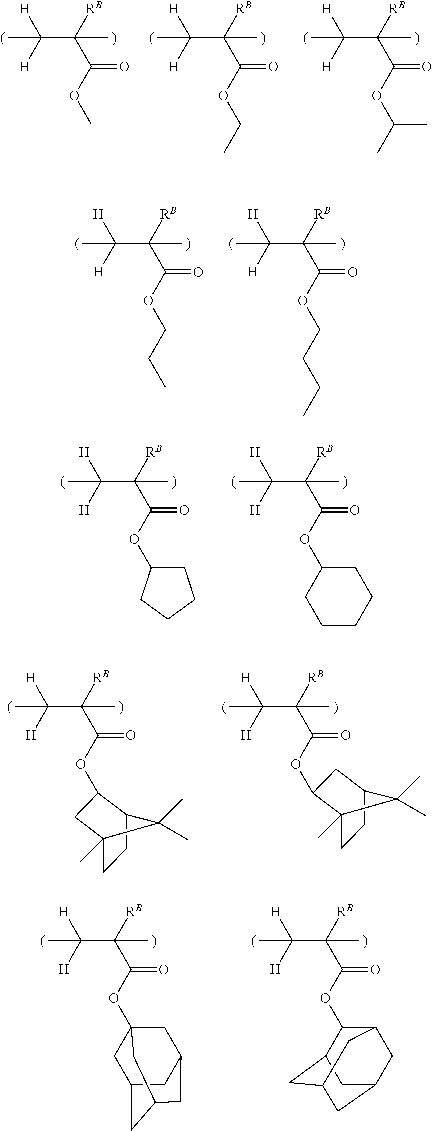

The polymeric surfactant may further contain repeat units other than the repeat units having formulae (4A) to (4E). Examples of the other repeat units include repeat units derived from methacrylic acid, an α-trifluoromethylacrylic acid derivative, or the like. In the polymeric surfactant, the content of the repeat units having formulae (4A) to (4E) is preferably 20 mol % or more, more preferably 60 mol % or more, and still more preferably 100 mol % of the overall repeat units.

The Mw of the polymeric surfactant is preferably 1000 to 500000, more preferably 3000 to 100000. Mw/Mn is preferably 1.0 to 2.0, more preferably 1.0 to 1.6.

Examples of the method for synthesizing the polymeric surfactant include a method of dissolving an unsaturated bond-containing monomer providing repeat units having formulae (4A) to (4E) and optionally other repeat units in an organic solvent, adding a radical initiator, and heating for polymerization. Examples of the organic solvent used in the polymerization include toluene, benzene, THF, diethyl ether, and dioxane. Examples of the polymerization initiator include AIBN, 2,2′-azobis(2,4-dimethylvaleronitrile), dimethyl 2,2-azobis(2-methylpropionate), benzoyl peroxide, and lauroyl peroxide. The reaction temperature is preferably 50 to 100° C. The reaction time is preferably 4 to 24 hours. The acid labile group that has been incorporated in the monomer may be kept as such, or polymerization may be followed by protection or partial protection.

In the case of synthesizing the polymeric surfactant, any known chain transfer agent such as dodecyl mercaptan or 2-mercaptoethanol may be used for molecular weight control purpose. In this case, these chain transfer agents are preferably added in an amount of 0.01 to 10 mol % based on the total number of moles of monomers to be polymerized.

When the resist composition of the present invention comprises the surfactant (F), the content thereof is preferably 0.1 to 50 parts by weight and more preferably 0.5 to 10 parts by weight per 80 parts by weight of the base polymer (C). When the content of the surfactant (F) is 0.1 parts by weight or more, the receding contact angle with water of the resist film at its surface is sufficiently improved, and when the content thereof is 50 parts by weight or less, the dissolution rate of the resist film at its surface in the developer is low, and the height of the formed fine pattern is sufficiently maintained. The surfactant (F) may be used alone or in combination of two or more kinds thereof.

### Other Components (G)

The resist composition of the present invention may comprise a compound which is decomposed with an acid to generate another acid (acid amplifier compound), an organic acid derivative, a fluorinated alcohol, a compound having a Mw of 3000 or less which changes its solubility in a developer under the action of acid (dissolution inhibitor), and the like as other components (G). As the acid amplifier compound, a compound described in JP-A 2009-269953 or JP-A 2010-215608 can be referred to. When the resist composition comprises the acid amplifier compound, the content thereof is preferably 0 to 5 parts by weight and more preferably 0 to 3 parts by weight per 80 parts by weight of the base polymer (C). When the content thereof is too large, it is difficult to control acid diffusion, and resolution and pattern profile may be deteriorated. As the organic acid derivative, the fluorinated alcohol, and the dissolution inhibitor, compounds described in JP-A 2009-269953 or JP-A 2010-215608 can be referred to.

### [Pattern Forming Process]

A pattern forming process of the present invention comprises the steps of: applying the resist composition defined above onto a substrate to form a resist film thereon; exposing the resist film to high-energy radiation; and developing the exposed resist film in a developer.

As the substrate, for example, substrates for integrated circuit fabrication (such as Si, SiO₂, SiN, SION, TIN, WSi, BPSG, SOG, and organic antireflective coating), or substrates for mask circuit fabrication (such as Cr, CrO, CrON, MoSi₂, and SiO₂) can be used.

The resist film can be formed by, for example, applying the resist composition by a method such as spin coating so that the film thickness is preferably 0.05 to 2 μm, and prebaking the resist composition on a hotplate at preferably 60 to 150° C. for 1 to 10 minutes, more preferably 80 to 140° C. for 1 to 5 minutes.

Examples of the high-energy radiation used for exposure of the resist film include i-ray, KrF excimer laser, ArF excimer laser, EB, and EUV. In the case of using KrF excimer laser, ArF excimer laser, or EUV, exposure can be performed by using a mask for forming a target pattern and performing irradiation so that the exposure dose is preferably 1 to 200 mJ/cm², more preferably 10 to 100 mJ/cm². In the case of using EB, irradiation is performed using a mask for forming a target pattern or directly so that the exposure dose is preferably 1 to 300 μC/cm², more preferably 10 to 200 μC/cm².

In addition to a normal exposure method, it is also possible to use an immersion method in which exposure is performed by interposing a liquid having a refractive index of 1.0 or more between a resist film and a projection lens. In this case, it is also possible to use a protective film insoluble in water.

The water-insoluble protective film is used to prevent an eluate from the resist film and to increase the lubricity of the film surface, and is generally divided into two types. The first type is an organic solvent-strippable protective film in which peeling is required before alkaline aqueous solution development by an organic solvent that does not dissolve a resist film, and the second type is an alkaline aqueous solution-soluble protective film which is soluble in an alkaline developer so that the protective film is removed simultaneously with the removal of solubilized regions of the resist film. The protective film of the second type is particularly preferably of a material comprising a polymer having a 1,1,1,3,3,3-hexafluoro-2-propanol residue, which is insoluble in water and soluble in an alkaline developer, as a base and dissolved in an alcohol-based solvent of 4 or more carbon atoms, an ether-based solvent of 8 to 12 carbon atoms, and a mixed solvent thereof. A material obtained by dissolving the surfactant, which is insoluble in water and soluble in an alkaline developer, in an alcohol-based solvent of 4 or more carbon atoms, an ether-based solvent of 8 to 12 carbon atoms, or a mixed solvent thereof can also be used.

After the exposure, PEB may be performed. PEB can be performed, for example, by heating on a hotplate at preferably 60 to 150° C. for 1 to 5 minutes, more preferably 80 to 140° C. for 1 to 3 minutes.

For example, the resist film is developed with a developer in the form of an alkaline aqueous solution such as tetramethylammonium hydroxide (TMAH) in an amount of preferably 0.1 to 5 wt %, more preferably 2 to 3 wt % for preferably 0.1 to 3 minutes, more preferably 0.5 to 2 minutes by conventional techniques such as dip, puddle, and spray techniques. In this way, the exposed area is dissolved and a target pattern is formed on the substrate.

After the resist film is formed, the acid generator or the like may be extracted from the film surface by performing rinsing with pure water, or particles may be washed off, or rinsing for removing water remaining on the film after exposure may be performed.

Pattern formation may be performed by a double patterning process. Examples of the double patterning process include a trench process of processing an underlay to 1:3 trench pattern by a first step of exposure and etching, shifting the position, and forming a 1:3 trench pattern by a second step of exposure, for forming a 1:1 pattern, and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the pattern forming process of the present invention, a method of negative tone development in which an organic solvent is used instead of the alkaline aqueous solution as the developer for dissolving away the unexposed area may be used. For the organic solvent development, as the developer, 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, 2-phenylethyl acetate, and the like can be used. These organic solvents may be used alone or in admixture of two or more kinds thereof.

### EXAMPLES

Hereinafter, the present invention is specifically described with reference to Synthesis Examples, Examples, and Comparative Examples, but the present invention is not limited to the following Examples. The devices used are as follows.
IR: NICOLET 6700 manufactured by Thermo Fisher Scientific Inc.
¹H-NMR: ECA-500 manufactured by JEOL Ltd.
MALDI TOF-MS: S3000 manufactured by JEOL Ltd.

### [1] Synthesis of Onium Salt

### [Example 1-1] Synthesis of Onium Salt NSQ-1

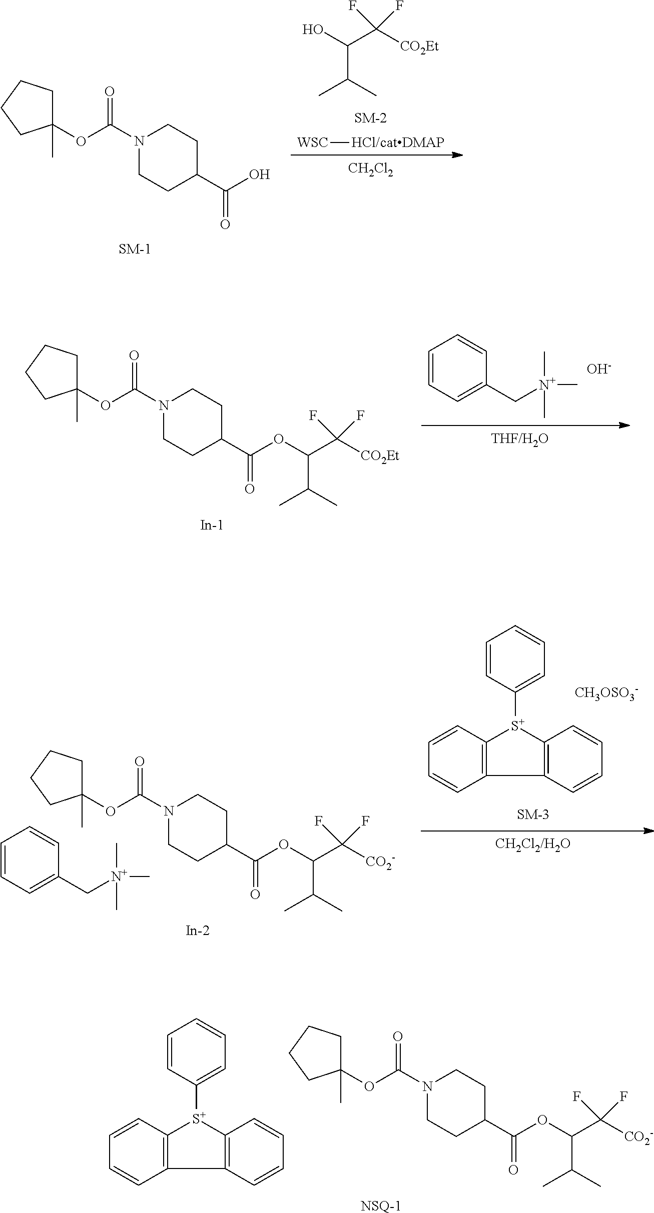

### (1) Synthesis of Intermediate In-1

In a nitrogen atmosphere, a reaction vessel was charged with Compound SM-1 (10.2 g), Compound SM-2 (7.9 g), 4-dimethylaminopyridine (DMAP) (0.5 g), and methylene chloride (50 g), and cooled in an ice bath. 1-ethyl-3-(3-dimethylaminopropyl) carbodiimide hydrochloride (9.2 g) was added as a powder while the temperature in the reaction vessel was kept at 20° C. or lower. After addition, the reaction mixture was warmed up to room temperature, and aged for 12 hours. After aging, water was added to stop the reaction, followed by ordinary aqueous work-up and solvent distillation to obtain Intermediate In-1 as oily matter (yield: 16.4 g, yield percentage: 95%).

### (2) Synthesis of Intermediate In-2

In a nitrogen atmosphere, Intermediate In-1 (16.2 g) was dissolved in THF (40 g). Thereafter, a 25 wt % benzyltrimethylammonium hydroxide aqueous solution (23.9 g) was added dropwise thereto. After the dropwise addition, the reaction solution was heated to 40° C. and aged for 4 hours. After aging, the reaction system was cooled to room temperature, and the solvent was distilled off, then diisopropyl ether was added thereto, and washing was performed to obtain Intermediate In-2 as oily matter (yield: 19.9 g, yield percentage: 90%).

### (3) Synthesis of Onium Salt NSQ-1

In a nitrogen atmosphere, Intermediate In-2 (19.9 g) and Compound SM-3 (13.4 g) were mixed with methylene chloride (100 g) and water (80 g), and the mixture was stirred at room temperature for 2 hours. After stirring, ordinary aqueous work-up was performed, the solvent was distilled off, then diisopropyl ether was added thereto, and washing was performed to obtain Onium Salt NSQ-1 as oily matter (yield: 20.6 g, yield percentage: 86%).

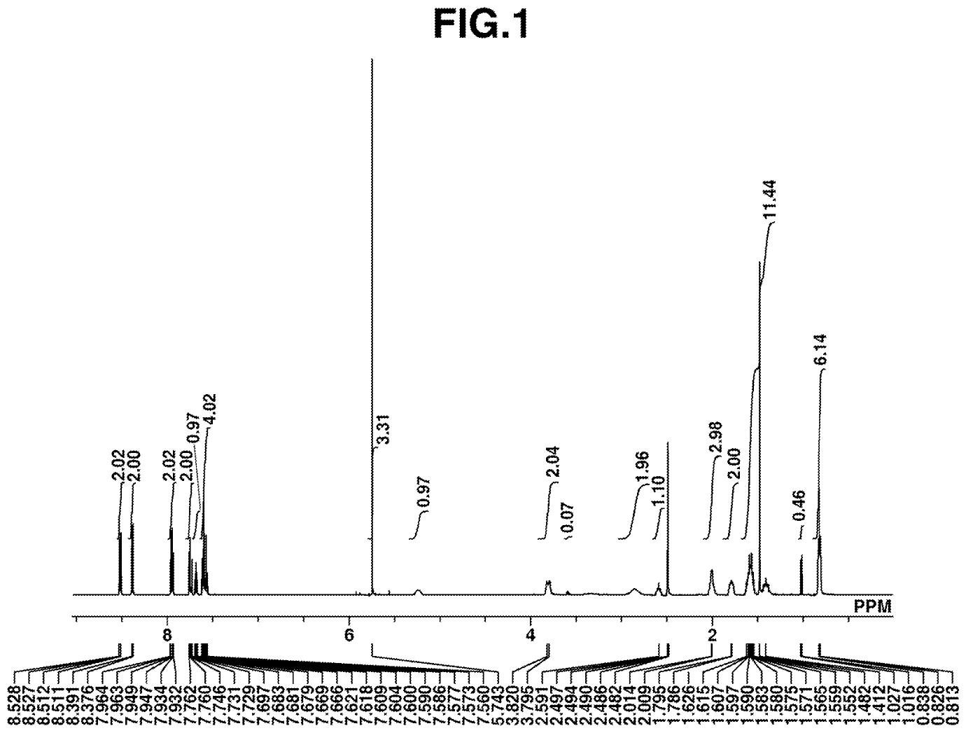

The IR spectrum data and results of TOF-MS of Onium Salt NSQ-1 are shown below. The results of the nuclear magnetic resonance spectrum (¹H-NMR/DMSO-d₆) is shown in FIG. 1. In the results of the nuclear magnetic resonance spectrum (¹H-NMR/DMSO-d₆), methylene chloride used as a solvent and diisopropyl ether used for washing were detected.
IR (D-ATR): v=3409, 3086, 3059, 2985, 2873, 1736, 1687, 1652, 1469, 1448, 1427, 1374, 1313, 1292, 1274, 1175, 1126, 1090, 1037, 1000, 933, 866, 800, 764, 735, 707, 681, 613, 526, 490, 424 cm-1.
MALDI TOF-MS:
POSITIVE M⁺261 (corresponding to C₁₈H₁₃S⁺)
NEGATIVE M⁻404 (corresponding to C₁₉H₂₈F₂NO₆⁻)

### [Examples 1-2 to 1-6] Synthesis of Onium Salts NSQ-2 to NSQ-7

Onium Salts NSQ-2 to NSQ-7 having the following formulae were synthesized using corresponding raw materials and known organic chemical reactions.

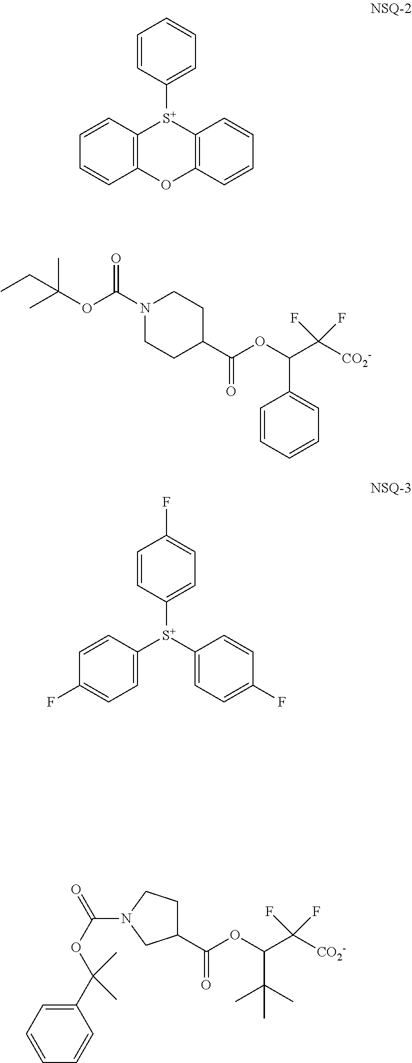

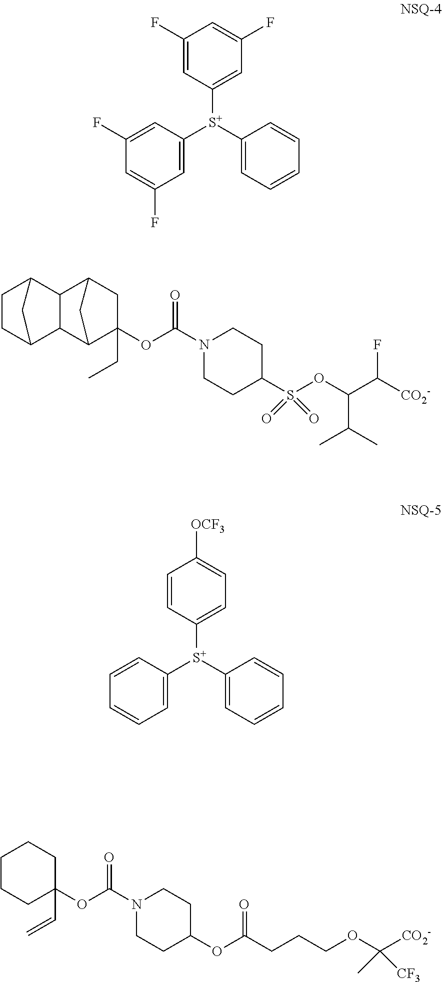

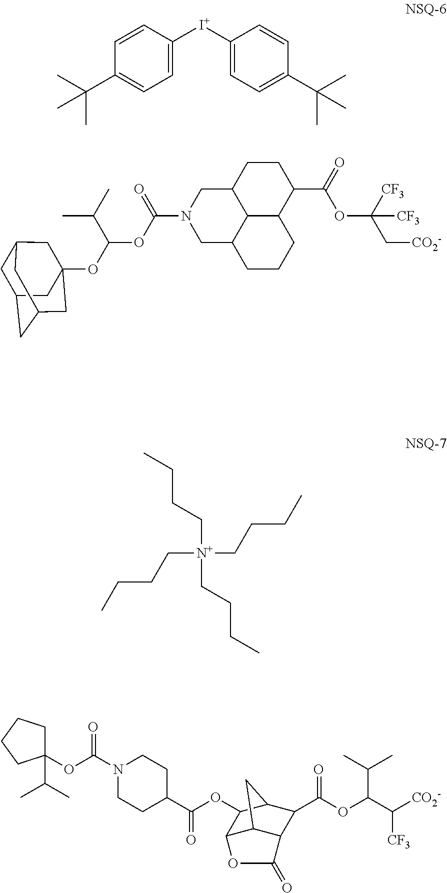

### [Synthesis Examples] Synthesis of Base Polymers (Polymers P-1 to P-5)

Base polymers (Polymers P-1 to P-5) of the composition shown below were synthesized by combining respective monomers, effecting copolymerization reaction in MEK as a solvent, pouring the reaction solution into hexane, washing the solid precipitate with hexane, isolation, and drying. The obtained base polymers were analyzed for composition by ¹H-NMR spectroscopy and for Mw and Mw/Mn by GPC (solvent: DMF, standard: polystyrene).

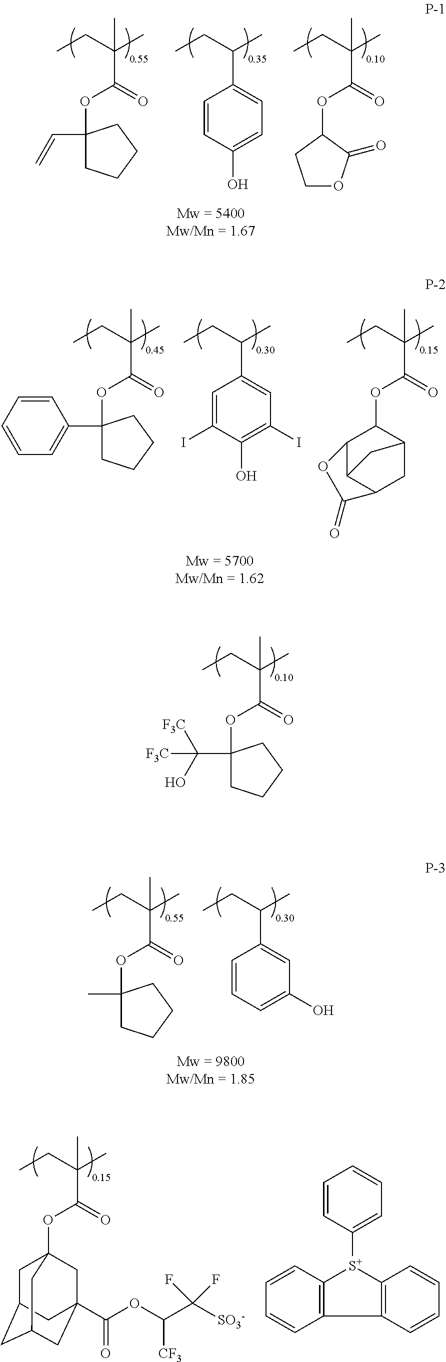

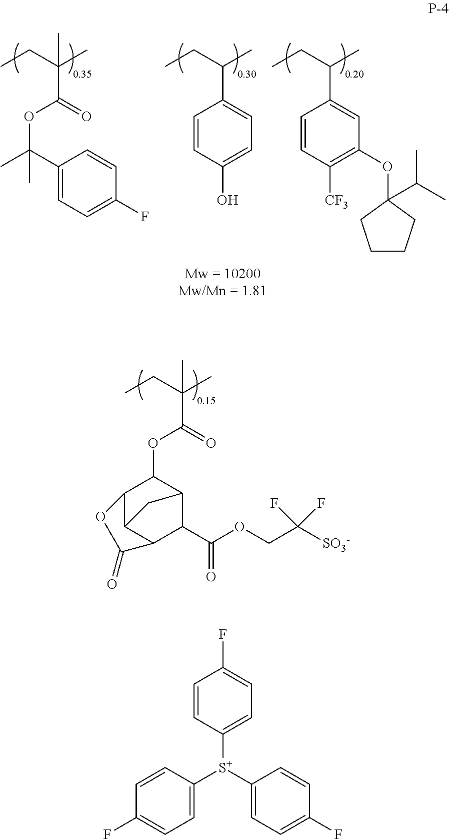

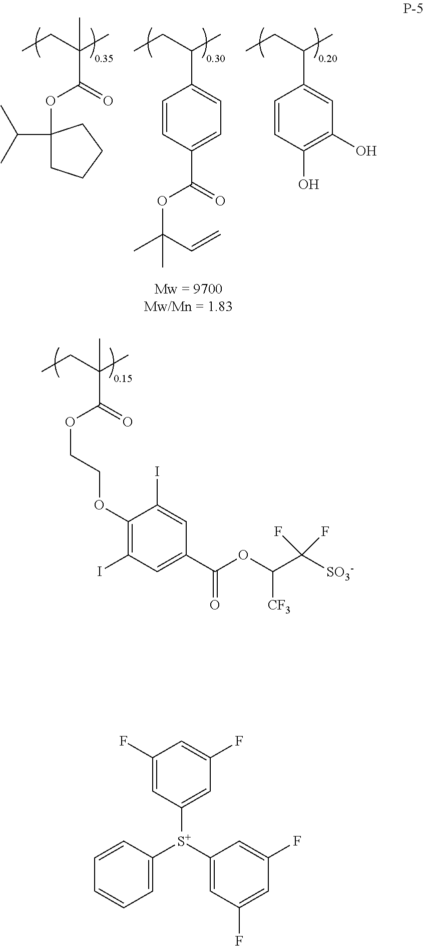

### [3] Preparation of Resist Composition

### Examples 2-1 to 2-20 and Comparative Examples 1-1 to 1-12

Resist compositions were prepared by dissolving the onium salt (NSQ-1 to NSQ-6) of the present invention, a comparative quencher (SQ-A to SQ-D, AQ-A), a base polymer (P-1 to P-5), and a photoacid generator (PAG-1, PAG-2) in a solvent in which 100 ppm of FC-4430 manufactured by 3M as a surfactant was dissolved, in accordance with the formulation shown in Tables 1 and 2 below to prepare a solution, and filtering the solution through a 0.2 μm Teflon® filter.

The components in Tables 1 and 2 are identified below.

### Organic Solvents:

PGMEA (propylene glycol monomethyl ether acetate)
DAA (diacetone alcohol)
Photoacid generators: PAG-1 and PAG-2

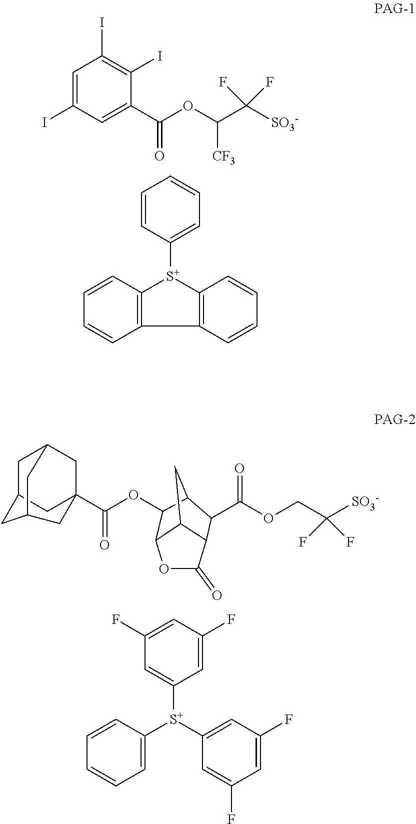

### Comparative Quenchers: SQ-A to SQ-D and AQ-A

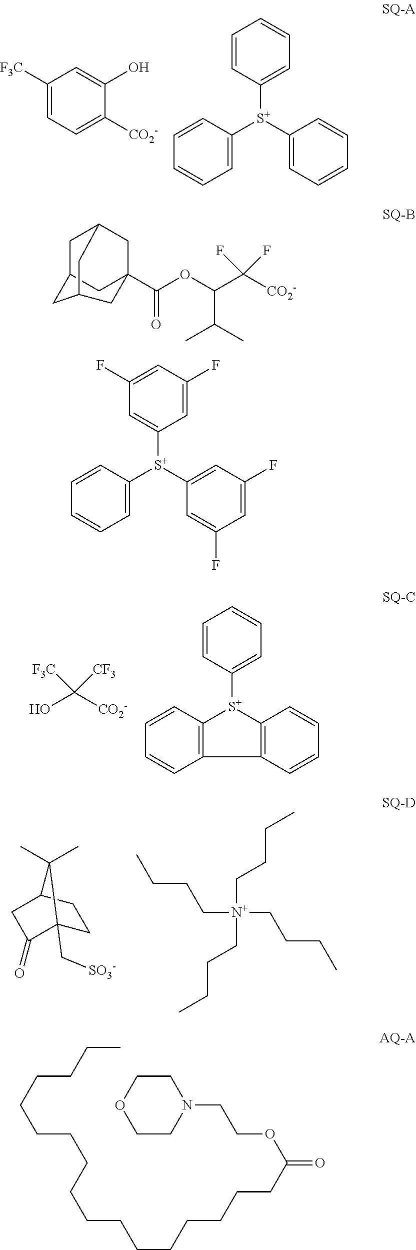

TABLE 1

| | | Resist | Base polymer | Photoacid generator | Quencher | Organic solvent 1 | Organic solvent 2 |
|---|---|---|---|---|---|---|---|
| | | composition | (pbw) | (pbw) | (pbw) | (pbw) | (pbw) |
| Example | 2-1 | R-1 | P-1 (80) | PAG-1 (30.4) | NSQ-1 (4.5) | PGMEA (3000) | DAA (900) |
| | 2-2 | R-2 | P-1 (80) | PAG-2 (25.8) | NSQ-2 (4.3) | PGMEA (3000) | DAA (900) |
| | 2-3 | R-3 | P-1 (80) | PAG-1 (28.4) | NSQ-3 (4.5) | PGMEA (3000) | DAA (900) |
| | 2-4 | R-4 | P-1 (80) | PAG-2 (24.8) | NSQ-4 (4.5) | PGMEA (3000) | DAA (900) |
| | 2-5 | R-5 | P-2 (80) | PAG-1 (30.4) | NSQ-1 (4.5) | PGMEA (3000) | DAA (900) |
| | 2-6 | R-6 | P-2 (80) | PAG-1 (29.4) | NSQ-2 (4.3) | PGMEA (3000) | DAA (900) |
| | 2-7 | R-7 | P-2 (80) | PAG-2 (25.8) | NSQ-5 (4.2) | PGMEA (3000) | DAA (900) |
| | 2-8 | R-8 | P-2 (80) | PAG-2 (24.8) | NSQ-6 (4.1) | PGMEA (3000) | DAA (900) |
| | 2-9 | R-9 | P-3 (80) | — | NSQ-1 (4.5) | PGMEA (3000) | DAA (900) |
| | 2-10 | R-10 | P-3 (80) | — | NSQ-4 (4.2) | PGMEA (3000) | DAA (900) |
| | 2-11 | R-11 | P-3 (80) | — | NSQ-5 (4.2) | PGMEA (3000) | DAA (900) |
| | 2-12 | R-12 | P-3 (80) | PAG-1 (10.4) | NSQ-1 (4.4) | PGMEA (3000) | DAA (900) |
| | 2-13 | R-13 | P-4 (80) | — | NSQ-1 (4.5) | PGMEA (3000) | DAA (900) |
| | 2-14 | R-14 | P-4 (80) | — | NSQ-2 (4.7) | PGMEA (3000) | DAA (900) |
| | 2-15 | R-15 | P-4 (80) | — | NSQ-4 (4.1) | PGMEA (3000) | DAA (900) |
| | 2-16 | R-16 | P-4 (80) | PAG-2 (8.4) | NSQ-1 (4.3) | PGMEA (3000) | DAA (900) |
| | 2-17 | R-17 | P-5 (80) | — | NSQ-1 (4.5) | PGMEA (3000) | DAA (900) |
| | 2-18 | R-18 | P-5 (80) | — | NSQ-2 (4.3) | PGMEA (3000) | DAA (900) |
| | 2-19 | R-19 | P-5 (80) | — | NSQ-7 (4.7) | PGMEA (3000) | DAA (900) |
| | 2-20 | R-20 | P-5 (80) | PAG-1 (6.4) | NSQ-1 (4.6) | PGMEA (3000) | DAA (900) |

TABLE 2

| | | Resist | Base polymer | Photoacid generator | Quencher | Organic solvent 1 | Organic solvent 2 |
|---|---|---|---|---|---|---|---|
| | | composition | (pbw) | (pbw) | (pbw) | (pbw) | (pbw) |
| Comparative | 1-1 | CR-1 | P-1 (80) | PAG-1 (30.4) | SQ-A (4.4) | PGMEA (3000) | DAA (900) |
| Example | 1-2 | CR-2 | P-1 (80) | PAG-2 (25.8) | SQ-B (4.3) | PGMEA (3000) | DAA (900) |
| | 1-3 | CR-3 | P-1 (80) | PAG-1 (28.4) | SQ-C (4.6) | PGMEA (3000) | DAA (900) |
| | 1-4 | CR-4 | P-1 (80) | PAG-2 (24.8) | AQ-A (4.8) | PGMEA (3000) | DAA (900) |
| | 1-5 | CR-5 | P-2 (80) | PAG-1 (29.4) | SQ-B (4.3) | PGMEA (3000) | DAA (900) |
| | 1-6 | CR-6 | P-2 (80) | PAG-2 (25.8) | SQ-D (3.3) | PGMEA (3000) | DAA (900) |
| | 1-7 | CR-7 | P-3 (80) | — | SQ-B (4.3) | PGMEA (3000) | DAA (900) |
| | 1-8 | CR-8 | P-3 (80) | PAG-1 (10.4) | SQ-A (4.6) | PGMEA (3000) | DAA (900) |
| | 1-9 | CR-9 | P-4 (80) | — | SQ-A (4.8) | PGMEA (3000) | DAA (900) |
| | 1-10 | CR-10 | P-4 (80) | PAG-2 (8.4) | AQ-A (4.5) | PGMEA (3000) | DAA (900) |
| | 1-11 | CR-11 | P-5 (80) | — | SQ-A (4.8) | PGMEA (3000) | DAA (900) |
| | 1-12 | CR-12 | P-5 (80) | PAG-1 (6.4) | SQ-B (4.3) | PGMEA (3000) | DAA (900) |

### [4] EUV Lithography Test (1)

### Examples 3-1 to 3-20 and Comparative Examples 2-1 to 2-12

Each chemically amplified resist composition (R-1 to R-20 and CR-1 to CR-12) was spin coated on a Si substrate having a 20-nm coating of silicon-containing spin-on hard mask SHB-A940 manufactured by Shin-Etsu Chemical Co., Ltd. (content of silicon: 43 wt %) and prebaked on a hotplate at 100° C. for 60 seconds to form a resist film of 50 nm thick. Using an EUV scanner NXE3300 (NA 0.33, σ0.9/0.6, dipole illumination) manufactured by ASML, the resist film was exposed to EUV through a LS pattern having a size of 18 nm and a pitch of 36 nm (on-wafer size) while varying the exposure dose and focus (exposure dose pitch: 1 mJ/cm², focus pitch: 0.020 μm), and after the exposure, the resist film was subjected to PEB at the temperature shown in Tables 3 and 4 for 60 seconds. Thereafter, the resist film was puddle developed in a 2.38 wt % TMAH aqueous solution for 30 seconds, rinsed with a surfactant-containing rinse material, and spin-dried to obtain a positive pattern.

The LS pattern after development was observed with a critical dimension SEM (CG6300) manufactured by Hitachi High-Technologies Corp. and evaluated for sensitivity, EL, LWR, DOF, and collapse limit by the following methods. The results are shown in Tables 5 and 6.

### [Sensitivity Evaluation]

An optimum exposure dose Eₒₚ (mJ/cm²) which provided a LS pattern with a line width of 18 nm and a pitch of 36 nm was determined and taken as sensitivity.

### [EL Evaluation]

EL (unit: %) was determined from the exposure dose which provided a LS pattern with a space width of 18 nm±10% (16.2 to 19.8 nm) according to the following equation. A greater value indicates better performance.

$$EL ( \% ) = ( \vert {E}_{1} - {E}_{2} \vert / {E}_{o p} ) \times 100$$

E₁: optimum exposure dose which provides a LS pattern with a line width of 16.2 nm and a pitch of 36 nm
E₂: optimum exposure dose which provides a LS pattern with a line width of 19.8 nm and a pitch of 36 nm
Eₒₚ: optimum exposure dose which provides a LS pattern with a line width of 18 nm and a pitch of 36 nm

### [Lwr Evaluation]

For the LS pattern formed by exposure at the optimum dose Eₒₚ, the line width was measured at 10 longitudinally spaced apart points, from which a 3-fold value (3σ) of the standard deviation (σ) was determined as LWR. As this value is smaller, a pattern having small roughness and uniform line width can be obtained.

### [DOF Evaluation]

As evaluation of the depth of focus, a range of focus which provided a LS pattern with a size of 18 nm±10% (16.2 to 19.8 nm) was determined. A greater value indicates a wider depth of focus.

### [Evaluation of Collapse Limit of Line Pattern]

For the LS pattern formed by exposure at the exposure dose corresponding to the optimum focus, the line width was measured at 10 longitudinally spaced apart points. The minimum line size above which lines could be resolved without collapse was determined and reported as collapse limit. A smaller value indicates better collapse limit.

TABLE 3

| | | Resist | PEB temperature | Optimum exposure dose | EL | LWR | DOF | Collapse limit |
|---|---|---|---|---|---|---|---|---|
| | | composition | (° C.) | (mJ/cm²) | (%) | (nm) | (nm) | (nm) |
| Example | 3-1 | R-1 | 105 | 32 | 18 | 2.7 | 120 | 10.4 |
| | 3-2 | R-2 | 100 | 31 | 17 | 2.8 | 110 | 10.7 |
| | 3-3 | R-3 | 100 | 32 | 18 | 2.8 | 110 | 10.5 |
| | 3-4 | R-4 | 105 | 32 | 17 | 2.8 | 110 | 10.6 |
| | 3-5 | R-5 | 100 | 32 | 18 | 2.9 | 110 | 10.7 |
| | 3-6 | R-6 | 95 | 31 | 18 | 3 | 120 | 11.3 |
| | 3-7 | R-7 | 100 | 31 | 19 | 2.7 | 100 | 11.2 |
| | 3-8 | R-8 | 100 | 30 | 18 | 2.8 | 120 | 11.0 |
| | 3-9 | R-9 | 95 | 26 | 18 | 2.9 | 110 | 11.3 |
| | 3-10 | R-10 | 105 | 27 | 18 | 2.8 | 110 | 10.7 |
| | 3-11 | R-11 | 95 | 27 | 19 | 2.8 | 120 | 10.7 |
| | 3-12 | R-12 | 100 | 28 | 17 | 2.7 | 100 | 11.3 |
| | 3-13 | R-13 | 100 | 27 | 18 | 2.9 | 110 | 11.2 |
| | 3-14 | R-14 | 105 | 27 | 19 | 2.7 | 120 | 10.8 |
| | 3-15 | R-15 | 100 | 26 | 18 | 2.7 | 120 | 11.1 |
| | 3-16 | R-16 | 100 | 27 | 18 | 2.9 | 110 | 10.9 |
| | 3-17 | R-17 | 105 | 26 | 17 | 2.6 | 120 | 11.5 |
| | 3-18 | R-18 | 100 | 27 | 18 | 2.8 | 110 | 10.8 |
| | 3-19 | R-19 | 105 | 27 | 18 | 2.6 | 110 | 11.2 |
| | 3-20 | R-20 | 95 | 26 | 17 | 2.8 | 110 | 11.1 |

TABLE 4

| | | Resist | PEB temperature | Optimum exposure dose | EL | LWR | DOF | Collapse limit |
|---|---|---|---|---|---|---|---|---|
| | | composition | (° C.) | (mJ/cm²) | (%) | (nm) | (nm) | (nm) |
| Comparative | 2-1 | CR-1 | 105 | 35 | 13 | 3.7 | 90 | 13.8 |
| Example | 2-2 | CR-2 | 105 | 37 | 15 | 3.9 | 80 | 14.6 |
| | 2-3 | CR-3 | 100 | 37 | 14 | 3.8 | 70 | 14.6 |
| | 2-4 | CR-4 | 105 | 37 | 13 | 3.8 | 70 | 14.1 |
| | 2-5 | CR-5 | 105 | 36 | 14 | 4 | 80 | 14.2 |
| | 2-6 | CR-6 | 100 | 36 | 13 | 3.8 | 90 | 14 |
| | 2-7 | CR-7 | 105 | 29 | 14 | 3.9 | 70 | 14.5 |
| | 2-8 | CR-8 | 95 | 28 | 14 | 4.1 | 80 | 13.7 |
| | 2-9 | CR-9 | 105 | 27 | 15 | 3.8 | 90 | 13.9 |
| | 2-10 | CR-10 | 100 | 29 | 14 | 3.9 | 70 | 13.6 |
| | 2-11 | CR-11 | 100 | 28 | 14 | 3.9 | 90 | 13.5 |
| | 2-12 | CR-12 | 110 | 29 | 15 | 3.8 | 80 | 14 |

From the results shown in Tables 3 and 4, it has been confirmed that the resist composition of the present invention has favorable sensitivity, is excellent in various lithographic performances, and exhibits performance resistant to pattern collapse.

### [5] EUV Lithography Test (2)

### Examples 4-1 to 4-20 and Comparative Examples 3-1 to 3-12

Each chemically amplified resist composition (R-1 to R-20 and CR-1 to CR-12) was spin coated on a Si substrate having a 20-nm coating of silicon-containing spin-on hard mask SHB-A940 manufactured by Shin-Etsu Chemical Co., Ltd. (content of silicon: 43 wt %) and prebaked on a hotplate at 100° C. for 60 seconds to form a resist film of 60 nm thick. Using an EUV scanner NXE3400 (NA 0.33, σ 0.9/0.6, quadrupole illumination, mask bearing a hole pattern at a pitch 44 nm (on-wafer size) and +20% bias) manufactured by ASML, the resist film was exposed to EUV, the resist film was baked (PEB) on a hotplate at the temperature shown in Tables 5 and 6 for 60 seconds and developed in a 2.38 wt % TMAH aqueous solution for 30 seconds to form a hole pattern having a size of 22 nm in Examples 4-1 to 4-18 and Comparative Examples 3-1 to 3-10 or a hole pattern having a size of 22 nm in Examples 4-19 and 4-20 and Comparative Examples 3-11 and 3-12.

The exposure dose that provides a hole or dot pattern having a size of 22 nm was measured using length measurement SEM (CG6300) manufactured by Hitachi High-Tech Corporation and taken as sensitivity, the size of 50 holes or dots at that dose was measured, from which a 3-fold value (3σ) of the standard deviation (σ) was computed and taken as CDU. The results are shown in Tables 5 and 6.

TABLE 5

| | | | PEB | Optimum | |
|---|---|---|---|---|---|
| | | Resist | temperature | exposure dose | CDU |
| | | composition | (° C.) | (mJ/cm²) | (nm) |
| Example | 4-1 | R-1 | 90 | 30 | 3.0 |
| | 4-2 | R-2 | 90 | 31 | 3.2 |
| | 4-3 | R-3 | 85 | 31 | 3.1 |
| | 4-4 | R-4 | 90 | 30 | 2.8 |
| | 4-5 | R-5 | 90 | 28 | 3.0 |
| | 4-6 | R-6 | 85 | 30 | 2.9 |
| | 4-7 | R-7 | 95 | 32 | 2.8 |
| | 4-8 | R-8 | 95 | 29 | 2.8 |
| | 4-9 | R-9 | 90 | 27 | 3.0 |
| | 4-10 | R-10 | 85 | 26 | 3.2 |
| | 4-11 | R-11 | 90 | 27 | 2.8 |
| | 4-12 | R-12 | 90 | 28 | 2.8 |
| | 4-13 | R-13 | 95 | 27 | 2.9 |
| | 4-14 | R-14 | 95 | 26 | 2.8 |
| | 4-15 | R-15 | 90 | 26 | 2.7 |
| | 4-16 | R-16 | 85 | 27 | 3.0 |
| | 4-17 | R-17 | 90 | 26 | 2.8 |
| | 4-18 | R-18 | 90 | 27 | 2.9 |
| | 4-19 | R-19 | 95 | 26 | 2.8 |
| | 4-20 | R-20 | 90 | 27 | 2.7 |

TABLE 6

| | | | PEB | Optimum | |
|---|---|---|---|---|---|
| | | Resist | temperature | exposure dose | CDU |
| | | composition | (° C.) | (mJ/cm²) | (nm) |
| Comparative | 3-1 | CR-1 | 90 | 34 | 3.6 |
| Example | 3-2 | CR-2 | 95 | 33 | 4.0 |
| | 3-3 | CR-3 | 85 | 35 | 3.8 |
| | 3-4 | CR-4 | 90 | 34 | 3.9 |
| | 3-5 | CR-5 | 85 | 35 | 4.2 |
| | 3-6 | CR-6 | 90 | 31 | 3.9 |
| | 3-7 | CR-7 | 90 | 32 | 4.1 |
| | 3-8 | CR-8 | 90 | 30 | 4.2 |
| | 3-9 | CR-9 | 95 | 29 | 4.3 |
| | 3-10 | CR-10 | 90 | 30 | 4.1 |
| | 3-11 | CR-11 | 85 | 31 | 4.0 |
| | 3-12 | CR-12 | 90 | 30 | 3.9 |

From the results shown in Tables 5 and 6, it was confirmed that the chemically amplified resist composition comprising a quencher composed of an onium salt of the present invention has favorable sensitivity and is excellent in CDU.

Japanese Patent Application No. 2023-064766 is incorporated herein by reference. Although some preferred embodiments have been described, many modifications and variations may be made thereto in light of the above teachings. It is therefore to be understood that the invention may be practiced otherwise than as specifically described without departing from the scope of the appended claims.

1. An onium salt having the following formula (1): 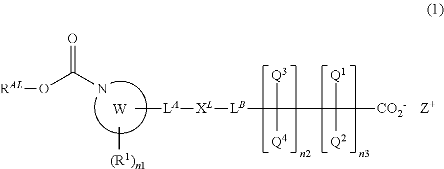
wherein n1 is an integer of 0 to 6, n2 is an integer of 0 to 3, n3 is an integer of 1 to 4,
W is a C₂-C₂₀ nitrogen atom-containing aliphatic heterocyclic ring which may contain a heteroatom,
L^{A} and L^{B} are each independently a single bond, an ether bond, an ester bond, an amide bond, a sulfonic acid ester bond, a carbonate bond, or a carbamate bond,
X^{L} is a single bond or a C₁-C₄₀ hydrocarbylene group which may contain a heteroatom,
R¹ is a C₁-C₂₀ hydrocarbyl group which may contain a heteroatom, when n1≥2, a plurality of R¹'s may bond together to form a ring with the carbon atom on W to which they are attached,
Q¹ to Q⁴ are each independently a hydrogen atom, a fluorine atom, a C₁-C₆ hydrocarbyl group, or a C₁-C₆ fluorinated hydrocarbyl group, provided that at least one of Q¹ to Q⁴ is a fluorine atom or a C₁-C₆ fluorinated hydrocarbyl group, Q³ and Q⁴ may bond together to form a ring with the carbon atom to which they are attached,
R^{AL} is an acid labile group, and
Z⁺ is an onium cation.

2. The onium salt according to claim 1, wherein R^{AL} is a group having the following formula (AL-1) or (AL-2): 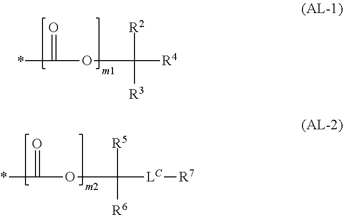
wherein LC is-O—or —S—,
R², R³, and R⁴ are each independently a C₁-C₁₀ hydrocarbyl group, any two of R², R³, and R⁴ may bond together to form a ring with the carbon atom to which they are attached,
R⁵ and R⁶ are each independently a hydrogen atom or a C₁-C₁₀ hydrocarbyl group, R⁷ is a C₁-C₂₀ hydrocarbyl group, some of —CH₂—in the hydrocarbyl group may be replaced by —O— or —S—, R⁶ and R⁷ may bond together to form a C₃-C₂₀ heterocyclic group with the carbon atom and LC to which they are attached, some of —CH₂—in the heterocyclic group may be replaced by —O—or —S—,
m1 and m2 are each independently 0 or 1, and
* designates a point of attachment to the adjacent —O—.

3. The onium salt according to claim 1, wherein Z⁺ is a sulfonium cation having the following formula (cation-1), an iodonium cation having the following formula (cation-2), or an ammonium cation having the following formula (cation-3): 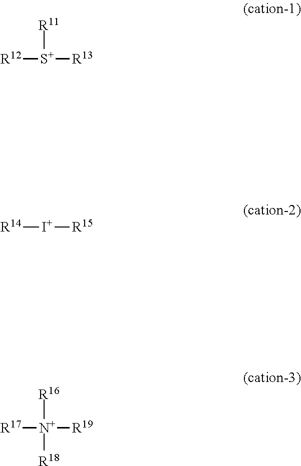
wherein R¹¹ to R¹⁹ are each independently a C₁-C₃₀ hydrocarbyl group which may contain a heteroatom, and R¹¹ and R¹² may bond together to form a ring with the sulfur atom to which they are attached.

4. The onium salt according to claim 1, which has the following formula (1A): 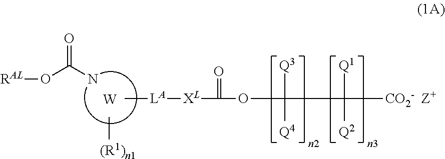
wherein n1 to n3, W, L^{A}, X^{L}, R¹, Q′ to Q⁴, R^{AL}, and Z⁺ are as defined above.

5. The onium salt according to claim 4, which has the following formula (1B): 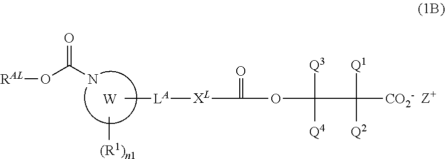
wherein n1, W, L^{A}, X^{L}, R¹, Q′ to Q³, R^{AL}, and Z⁺ are as defined above.

6. A quencher comprising the onium salt according to claim 1.

7. A resist composition comprising the quencher according to claim 6.

8. The resist composition according to claim 7, further comprising an organic solvent.

9. The resist composition according to claim 7, further comprising a base polymer comprising repeat units having the following formula (a1): 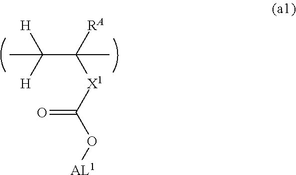
wherein R^{A} is a hydrogen atom, a fluorine atom, a methyl group, or a trifluoromethyl group,
X¹ is a single bond, a phenylene group, a naphthylene group, or *—C(═O)—O—X¹¹—, the phenylene group or the naphthylene group may be substituted with a C₁-C₁₀ alkoxy group which may contain a fluorine atom, or a halogen atom, X¹¹ is a C₁-C₁₀ saturated hydrocarbylene group, a phenylene group, or a naphthylene group, the saturated hydrocarbylene group may contain a hydroxy group, an ether bond, an ester bond, or a lactone ring, * designates a point of attachment to the carbon atom in the backbone, and
AL¹ is an acid labile group.

10. The resist composition according to claim 9, wherein the base polymer further comprises repeat units having the following formula (a2): 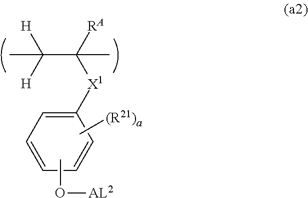
wherein R^{A} is a hydrogen atom, a fluorine atom, a methyl group, or a trifluoromethyl group,
X² is a single bond or *—C(═O)—O—, * designates a point of attachment to the carbon atom in the backbone,
R²¹ is a halogen atom, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
AL² is an acid labile group, and
a is an integer of 0 to 4.

11. The resist composition according to claim 9, wherein the base polymer further comprises repeat units having the following formula (b1) or (b2): 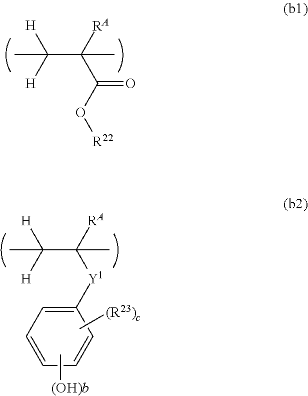
wherein R^{A} is each independently a hydrogen atom, a fluorine atom, a methyl group, or a trifluoromethyl group,
Y¹ is a single bond or *—C(═O)—O—, * designates a point of attachment to the carbon atom in the backbone,
R²² is a hydrogen atom or a C₁-C₂₀ group containing at least one or more structures selected from a hydroxy group other than a phenolic hydroxy group, a cyano group, a carbonyl group, a carboxy group, an ether bond, an ester bond, a sulfonic acid ester bond, a carbonate bond, a lactone ring, a sultone ring, and a carboxylic anhydride (—C(═O)—O—C(═O)—),
R²³ is a halogen atom, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
b is an integer of 1 to 4, and c is an integer of 0 to 4, provided that b+c is from 1 to 5.

12. The resist composition according to claim 9, wherein the base polymer further comprises repeat units having any one of the following formulae (c1) to (c4): 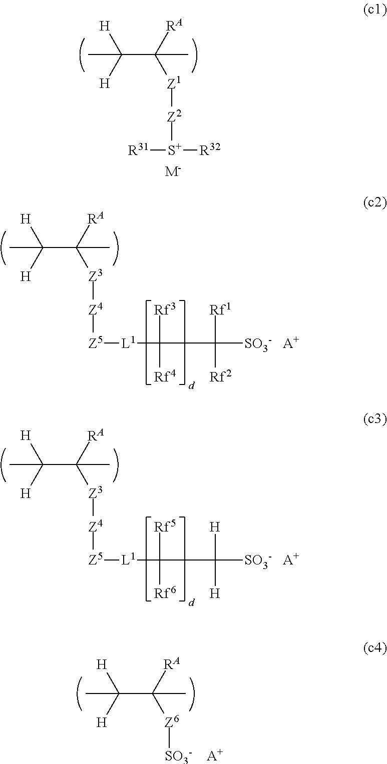
wherein R^{A} is each independently a hydrogen atom, a fluorine atom, a methyl group, or a trifluoromethyl group,
Z¹ is a single bond or a phenylene group,
Z² is *—C(═O)—O—Z²¹—, *—C(═O)—NH—Z²¹—, or *—O—Z²¹—, Z²¹ is a C₁-C₆ aliphatic hydrocarbylene group, a phenylene group, or a divalent group obtained by combining the foregoing, which may contain a carbonyl group, an ester bond, an ether bond, or a hydroxy group,
Z³ is each independently a single bond, a phenylene group, a naphthylene group, or *—C(═O)—O—Z³¹—, Z³¹ is a C₁-C₁₀ aliphatic hydrocarbylene group, a phenylene group, or a naphthylene group, the aliphatic hydrocarbylene group may contain a hydroxy group, an ether bond, an ester bond, or a lactone ring,
Z⁴ is each independently a single bond, *—Z⁴¹—C(═O)—O—, *—C(═O)—NH—Z⁴¹—, or *—O—Z⁴¹—, Z⁴¹ is a C₁-C₂₀ hydrocarbylene group which may contain a heteroatom,
Z⁵ is each independently a single bond, *—Z³¹—C(═O)—O—, *—C(═O)—NH—Z⁵¹—, or *—O—Z⁵¹—, Z⁵¹ is a C₁-C₂₀ hydrocarbylene group which may contain a heteroatom,
Z⁶ is a single bond, a methylene group, an ethylene group, a phenylene group, a fluorinated phenylene group, a trifluoromethyl group-substituted phenylene group, *—C(═O)—O—Z⁶¹—, *—C(═O)—N(H)—Z⁶¹—, or *—O—Z⁶¹—, Z⁶¹ is a C₁-C₆ aliphatic hydrocarbylene group, a phenylene group, a fluorinated phenylene group, or a trifluoromethyl group-substituted phenylene group, which may contain a carbonyl group, an ester bond, an ether bond, or a hydroxy group,
* designates a point of attachment to the carbon atom in the backbone,
R³¹ and R³² are each independently a C₁-C₂₀ hydrocarbyl group which may contain a heteroatom, R³¹ and R³² may bond together to form a ring with the sulfur atom to which they are attached,
L¹ is a single bond, an ether bond, an ester bond, a carbonyl group, a sulfonic acid ester bond, a carbonate bond, or a carbamate bond,
Rf¹ and Rf² are each independently a fluorine atom or a C₁-C₆ fluorinated saturated hydrocarbyl group,
Rf³ and Rf⁴ are each independently a hydrogen atom, a fluorine atom, or a C₁-C₆ fluorinated saturated hydrocarbyl group,
Rf⁵ and Rf⁶ are each independently a hydrogen atom, a fluorine atom, or a C₁-C₆ fluorinated saturated hydrocarbyl group, provided that all Rf⁵ and Rf⁶ are not a hydrogen atom at the same time,
M⁻ is a non-nucleophilic counter ion,
A⁺ is an onium cation, and
d is an integer of 0 to 3.

13. The resist composition according to claim 7, further comprising a photoacid generator.

14. The resist composition according to claim 7, further comprising a quencher other than the quencher.

15. The resist composition according to claim 7, further comprising a surfactant.

16. A pattern forming process comprising the steps of: applying the resist composition according to claim 7 onto a substrate to form a resist film thereon; exposing the resist film to high-energy radiation; and developing the exposed resist film in a developer.

17. The pattern forming process according to claim 16, wherein the high-energy radiation is KrF excimer laser, ArF excimer laser, EB, or EUV of wavelength 3 to 15 nm.
